(19) United States
(12) Patent Application Publication
(10) Pub. No.: US 2026/0158029 A1
(43) Pub. Date: Jun. 11, 2026

(54) FIVE-MEMBERED RING-FUSED SIX-MEMBERED RING COMPOUND, PREPARATION METHOD THEREFOR, AND PHARMACEUTICAL COMPOSITION AND USE THEREOF

(71) Applicant: HANGZHOU POLYMED BIOPHARMACEUTICALS, INC., Hangzhou (CN)

(72) Inventors: Jason Shaoyun Xiang, Hangzhou (CN); Lei Wu, Hangzhou (CN); Bing Zhang, Hangzhou (CN); Qiang Zhang, Hangzhou (CN); Gang Yang, Hangzhou (CN); Rui Xu, Hangzhou (CN); Shuai Yang, Hangzhou (CN); Yue Wu, Hangzhou (CN); Suyue Wang, Hangzhou (CN); Rui Yang, Hangzhou (CN)

(21) Appl. No.: 19/189,125

(22) Filed: Apr. 24, 2025

Related U.S. Application Data
(63) Continuation of application No. 18/651,214, filed on Apr. 30, 2024, now Pat. No. 12,343,343.
Application No. 18/651,214 is a continuation of application No. PCT/CN2023/100361, filed on Jun. 15, 2023.

(30) Foreign Application Priority Data
Jun. 15, 2022 (CN) 202210675599.3
Dec. 16, 2022 (CN) 202211627919.4
Jun. 5, 2023 (CN) 202310658843.X

(51) Int. Cl. A61K 31/496; A61K 31/4545; A61K 31/519; A61K 31/5377; A61K 31/5386; C07D 401/14; C07D 413/14; C07D 417/14; C07D 487/04; C07D 498/08; C07D 519/00

(52) U.S. Cl. CPC A61K 31/496; A61K 31/4545; A61K 31/519; A61K 31/5377; A61K 31/5386; C07D 401/14; C07D 413/14; C07D 417/14; C07D 487/04; C07D 498/08; C07D 519/00

(57) ABSTRACT

Disclosed in the present invention are a five-membered ring-fused six-membered ring compound, a preparation method therefor, and a pharmaceutical composition and the use thereof. Provided in the present invention is a compound as represented by formula (I), or a pharmaceutically acceptable salt or an isotope compound thereof. The compound of the present invention has an inhibition and/or degradation effect on IRAK4.

This application is a continuation of U.S. application Ser. No. 18/651,214, filed on Apr. 30, 2024, which is a continuation of International Patent Application No. PCT/CN 2023/100361, filed Jun. 15, 2023, which claims the priorities of Chinese Patent Application No. 202210675599.3, filed on Jun. 15, 2022, Chinese Patent Application No. 202211627919.4, filed on Dec. 16, 2022, and Chinese patent application No. 202310658843.X, filed on Jun. 5, 2023, the entire disclosure of each of which is incorporated herein by reference for all purposes.

### FIELD OF THE DISCLOSURE

The present disclosure relates to a five-membered ring-fused six-membered ring compound, preparation method therefor, and pharmaceutical composition and use thereof.

### BACKGROUND OF THE DISCLOSURE

Kinases have always been important therapeutic targets for the development of anti-inflammatory drugs (Current Opinion in Cell Biology 2009 21, 1-8). Interleukin-1 receptor-associated kinases (IRAKs) are serine/threonine protein kinases that belong to tyrosine-like kinase (TLK) family. IRAKs are located downstream of toll-like receptors and IL-1R pathways, and IRAK1 and IRAK4 have kinase activity. IRAK4 acts upstream of the IRAKs family kinase activation pathway and plays an important role in innate immune signaling (Science 1996, 271(5252): 1128-31). Stimulation of TLR can recruit myeloid differentiation primary response 88 (MYD88) and activate receptors to form a complex Myddosome, which then forms a complex with IRAK4 to activate IRAK1. Subsequently, TRAF6 is activated by IRAK1, leading to the activation of NF-κB and A M PK signaling pathways, ultimately leading to the expression of inflammatory cytokines (Molecules 2016, 21, 1529, J Biol Chem. 2018 Sep. 28; 293(39): 15195-15207, Eur J. Immunol. 2008. 38:614-618).

As a very important property, IRAK4 has two functions of scaffolding and kinase phosphorylation in the TLR and IL-1R signaling pathways. The kinase domain (KD) provides the kinase function, and the death domain (DD) provides the scaffolding function for Myddosome (Molecules 2016, 21(11), 1529). Myddosomes are related to a variety of diseases, not only to autoimmune and inflammatory diseases but also to cancer. For example, 39% of patients with active B-cell-like diffuse large B-cell lymphoma (ABC DLBCL) and 86%-100% of patients with several other types of B-cell malignancies and primary central nervous system lymphomas have MYD88 mutations (Cell Chemical Biology 27, 1-10, Dec. 17, 2020).

IRAK4 gene knockout mice and clinical pathology studies have shown that IRAK4 deficiency itself is nonlethal, and individuals with IRAK4 mutations are also been protected from chronic lung disease and inflammatory bowel disease (Eur. J. Immunol. 2008. 38: 614-618). IRAK4 inhibitors have been considered as targets for the treatment of immune diseases such as autoimmune diseases rheumatoid arthritis (RA), systemic lupus erythematosus (SLE) and psoriasis (Expert Opinion on Therapeutic Patents Volume 29, 2019-Issue 4). Meanwhile, IRAK4 is also a popular target for treating tumors, and there are currently several IRAK4 kinase inhibitors entering the clinical stage. However, these drugs underdevelopment in clinical stages are all inhibitors having IRAK4 kinase function to kinase domain (K D) and have no direct inhibitory effect on the scaffolding function of IRAK4. Protein degraders (PROTACs) targeting IRAK4 are expected to simultaneously eliminate its kinase activity and scaffolding functions, resulting in a better and broader efficacy (Nature Biotechnology 2020, volume 38, pages 1221-1223, ACS M ed Chem Lett. 2019 Jul. 11; 10(7): 1081-1085).

### DESCRIPTION OF THE DISCLOSURE

The present disclosure provides a five-membered ring-fused six-membered ring compound, preparation method therefor, and pharmaceutical composition and use thereof. The compound of the present disclosure has an inhibitory or/and degradative effect on IRAK4, has potential clinical application value, and is expected to improve the prognosis of patients and reduce the possibility of drug resistance.

The present disclosure provides a compound of formula I, a pharmaceutically acceptable salt or an isotope compound thereof:

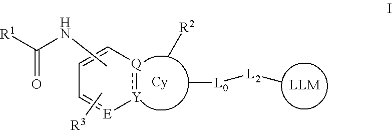

Ring Cy is a 5-membered heterocycle or a 5-membered heteroaromatic ring; the heteroatoms of the 5-membered heterocycle are selected from one or two of N, S and O, and the number of heteroatoms is 1 or 2; the heteroatoms of the 5-membered heteroaromatic ring are selected from one or more of N, S and O, and the number of heteroatoms is 1, 2 or 3;
[symbol] is | or ∥;
Q is C or N;
E is CH or N;
Y is C or N;
R¹ is a 5- to 10-membered heteroaryl that is unsubstituted or substituted by one or more R¹⁻¹, or a 6- to 10-membered aryl that is unsubstituted or substituted by one or more R¹⁻²; the heteroatoms of the 5- to 10-membered heteroaryl are selected from one or more of N, S and O, and the number of heteroatoms is 1, 2 or 3;
Each R¹⁻¹ and R¹⁻² are independently halogen, hydroxyl,

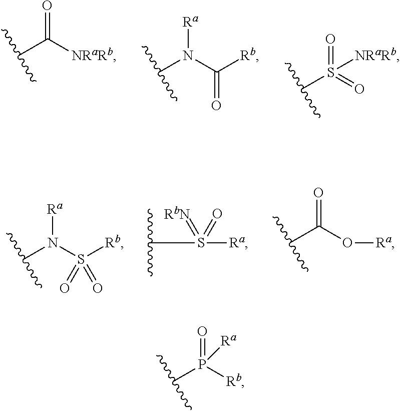

—SO₂R^{a}, —SO₂R^{a}, cyano, nitro, a 3- to 11-membered heterocycloalkyl that is unsubstituted or substituted by one or more R¹⁻¹⁻¹, C₁-C₆ alkoxy that is unsubstituted or substituted by one or more R¹⁻¹⁻³, C₁-C₆alkyl that is unsubstituted or substituted by one or more R¹⁻¹⁻⁴,

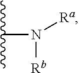

a 3- to 10-membered cycloalkyl that is unsubstituted or substituted by one or more R¹⁻¹⁻⁵, a 6- to 10-membered aryl that is unsubstituted or substituted by one or more R¹⁻¹⁻⁸, or a 5- to 10-membered heteroaryl that is unsubstituted or substituted by one or more R¹⁻¹⁻⁷; the heteroatoms of the 3- to 11-membered heterocycloalkyl are selected from one or more of N, S and O, and the number of heteroatoms is 1, 2 or 3; the heteroatoms of the 5- to 10-membered heteroaryl are selected from one or more of N, S and O, and the number of heteroatoms is 1, 2 or 3;
Each R¹⁻¹⁻¹, R¹⁻¹⁻³, R¹⁻¹⁻⁴, R¹⁻¹⁻⁵, R¹⁻¹⁻⁷ and R¹⁻¹⁻⁸ are independently deuterium, halogen, oxo, hydroxyl,

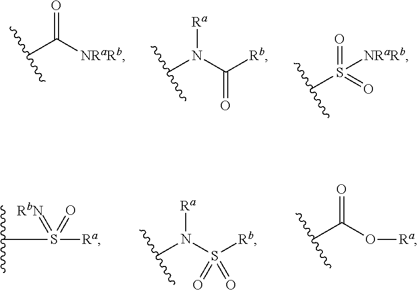

—SO₂—R^{a}, —SO—R^{a}, C₁-C₆ alkoxy that is unsubstituted or substituted by one or more halogens, cyano, nitro, a 3- to 11-membered heterocycloalkyl that is unsubstituted or substituted by one or more R¹⁻¹⁻¹⁻¹, C₁-C₆ alkyl that is unsubstituted or substituted by one or more halogens or deuterium, a 3- to 10-membered cycloalkyl, a 6- to 10-membered aryl that is unsubstituted or substituted by one or more R¹⁻¹⁻¹⁻², or a 5- to 10-membered heteroaryl that is unsubstituted or substituted by one or more R¹⁻¹⁻¹⁻³; the heteroatoms of the 3- to 11-membered heterocycloalkyl are selected from one or more of N, S and O, and the number of heteroatoms is 1, 2 or 3; the heteroatoms of the 5- to 10-membered heteroaryl are selected from one or more of N, S and O, and the number of heteroatoms is 1, 2 or 3;
Each R¹⁻¹⁻¹⁻¹, R¹⁻¹⁻¹⁻², and R¹⁻¹⁻¹⁻³ are independently halogen, oxo, hydroxyl, C₁-C₆ alkoxy or C₁-C₆ alkyl;
R² is hydrogen, hydroxyl, cyano, halogen, C₁-C₆ alkyl that is unsubstituted or substituted by one or more R²⁻³, C₁-C₆ alkoxy that is unsubstituted or substituted by one or more R²⁻²,

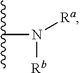

a 5- to 10-membered heteroaryl that is unsubstituted or substituted by one or more R²⁻¹ or a 4- to 10-membered heterocycloalkyl that is unsubstituted or substituted by one or more R²⁴; the heteroatoms of the 5- to 10-membered heteroaryl are selected from one or more of N, S and O, and the number of heteroatoms is 1, 2 or 3; the heteroatoms of the 4- to 10-membered heterocycloalkyl are selected from one or more N, S and O, and the number of heteroatoms is 1, 2 or 3;
Each R²⁻¹, R²⁻², R²⁻³ and R²⁻⁴ are independently halogen, hydroxyl, cyano, nitro, C₁-C₆ alkyl that is unsubstituted or substituted by one or more halogens,

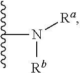

or C₁-C₆ alkoxy that is unsubstituted or substituted by one or more halogens;
R³ is hydrogen, deuterium, halogen, cyano, hydroxyl, nitro,

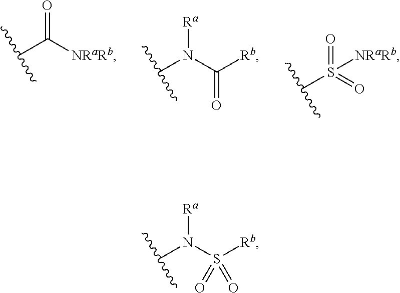

—SO—R^{a}, —SO—R^{a},

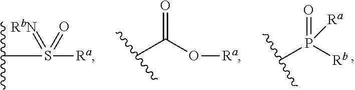

a 3- to 11-membered heterocycloalkyl that is unsubstituted or substituted by one or more R³⁻¹, a 3- to 10-membered cycloalkyl that is unsubstituted or substituted by one or more R³⁻²,

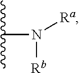

C₁-C₆ alkyl that is unsubstituted or substituted by one or more R¹³-4, a 6- to 10-membered aryl that is unsubstituted or substituted by one or more R³⁻⁵, a 5- to 10-membered heteroaryl that is unsubstituted or substituted by one or more R³⁻⁶, a hydroxyl substituted by R³⁻⁸, C₁-C₆ alkoxy that is unsubstituted or substituted by one or more R³⁻⁷ or —O—COR^{a}; the heteroatoms of the 3- to 11-membered heterocycloalkyl are selected from one or more of N, S and O, and the number of the heteroatoms is 1, 2 or 3; the heteroatoms of the 5- to 10-membered heteroaryl are selected from one or more of N, S and O, and the number of heteroatoms is 1, 2 or 3;
Each R³⁻¹, R³⁻², R³⁻⁴, R³⁻⁵, R³⁻⁶ and R³⁻⁷ are independently deuterium, halogen, oxo, hydroxyl, a 3- to 11-membered heterocycloalkyl that is unsubstituted or substituted by one or more R³⁻¹⁻¹,

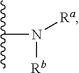

cyano, alkoxy that is unsubstituted or substituted by one or more R³⁻¹⁻³,

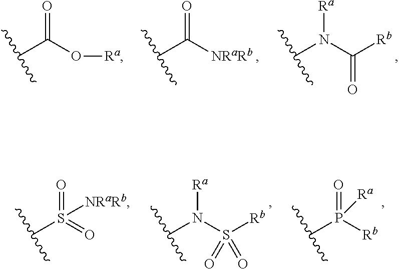

C₁-C₆ alkyl that is unsubstituted or substituted by one or more R³⁻¹⁻⁴, a 3- to 10-membered cycloalkyl that is unsubstituted or substituted by one or more R³⁻¹⁻⁵, —SO₂—R^{a}, —SO—R^{a},

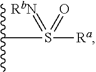

a 6- to 10-membered aryl, or a 5- to 10-membered heteroaryl; the heteroatoms of the 3- to 11-membered heterocycloalkyl are selected from one or more of N, S and O, and the number of heteroatoms is 1, 2 or 3; the heteroatoms of the 5- to 10-membered heteroaryl are selected from one or more of N, S and O, and the number of heteroatoms is 1, 2 or 3;
R³⁻⁸ is a 3- to 10-membered cycloalkyl, a 6- to 10-membered aryl, a 3- to 11-membered heterocycloalkyl, or a 5- to 10-membered heteroaryl; the heteroatoms of the 3- to 11-membered heterocycloalkyl are selected from one or more of N, S and O, and the number of heteroatoms is 1, 2 or 3; the heteroatoms of the 5- to 10-membered heteroaryl are selected from one or more N, S and O, and the number of heteroatoms is 1, 2 or 3;
Each R³⁻¹⁻¹, R³⁻¹⁻³, R³⁻¹⁻⁴ and R³⁻¹⁻⁵ are independently C₁-C₆ alkyl that is unsubstituted or substituted by one or more halogens, halogen, oxo or hydroxyl;
Each R^{a} and each R^{b} are independently H, C₁-C₆ alkyl that is unsubstituted or substituted by one or more R^{a-1}, a 3- to 10-membered cycloalkyl that is unsubstituted or substituted by one or more R^{a-2}, a 3- to 11-membered heterocycloalkyl that is unsubstituted or substituted by one or more R^{a-3}, a 6- to 10-membered aryl that is unsubstituted or substituted by one or more R^{a-4}, or a 5- to 10-membered heteroaryl that is unsubstituted or substituted by one or more R^{a-5};
Or, R^{a} and R^{b} and the atoms connected thereto together form a 3- to 11-membered heterocycle; the heteroatoms of the 3- to 11-membered heterocycle are selected from one or more of N, S and O, and the number of heteroatoms is 1, 2 or 3;
Each R^{a-1}, R^{a-2}, R^{a-3}, R^{a-4} and R^{a-5} are independently halogen, cyano, hydroxyl, nitro, C₁-C₆ alkyl, C₁-C₆ alkoxy, a 3- to 10-membered cycloalkyl, a 3- to 11-membered heterocycloalkyl, a 6- to 10-membered aryl, or a 5- to 10-membered heteroaryl; the heteroatoms of the 3 to 11-membered heterocycloalkyl are selected from one or more of N, S and O, and the number of heteroatoms is 1, 2 or 3; the heteroatoms of the 5- to 10-membered heteroaryl are selected from one or more of N, S and O, and the number of heteroatoms is 1, 2 or 3;
L₀ is a 5- to 12-membered cycloalkylene group that is unsubstituted or substituted by one or more L₀⁻², or a 5- to 12-membered heterocycloalkylene group that is unsubstituted or substituted by one or more L₀⁻³; the 5- to 12-membered cycloalkylene group is bicyclic or polycyclic; the 5- to 12-membered heterocycloalkylene group is a monocyclic, bicyclic or polycyclic compound; the heteroatoms of the 5- to 12-membered cycloalkylene group are selected from one or more of N, S and O, and the number of heteroatoms is 1, 2 or 3; each L₀⁻² is independently deuterium, hydroxyl, halogen, C₁-C₆ alkoxy that is unsubstituted or substituted by one or more halogens or deuterium,

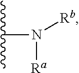

or C₁-C₆ alkyl that is unsubstituted or substituted by one or more halogens or deuterium; each L₀⁻³ is independently deuterium, hydroxyl, halogen, C₁-C₆ alkoxy that is unsubstituted or substituted by one or more halogens or deuterium,

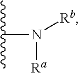

or C₁-C₆ alkyl that is unsubstituted or substituted by one or more halogens or deuterium;
L₂ is linker unit (connecting LLM to L₀);
When L₀ is

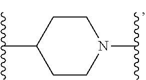 [SMILES: *C1CCN(*)CC1]

L₂ comprises at least one or more of the following fragments:

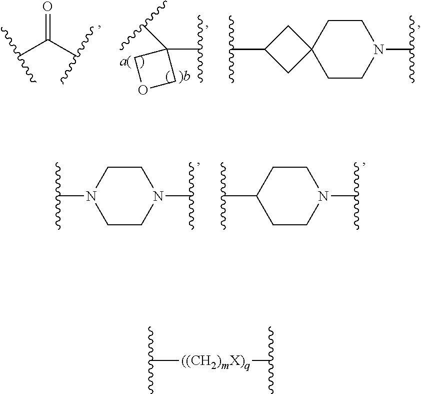

that is unsubstituted or substituted by one or more L₂¹⁻¹, or

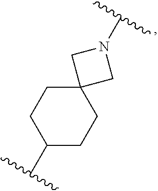 [SMILES: *C1CCC2(CC1)CN(*)C2]

a and b are independently 1 or 2, m is an integer from 1 to 4, q is an integer from 1 to 6, X is 0; L₂¹⁻¹ is halogen, C₁-C₆ alkyl that is unsubstituted or substituted by one or more halogens, C₁-C₆ alkoxy that is unsubstituted or substituted by one or more halogens,

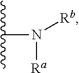

hydroxyl, oxo or

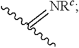

Each R^{c} is independently H, C₁-C₆ alkyl that is unsubstituted or substituted by one or more R^{c-1}, a 3- to 10-membered cycloalkyl that is unsubstituted or substituted by one or more R^{c-2}, a 3- to 11-membered heterocycloalkyl that is unsubstituted or substituted by one or more R^{c-3}, a 6- to 10-membered aryl that is unsubstituted or substituted by one or more R^{c-4}, or a 5- to 10-membered heteroaryl that is unsubstituted or substituted by one or more R^{c-5};
Each R^{c-1}, R^{c-2}, R^{c-3}, R^{c-4} and R^{c-5} are independently halogen, cyano, hydroxyl, nitro, C₁-C₆ alkyl, C₁-C₆ alkoxy, a 3- to 10-membered cycloalkyl, a 3- to 11-membered heterocycloalkyl, a 6- to 10-membered aryl, or a 5- to 10-membered heteroaryl; the heteroatoms of the 3- to 11-membered heterocycloalkyl are selected from one or more of N, S and O, and the number of heteroatoms is 1, 2 or 3; the heteroatoms of the 5- to 10-membered heteroaryl are selected from one or more of N, S and O, and the number of heteroatoms is 1, 2 or 3;
LLM is

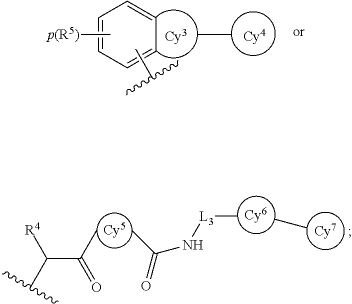

Ring Cy³ is a 5- to 12-membered heterocycle that is unsubstituted or substituted by one or more Cy³⁻¹, the heteroatoms of the 5- to 12-membered heterocycle are selected from one or more of N, S and O, and the number of heteroatoms is 1, 2 or 3; Cy³⁻¹ is independently C₁-C₆ alkyl, halogen, hydroxyl or oxo;
Ring Cy⁴ is a 5- to 12-membered heterocycloalkyl that is unsubstituted or substituted by one or more Cy⁴⁻¹, the heteroatoms of the 5- to 12-membered heterocycloalkyl are selected from one or more of N, S and O, and the number of heteroatoms is 1, 2 or 3; Cy⁴⁻¹ is independently C₁-C₆ alkyl, halogen, hydroxyl or oxo;
Ring Cy⁵ is a 5- to 12-membered heterocycle that is unsubstituted or substituted by one or more Cy⁵⁻¹, the heteroatoms of the 5- to 12-membered heterocycle are selected from one or more of N, S and O, and the number of heteroatoms is 1, 2 or 3; Cy⁵⁻¹ is independently C₁-C₆ alkyl, hydroxyl or oxo;
Ring Cy⁶ is a 6- to 10-membered aromatic ring that is unsubstituted or substituted by one or more Cy⁶⁻¹; Cy⁶⁻¹ is independently C₁-C₆ alkyl, hydroxyl or halogen;
Ring Cy⁷ is a 5- to 9-membered heteroaromatic ring that is unsubstituted or substituted by one or more Cy⁷⁻¹, the heteroatoms of the 5- to 9-membered heteroaromatic ring are selected from one or more of N, S and O, and the number of heteroatoms is 1, 2 or 3; Cy⁷⁻¹ is independently C₁-C₆ alkyl, hydroxyl or halogen;
R⁴ is independently hydrogen, halogen, hydroxyl or C₁-C₆ alkyl that is unsubstituted or substituted by one or more halogens;
P is 0, 1, 2 or 3;
Each R⁵ is independently halogen;
L₃ is

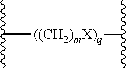

that is unsubstituted or substituted by one or more L₃⁻¹; where m is an integer from 1 to 4, q is an integer from 1to 6, and X is absent or O; L₃⁻¹ is independently halogen, or C₁-C₆ alkyl that is unsubstituted or substituted by one or more halogens.

The present disclosure provides a compound of formula I, a pharmaceutically acceptable salt or an isotope compound thereof:

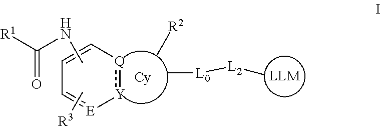

Ring Cy is a 5-membered heterocycle or a 5-membered heteroaromatic ring; the heteroatoms of the 5-membered heterocycle are selected from one or two of N, S and O, and the number of heteroatoms is 1 or 2; the heteroatoms of the 5-membered heteroaromatic ring are selected from one or more of N, S and O, and the number of heteroatoms is 1, 2 or 3;
[symbol] is | or ∥;
Q is C or N;
E is CH or N;
Y is C or N;
R¹ is a 5- to 10-membered heteroaryl that is unsubstituted or substituted by one or more R¹⁻¹, or a 6- to 10-membered aryl that is unsubstituted or substituted by one or more R¹⁻²; the heteroatoms of the 5- to 10-membered heteroaryl are selected from one or more of N, S and O, and the number of heteroatoms is 1, 2 or 3;
Each R¹⁻¹ and R¹⁻² are independently halogen, hydroxyl,

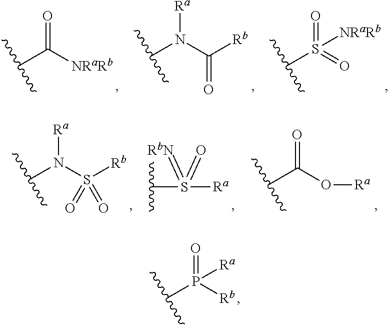

—SO₂—R^{a}, —SO—R^{a}, cyano, nitro, a 3- to 11-membered heterocycloalkyl that is unsubstituted or substituted by one or more R¹⁻¹⁻¹, C₁-C₆ alkoxy that is unsubstituted or substituted by one or more R¹⁻¹⁻³, C₁-C₆ alkyl that is unsubstituted or substituted by one or more R¹⁻¹⁻⁴,

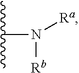

a 3- to 10-membered cycloalkyl that is unsubstituted or substituted by one or more R¹⁻¹⁻⁵, a 6- to 10-membered aryl that is unsubstituted or substituted by one or more R¹⁻¹⁻⁸, or a 5- to 10-membered heteroaryl that is unsubstituted or substituted by one or more R¹⁻¹⁻⁷; the heteroatoms of the 3- to 11-membered heterocycloalkyl are selected from one or more of N, S and O, and the number of heteroatoms is 1, 2 or 3; the heteroatoms of the 5- to 10-membered heteroaryl are selected from one or more of N, S and O, and the number of heteroatoms is 1, 2 or 3;
Each R¹⁻¹⁻¹, R¹⁻¹⁻³, R¹⁻¹⁻⁴, R¹⁻¹⁻⁵, R¹⁻¹⁻⁷ and R¹⁻¹⁻⁸ are independently halogen, oxo, hydroxyl,

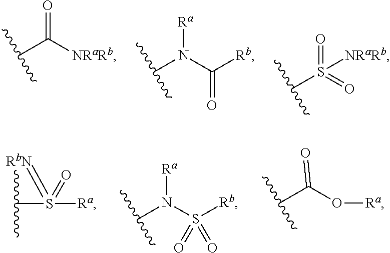

—SO₂—R^{a}, —SO—R^{a}, C₁-C₆ alkoxy that is unsubstituted or substituted by one or more halogens, cyano, nitro, a 3- to 11-membered heterocycloalkyl that is unsubstituted or substituted by one or more R¹⁻¹⁻¹⁻¹, C₁-C₆ alkyl that is unsubstituted or substituted by one or more halogens, a 3- to 10-membered cycloalkyl, a 6- to 10-membered aryl that is unsubstituted or substituted by one or more R¹⁻¹⁻¹⁻², or a 5- to 10-membered heteroaryl that is unsubstituted or substituted by one or more R¹⁻¹⁻¹⁻³; the heteroatoms of the 3- to 11-membered heterocycloalkyl are selected from one or more of N, S and O, and the number of heteroatoms is 1, 2 or 3; the heteroatoms of the 5- to 10-membered heteroaryl are selected from one or more of N, S and O, and the number of heteroatoms is 1, 2 or 3;
Each R¹⁻¹⁻¹⁻¹, R¹⁻¹⁻¹⁻², and R¹⁻¹⁻¹⁻³ are independently halogen, oxo, hydroxyl, C₁-C₆ alkoxy or C₁-C₆ alkyl;
R² is hydrogen, hydroxyl, cyano, halogen, C₁-C₆ alkyl that is unsubstituted or substituted by one or more R²⁻³, C₁-C₆ alkoxy that is unsubstituted or substituted by one or more R²⁻²,

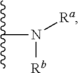

a 5-to 10-membered heteroaryl that is unsubstituted or substituted by one or more R²⁻¹or a 4- to 10-membered heterocycloalkyl that is unsubstituted or substituted by one or more R²⁻⁴; the heteroatoms of the 5- to 10-membered heteroaryl are selected from one or more of N, S and O, and the number of heteroatoms is 1, 2 or 3; the heteroatoms of the 4- to 10-membered heterocycloalkyl are selected from one or more N, S and O, and the number of heteroatoms is 1, 2 or 3;
Each R²⁻¹, R²⁻², R²⁻³ and R²⁻⁴ are independently halogen, hydroxyl, cyano, nitro, C₁-C₆ alkyl that is unsubstituted or substituted by one or more halogens,

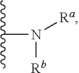

or C₁-C₆ alkoxy that is unsubstituted or substituted by one or more halogens;
R³ is hydrogen, deuterium, halogen, cyano, hydroxyl, nitro,

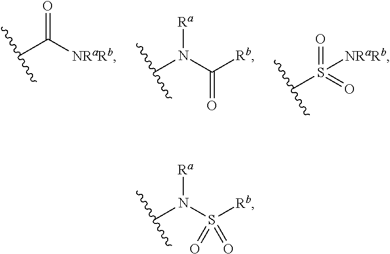

SO₂—R^{a}, —SO—R^{a},

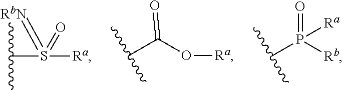

a 3- to 11-membered heterocycloalkyl that is unsubstituted or substituted by one or more R³⁻¹, a 3- to 10-membered cycloalkyl that is unsubstituted or substituted by one or more R³⁻²,

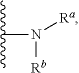

C₁-C₆ alkyl that is unsubstituted or substituted by one or more R³⁻⁴, a 6- to 10-membered aryl that is unsubstituted or substituted by one or more R³⁻⁵, a 5- to 10-membered heteroaryl that is unsubstituted or substituted by one or more R³⁻⁶, a hydroxyl substituted by R³⁻⁸, C₁-C₆ alkoxy that is unsubstituted or substituted by one or more R³⁻⁷ or —O—COR^{a}; the heteroatoms of the 3- to 11-membered heterocycloalkyl are selected from one or more of N, S and O, and the number of the heteroatoms is 1, 2 or 3; the heteroatoms of the 5- to 10-membered heteroaryl are selected from one or more of N, S and O, and the number of heteroatoms is 1, 2 or 3;
Each R³⁻¹, R³⁻², R³⁻⁴, R³⁻⁵, R³⁻⁶ and R³⁻⁷ are independently deuterium, halogen, oxo, hydroxyl, a 3- to 11-membered heterocycloalkyl that is unsubstituted or substituted by one or more R³⁻¹⁻¹,

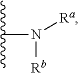

cyano, alkoxy that is unsubstituted or substituted by one or more R³⁻¹⁻³,

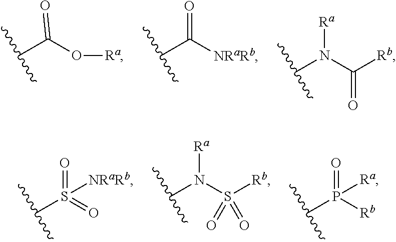

C₁-C₆ alkyl that is unsubstituted or substituted by one or more R³⁻¹⁻⁴, a 3- to 10-membered cycloalkyl that is unsubstituted or substituted by one or more R³⁻¹⁻⁵, —SO₂—R^{a}, —SO—R^{a},

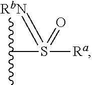

a 6- to 10-membered aryl, or a 5- to 10-membered heteroaryl; the heteroatoms of the 3- to 11-membered heterocycloalkyl are selected from one or more of N, S and O, and the number of heteroatoms is 1, 2 or 3; the heteroatoms of the 5- to 10-membered heteroaryl are selected from one or more of N, S and O, and the number of heteroatoms is 1, 2 or 3;
R³⁻⁸ is a 3- to 10-membered cycloalkyl, a 6- to 10-membered aryl, a 3- to 11-membered heterocycloalkyl, or a 5- to 10-membered heteroaryl; the heteroatoms of the 3- to 11-membered heterocycloalkyl are selected from one or more of N, S and O, and the number of heteroatoms is 1, 2 or 3; the heteroatoms of the 5- to 10-membered heteroaryl are selected from one or more N, S and O, and the number of heteroatoms is 1, 2 or 3;
Each R³⁻¹⁻¹, R³⁻¹⁻³, R³⁻¹⁻⁴ and R³⁻¹⁻⁵ are independently C₁-C₆ alkyl that is unsubstituted or substituted by one or more halogens, halogen, oxo or hydroxyl;
Each R^{a} and each R^{b} are independently H, C₁-C₆ alkyl that is unsubstituted or substituted by one or more R^{a-1}, a 3- to 10-membered cycloalkyl that is unsubstituted or substituted by one or more R^{a-2}, a 3- to 11-membered heterocycloalkyl that is unsubstituted or substituted by one or more R^{a-3}, a 6- to 10-membered aryl that is unsubstituted or substituted by one or more R^{a-4}, or a 5- to 10-membered heteroaryl that is unsubstituted or substituted by one or more R^{a-5};
Or, R^{a} and R^{b} and the atoms connected thereto together form a 3- to 11-membered heterocycle; the heteroatoms of the 3- to 11-membered heterocycle are selected from one or more of N, S and O, and the number of heteroatoms is 1, 2 or 3;
Each R^{a-1}, R^{a-2}, R^{a-3}, R^{a-4} and R^{a-5} are independently halogen, cyano, hydroxyl, nitro, C₁-C₆ alkyl, C₁-C₆ alkoxy, a 3- to 10-membered cycloalkyl, a 3- to 11-membered heterocycloalkyl, a 6- to 10-membered aryl, or a 5- to 10-membered heteroaryl; the heteroatoms of the 3 to 11-membered heterocycloalkyl are selected from one or more of N, S and O, and the number of heteroatoms is 1, 2 or 3; the heteroatoms of the 5- to 10-membered heteroaryl are selected from one or more of N, S and O, and the number of heteroatoms is 1, 2 or 3;
L₀ is a 5- to 12-membered cycloalkylene group that is unsubstituted or substituted by one or more L₀⁻², or a 5- to 12-membered heterocycloalkylene group that is unsubstituted or substituted by one or more L₀⁻³; the 5- to 12-membered cycloalkylene group is bicyclic or polycyclic; the 5- to 12-membered heterocycloalkylene group is a monocyclic, bicyclic or polycyclic compound; the heteroatoms of the 5- to 12-membered cycloalkylene group are selected from one or more of N, S and O, and the number of heteroatoms is 1, 2 or 3; each L₀⁻² is independently hydroxyl, halogen, C₁-C₆ alkoxy that is unsubstituted or substituted by one or more halogens,

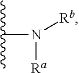

or C₁-C₆ alkyl that is unsubstituted or substituted by one or more halogens; each L₀⁻³ is independently hydroxyl, halogen, C₁-C₆ alkoxy that is unsubstituted or substituted by one or more halogens,

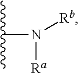

or C₁-C₆ alkyl that is unsubstituted or substituted by one or more halogens;
L₂ is linker unit (connecting LLM to L₀);
When L₀ is

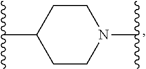 [SMILES: *C1CCN(*)CC1]

L₂ comprises at least one or more of the following fragments:

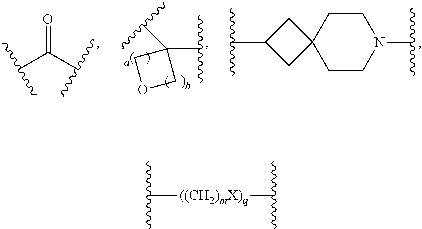

that is unsubstituted or substituted by one or more L₂¹⁻¹, or

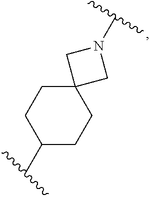 [SMILES: *C1CCC2(CC1)CN(*)C2]

a and b are independently 1or 2, m is an integer from 1to 4, q is an integer from 1to 6, X is 0; L₂¹⁻¹ is halogen, C₁-C₆ alkyl that is unsubstituted or substituted by one or more halogens, C₁-C₆ alkoxy that is unsubstituted or substituted by one or more halogens,

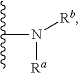

hydroxyl, oxo or

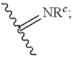

Each R^{c} is independently H, C₁-C₆ alkyl that is unsubstituted or substituted by one or more R^{c-1}, a 3- to 10-membered cycloalkyl that is unsubstituted or substituted by one or more R^{c-2}, a 3- to 11-membered heterocycloalkyl that is unsubstituted or substituted by one or more R^{c-3}, a 6- to 10-membered aryl that is unsubstituted or substituted by one or more R^{c-4}, or a 5- to 10-membered heteroaryl that is unsubstituted or substituted by one or more R^{c-5;}
Each R^{c-1}, R^{c-2}, R^{c-3}, R^{c-4} and R^{c-S} are independently halogen, cyano, hydroxyl, nitro, C₁-C₆ alkyl, C₁-C₆ alkoxy, a 3- to 10-membered cycloalkyl, a 3- to 11-membered heterocycloalkyl, a 6- to 10-membered aryl, or a 5 to 10-membered heteroaryl; the heteroatoms of the 3- to 11-membered heterocycloalkyl are selected from one or more of N, S and O, and the number of heteroatoms is 1, 2 or 3; the heteroatoms of the 5- to 10-membered heteroaryl are selected from one or more of N, S and O, and the number of heteroatoms is 1, 2 or 3;
LLM is

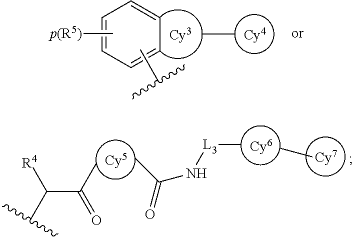

Ring Cy³ is a 5- to 12-membered heterocycle that is unsubstituted or substituted by one or more Cy³⁻¹, the heteroatoms of the 5- to 12-membered heterocycle are selected from one or more of N, S and O, and the number of heteroatoms is 1, 2 or 3; Cy³⁻¹ is independently C₁-C₆ alkyl, halogen, hydroxyl or oxo;
Ring Cy⁴ is a 5- to 12-membered heterocycloalkyl that is unsubstituted or substituted by one or more Cy⁴⁻¹, the heteroatoms of the 5- to 12-membered heterocycloalkyl are selected from one or more of N, S and O, and the number of heteroatoms is 1, 2 or 3; Cy⁴¹ is independently C₁-C₆ alkyl, halogen, hydroxyl or oxo;
Ring Cy⁵ is a 5- to 12-membered heterocycle that is unsubstituted or substituted by one or more Cy⁵-1, the heteroatoms of the 5- to 12-membered heterocycle are selected from one or more of N, S and O, and the number of heteroatoms is 1, 2 or 3; Cy⁵⁻¹ is independently C₁-C₆ alkyl, hydroxyl or oxo;
Ring Cy⁶ is a 6- to 10-membered aromatic ring that is unsubstituted or substituted by one or more Cy⁶⁻¹; Cy⁶⁻¹ is independently C₁-C₆ alkyl, hydroxyl or halogen;
Ring Cy⁷ is a 5- to 9-membered heteroaromatic ring that is unsubstituted or substituted by one or more Cy⁷⁻¹, the heteroatoms of the 5- to 9-membered heteroaromatic ring are selected from one or more of N, S and O, and the number of heteroatoms is 1, 2 or 3; Cy⁷⁻¹ is independently C₁-C₆ alkyl, hydroxyl or halogen;
R⁴ is independently hydrogen, halogen, hydroxyl or C₁-C₆ alkyl that is unsubstituted or substituted by one or more halogens;
P is 0, 1, 2 or 3;
Each R⁵ is independently halogen;
L₃ is

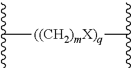

that is unsubstituted or substituted by one or more L₃⁻¹; where m is an integer from 1to 4, q is an integer from 1to 6, and X is absent or O; L₃⁻¹ is independently halogen, or C₁-C₆ alkyl that is unsubstituted or substituted by one or more halogens.

In a preferred embodiment, in the five-membered ring-fused six-membered ring compound as shown in Formula I, the pharmaceutically acceptable salt or isotope compound thereof; the definitions of some groups can be as follows, and the definitions of other groups can be as described in any one of the above embodiments (hereinafter referred to as “in a certain embodiment”): LLM is a group that binds to a ligase; and the ligase can be an E 3 ligase, preferably VHL, CRBN, MDM2, cIAP, Cereblon, XIAP, E3A, APC, UBR5(EDD1), SOCS/BC-box/eloBC/CUL5/RING, LNXp80, CBX4, CBLL1, HACE1, HECTD1, HECTD2, HECTD3, HECW1, HECW2, HERC1, HERC2, HERC3, HERC4, HUWE1, ITCH, NEDD4, NEDD4L, cPPIL2, PRPF19, PIAS1, PIAS2, PIAS3, PIAS4, RANBP2, RNF4, RBX1, SMURF1, SMURF2, STUB1, TOPORS, TRIP12, UBE3A, UBE3B, UBE3C, UBE4A, UBE4B, UBOX5, UBR5, WWP1, WWP2, Parkin, A20/TNFAIP3, AMFR/gp78, ARA54, β-TrCP1/BTRC, BRCA1, CBL, CHIP/STUB1, E6, E6AP/UBE3A, F-box protein 15/FBXO15, FBXW7/Cdc4, GRAIL/RNF128, HOIP/RNF31, cIAP-1/HIAP-2, cIAP-2/HIAP-1, cIAP(pan), ITCH/AIP4, KAP1, MARCH8, Mind Bomb 1/MIB1, Mind Bomb 2/MIB2, MuRF1/TRIM63, NDFIP1, NEDD4, NleL, Parkin, RNF2, RNF4, RNF8, RNF168, RNF43, SART1, Skp2, SMURF2, TRAF-1, TRAF-2, TRAF-3, TRAF-4, TRAF-5, TRAF-6, TRIM5, TRIM21, TRIM32, UBR5 or ZNRF3, more preferably VHL, CRBN, MDM2 or cIAP.

In a preferred embodiment, the linker unit can be a conventional linker unit in the art. Preferably, L₂ is -L₂⁻¹-L₂⁻²L₂⁻³-L₂⁻⁴-; L₂⁻¹, L₂⁻², L₂⁻³ and L₂⁻⁴ are independently absent,

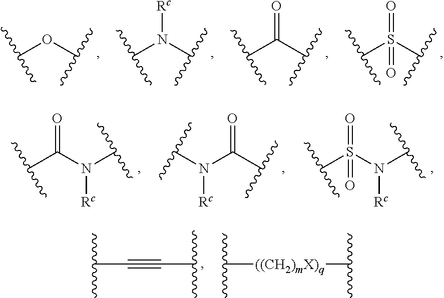

that is unsubstituted or substituted by one or more L₂¹⁻¹,

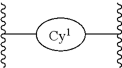

is unsubstituted or substituted by one or more L₂¹⁻², or

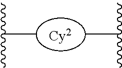

that is unsubstituted or substituted by one or more L₂¹⁻³, m is an integer from 1 to 4, q is an integer from 1 to 6, X is absent or O; Ring Cy¹ is a 4- to 12-membered heterocycle or 3- to 12-membered cycloalkyl; ring Cy² is a 5- to 10-membered heteroaromatic ring or a 6- to 10-membered aromatic ring; the heteroatoms of the 4- to 12-membered heterocycle are selected from one or more of N, S and O, and the number of heteroatoms is 1, 2 or 3; the heteroatoms of the 5- to 10-membered heteroaromatic ring are selected from one or more of N, S and O, and the number of heteroatoms is 1, 2 or 3;
L₂¹⁻¹ and L₂¹⁻² are independently halogen, C₁-C₆ alkyl that is unsubstituted or substituted by one or more halogens, C₁-C₆ alkoxy that is unsubstituted or substituted by one or more halogens,

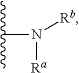

hydroxyl, oxo, or

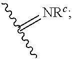

L₂¹⁻³ is independently halogen, C₁-C₆ alkyl that is unsubstituted or substituted by one or more halogens, C₁-C₆ alkoxy that is unsubstituted or substituted by one or more halogens, cyano,

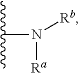

a 3- to 10-membered cycloalkyl that is unsubstituted or substituted by one or more L₂¹⁻³⁻¹, a 3- to 11-membered heterocycloalkyl that is unsubstituted or substituted by one or more L₂¹⁻³⁻¹,

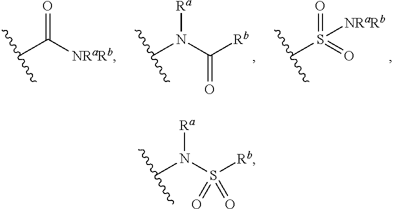

—SO₂—R^{a}, —SO—R^{a},

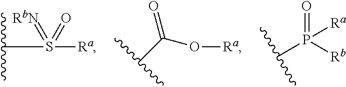

or hydroxyl; the heteroatoms of the 3- to 11-membered heterocycloalkyl are selected from one or more of N, S and O, and the number of heteroatoms is 1, 2 one or 3;
L₂¹⁻³⁻¹ and L₂¹⁻³⁻² are independently halogen, C₁-C₆ alkyl that is unsubstituted or substituted by one or more halogens, C₁-C₆ alkoxy that is unsubstituted or substituted by one or more halogens, hydroxyl, oxo, or

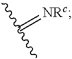

Each R^{c} is independently H, C₁-C₆ alkyl that is unsubstituted or substituted by one or more R^{c-1}, a 3- to 10-membered cycloalkyl that is unsubstituted or substituted by one or more R^{c-2}, a 3- to 11-membered heterocycloalkyl that is unsubstituted or substituted by one or more R^{c-3}, a 6- to 10-membered aryl that is unsubstituted or substituted by one or more R^{c-4}, or a 5- to 10-membered heteroaryl that is unsubstituted or substituted by one or more R^{c-5;}
Each R^{c-1}, R^{c-2}, R^{c-3}, R^{c-4} and R^{c-5} are independently halogen, cyano, hydroxyl, nitro, C₁-C₆ alkyl, C₁-C₆ alkoxy, a 3- to 10-membered cycloalkyl, a 3- to 11-membered heterocycloalkyl, a 6- to 10-membered aryl, or a 5- to 10-membered heteroaryl; the heteroatoms of the 3- to 11-membered heterocycloalkyl are selected from one or more of N, S and O, and the number of heteroatoms is 1, 2 or 3; the heteroatoms of the 5- to 10-membered heteroaryl are selected from one or more of N, S and O, and the number of heteroatoms is 1, 2 or 3;
Each R^{a} and each R^{b} are independently H, C₁-C₆ alkyl that is unsubstituted or substituted by one or more R^{a-1}, a 3- to 10-membered cycloalkyl that is unsubstituted or substituted by one or more R^{a-2}, a 3- to 11-membered heterocycloalkyl that is unsubstituted or substituted by one or more R^{a-3}, a 6- to 10-membered aryl that is unsubstituted or substituted by one or more R^{a-4}, or a 5- to 10-membered heteroaryl that is unsubstituted or substituted by one or more R^{a-5};
Each R^{a-1}, R^{a-2}, R^{a-3}, R^{a-4} and R^{a-5} are independently halogen, cyano, hydroxyl, nitro, C₁-C₆ alkyl, C₁-C₆ alkoxy, a 3- to 10-membered cycloalkyl, a 3- to 11-membered heterocycloalkyl, a 6- to 10-membered aryl, or a 5- to 10-membered heteroaryl; the heteroatoms of the 3 to 11-membered heterocycloalkyl are selected from one or more of N, S and O, and the number of heteroatoms is 1, 2 or 3; the heteroatoms of the 5- to 10-membered heteroaryl are selected from one or more of N, S and O, and the number of heteroatoms is 1, 2 or 3.

In a preferred embodiment, when ring Cy is a 5-membered heterocycle, the heteroatom of the 5-membered heterocycle is 0 and the number is 1. The 5-membered heterocycle is preferably a tetrahydrofuran ring.

In a preferred embodiment, when ring Cy is a 5-membered heteroaromatic ring, the heteroatoms of the 5-membered heteroaromatic ring are selected from one or two of N, S and O, and the number of heteroatoms is 1 or 2. The 5-membered heteroaromatic ring is preferably a pyrrole ring, pyrazole ring, thiazole ring, oxazole ring or imidazole ring.

In a preferred embodiment, when R¹is a 5- to 10-membered heteroaryl that is unsubstituted or substituted by one or more R¹⁻¹, the heteroatoms of the 5- to 10-membered heteroaryl are selected from one or two of N, O and S, and the number of heteroatoms is 1, 2 or 3; the 5- to 10-membered heteroaryl can be oxazolyl, pyrazolyl, thiazolyl, imidazolyl, pyridyl, pyridazinyl, pyrazinyl, pyrimidinyl, triazinyl or pyrazolopyrimidinyl, such as pyridyl, oxazolyl, pyrazolyl or

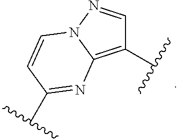 [SMILES: *c1ccn2ncc(*)c2n1]

In a preferred embodiment, when R¹ is a 6- to 10-membered aryl that is unsubstituted or substituted by one or more R¹⁻², the 6- to 10-membered aryl group is preferably phenyl or naphthyl.

In a preferred embodiment, when each R¹⁻¹ and R¹⁻² are independently a halogen, the halogen can be fluorine, chlorine, bromine or iodine, such as fluorine.

In a preferred embodiment, when each R¹⁻¹ and R¹⁻² are independently a 3- to 11-membered heterocycloalkyl that is unsubstituted or substituted by one or more R¹⁻¹⁻¹, the 3- to 11-membered heterocycloalkyl may be 4- to 8-membered heterocycloalkyl, the heteroatoms of the 3- to 11-membered heterocycloalkyl are selected from one or two of N, S and O, and the number of heteroatoms is 1 or 2; preferably, the heteroatom of the 3- to 11-membered heterocycloalkyl is N, S or O, and the number of heteroatoms is 1or 2; preferably, each R¹⁻¹⁻¹ is independently halogen, hydroxyl, oxo, C₁-C₆ alkoxy that is unsubstituted or substituted by one or more halogens, or C₁-C₆ alkyl that is unsubstituted or substituted by one or more halogens; the 3- to 11-membered heterocycloalkyl is preferably pyridazinyl, piperazinyl, piperidinyl, tetrahydropyrrolyl, oxacyclobutyl, azabicyclo[2.2.2]octyl, azabicyclo[3.2.1]octyl, azaspiro[3.3]heptyl or azabicyclo[2.2.1]heptyl, such as

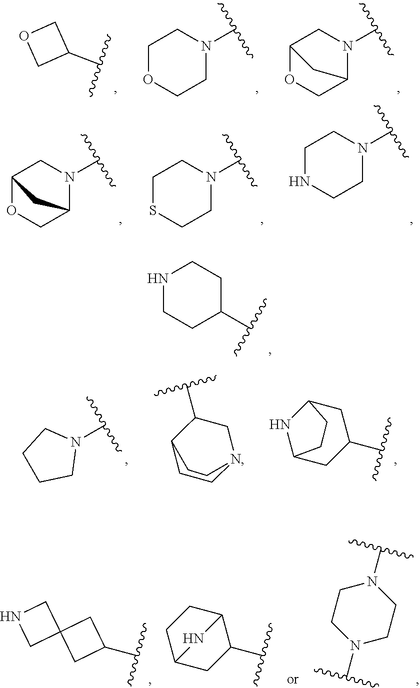

In a preferred embodiment, when each R¹⁻¹ and R¹⁻² are independently C₁-C₆ alkoxy that is unsubstituted or substituted by one or more R¹⁻¹⁻³, the C₁-C₆ alkoxy can be C₁-C₄ alkoxy, such as methoxy, ethoxy, n-propoxy, isopropoxy, n-butoxy, isobutoxy, sec-butoxy or tert-butoxy; preferably, each R¹⁻¹⁻³ is independently halogen, hydroxyl, C₁-C₆ alkoxy that is unsubstituted or substituted by one or more halogens or C₁-C₆ alkyl that is unsubstituted or substituted by one or more halogens.

In a preferred embodiment, when each R¹⁻¹ and R¹⁻² are independently C₁-C₆ alkyl that is unsubstituted or substituted by one or more R¹⁻¹⁻⁴, the C₁-C₆ alkyl can be C₁-C₄ alkyl, such as methyl, ethyl, n-propyl, isopropyl, n-butyl, isobutyl, sec-butyl or tert-butyl; preferably, each R¹⁻¹⁻⁴ is independently halogen, hydroxyl, C₁-C₆ alkoxy that is unsubstituted or substituted by one or more halogens or C₁-C₆ alkyl that is unsubstituted or substituted by one or more halogens.

In a preferred embodiment, when each R¹⁻¹ and R¹⁻² are independently a 3- to 10-membered cycloalkyl that is unsubstituted or substituted by one or more R¹⁻¹⁻⁵, the 3- to 10-membered cycloalkyl can be a 3- to 6-membered cycloalkyl, such as cyclopropyl, cyclobutyl, cyclopentyl or cyclohexyl; preferably, each R¹⁻¹⁻⁵ is independently halogen, hydroxyl, C₁-C₆ alkoxy that is unsubstituted or substituted by one or more halogens or C₁-C₆ alkyl that is unsubstituted or substituted by one or more halogens.

In a preferred embodiment, when each R¹⁻¹ and R¹⁻² are independently a 5- to 10-membered heteroaryl that is unsubstituted or substituted by one or more R¹⁻¹⁻⁷, the heteroatom of the 5- to 10-membered heteroaryl is N, and the number of heteroatoms is 1, 2 or 3; preferably, each R¹⁻¹⁻⁷ is independently halogen, hydroxyl, C₁-C₆ alkoxy that is unsubstituted or substituted by one or more halogens or C₁-C₆ alkyl that is unsubstituted or substituted by one or more halogens; the 5- to 10-membered heteroaryl may be pyridyl, pyridylene, pyridazinyl, pyrazinyl, pyrimidinyl or triazinyl, such as pyridyl or pyridylene.

In a preferred embodiment, when each R¹⁻¹ and R¹⁻² are independently a 6- to 10-membered aryl that is unsubstituted or substituted by one or more R¹⁻¹⁻⁸, the 6- to 10-membered aryl is preferably phenyl or naphthyl; preferably, each R¹⁻¹⁻⁸ is independently halogen, hydroxyl, C₁-C₆ alkoxy that is unsubstituted or substituted by one or more halogens or C₁-C₆ alkyl that is unsubstituted or substituted by one or more halogens.

In a preferred embodiment, when each R¹⁻¹⁻¹ and R¹⁻¹⁻⁷ are independently C₁-C₆ alkyl that is unsubstituted or substituted by one or more halogens, the C₁-C₆ alkyl can be C₁-C₄ alkyl, such as methyl, ethyl, n-propyl, isopropyl, n-butyl, isobutyl, sec-butyl or tert-butyl.

In a preferred embodiment, when each R¹⁻¹⁻⁴ is independently halogen, the halogen can be fluorine, chlorine, bromine or iodine, such as fluorine.

In a preferred embodiment, when R² is halogen, the halogen can be fluorine, chlorine, bromine or iodine, such as fluorine or chlorine.

In a preferred embodiment, when R² is a 5- to 10-membered heteroaryl that is unsubstituted or substituted by one or more R²⁻¹, the heteroatom of the 5- to 10-membered heteroaryl is N, and the number of heteroatoms is 1, 2 or 3; the 5- to 10-membered heteroaryl is preferably pyridyl, pyridazinyl, pyrazinyl, pyrimidinyl or triazinyl, such as pyridyl.

In a preferred embodiment, when R² is C₁-C₆ alkoxy that is unsubstituted or substituted by one or more R²⁻², the C₁-C₆ alkoxy can be C₁-C₄ alkoxy, such as methoxy, ethoxy, n-propoxy, isopropoxy, n-butoxy, isobutoxy, sec-butoxy or tert-butoxy.

In a preferred embodiment, when R² is C₁-C₆ alkyl that is unsubstituted or substituted by one or more R²⁻³, the C₁-C₆ alkyl can be C₁-C₄ alkyl, such as methyl, ethyl, n-propyl, isopropyl, n-butyl, isobutyl, sec-butyl or tert-butyl.

In a preferred embodiment, when R² is independently a 4- to 10-membered heterocycloalkyl that is unsubstituted or substituted by one or more R²⁻⁴, the 4- to 10-membered heterocycloalkyl may be 5- to 8-membered heterocycloalkyl, the heteroatoms of the 4- to 10-membered heterocycloalkyl are selected from one or two of N, S and O, and the number of heteroatoms is 1 or 2; preferably, the heteroatom of the 4- to 10-membered heterocycloalkyl is N, S or O, and the number of heteroatoms is 1 or 2; the 4- to 10-membered heterocycloalkyl is preferably piperazinyl, piperidinyl, tetrahydropyrrolyl, azabicyclo[2.2.2]octyl, azabicyclo[3.2.1]octyl, azaspiro[3.3]heptyl or azabicyclo[2.2.1]heptyl, such as

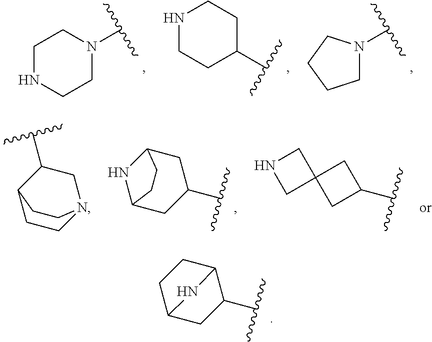

In a preferred embodiment, when each R²⁻¹, R²⁻², R²⁻³ and R²⁻⁴ are independently halogen, the halogen can be fluorine, chlorine, bromine or iodine; such as fluorine.

In a preferred embodiment, when each R²⁻¹, R²⁻², R²⁻³ and R²⁻⁴ are independently C₁-C₆ alkyl that is unsubstituted or substituted by one or more halogens, the C₁-C₆ alkyl can be C₁-C₄ alkyl, such as methyl, ethyl, n-propyl, isopropyl, n-butyl, isobutyl, sec-butyl or tert-butyl; and the halogen can be fluorine, chlorine, bromine or iodine; such as fluorine.

In a preferred embodiment, when each R²⁻¹, R²⁻², R²⁻³ and R²⁻⁴ are independently C₁-C₆ alkoxy, the C₁-C₆ alkoxy can be C₁-C₄ alkoxy, such as methoxy, ethoxy, n-propoxy, isopropoxy, n-butoxy, isobutoxy, sec-butoxy or tert-butoxy, further such as methoxy; and the halogen can be fluorine, chlorine, bromine or iodine; such as fluorine.

In a preferred embodiment, when R³ is halogen, the halogen can be fluorine, chlorine, bromine or iodine; such as fluorine.

In a preferred embodiment, when R³ is a 3- to 11-membered heterocycloalkyl that is unsubstituted or substituted by one or more R³⁻¹, the 3- to 11-membered heterocycloalkyl may be 3- to 8-membered heterocycloalkyl, the heteroatoms of the 3- to 11-membered heterocycloalkyl are preferably N and/or O, and the number of heteroatoms is 1 or 2; the 3- to 11-membered heterocycloalkyl is preferably piperidinyl, tetrahydropyrrolyl, tetrahydropyrrolidene, 2-azaspiro[3.3]heptyl, 2-oxaspiro[3.3]heptyl, morpholinyl, tetrahydropyranyl, oxa cyclobutyl, azabicyclo[2.2.1]heptyl or diazabicyclo[2.2.1]heptyl; preferably, each R³⁻¹ is independently halogen, oxo or hydroxyl, such as hydroxyl; the 3- to 8-membered heterocycloalkyl that is unsubstituted or substituted by one or more R³⁻¹ is preferably

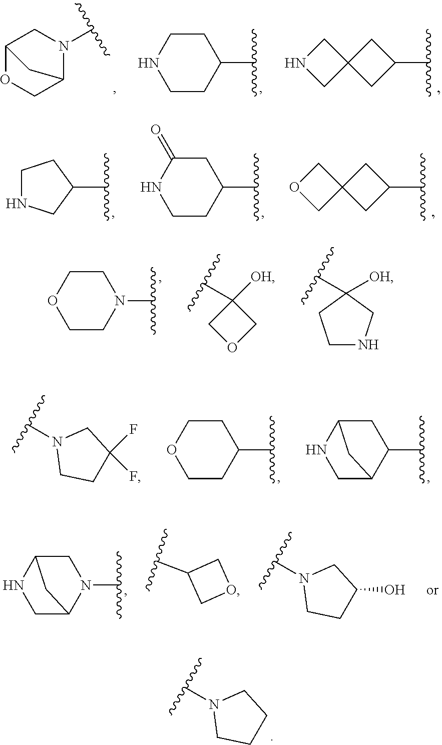

In a preferred embodiment, when R³ is a 3- to 10-membered cycloalkyl that is unsubstituted or substituted by one or more R³⁻², the 3- to 10-membered cycloalkyl may be C₃-C₆ cycloalkyl, can also be cyclopropyl, cyclobutyl, cyclopentyl or cyclohexyl, such as cyclopropyl, cyclobutyl or cyclohexyl; preferably, each R³⁻² is independently halogen or hydroxyl; the 3- to 10-membered cycloalkyl that is unsubstituted or substituted by one or more R³⁻² is preferably

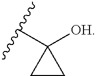 [SMILES: *C1(O)CC1]

In a preferred embodiment, when R³ is C₁-C₆ alkyl that is unsubstituted or substituted by one or more R³⁻⁴, the C₁-C₆ alkyl can be methyl, ethyl, n-propyl, isopropyl, n-butyl, isobutyl, primary butyl, sec-butyl, tert-butyl, n-pentyl, isopentyl, primary pentyl, sec-pentyl, tert-pentyl or neopentyl, preferably can be methyl, ethyl, propyl, isopropyl or isopentyl; preferably, each R³⁻⁴ is independently halogen, hydroxyl, —SO₂—R^{a} or

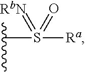

wherein R^{a} is C₁-C₆ alkyl and R^{b} is hydrogen; and the C₁-C₆ alkyl that is unsubstituted or substituted by one or more R³⁻⁴ can be

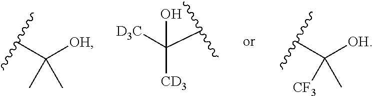

In a preferred embodiment, when R³ is C₁-C₆ alkyl that is unsubstituted or substituted by one or more R³⁻⁴, the C₁-C₆ alkyl can be methyl, ethyl, n-propyl, isopropyl, n-butyl, isobutyl, primary butyl, sec-butyl, tert-butyl, n-pentyl, isopentyl, primary pentyl, sec-pentyl, tert-pentyl or neopentyl, and can also be methyl, ethyl, propyl, isopropyl or isopentyl; preferably, each R³⁻⁴ is independently deuterium, halogen, hydroxyl, —SO₂—R^{a} or

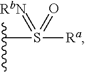

R^{a} is C₁-C₆ alkyl and R^{b} is hydrogen; and the C₁-C₆ alkyl that is unsubstituted or substituted by one or more R³⁻⁴ can be

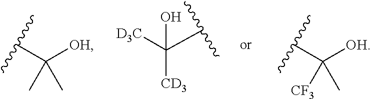

In a preferred embodiment, when R³ is C₁-C₆ alkoxy that is unsubstituted or substituted by one or more R³⁻⁷, the C₁-C₆ alkoxy can be C₁-C₄ alkoxy, such as methoxy, ethoxy, n-propoxy, isopropoxy, n-butoxy, isobutoxy, primary butoxy, sec-butoxy or tert-butoxy; preferably can be methoxy, ethoxy or isopropoxy; preferably, each R³⁻⁷ is independently halogen, the C₁-C₆ alkoxy that is unsubstituted or substituted by one or more R³⁻⁷ is preferably methoxy,

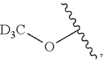 [SMILES: *OC([2H])([2H])[2H]]

isopropoxy or trifluoromethoxy.

In a preferred embodiment, when R³ is C₁-C₆ alkoxy that is unsubstituted or substituted by one or more R³⁻⁷, the C₁-C₆ alkoxy can be C₁-C₄ alkoxy, such as methoxy, ethoxy, n-propoxy, isopropoxy, n-butoxy, isobutoxy, primary butoxy, sec-butoxy or tert-butoxy; preferably can be methoxy, ethoxy or isopropoxy; preferably, each R³⁻⁷ is independently deuterium or halogen, the C₁-C₆ alkoxy that is unsubstituted or substituted by one or more R³⁻⁷ is preferably methoxy,

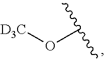 [SMILES: *OC([2H])([2H])[2H]]

isopropoxy or trifluoromethoxy.

In a preferred embodiment, when R³ is a hydroxyl group substituted by R³⁻⁸, R³⁻⁸ can be a 3- to 6-membered cycloalkyl or a 3- to 6-membered heterocycloalkyl, the heteroatom of the 3- to 6-membered heterocycloalkyl is oxygen, and the number of heteroatoms is 1; the hydroxyl group substituted by R³⁻⁸ is preferably

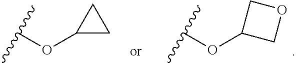 [SMILES: *OC1CC1 ; *OC1COC1]

In a preferred embodiment, when R³ is

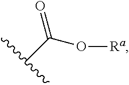

### R^{a} can be H.

In a preferred embodiment, when each R³⁻¹, R³⁻², R³⁻⁴, R³⁻⁵, R³⁻⁶ and R³⁻⁷ are independently a halogen, the halogen can be fluorine, chlorine, bromine or iodine, such as fluorine.

In a preferred embodiment, when each R³⁻¹, R³⁻², R³⁻⁴, R³⁻⁵, R³⁻⁶ and R³⁻⁷ are independently a 3- to 11-membered heterocycloalkyl that is unsubstituted or substituted by one or more R³⁻¹⁻¹, the 3- to 11-membered heterocycloalkyl may be 5 to 8-membered heterocycloalkyl, the heteroatoms of the 3- to 11-membered heterocycloalkyl are preferably N and/or O, and the number of heteroatoms is 1 or 2; the 3- to 11-membered heterocycloalkyl that is unsubstituted or substituted by one or more R³⁻¹⁻¹ is preferably tetrahydropyrrolyl, oxacyclobutyl or contains one oxygen and/or one azospiroheptyl group, such as

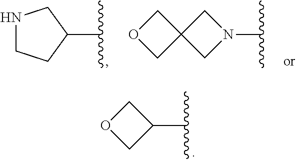

In a preferred embodiment, when each R³⁻¹⁻¹, R³⁻¹⁻³, R³⁻¹⁻⁴ and R³⁻¹⁻⁵ are independently C₁-C₆ alkyl that is unsubstituted or substituted by one or more halogens, the C₁-C₆ alkyl can be C₁-C₄ alkyl, and can be methyl, ethyl, n-propyl, isopropyl, n-butyl, isobutyl, primary butyl, sec-butyl, or tert-butyl, preferably can be methyl or ethyl.

In a preferred embodiment, when each R³⁻¹⁻¹, R³⁻¹⁻³, R³⁻¹⁻⁴ and R³⁻¹⁻⁵ are independently halogen, the halogen can be fluorine, chlorine, bromine or iodine, such as fluorine.

In a preferred embodiment, when L₀ is a 5- to 12-membered cycloalkylene group that is unsubstituted or substituted by one or more L₀⁻², the 5- to 12-membered cycloalkylene group can be subspiro ring, subbridged ring or subfused ring, preferably can be

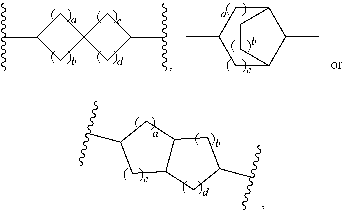

wherein a, b, c and d are independently 0, 1 or 2; such as

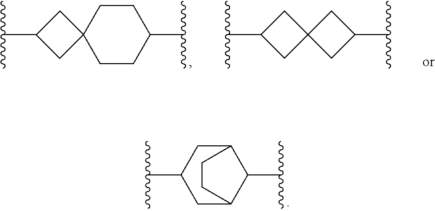

In a preferred embodiment, when L₀ is a 5- to 12-membered cycloalkylene group that is unsubstituted or substituted by one or more L₀⁻², the 5- to 12-membered cycloalkylene group can be subspiro ring, subbridged ring or subfused ring, preferably can be

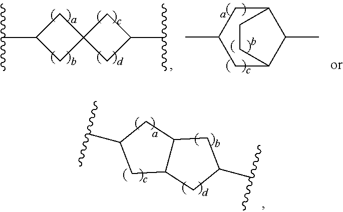

wherein a, b, c and d are independently 0, 1 or 2; such as

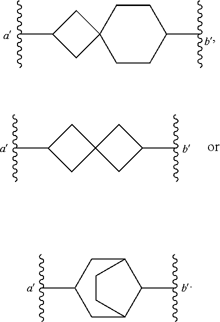 [SMILES: *C1CCC2(CC1)CC(*)C2 ; *C1CC2(C1)CC(*)C2 ; *C1CC2CCC(C1)C2*]

wherein the a′ end is connected to ring Cy, and the b′ end is connected to L².

In a preferred embodiment, when L₀ is a 5- to 12-membered heterocycloalkylene group that is unsubstituted or substituted by one or more L₀⁻³, the 5- to 12-membered heterocycloalkylene group can be a single ring, subspiro ring, subbridged ring or subfused ring, preferably can be

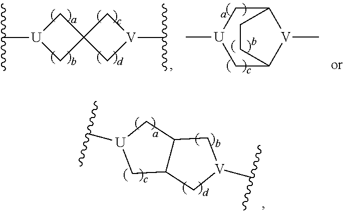

wherein U and V are independently CH or N, and at least one of U and V is N, a, b, c and d are independently 0, 1 or 2; such as

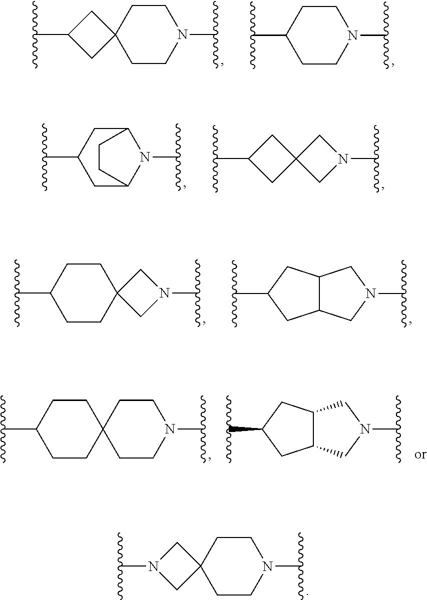

In a preferred embodiment, when L₀ is a 5- to 12-membered heterocycloalkylene group that is unsubstituted or substituted by one or more L₀⁻³, the 5- to 12-membered heterocycloalkylene group can be a single ring, subspiro ring, subbridged ring or subfused ring, preferably can be

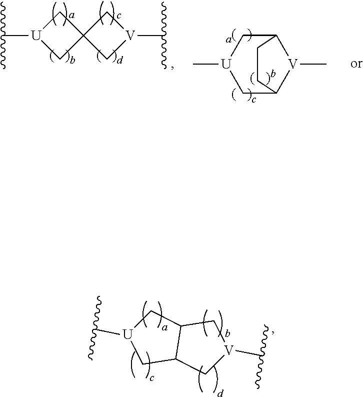

wherein U and V are independently CH or N, and at least one of U and V is N, a, b, c and d are independently 0, 1 or 2; such as

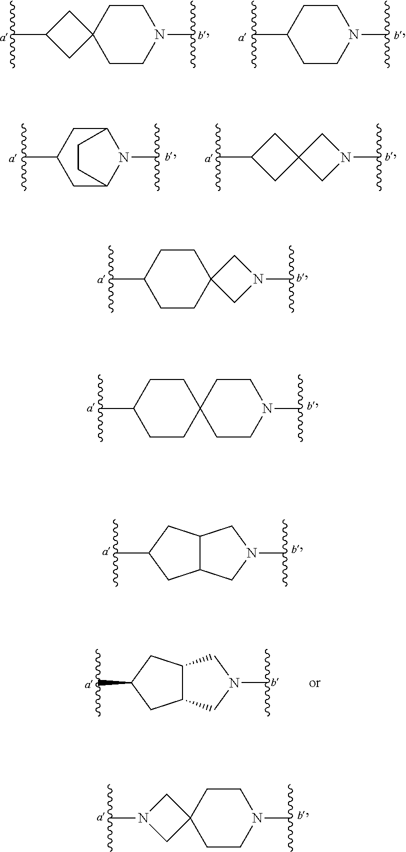

where the a′ end is connected to ring Cy, and the b′ end is connected to L².

In a preferred embodiment, when each L₀⁻² is independently C₁-C₆ alkyl that is unsubstituted or substituted by one or more halogens, the C₁-C₆ alkyl can be C₁-C₄ alkyl, preferably, can be methyl, ethyl, n-propyl, isopropyl, n-butyl, isobutyl, primary butyl, sec-butyl or tert-butyl, and preferably methyl, ethyl or isopropyl; and the halogen can be fluorine, chlorine, bromine or iodine, such as fluorine.

In a preferred embodiment, when each L₀⁻² is independently halogen, the halogen can be fluorine, chlorine, bromine or iodine, such as fluorine.

In a preferred embodiment, when each L₀⁻² is independently C₁-C₆ alkoxy that is unsubstituted or substituted by one or more halogens, the halogen can be fluorine, chlorine, bromine or iodine, such as fluorine.

In a preferred embodiment, when each L₀⁻² is independently C₁-C₆ alkoxy that is unsubstituted or substituted by one or more halogens, the C₁-C₆ alkoxy can be C₁-C₄ alkoxy, such as methoxy, ethoxy, n-propoxy, isopropoxy, n-butoxy, isobutoxy, primary butoxy, sec-butoxy or tert-butoxy; preferably methoxy, ethoxy or isopropoxy.

In a preferred embodiment, when each L₀⁻³ is independently C₁-C₆ alkyl that is unsubstituted or substituted by one or more halogens, the C₁-C₆ alkyl can be C₁-C₄ alkyl, preferably, can be methyl, ethyl, n-propyl, isopropyl, n-butyl, isobutyl, primary butyl, sec-butyl or tert-butyl, and preferably methyl, ethyl or isopropyl; and the halogen can be fluorine, chlorine, bromine or iodine, such as fluorine.

In a preferred embodiment, when each L₀⁻³ is independently halogen, the halogen can be fluorine, chlorine, bromine or iodine, such as fluorine.

In a preferred embodiment, when each L₀⁻³ is independently C₁-C₆ alkoxy that is unsubstituted or substituted by one or more halogens, the halogen can be fluorine, chlorine, bromine or iodine, such as fluorine.

In a preferred embodiment, when each L₀⁻³ is independently C₁-C₆ alkoxy that is unsubstituted or substituted by one or more halogens, the C₁-C₆ alkoxy can be C₁-C₄ alkoxy, such as methoxy, ethoxy, n-propoxy, isopropoxy, n-butoxy, isobutoxy, primary butoxy, sec-butoxy or tert-butoxy; preferably methoxy, ethoxy or isopropoxy.

In a preferred embodiment, when each R^{a} and each R^{b} are independently C₁-C₆ alkyl that is unsubstituted or substituted by one or more R^{a-1}, the C₁-C₆ alkyl can be C₁-C₄ alkyl, preferably, can be methyl, ethyl, n-propyl, isopropyl, n-butyl, isobutyl, primary butyl, sec-butyl or tert-butyl, and preferably methyl, ethyl or isopropyl.

In a preferred embodiment, when each R^{a} and each R^{b} are independently a 3- to 10-membered cycloalkyl that is unsubstituted or substituted by one or more R^{a-2}, the 3- to 10-membered cycloalkyl can be a 3- to 6-membered cycloalkyl, such as cyclopropyl, cyclobutyl, cyclopentyl or cyclohexyl.

In a preferred embodiment, when each R^{a} and each R^{b} are independently a 3- to 11-membered heterocycloalkyl that is unsubstituted or substituted by one or more R^{a-3}, the 3- to 11-membered heterocycloalkyl may be a 5 to 8-membered heterocycloalkyl, the heteroatoms of the 3- to 11-membered heterocycloalkyl are selected from one or two of N, S and O, and the number of heteroatoms is 1 or 2; preferably, the heteroatom of the 3- to 11-membered heterocycloalkyl is N, S or O, and the number of heteroatoms is 1 or 2; the 3- to 11-membered heterocycloalkyl is preferably piperazinyl, piperidinyl, tetrahydropyrrolyl, azabicyclo[2.2.2]octyl, azabicyclo[3.2.1]octyl, azaspiro[3.3]heptyl or azabicyclo[2.2.1]heptyl, such as

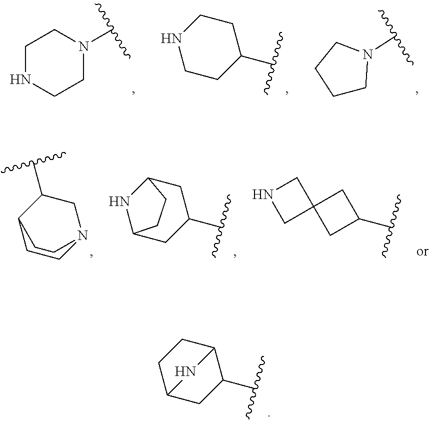

In a preferred embodiment, when each R^{a} and each R^{b} are independently a 3- to 11-membered heterocycloalkyl that is unsubstituted or substituted by one or more R^{a-3}, the 3- to 11-membered heterocycloalkyl may be a 5 to 8-membered heterocycloalkyl, the heteroatoms of the 3- to 11-membered heterocycloalkyl are selected from one or two of N, S and O, and the number of heteroatoms is 1 or 2; preferably, the heteroatom of the 3- to 11-membered heterocycloalkyl is N, S or O, and the number of heteroatoms is 1 or 2; the 3- to 11-membered heterocycloalkyl is preferably piperazinyl, piperidinyl, tetrahydropyrrolyl, azabicyclo[2.2.2]octyl, azabicyclo[3.2.1]octyl, azaspiro[3.3]heptyl or azabicyclo[2.2.1]heptyl.

In a preferred embodiment, when each R^{a} and each R^{b} are independently a 6- to 10-membered aryl that is unsubstituted or substituted by one or more R^{a-4}, the 6- to 10-membered aryl can be benzene ring or naphthalene ring.

In a preferred embodiment, when each R^{a} and each R^{b} are independently a 5- to 10-membered heteroaryl that is unsubstituted or substituted by one or more R^{a}-s; the 5- to 10-membered heteroaryl can be a 5-membered heteroaryl or a 6-membered heteroaryl; the 6-membered heteroaryl group is preferably pyridyl, pyridazinyl, pyrazinyl, pyrimidinyl or triazinyl; and the 5-membered heteroaryl group is preferably pyrazolyl, thiazolyl or imidazolyl.

In a preferred embodiment, in ring Cy³, the 5- to 12-membered heterocycle may be a 5 to 6-membered heterocycle, and the heteroatom of the 5- to 12-membered heterocycle is preferably N, S or O, and the number of the heteroatom is 1 or 2; the 5- to 12-membered heterocycle may be a tetrahydropyrrole ring, a piperidine ring, a tetrahydrofuran ring or a tetrahydrothiophene ring, such as a tetrahydropyrrole ring.

In a preferred embodiment, in Cy³⁻¹, the C₁-C₆ alkyl can be C₁-C₄ alkyl, such as methyl, ethyl, n-propyl, isopropyl, n-butyl, isobutyl, primary butyl, sec-butyl or tert-butyl; and can also be methyl, ethyl or isopropyl.

In a preferred embodiment, in ring Cy⁴, the 5- to 12-membered heterocycle may be a 5 to 6-membered heterocycle, and the heteroatom of the 5- to 6-membered heterocycle is preferably N, S or O, and the number of the heteroatom is 1 or 2; the 5- to 12-membered heterocycle may be a tetrahydropyrrole ring, a piperidine ring, a tetrahydrofuran ring or a tetrahydrothiophene ring, such as a piperidine ring.

In a preferred embodiment, in Cy⁴⁻¹, the C₁-C₆ alkyl can be C₁-C₄ alkyl, such as methyl, ethyl, n-propyl, isopropyl, n-butyl, isobutyl, primary butyl, sec-butyl or tert-butyl; and can also be methyl, ethyl or isopropyl.

In a preferred embodiment, in ring Cy⁵, the 5- to 12-membered heterocycle may be a 5 to 6-membered heterocycle, and the heteroatom of the 5- to 12-membered heterocycle is preferably N, S or O, and the number of the heteroatom is 1 or 2; the 5- to 12-membered heterocycle may be a tetrahydropyrrole ring, a piperidine ring, a tetrahydrofuran ring or a tetrahydrothiophene ring, such as a tetrahydropyrrole ring.

In a preferred embodiment, in Cy⁵⁻¹, the C₁-C₆ alkyl can be C₁-C₄ alkyl, such as methyl, ethyl, n-propyl, isopropyl, n-butyl, isobutyl, primary butyl, sec-butyl or tert-butyl; and can also be methyl, ethyl or isopropyl.

In a preferred embodiment, in ring Cy⁶, the 6- to 10-membered aromatic ring can be a benzene ring or a naphthalene ring.

In a preferred embodiment, in Cy⁶⁻¹, the halogen can be fluorine, chlorine, bromine or iodine, such as fluorine or chlorine.

In a preferred embodiment, in Cy⁶⁻¹, the C₁-C₆ alkyl can be C₁-C₄ alkyl, such as methyl, ethyl, n-propyl, isopropyl, n-butyl, isobutyl, primary butyl, sec-butyl or tert-butyl; and can also be methyl, ethyl or isopropyl.

In a preferred embodiment, in ring Cy⁷, the 5- to 9-membered heteroaromatic ring may be a 5-membered heteroaromatic ring or a 6-membered heteroaromatic ring; the 6-membered heteroaromatic ring is preferably a pyridine ring, a pyridazine ring, a pyrazine ring, a pyrimidine ring or a triazine ring; and the 5-membered heteroaromatic ring is preferably a pyrazole ring, an oxazole ring, a thiazole ring or an imidazole ring, such as

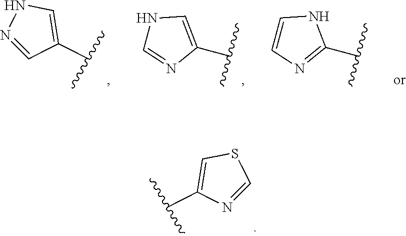

In a preferred embodiment, in Cy⁷⁻¹, the halogen can be fluorine, chlorine, bromine or iodine, such as fluorine or chlorine.

In a preferred embodiment, in Cy⁷⁻¹, the C₁-C₆ alkyl can be C₁-C₄ alkyl, such as methyl, ethyl, n-propyl, isopropyl, n-butyl, isobutyl, primary butyl, sec-butyl or tert-butyl; and can also be methyl.

In a preferred embodiment, in R⁴, the C₁-C₆ alkyl can be C₁-C₄ alkyl, such as methyl, ethyl, n-propyl, isopropyl, n-butyl, isobutyl, primary butyl, sec-butyl or tert-butyl; and can also be tert-butyl.

In a preferred embodiment, in R⁴, the halogen can be fluorine, chlorine, bromine or iodine, such as fluorine or chlorine.

In a preferred embodiment, in R⁵, the halogen can be fluorine, chlorine, bromine or iodine, such as fluorine or chlorine.

In a preferred embodiment, in L₃⁻¹, the C₁-C₆ alkyl can be C₁-C₄ alkyl, such as methyl, ethyl, n-propyl, isopropyl, n-butyl, isobutyl, primary butyl, sec-butyl or tert-butyl; such as methyl.

In a preferred embodiment, when the ring Cy¹ is a 5- to 12-membered heterocycle, the heteroatom of the 5- to 12-membered heterocycle is N, S or O, and the number of heteroatoms is 1 or 2; the 5- to 12-membered heterocycle can be oxacyclobutyl ring, diazaspiro[3.3]heptane, tetrahydrofuran ring, piperidine ring, piperazine ring, diazaspiro[3.5]nonane, azaspiro[3.3]heptane, diazaspiro[5.5]undecane, azaspiro[3.5]nonaneorazaspiro[5.5]undecane.

In a preferred embodiment, when the ring Cy¹ is a 3- to 12-membered cycloalkyl, the 3- to 12-membered cycloalkyl may be a 3- to 6-membered cycloalkyl, such as cyclohexyl.

In a preferred embodiment, when ring Cy² is a 5- to 10-membered heteroaromatic ring, the heteroatoms of the 5- to 10-membered heteroaromatic ring are selected from one or two of N, S and O, and the number of the heteroatoms is 1 or 2; the 5- to 10-membered heteroaromatic ring is preferably a 5 or 6-membered heteroaromatic ring; the 6-membered heteroaromatic ring is preferably a pyridine ring, a pyridazine ring, a pyrazine ring, a pyrimidine ring or a triazine ring; and the 5-membered heteroaromatic ring is preferably a pyrazole ring, a thiazole ring or an imidazole ring.

In a preferred embodiment, in L₂¹⁻¹ and L₂¹⁻², the C₁-C₆ alkyl can be C₁-C₄ alkyl, such as methyl, ethyl, n-propyl, isopropyl, n-butyl, isobutyl, primary butyl, sec-butyl or tert-butyl; and can also be methyl, ethyl or isopropyl.

In a preferred embodiment, in L₂¹⁻¹ and L₂¹⁻², the C₁-C₆ alkoxy can be C₁-C₄ alkoxy, such as methoxy, ethoxy, n-propoxy, isopropoxy, n-butoxy, isobutoxy, primary butoxy, sec-butoxy or tert-butoxy; and can also be methoxy, ethoxy, or isopropoxy.

In a preferred embodiment, in L₂¹⁻¹ and L₂¹⁻², the halogen can be fluorine, chlorine, bromine or iodine, such as fluorine.

In a preferred embodiment, in L₂¹⁻³, the halogen can be fluorine, chlorine, bromine or iodine, such as fluorine or chlorine.

In a preferred embodiment, in L₂¹⁻³, in the C₁-C₆ alkyl that is unsubstituted or substituted by one or more halogens, the C₁-C₆ alkyl can be C₁-C₄ alkyl, such as methyl, ethyl, n-propyl, isopropyl, n-butyl, isobutyl, primary butyl, sec-butyl or tert-butyl; and can also be methyl, ethyl or isopropyl; and the halogen is preferably fluorine, chlorine, bromine or iodine, such as fluorine or chlorine.

In a preferred embodiment, in L₂¹⁻³, in the C₁-C₆ alkoxy that is unsubstituted or substituted by one or more halogens, the C₁-C₆ alkoxy can be C₁-C₄ alkoxy, such as methoxy, ethoxy, n-propoxy, isopropoxy, n-butoxy, isobutoxy, primary butoxy, sec-butoxy or tert-butoxy; and can also be methoxy, ethoxy, or isopropoxy; and the halogen is preferably fluorine, chlorine, bromine or iodine, such as fluorine or chlorine.

In a preferred embodiment, in L₂¹⁻³, in the 3- to 10-membered cycloalkyl that is unsubstituted or substituted by one or more L₂¹⁻³⁻¹, the 3- to 10-membered cycloalkyl can be C₃-C₆ cycloalkyl, can also be cyclopropyl, cyclobutyl, cyclopentyl or cyclohexyl, such as cyclopropyl, cyclobutyl or cyclohexyl; and preferably, each L₂¹⁻³⁻¹ is independently halogen or hydroxyl.

In a preferred embodiment, in L₂¹⁻³, in the 3- to 11-membered heterocycloalkyl that is unsubstituted or substituted by one or more L₂¹⁻³⁻², the 3- to 11-membered heterocycloalkyl can be 4 to 9-membered heterocycloalkyl, the heteroatoms of the 4 to 9-membered heterocycloalkyl are preferably selected from one or more of N, S and O, and the number of heteroatoms is 1 or 2; the 3- to 11-membered heterocycloalkyl may be a tetrahydropyrrole ring, a piperidine ring, a tetrahydrofuran ring or a tetrahydrothiophene ring, such as a piperidine ring.

In a preferred embodiment, in L₂¹⁻³⁻¹ and L₂¹⁻³⁻², the C₁-C₆ alkoxy can be C₁-C₄ alkoxy, such as methoxy, ethoxy, n-propoxy, isopropoxy, n-butoxy, isobutoxy, primary butoxy, sec-butoxy or tert-butoxy; and can also be methoxy, ethoxy, or isopropoxy.

In a preferred embodiment, when R^{c} is C₁-C₆ alkyl that is unsubstituted or substituted by one or more R^{c-1}, the C₁-C₆ alkyl can be C₁-C₄ alkyl, such as methyl, ethyl, n-propyl, isopropyl, n-butyl, isobutyl, primary butyl, sec-butyl or tert-butyl; and can also be methyl, ethyl or isopropyl.

In a preferred embodiment, in LLM, p is 0 or 1.

In a preferred embodiment, in L₂, m is 1 or 2.

In a preferred embodiment, in L₂, q is 1 or 2.

In a preferred embodiment, in L₃, m is 1 or 2.

In a preferred embodiment, in L₃, q is 1 or 2.

In the present disclosure, in R^{c}, R¹⁻¹⁻¹, R¹⁻¹⁻³, R¹⁻¹⁻⁴, R¹⁻¹⁻⁵, R³⁻¹⁻¹, R³⁻¹⁻⁴, R³⁻¹⁻⁵, R¹⁻¹⁻¹⁻¹, R¹⁻¹⁻¹⁻², R¹⁻¹⁻¹⁻³, R^{a-1}, R^{a-2}, R^{a-3}, R^{a-4}, R^{a-5}, R^{c-1}, R^{c-2}, R^{c-3}, R^{c-4} and R^{c-5}, the 6- to 10-membered aryl may independently be phenyl or naphthyl.

In the present disclosure, in R¹⁻¹⁻¹, R¹⁻¹⁻³, R¹⁻¹⁻⁴, R¹⁻¹⁻⁵, R³⁻¹⁻¹, R³⁻¹⁻⁴, R³⁻¹⁻⁵, R¹⁻¹⁻¹⁻¹, R¹⁻¹⁻¹⁻², R¹⁻¹⁻¹⁻³, R^{a-1}, R^{a-2}, R^{a-3}, R^{a-4}, R^{a-5}, R^{c-1}, R^{c-2}, R^{c-3}, R^{c-4} and R^{c-5}, the C₁-C₆ alkoxy can independently be C₁-C₄ alkoxy, such as methoxy, ethoxy, n-propoxy, isopropoxy, n-butoxy, isobutoxy, sec-butoxy or tert-butoxy; also such as methoxy.

In the present disclosure, in R¹⁻¹⁻¹, R¹⁻¹⁻³, R¹⁻¹⁻⁴, R¹⁻¹⁻⁵, R³⁻¹⁻¹, R³⁻¹⁻⁴, R³⁻¹⁻⁵, R¹⁻¹⁻¹⁻¹, R¹⁻¹⁻¹⁻², R¹⁻¹⁻¹⁻³, R^{a-1}, R^{a-2}, R^{a-3}, R^{a-4}, R^{a-5}, R^{c-1}, R^{c-2}, R^{c-3}, R^{c-4} and R^{c-5}, the halogen can independently be fluorine, chlorine, bromine or iodine, such as fluorine or chlorine.

In the present disclosure, in R^{c}, R¹⁻¹⁻¹, R¹⁻¹⁻³, R¹⁻¹⁻⁴, R¹⁻¹⁻⁵, R³⁻¹⁻¹, R³⁻¹⁻⁴, R³⁻¹⁻⁵, R¹⁻¹⁻¹⁻¹, R¹⁻¹⁻¹⁻², R¹⁻¹⁻¹⁻³, R^{a-1}, R^{a-2}, R^{a-3}, R^{a-4}, R^{a-5}, R^{c-1}, R^{c-2}, R^{c-3}, R^{c-4} and R^{c-5}, the C₁-C₆ alkyl can independently be C₁-C₄ alkyl, such as methyl, ethyl, n-propyl, isopropyl, n-butyl, isobutyl, primary butyl, sec-butyl or tert-butyl; and can also be methyl, ethyl or isopropyl.

In the present disclosure, in R^{c}, R¹⁻¹⁻¹, R¹⁻¹⁻³, R¹⁻¹⁻⁴, R¹⁻¹⁻⁵, R³⁻¹⁻¹, R³⁻¹⁻⁴, R³⁻¹⁻⁵, R¹⁻¹⁻¹⁻¹, R¹⁻¹⁻¹⁻², R¹⁻¹⁻¹⁻³, R^{a-1}, R^{a-2}, R^{a-3}, R^{a-4}, R^{a-5}, R^{c-1}, R^{c-2}, R^{c-3}, R^{c-4} and R^{c-5}, the 3- to 11-membered heterocycloalkyl can be independently a 6-membered heterocycloalkyl, a 5-membered heterocycloalkyl, an 8-membered heterocycloalkyl or a 7-membered heterocycloalkyl. The heteroatoms of the 3- to 11-membered heterocycloalkyl are selected from one or two of N and O, and the number of heteroatoms is 1 or 2; the 6-membered heterocycloalkyl is preferably piperazinyl, morpholinyl or piperidinyl, such as

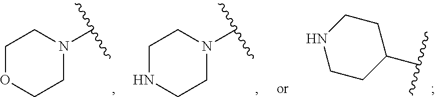 [SMILES: *N1CCOCC1 ; *N1CCNCC1 ; *C1CCNCC1]

the 5-membered heterocycloalkyl is preferably tetrahydropyrrolyl, such as

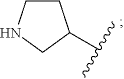 [SMILES: *C1CCNC1]

the 8-membered heterocycloalkyl is preferably azabicyclo[2.2.2]octyl or azabicyclo[3.2.1]octyl, such as

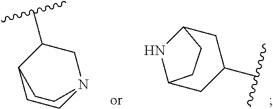 [SMILES: *C1CN2CCC1CC2 ; *C1CC2CCC(C1)N2]

the 7-membered heterocycloalkyl is preferably azaspiro[3.3]heptyl or azabicyclo[2.2.1]heptyl, such as

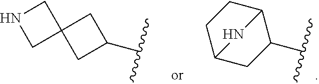 [SMILES: *C1CC2(CNC2)C1 ; *C1CC2CCC1N2]

In the present disclosure, in R^{c}, R¹⁻¹⁻¹, R¹⁻¹⁻³, R¹⁻¹⁻⁴, R¹⁻¹⁻⁵, R³⁻¹⁻¹, R³⁻¹⁻⁴, R³⁻¹⁻⁵, R¹⁻¹⁻¹⁻¹, R¹⁻¹⁻¹⁻², R¹⁻¹⁻¹⁻³, R^{a-1}, R^{a-2}, R^{a-3}, R^{a-4}, R^{a-5}, R^{c-1}, R^{c-2}, R^{c-3}, R^{c-4} and R^{c-5}, the 3- to 10-membered cycloalkyl can be independently a 3- to 6-membered cycloalkyl, such as cyclopropyl, cyclobutyl, cyclopentyl or cyclohexyl.

In the present disclosure, in R^{c}, R¹⁻¹⁻¹, R¹⁻¹⁻³, R¹⁻¹⁻⁴, R¹⁻¹⁻⁵, R³⁻¹⁻¹, R³⁻¹⁻⁴, R³⁻¹⁻⁵, R¹⁻¹⁻¹⁻¹, R¹⁻¹⁻¹⁻², R¹⁻¹⁻¹⁻³, R^{a-1}, R^{a-2}, R^{a-3}, R^{a-4}, R^{a-5}, R^{c-1}, R^{c-2}, R^{c-3}, R^{c-4} and R^{c-5}, the 5- to 10-membered heteroaryl can be independently a 5-membered heteroaryl, a 6-membered heteroaryl, a 5-membered-fused 5-membered heteroaryl; the 6-membered heteroaryl is preferably a pyridyl, a pyridazinyl, a pyrazinyl, a pyrimidinyl or a triazinyl; and the 5-membered heteroaryl ring is preferably pyrazolyl, oxazolyl, thiazolyl or imidazolyl, such as

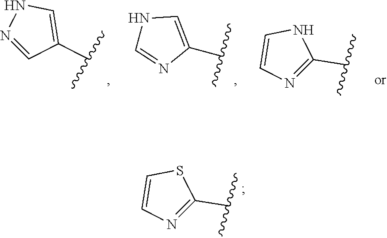

the 5- to 10-membered heteroaryl is preferably pyrazolyl, thiazolyl, imidazolyl, tetrahydropyrrolothiazolyl or tetrahydropyrrolopyrazolyl, such as

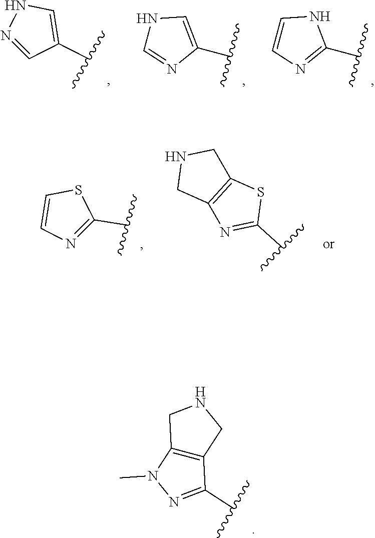

In a preferred embodiment, R¹ is a 5- to 10-membered heteroaryl that is unsubstituted or substituted by one or more R¹⁻¹ or a 6- to 10-membered aryl that is unsubstituted or substituted by one or more R¹⁻²; the heteroatoms of the 5- to 10-membered heteroaryl are selected from one or two of N, S and O, and the number of heteroatoms is 1 or 2. R¹ is preferably a 5- to 6-membered heteroaryl that is unsubstituted or substituted by one or more R¹⁻¹ or a 6- to 10-membered aryl that is unsubstituted or substituted by one or more R¹⁻²; the heteroatoms of the 5- to 6-membered heteroaryl are selected from one or two of N, S and O, and the number of heteroatoms is 1 or 2.

In a preferred embodiment, R¹ is a 5- to 10-membered heteroaryl that is unsubstituted or substituted by one or more R¹⁻¹ or a 6- to 10-membered aryl that is unsubstituted or substituted by one or more R¹⁻²; the heteroatoms of the 5- to 10-membered heteroaryl are selected from one or two of N, S and O, and the number of heteroatoms is 1, 2 or 3. R¹ is preferably a 5- to 9-membered heteroaryl that is unsubstituted or substituted by one or more R¹⁻¹ or a 6- to 10-membered aryl that is unsubstituted or substituted by one or more R¹⁻²; the heteroatoms of the 5- to 9-membered heteroaryl are selected from one or two of N, S and O, and the number of heteroatoms is 1, 2 or 3. R¹ is preferably a 5- to 9-membered heteroaryl that is unsubstituted or substituted by one or more R¹⁻¹, the 5- to 9-membered heteroaryl is pyridyl, oxazolyl, pyrazolyl or

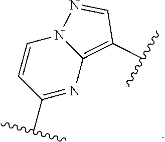 [SMILES: *c1ccn2ncc(*)c2n1]

R¹ is preferably pyridyl substituted by one or more R¹⁻¹, or

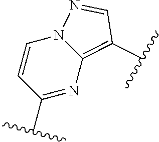 [SMILES: *c1ccn2ncc(*)c2n1]

substituted by one or more R^{1-1.}

In a preferred embodiment, each R¹⁻¹ and R¹⁻² are independently halogen, hydroxyl,

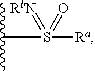

—SO₂—R^{a}, —SO—R^{a}, C₁-C₆ alkyl that is unsubstituted or substituted by one or more R¹⁻¹⁻⁴, or a 5- to 10-membered heteroaryl that is unsubstituted or substituted by one or more R¹⁻¹⁻⁷; the heteroatoms of the 5- to 10-membered heteroaryl are selected from one or more of N, S and O, and the number of heteroatoms is 1 or 2. Each R¹⁻¹ and R¹⁻² are independently halogen, a 5- to 6-membered heteroaryl that is unsubstituted or substituted by one or more R¹⁻¹⁻⁷ or C₁-C₆ alkyl that is unsubstituted or substituted by one or more R¹⁻¹⁻⁴, the heteroatoms of the 5- to 6-membered heteroaryl are selected from one or more of N, S and 0, and the number of heteroatoms is 1 or 2.

In a preferred embodiment, each R¹⁻¹ is C₁-C₆ alkyl that is unsubstituted or substituted by one or more R¹⁻¹⁻⁴, a 3- to 11-membered heterocycloalkyl that is unsubstituted or substituted by one or more R¹⁻¹⁻¹ or a 5- to 10-membered heteroaryl that is unsubstituted or substituted by one or more R¹⁻¹⁻⁷; the heteroatoms of the 5- to 10-membered heteroaryl are selected from one or more of N, S and O, and the number of heteroatoms is 1 or 2; the heteroatoms of the 3- to 11-membered heterocycloalkyl are selected from one or more of N, S and O, and the number of heteroatoms is 1, 2 or 3.

In a preferred embodiment, each R¹⁻¹⁻⁴ is independently halogen.

In a preferred embodiment, each R¹⁻¹⁻⁴ is independently halogen or C₁-C₆ alkyl that is unsubstituted or substituted by one or more halogens.

In a preferred embodiment, each R¹⁻¹⁻⁷ is independently halogen, hydroxy, C₁-C₆ alkoxy that is unsubstituted or substituted by one or more halogens, cyano, nitro, C₁-C₆ alkyl that is unsubstituted or substituted by one or more halogens, a 6- to 10-membered aryl that is unsubstituted or substituted by one or more R¹⁻¹⁻¹⁻², or a 5- to 10-membered heteroaryl that is unsubstituted or substituted by one or more R¹⁻¹⁻¹⁻³; the heteroatoms of the 3- to 11-membered heteroaryl are selected from one or more of N, S and O, and the number of heteroatoms is 1, 2 or 3; the heteroatoms of the 5- to 10-membered heteroaryl are selected from one or more of N, S and O, and the number of heteroatoms is 1, 2 or 3. Preferably, each R¹⁻¹⁻⁷ is independently C₁-C₆ alkyl that is unsubstituted or substituted by one or more halogens.

In a preferred embodiment, preferably, each R¹⁻¹⁻⁷ is independently halogen or C₁-C₆ alkyl that is unsubstituted or substituted by one or more halogens.

In a preferred embodiment, each R¹⁻¹⁻¹ is independently halogen or C₁-C₆ alkyl that is unsubstituted or substituted by one or more halogens. Preferably, each R¹⁻¹⁻¹ is independently C₁-C₆ alkyl that is unsubstituted or substituted by one or more halogens.

In a preferred embodiment, each R¹⁻¹⁻¹⁻¹, R¹⁻¹⁻¹⁻² and R¹⁻¹⁻¹⁻³ are independently halogen or C₁-C₆ alkyl.

In a preferred embodiment, R² is hydrogen.

In a preferred embodiment, R³ is hydrogen, hydroxy, halogen, cyano, a 3- to 10-membered heterocycloalkyl that is unsubstituted or substituted by one or more R³⁻¹, a 3- to 8-membered cycloalkyl that is unsubstituted or substituted by one or more R³⁻², C₁-C₆ alkyl that is unsubstituted or substituted by one or more R³⁻⁴, C₁-C₆ alkoxy that is unsubstituted or substituted by one or more R³⁻⁷ or hydroxyl substituted by R³⁻⁸; the heteroatoms of the 3- to 10-membered heterocycloalkyl are selected from one or two of N and O, and the number of heteroatoms is 1, 2 or 3. R³ is preferably a 3- to 6-membered heterocycloalkyl that is unsubstituted or substituted by one or more R³⁻¹, C₃-C₆ cycloalkyl that is unsubstituted or substituted by one or more R³⁻², C₁-C₆ alkoxy that is unsubstituted or substituted by one or more R³⁻⁷, C₁-C₆ alkyl that is unsubstituted or substituted by one or more R³⁻⁴, or hydroxyl substituted by R³⁻⁸; the heteroatoms of the 3- to 6-membered heterocycloalkyl are selected from one or two of N and O, and the number of heteroatoms is 1 or 2.

In a preferred embodiment, R³ is hydrogen, hydroxy, halogen, cyano,

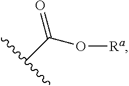

a 3- to 10-membered heterocycloalkyl that is unsubstituted or substituted by one or more R³⁻¹, a 3- to 8-membered cycloalkyl that is unsubstituted or substituted by one or more R³⁻², C₁-C₆ alkyl that is unsubstituted or substituted by one or more R³⁻⁴, C₁-C₆ alkoxy that is unsubstituted or substituted by one or more R³⁻⁷ or hydroxyl substituted by R³⁻⁸; the heteroatoms of the 3- to 10-membered heterocycloalkyl are selected from one or two of N and O, and the number of heteroatoms is 1, 2 or 3. R³ is preferably halogen,

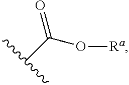

a 3- to 6-membered heterocycloalkyl that is unsubstituted or substituted by one or more R³⁻¹, C₃-C₆ cycloalkyl that is unsubstituted or substituted by one or more R³⁻², C₁-C₆ alkoxy that is unsubstituted or substituted by one or more R³⁻⁷, C₁-C₆ alkyl that is unsubstituted or substituted by one or more R³⁻⁴, or hydroxyl substituted by R³⁻⁸; the heteroatoms of the 3- to 6-membered heterocycloalkyl are selected from one or two of N and O, and the number of heteroatoms is 1 or 2. R³ is preferably C₁-C₆ alkyl that is unsubstituted or substituted by one or more R³⁻⁴, or C₁-C₆ alkoxy that is unsubstituted or substituted by one or more R^{3-7.}

R³ is preferably C₁-C₆ alkyl that is unsubstituted or substituted by one or more R³⁻⁴, C₁-C₆ alkoxy that is unsubstituted or substituted by one or more R³⁻⁷ or tetrahydropyrrolyl that is unsubstituted or substituted by one or more R^{3-1.}

In a preferred embodiment, each R³⁻¹, R³⁻², R³⁻⁴ and R³⁻⁷ are independently oxo, hydroxyl, halogen, a 3- to 6-membered cycloalkyl, a 3- to 6-membered heterocycloalkyl or C₁-C₆ alkoxy that is unsubstituted or substituted by one or more halogens; the heteroatoms of the 3- to 6-membered heterocycloalkyl are selected from one or two of N, S and O, and the number of heteroatoms is 1 or 2. Each R¹is independently preferably hydroxyl or halogen. Each R³⁻² is independently preferably hydroxyl. Each R³⁻⁴ is independently preferably hydroxyl; and each R³⁻⁷ is independently preferably halogen.

In a preferred embodiment, each R³⁻¹, R³⁻², R³⁻⁴ and R³⁻⁷ are independently deuterium, oxo, hydroxyl, halogen, a 3- to 6-membered cycloalkyl, a 3- to 6-membered heterocycloalkyl or C₁-C₆ alkoxy that is unsubstituted or substituted by one or more halogens; the heteroatoms of the 3- to 6-membered heterocycloalkyl are selected from one or two of N, S and O, and the number of heteroatoms is 1 or 2. Each R³⁻¹ is independently preferably hydroxyl or halogen. Each R³⁻² is independently preferably hydroxyl. Each R³⁻⁴ is independently preferably deuterium, halogen or hydroxyl; and each R³⁻⁷ is independently preferably deuterium or halogen.

In a preferred embodiment, each R³⁻¹ is independently deuterium, hydroxyl or halogen.

In a preferred embodiment, each R³⁻⁴ is independently deuterium, halogen or hydroxyl.

In a preferred embodiment, each R³⁻⁷ is independently deuterium or hydroxyl.

In a preferred embodiment, when R³ is C₁-C₆ alkyl substituted by multiple R³⁻⁴, at least one R³⁻⁴ is hydroxyl, deuterium and hydroxyl, or halogen and hydroxyl.

In a preferred embodiment, R³⁻⁸ is a 3- to 6-membered cycloalkyl or a 3- to 6-membered heterocycloalkyl; the heteroatoms of the 3- to 6-membered heterocycloalkyl are selected from one or two of N, S and O, and the number of heteroatoms is 1, 2 or 3. R³⁻⁸is preferably a 3- to 6-membered cycloalkyl or a 3- to 6-membered heterocycloalkyl; the heteroatoms of the 3- to 6-membered heterocycloalkyl are selected from one or two of N and O, and the number of heteroatoms is 1.

In a preferred embodiment, each R^{a} and each R^{b} are independently H or C₁-C₆ alkyl that is unsubstituted or substituted by one or more R^{a-1}. Preferably, each R^{a} and each R^{b} are independently H.

In a preferred embodiment, L₀ is a 5- to 12-membered cycloalkylene group that is unsubstituted or substituted by one or more L₀⁻², or a 5- to 12-membered heterocycloalkylene group that is unsubstituted or substituted by one or more L₀⁻³; the 5- to 12-membered cycloalkylene group is a spiro ring, a fused ring or a bridged ring; and the 5- to 12-membered heterocycloalkylene group is a monocyclic, a spiro ring, a fused ring or a bridged ring; the heteroatoms of the 5- to 12-membered heterocycloalkylene group are selected from one or more of N, S and O, and the number of heteroatoms is 1, 2 or 3; each L₀⁻² is independently halogen, hydroxyl, or C₁-C₆ alkyl that is unsubstituted or substituted by one or more halogens; and each L₀⁻³ is independently halogen, hydroxyl, or C₁-C₆ alkyl that is unsubstituted or substituted by one or more halogens.

In a preferred embodiment, L₀ is a 5- to 12-membered cycloalkylene group that is unsubstituted or substituted by one or more L₀⁻², or a 5- to 12-membered heterocycloalkylene group that is unsubstituted or substituted by one or more L₀⁻³; the 5- to 12-membered cycloalkylene group is a spiro ring, a fused ring or a bridged ring; and the 5- to 12-membered heterocycloalkylene group is a monocyclic, a spiro ring, a fused ring or a bridged ring; the heteroatoms of the 5- to 12-membered heterocycloalkylene group are selected from one or more of N, S and O, and the number of heteroatoms is 1, 2 or 3; each L₀⁻² is independently deuterium, halogen, hydroxyl, or C₁-C₆ alkyl that is unsubstituted or substituted by one or more halogens; and each L₀⁻³ is independently deuterium, halogen, hydroxyl, or C₁-C₆ alkyl that is unsubstituted or substituted by one or more halogens.

In a preferred embodiment, L₀ is a 7- to 11-membered cycloalkylene group that is unsubstituted or substituted by one or more L₀⁻², or a 7- to 11-membered heterocycloalkylene group that is unsubstituted or substituted by one or more L₀⁻³; the 7- to 11-membered cycloalkylene group is a spiro ring; and the 7- to 11-membered heterocycloalkylene group is a spiro ring; the heteroatom of the 7- to 11-membered heterocycloalkylene group is N, and the number of heteroatoms is 1 or 2; each L₀⁻² is independently halogen, hydroxyl, or C₁-C₆ alkyl that is unsubstituted or substituted by one or more halogens; and each L₀⁻³ is independently halogen, hydroxyl, or C₁-C₆ alkyl that is unsubstituted or substituted by one or more halogens.

In a preferred embodiment, L₀ is a 7- to 11-membered cycloalkylene group that is unsubstituted or substituted by one or more L₀⁻², or a 7- to 11-membered heterocycloalkylene group that is unsubstituted or substituted by one or more L₀⁻³; the 7- to 11-membered cycloalkylene group is a spiro ring; and the 7- to 11-membered heterocycloalkylene group is a spiro ring or a fused ring; the heteroatom of the 7- to 11-membered heterocycloalkylene group is N, and the number of heteroatoms is 1 or 2; each L₀⁻² is independently deuterium, halogen, hydroxyl, or C₁-C₆ alkyl that is unsubstituted or substituted by one or more halogens; and each L₀⁻³ is independently deuterium, halogen, hydroxyl, or C₁-C₆ alkyl that is unsubstituted or substituted by one or more halogens. In a preferred embodiment, L₀ is a 7- to 11-membered cycloalkylene group or a 7- to 11-membered heterocycloalkylene group, the 7- to 11-membered cycloalkylene group is a spiro ring, the 1- to 11-membered heterocycloalkylene group is a spiro ring, the heteroatom of the 7- to 11-membered heterocycloalkylene group is N, and the number of heteroatoms is 1.

In a preferred embodiment, L₀ is a 7- to 11-membered cycloalkylene group or a 7- to 11-membered heterocycloalkylene group, the 7- to 11-membered cycloalkylene group is a spiro ring, the 7- to 11-membered heterocycloalkylene group is a spiro ring or a fused ring, the heteroatom of the 7- to 11-membered heterocycloalkylene group is N, and the number of heteroatoms is 1 or 2.

In a preferred embodiment, L₀ is a 8- to 11-membered cycloalkylene group or a 8- to 11-membered heterocycloalkylene group, the 8- to 11-membered cycloalkylene group is a spiro ring, the 8- to 11-membered heterocycloalkylene group is a spiro ring or a fused ring, the heteroatom of the 8- to 11-membered heterocycloalkylene group is N, and the number of heteroatoms is 1. Preferably, the 8- to 11-membered cycloalkylene group can be

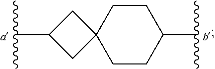 [SMILES: *C1CCC2(CC1)CC(*)C2]

and the 8- to 11-membered heterocycloalkylene group can be

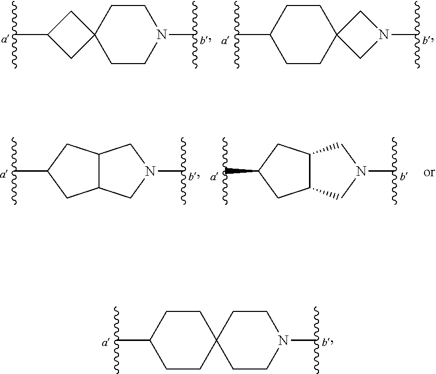

wherein the a′ end is connected to ring Cy, and the b′ end is connected to L².

In a preferred embodiment, L₀ is a 9- to 11-membered heterospirylene group; the heteroatom of the 9- to 11-membered heterospirylene group is N, and the number of heteroatoms is 1. Preferably, the 9- to 11-membered heterospirylene group is

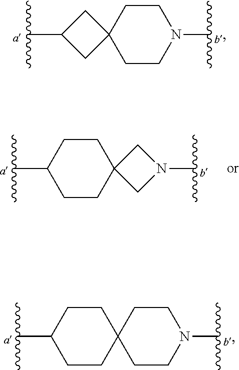 [SMILES: *C1CC2(CCN(*)CC2)C1 ; *C1CCC2(CC1)CN(*)C2 ; *C1CCC2(CC1)CCN(*)CC2]

wherein the a′ end is connected to ring Cy, and the b′ end is connected to L².

In a preferred embodiment, L₂⁻¹, L₂⁻², L₂⁻³ and L₂⁻⁴ are independently absent,

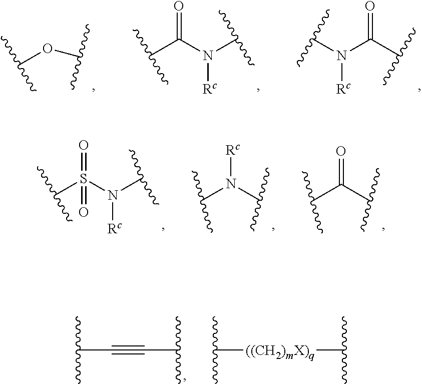

that is unsubstituted or substituted by one or more L₂¹⁻¹,

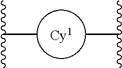

that is unsubstituted or substituted by one or more L₂¹⁻², or

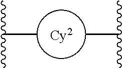

that is unsubstituted or substituted by one or more L₂¹⁻³, m is an integer from 1 to 4, q is an integer from 1 to 6, and X is absent or O; Ring Cy¹ is a 4- to 11-membered heterocycle or 4- to 11-membered cycloalkyl; ring Cy² is a 5- to 10-membered heteroaromatic ring or a 6- to 10-membered aromatic ring; the heteroatoms of the 4- to 11-membered heterocycle are selected from one or more of N, S and O, and the number of heteroatoms is 1, 2 or 3; the heteroatoms of the 5- to 10-membered heteroaromatic ring are selected from one or more of N, S and O, and the number of heteroatoms is 1, 2 or 3.

In a preferred embodiment, L₂⁻¹, L₂⁻², L₂⁻³ and L₂⁻⁴ are independently absent,

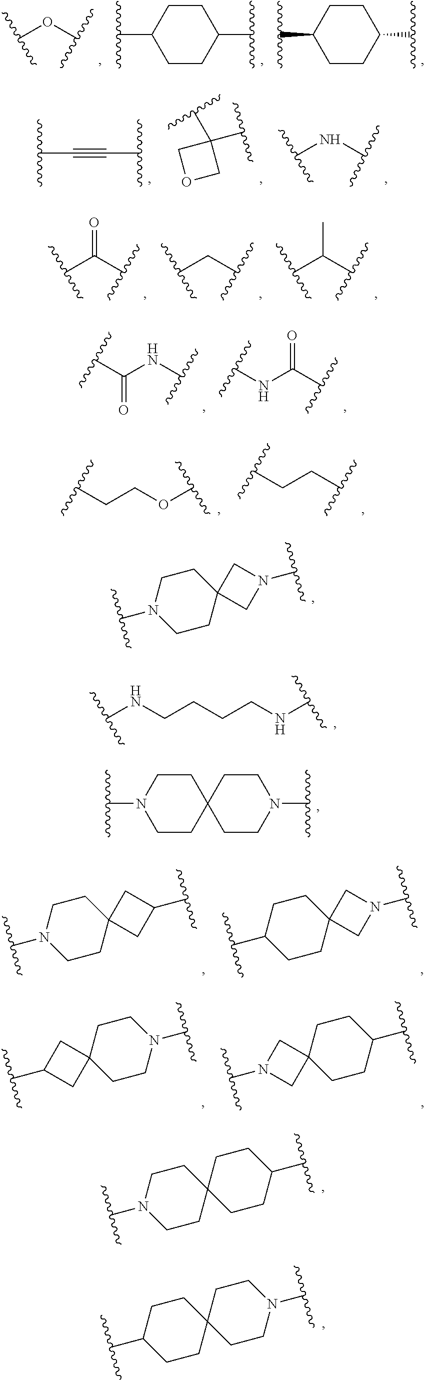

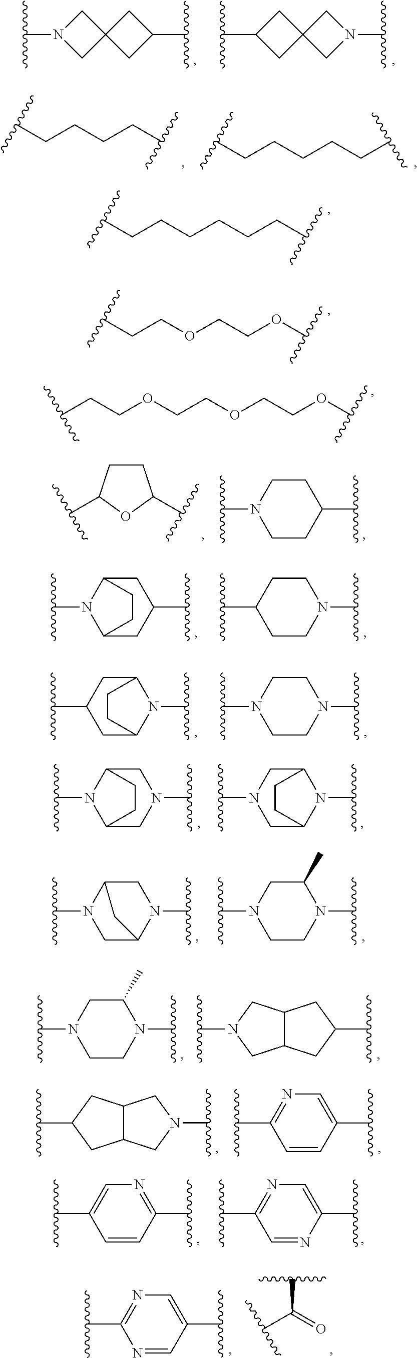

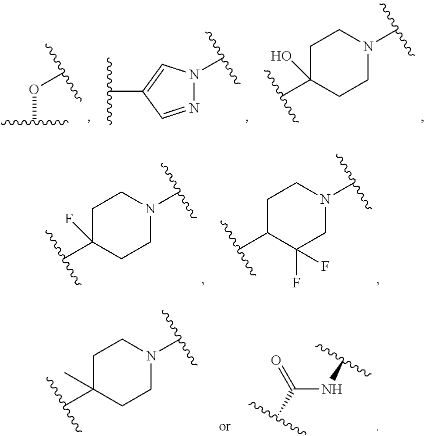

In a preferred embodiment, preferably, L₂⁻¹, L₂⁻², L₂⁻³ and L₂⁻⁴ are independently absent,

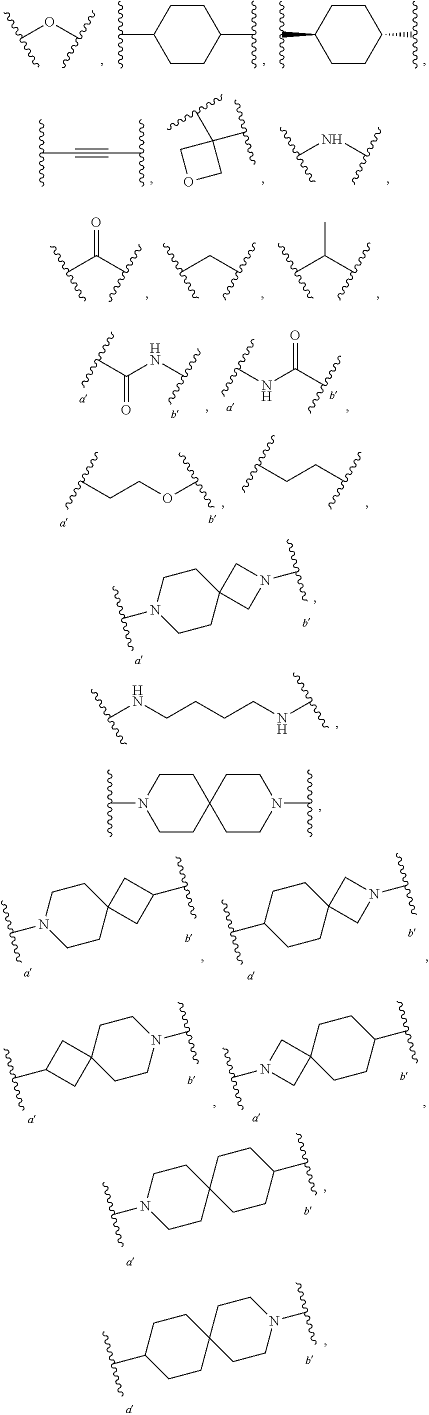

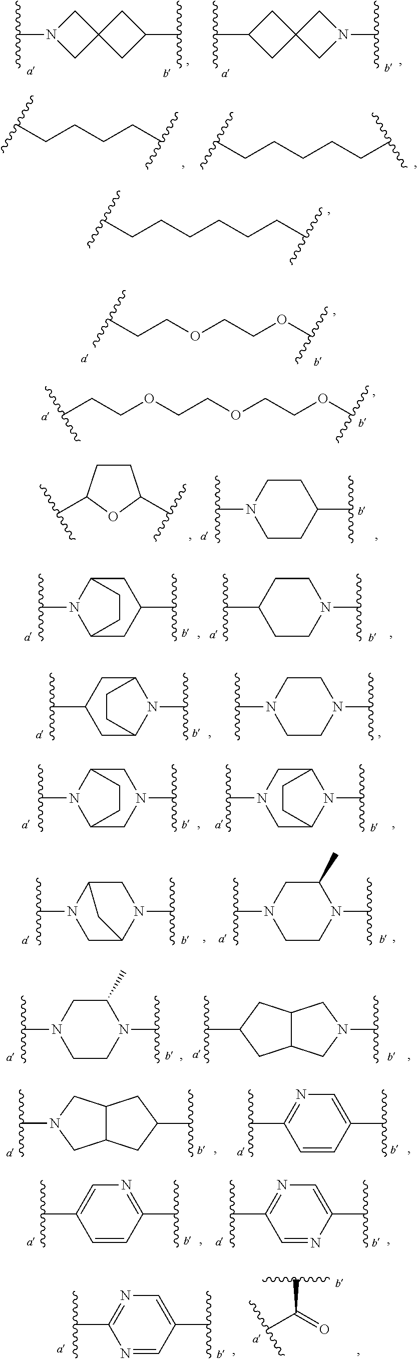

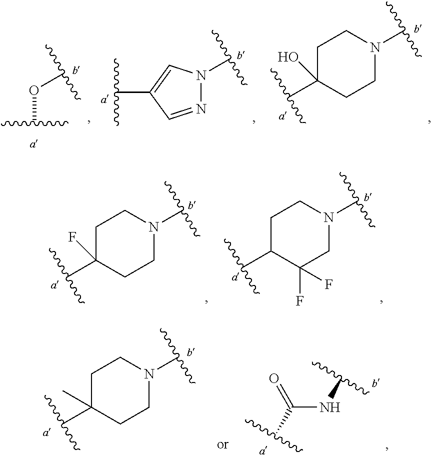

wherein the a′ end towards L₀ and the b′ end towards LLM.

In a preferred embodiment, L₂⁻¹, L₂⁻², L₂⁻³ and L₂⁻⁴ are independently absent,

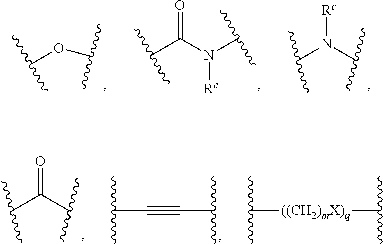

that is unsubstituted or
substituted by one or more L₂¹⁻¹, or

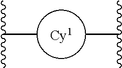

that is unsubstituted or substituted by one or more L₂¹⁻², m is 1 or 2, q is 1or 2, and X is absent or O; Ring Cy¹ is a 4- to 11-membered heterocycle or 4- to 6-membered cycloalkyl; the heteroatoms of the 4- to 11-membered heterocycle are N and/or O, and the number of heteroatoms is 1 or 2; the 4- to 11-membered heteroaromatic ring is a monocyclic ring, a bridged ring or a spiro ring.

In a preferred embodiment, L₂⁻¹, L₂⁻², L₂⁻³ and L₂⁻⁴ are independently absent,

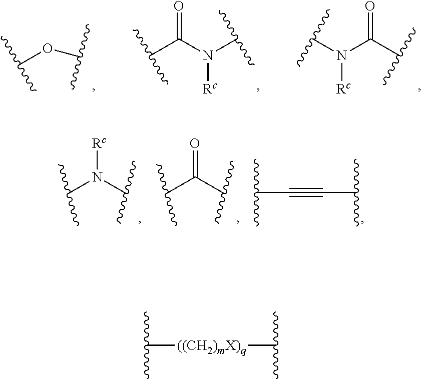

that is unsubstituted or substituted by one or more L₂¹⁻¹,

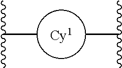

that is unsubstituted or substituted by one or more L₂¹⁻², or

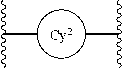

that is unsubstituted or substituted by one or more L₂¹⁻³, m is 1 or 2, q is 1 or 2, and X is absent or O; Ring Cy¹ is a 4- to 11-membered heterocycle or 4- to 6-membered cycloalkyl; the heteroatoms of the 4- to 11-membered heterocycle are N and/or O, and the number of heteroatoms is 1 or 2; the 4- to 11-membered heterocycle is a monocyclic ring, a bridged ring or a spiro ring; and the 4- to 6-membered cycloalkyl is a monocyclic ring. Ring Cy² is a 5- to 6-membered heteroaromatic ring; the heteroatoms of the 5- to 6-membered heteroaromatic ring are N and/or O, and the number of heteroatoms is 1 or 2. In a preferred embodiment, when the 4- to 11-membered heterocycle is a monocyclic ring, the 4- to 11-membered heterocycle is a 3- to 6-membered heterocycle, the heteroatom of the 4- to 11-membered heterocycle is N or O, and the number of heteroatoms is 1 or 2.

In a preferred embodiment, when the 4- to 11-membered heterocycle is a spiro ring, the 4- to 11-membered heterocycle is a 4,6-azaspirocycle, a 6,6-azaspirocycle or a 4, 4-azaspirocycle.

In a preferred embodiment, L₂⁻¹, L₂⁻², L₂⁻³ and L₂⁻⁴ are independently absent,

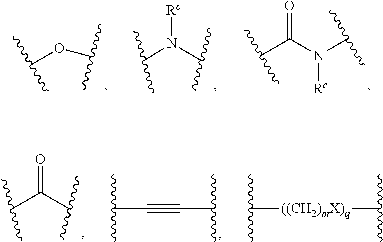

that is unsubstituted or substituted by one or more L₂¹-1, or

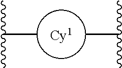

That is unsubstituted or substituted by one or more L₂¹⁻², m is 1 or 2, q is 1, and X is absent or O; Ring Cy¹ is a 4- to 10-membered heterocycle or 4- to 6-membered cycloalkyl; the heteroatoms of the 4- to 10-membered heterocycle are N, and the number of heteroatoms is 1 or 2; the 4 to 10-membered heterocycle is a 6-membered monocyclic ring or 4,6-azaspirocycle; and R^{c} is H.

In a preferred embodiment, L₂⁻¹, L₂⁻², L₂⁻³ and L₂⁻⁴ are independently absent,

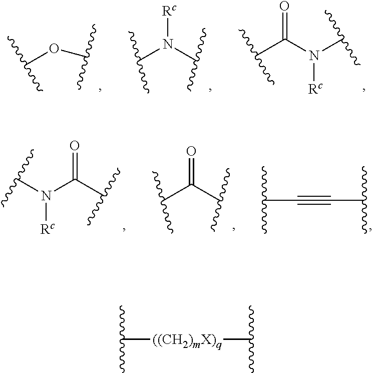

that is unsubstituted or substituted by one or more L₂¹⁻¹,

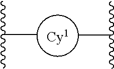

that is unsubstituted or substituted by one or more L₂¹⁻², or

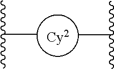

that is unsubstituted or substituted by one or more L₂¹⁻³, m is 1 or 2, q is 1, and X is absent or O; Ring Cy¹ is a 4- to 10-membered heterocycle or 4- to 6-membered cycloalkyl; the heteroatoms of the 4- to 10-membered heterocycle are N, and the number of heteroatoms is 1 or 2; the 4 to 10-membered heterocycle is a 6-membered monocyclic ring, 4,4-azaspirocycle or 4,6-azaspirocycle; R^{c} is H, ring Cy² is a 5-membered heteroaromatic ring; the heteroatoms of the 5-membered heteroaromatic ring are N, and the number of heteroatoms is 2, the 5-membered heteroaromatic ring is preferably a pyrazole ring.

In a preferred embodiment, L₂⁻¹, L₂⁻², L₂⁻³ and L₂⁻⁴ are independently absent,

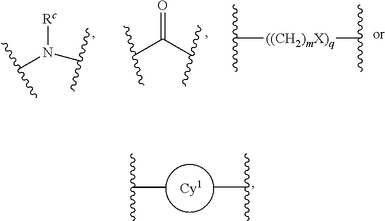

m is 1 or 2, q is 1 and X is absent; Ring Cy¹ is a 4- to 6-membered heterocycle or 4- to 6-membered cycloalkyl; the heteroatoms of the 4- to 10-membered heterocycle are N, and the number of heteroatoms is 1 or 2; the 4- to 6-membered cycloalkyl is a 6-membered monocyclic; R^{c} is H.

In a preferred embodiment, L₂⁻¹, L₂⁻², L₂⁻³ and L₂⁻⁴ are independently absent,

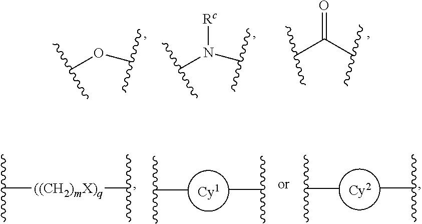

m is 1 or 2, q is 1 and X is absent; Ring Cy¹ is a 4- to 10-membered heterocycle or 4- to 6-membered cycloalkyl;

the heteroatoms of the 4- to 10-membered heterocycle are N, and the number of heteroatoms is 1 or 2; the 4- to 6-membered cycloalkyl is a 6-membered monocyclic; R^{c} is H, Ring Cy² is 5-membered heteroaromatic ring, the heteroatoms of the 5-membered heteroaromatic ring are N, and the number of heteroatoms is 2, such as a pyrazole ring.

In a preferred embodiment, L₂ is -L₂⁻¹-L₂⁻²-L₂⁻³-L₂⁻⁴-; L₂⁻¹ and L₂⁻² are absent, L₂⁻³is

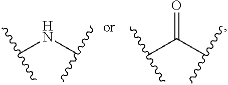

L₂⁻⁴ is

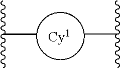

that is unsubstituted or substituted by one or more L₂¹⁻²; and ring Cy¹ is piperidinyl or piperazinyl. Preferably, L₂ is -L₂⁻¹-L₂⁻²-L₂⁻³-L₂⁻⁴-; L₂⁻¹ and L₂⁻² are absent, L₂⁻³ is

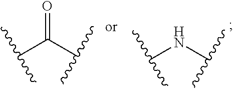

L₂⁻⁴ is unsubstituted ring Cy¹, and the ring Cy¹ is piperidinyl or piperazinyl.

In a preferred embodiment, L₂ is L₂⁻¹-L₂⁻²-L₂⁻³-L₂⁻⁴-; L₂⁻¹ is absent, L₂⁻², L₂⁻³ and L₂⁻⁴ are independently

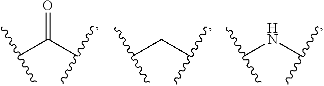

or ring Cy¹ that is unsubstituted or substituted by one or more L₂¹⁻², and the ring Cy¹ is cyclohexyl, piperidinyl or piperazinyl.

In a preferred embodiment, L₂ is -L₂⁻¹-L₂⁻²-L₂⁻³-L₂⁻⁴-; L₂⁻¹, L₂⁻², L₂⁻³ and L₂⁻⁴ are independently

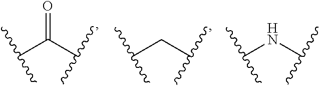

or ring Cy¹ that is unsubstituted or substituted by one or more L₂¹⁻², and the ring Cy¹ is cyclohexyl, piperidinyl or piperazinyl.

In a preferred embodiment, each L₂¹⁻² is independently halogen or hydroxyl.

In a preferred embodiment, L₂ is -L₂⁻¹-L₂⁻²-L₂⁻³-L₂⁻⁴- or

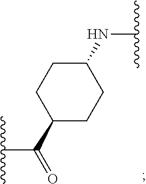 [SMILES: *N[C@H]1CC[C@H](C(*)=O)CC1]

L₂⁻¹ is

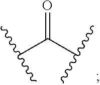 [SMILES: *C(*)=O]

L₂⁻², L₂⁻³ and L₂⁻⁴ are independently absent,

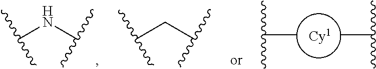

that is unsubstituted or substituted by one or more L₂¹⁻²; the ring Cy¹ is a 4- to 6-membered heterocycle; the heteroatom of the 4- to 6-membered heterocycle is N, and the number of heteroatoms is 1; and the 4- to 6-membered heterocycle is a monocyclic ring.

In a preferred embodiment, in L₂, R^{c} is H or a C₁-C₆ alkyl that is unsubstituted or substituted by one or more halogens, and is more preferably H.

In a preferred embodiment, ring Cy³ is a 5- to 6-membered heterocycle that is unsubstituted or substituted by one or more Cy³⁻¹; the heteroatom of the 5- to 6-membered heterocycle is N, S or O, and the number is 1. Ring Cy³ is preferably a 5- to 6-membered heterocycle that is unsubstituted or substituted by multiple Cy³⁻¹, the heteroatom of the 5- to 6-membered heterocycle is N, and the number is 1.

In a preferred embodiment, each Cy³⁻¹ is independently oxo.

In a preferred embodiment, Ring Cy⁴ is a 5- to 8-membered heterocycloalkyl that is unsubstituted or substituted by one or more Cy⁴⁻¹, the heteroatoms of the 5- to 8-membered heterocycle are selected from one or more of N, S and O, and the number of heteroatoms is 1, 2 or 3. Ring Cy⁴ is a 5- to 8-membered heterocycloalkyl that is unsubstituted or substituted by one or more Cy⁴⁻¹; the heteroatom of the 5- to 8-membered heterocycle is N, S or O, and the number of heteroatoms is 1.

In a preferred embodiment, each Cy⁴⁻¹ is independently C₁-C₆ alkyl, hydroxyl or oxo, preferably oxo.

In a preferred embodiment, the five-membered ring-fused six-membered ring compound represented by formula I is a compound represented by formula I-a, I-b, I-d, I-g, I-h or I-i:

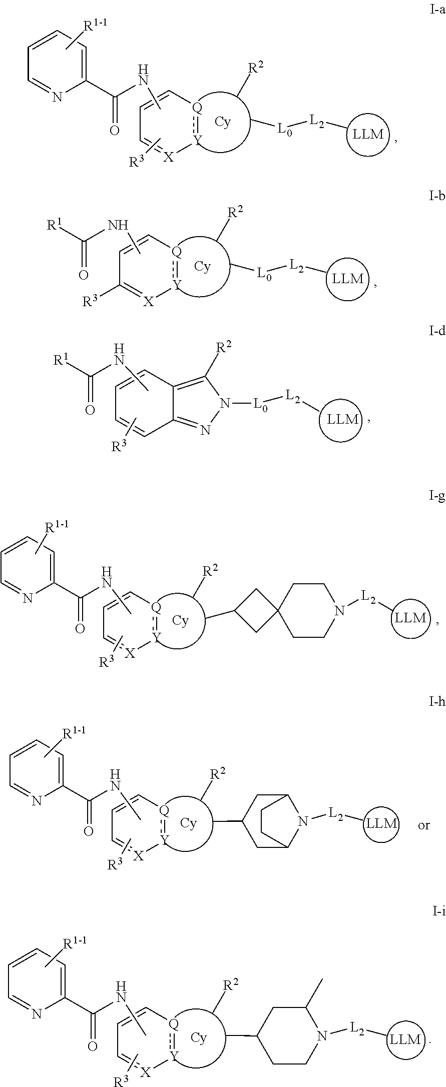

In a preferred embodiment, the five-membered ring-fused six-membered ring compound represented by formula I is a compound represented by formula I-a, I-b, I-d, I-g, I-h, I-i, I-k, I-m or I-n:

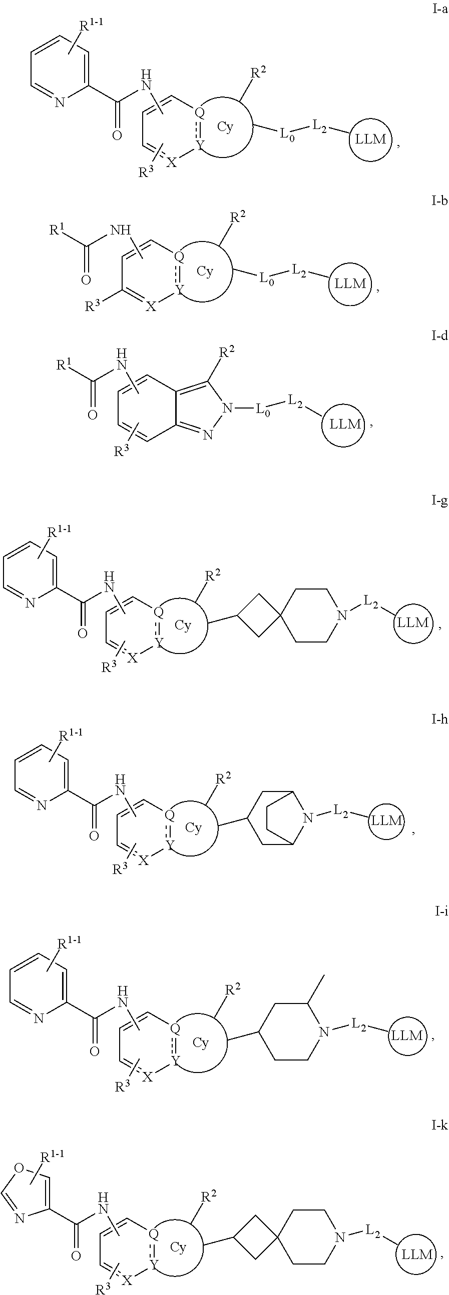

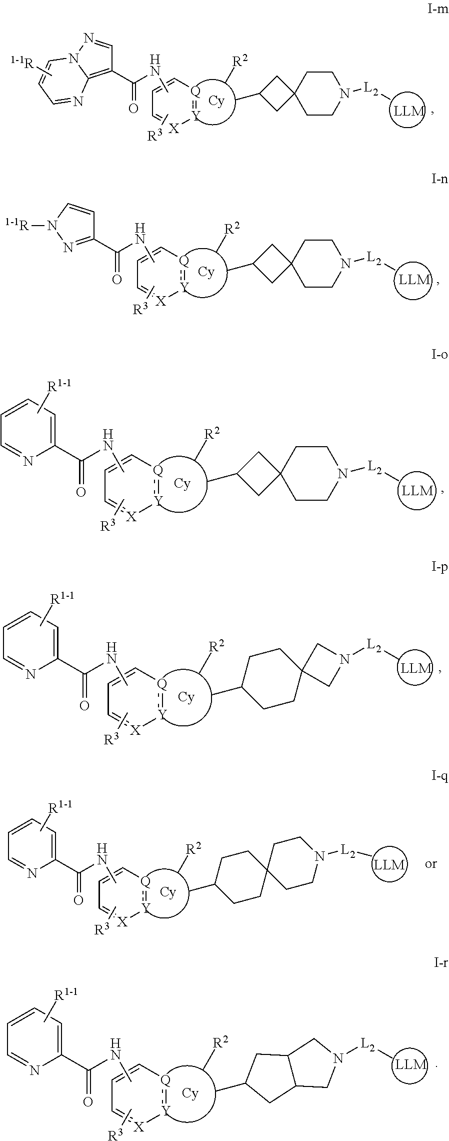

In a preferred embodiment, LLM is

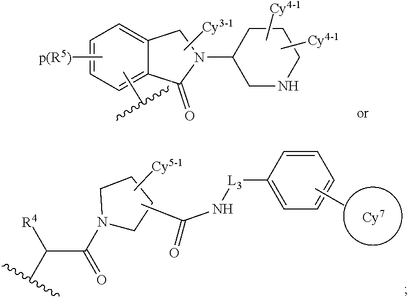

preferably is

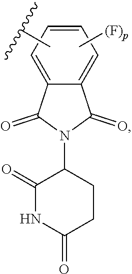

wherein p is 0 or 1;

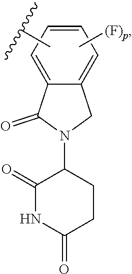

wherein p is 0 or 1; or

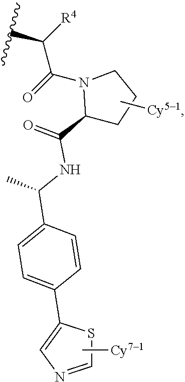

more preferably

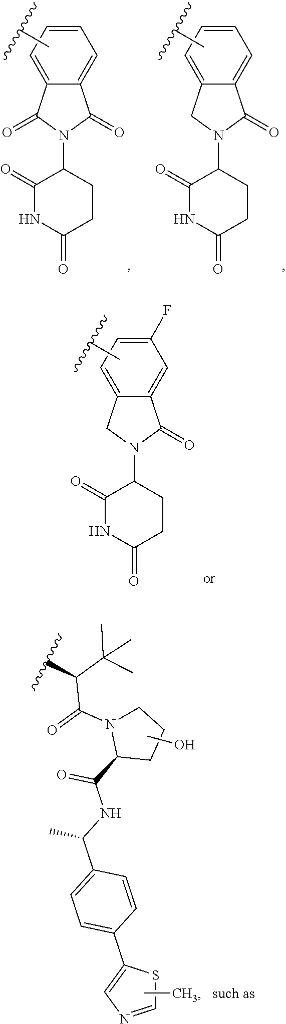

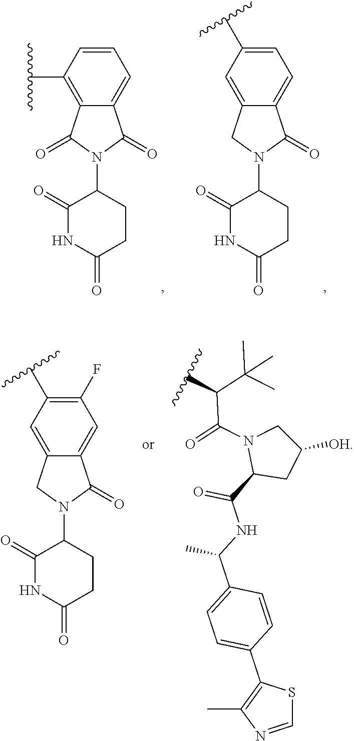 [SMILES: *c1cccc2c1C(=O)N(C1CCC(=O)NC1=O)C2=O ; *c1ccc2c(c1)CN(C1CCC(=O)NC1=O)C2=O ; *c1cc2c(cc1F)C(=O)N(C1CCC(=O)NC1=O)C2 ; *[C@H](C(=O)N1C[C@H](O)C[C@H]1C(=O)N[C@@H](C)c1ccc(-c2scnc2C)cc1)C(C)(C)C]

In a preferred embodiment, LLM is

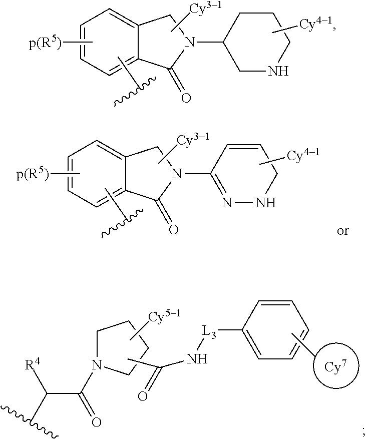

preferably is

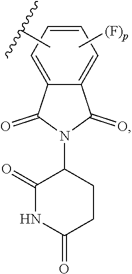

wherein p is 0 or 1;

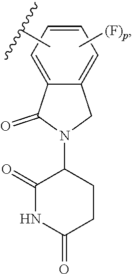

wherein p is 0 or 1;

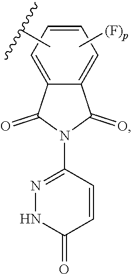

wherein
p is 0 or 1; or

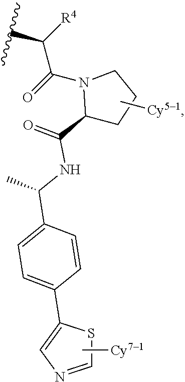

more preferably

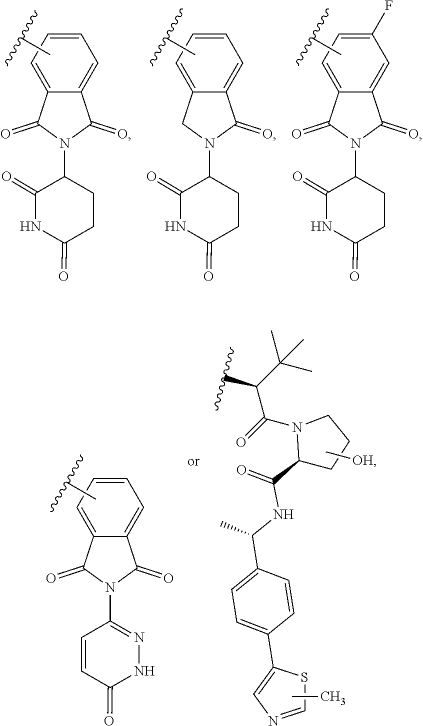

such as

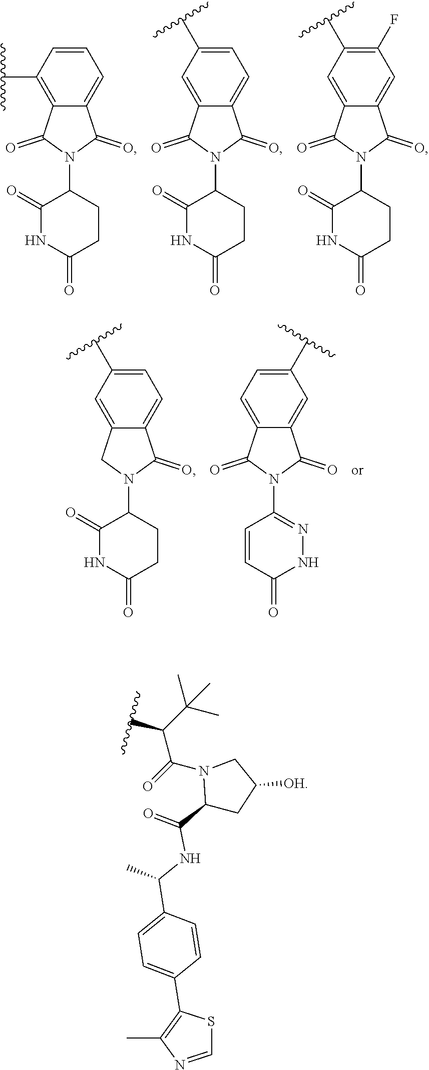

In a preferred embodiment, LLM is

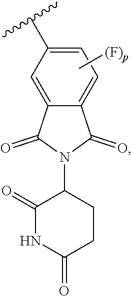

wherein p is 0 or 1; such as

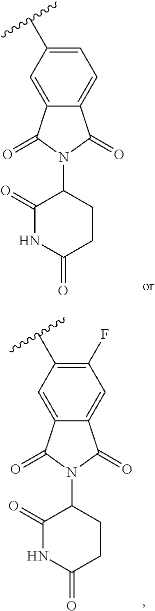 [SMILES: *c1ccc2c(c1)C(=O)N(C1CCC(=O)NC1=O)C2=O ; *c1cc2c(cc1F)C(=O)N(C1CCC(=O)NC1=O)C2=O]

also such as

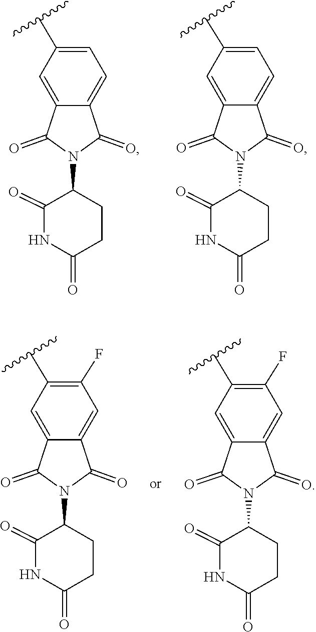 [SMILES: *c1ccc2c(c1)C(=O)N([C@H]1CCC(=O)NC1=O)C2=O ; *c1ccc2c(c1)C(=O)N([C@@H]1CCC(=O)NC1=O)C2=O ; *c1cc2c(cc1F)C(=O)N([C@H]1CCC(=O)NC1=O)C2=O ; "or" ; *c1cc2c(cc1F)C(=O)N([C@@H]1CCC(=O)NC1=O)C2=O]

In a preferred embodiment, R¹ is

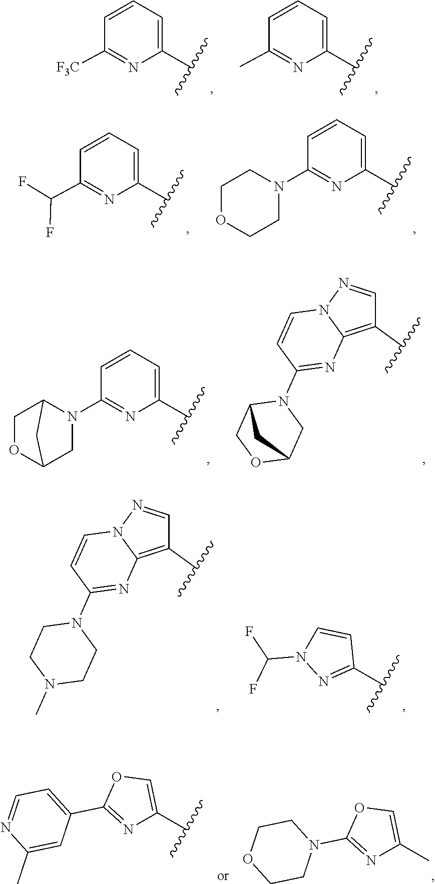

preferably

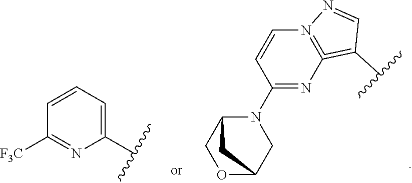

In a preferred embodiment, R³ is methoxy, isopropoxy, difluoromethoxy, trifluoromethoxy, carboxyl, fluorine,

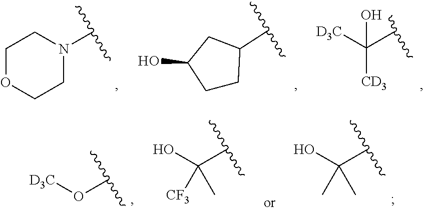

preferably methoxy,

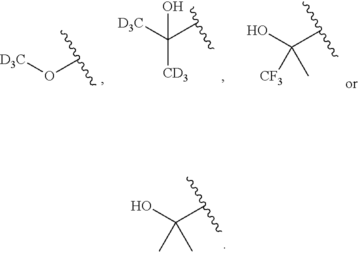

In a preferred embodiment,

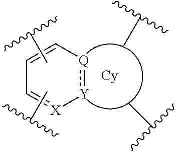

is

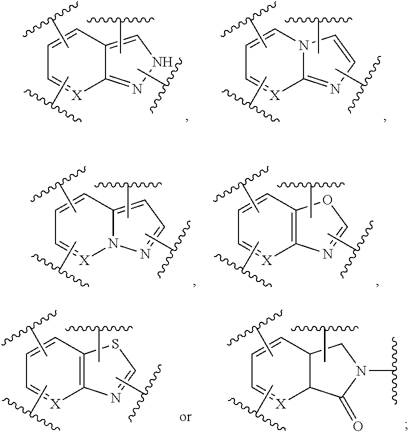

such as

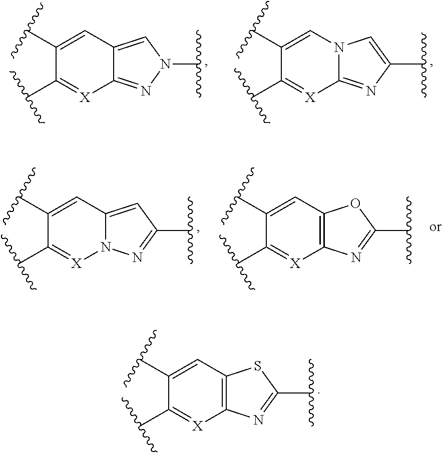

In a preferred embodiment, in the compounds of formula I,

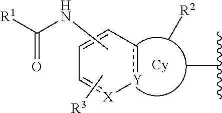

is

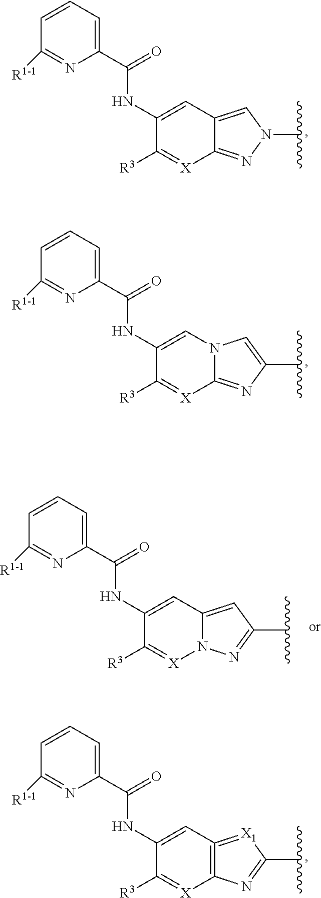

### X is No r CH, and X₁ is S or O.

In a preferred embodiment, in the compounds of formula I,

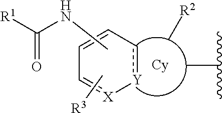

is

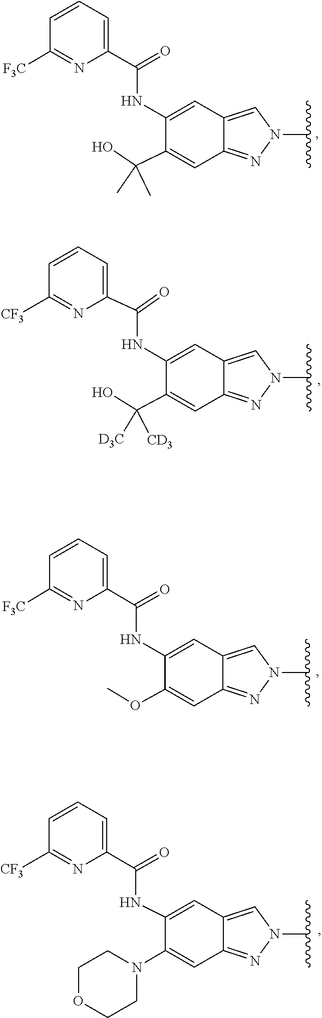 [SMILES: *n1cc2cc(NC(=O)c3cccc(C(F)(F)F)n3)c(C(C)(C)O)cc2n1 ; *n1cc2cc(NC(=O)c3cccc(C(F)(F)F)n3)c(C(O)(C([2H])([2H])[2H])C([2H])([2H])[2H])cc2n1 ; *n1cc2cc(NC(=O)c3cccc(C(F)(F)F)n3)c(OC)cc2n1 ; *n1cc2cc(NC(=O)c3cccc(C(F)(F)F)n3)c(N3CCOCC3)cc2n1]

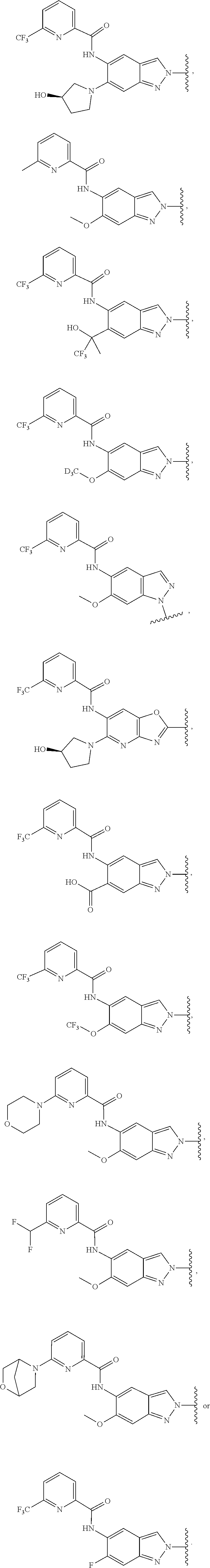

In a preferred embodiment, in the compounds of formula I,

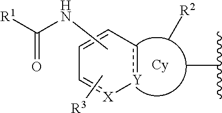

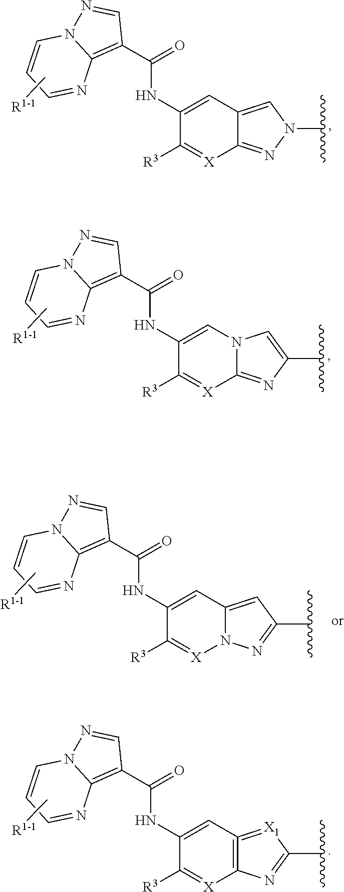

is

In a preferred embodiment, in the compounds of formula I,

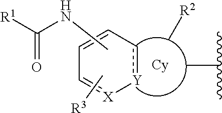

is

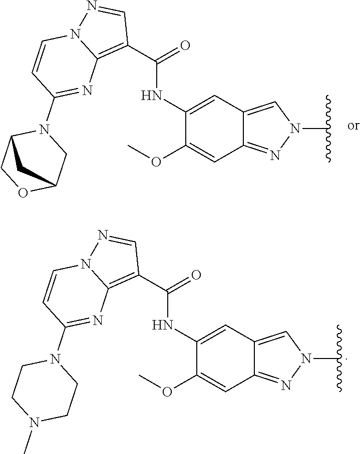 [SMILES: *n1cc2cc(NC(=O)c3cnn4ccc(N5C[C@H]6C[C@@H]5CO6)nc34)c(OC)cc2n1 ; *n1cc2cc(NC(=O)c3cnn4ccc(N5CCN(C)CC5)nc34)c(OC)cc2n1]

In a preferred embodiment, in the compounds of formula I,

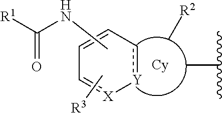

is

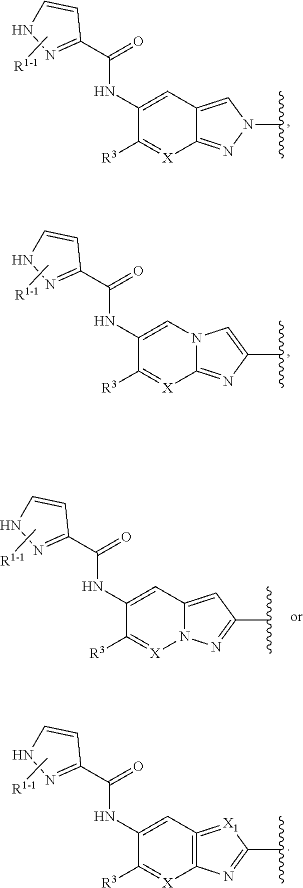

In a preferred embodiment, in the compounds of formula I,

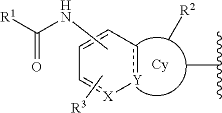

is

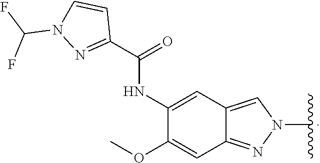 [SMILES: *n1cc2cc(NC(=O)c3ccn(C(F)F)n3)c(OC)cc2n1]

In a preferred embodiment, in the compounds of formula I,

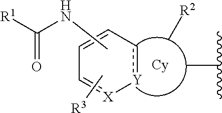

is

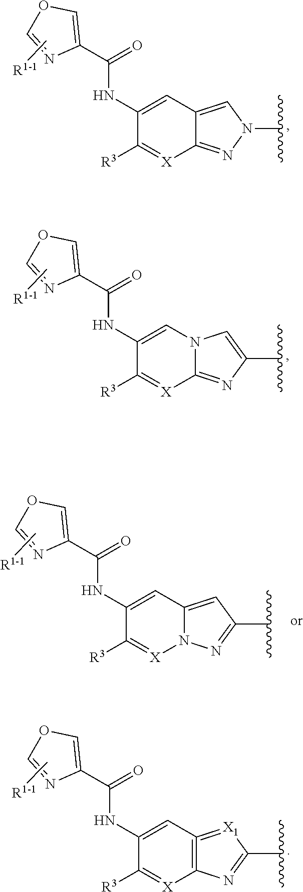

In a preferred embodiment, in the compounds of formula I,

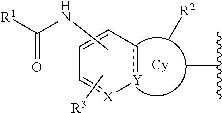

is

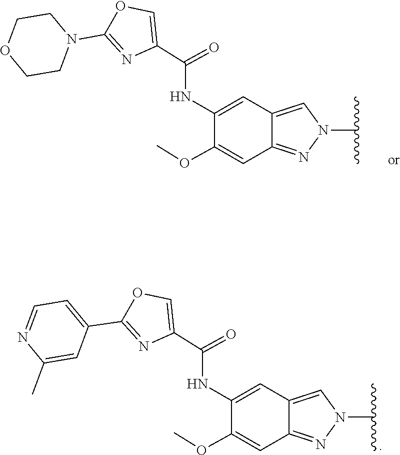 [SMILES: *n1cc2cc(NC(=O)c3coc(N4CCOCC4)n3)c(OC)cc2n1 ; *n1cc2cc(NC(=O)c3coc(-c4ccnc(C)c4)n3)c(OC)cc2n1]

In a preferred embodiment, L₀ is

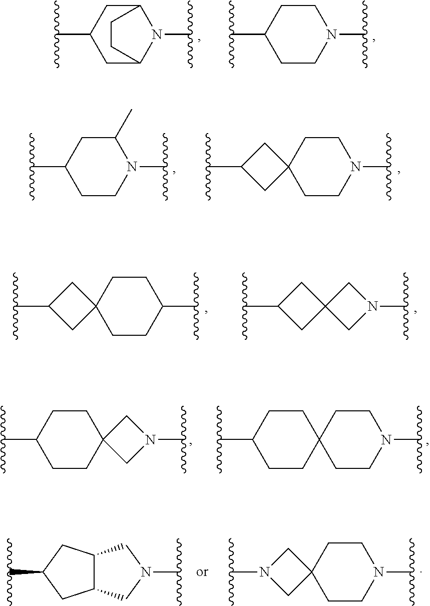

In a preferred embodiment L₀ is

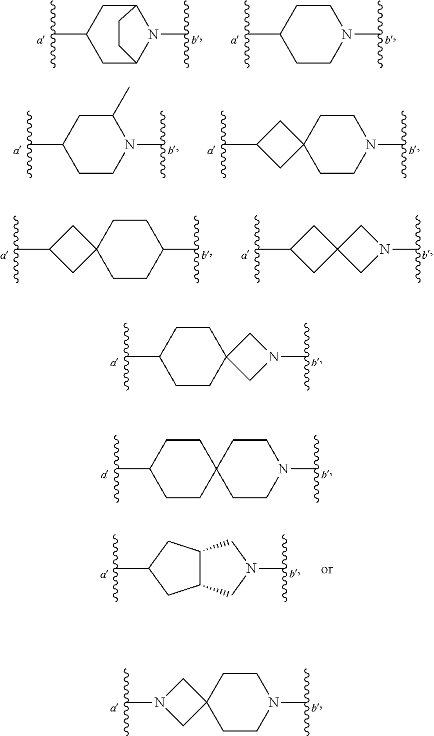

wherein the a′ end is connected to ring Cy, and the b′ end is connected to L².

In a preferred embodiment, L₀ is

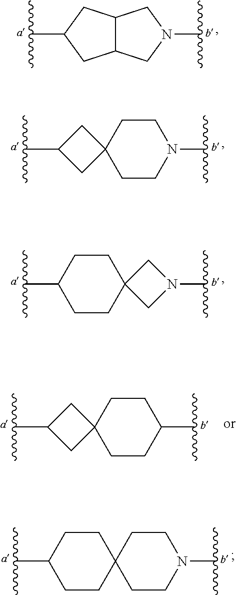 [SMILES: *C1CC2CN(*)CC2C1 ; *C1CC2(CCN(*)CC2)C1 ; *C1CCC2(CC1)CN(*)C2 ; *C1CCC2(CC1)CC(*)C2 ; *C1CCC2(CC1)CCN(*)CC2]

wherein the a′ end is connected to ring Cy, and the b′ end is connected to L².

In a preferred embodiment, L₀,

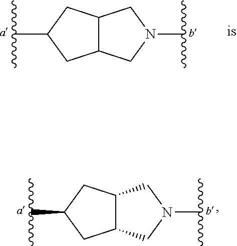 [SMILES: *C1CC2CN(*)CC2C1 ; *[C@H]1C[C@@H]2CN(*)C[C@@H]2C1]

wherein the a′ end is connected to ring Cy, and the b′ end is connected to L².

In a preferred embodiment L₂ is

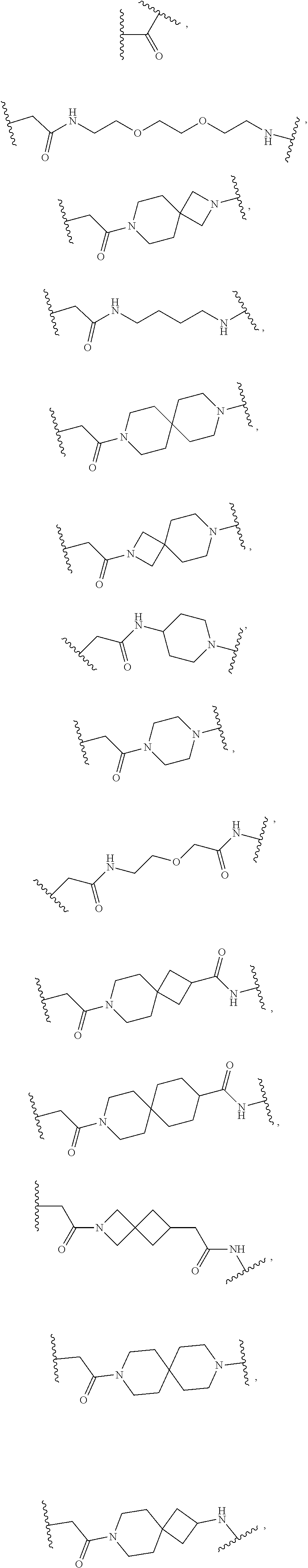

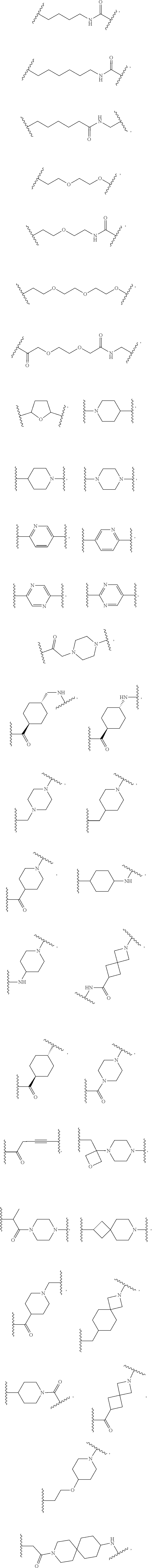

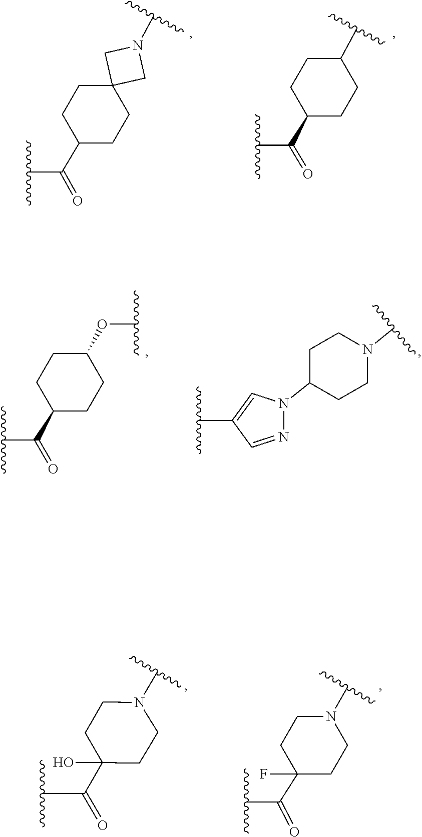

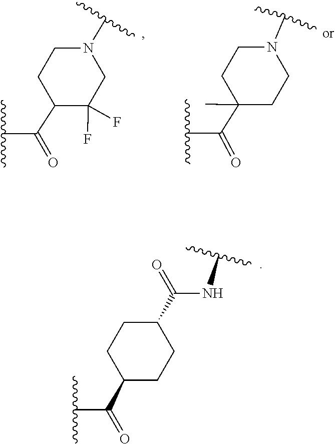

In a preferred embodiment, L₂ is

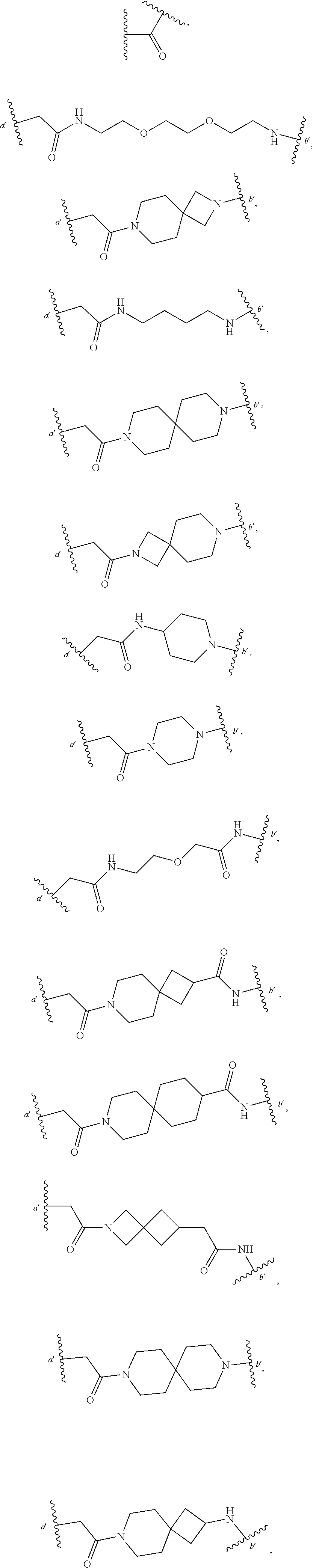

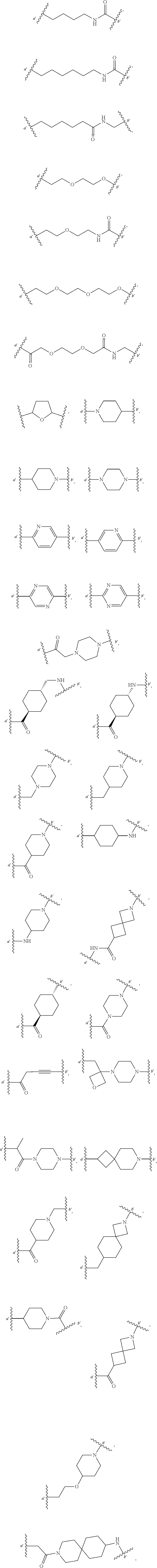

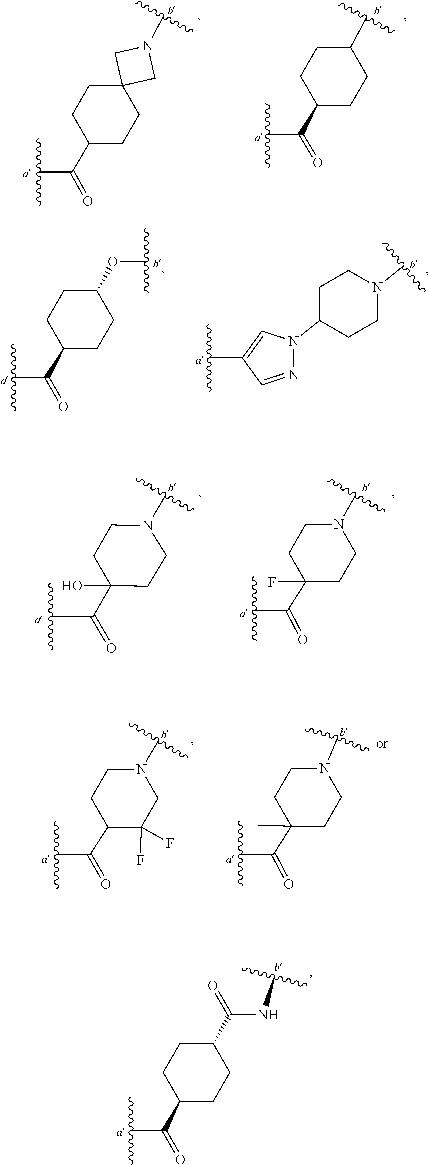

wherein the a′ end is connected to L₀, and the b′ end is connected to LLM.

In a preferred embodiment, when L₀ is

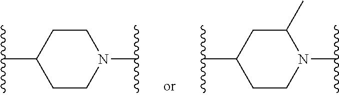

(Further, L₀ is a 5- to 12-membered heterocycloalkylene group that is unsubstituted or substituted by one or more L₀⁻³), L₂ is

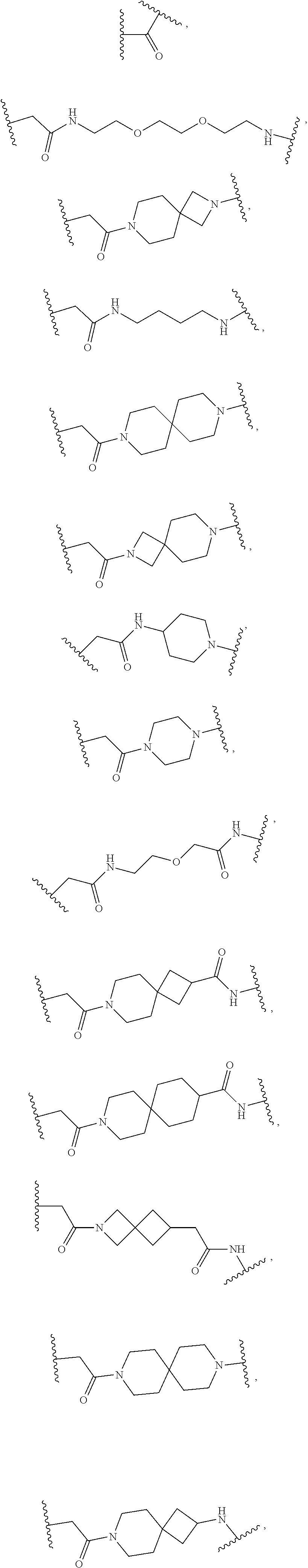

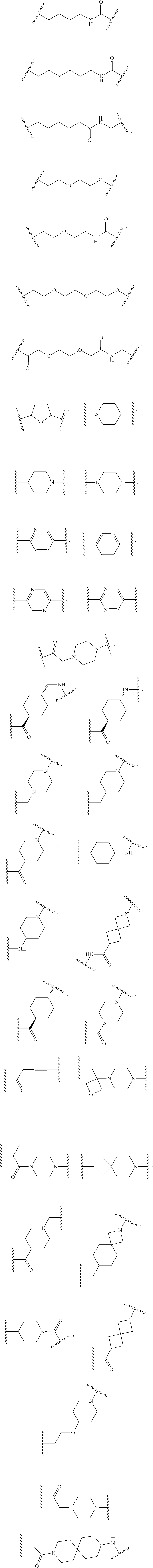

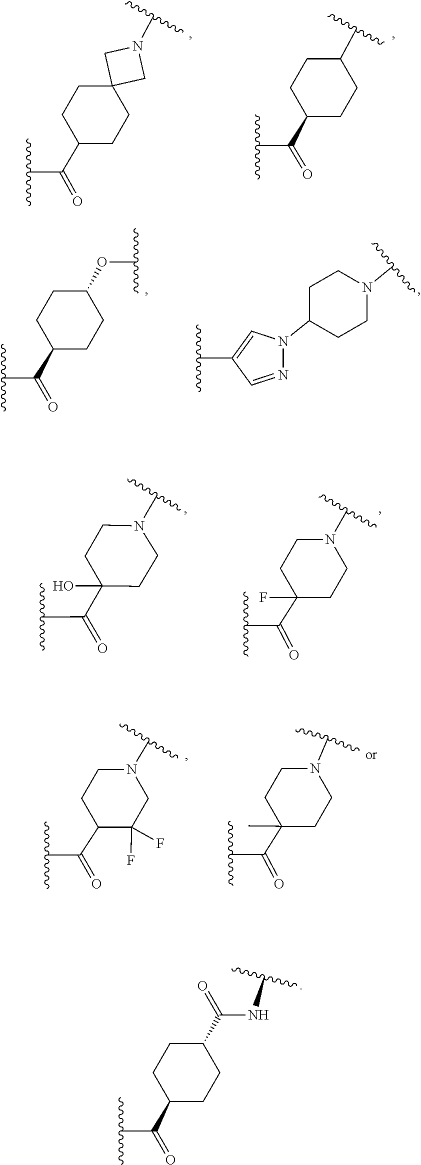

In a preferred embodiment, when L₀ is

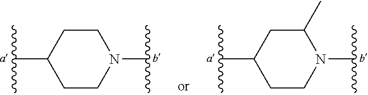

(Further, L₀ is a 5- to 12-membered heterocycloalkylene group that is unsubstituted or substituted by one or more L₀⁻³), L₂ is

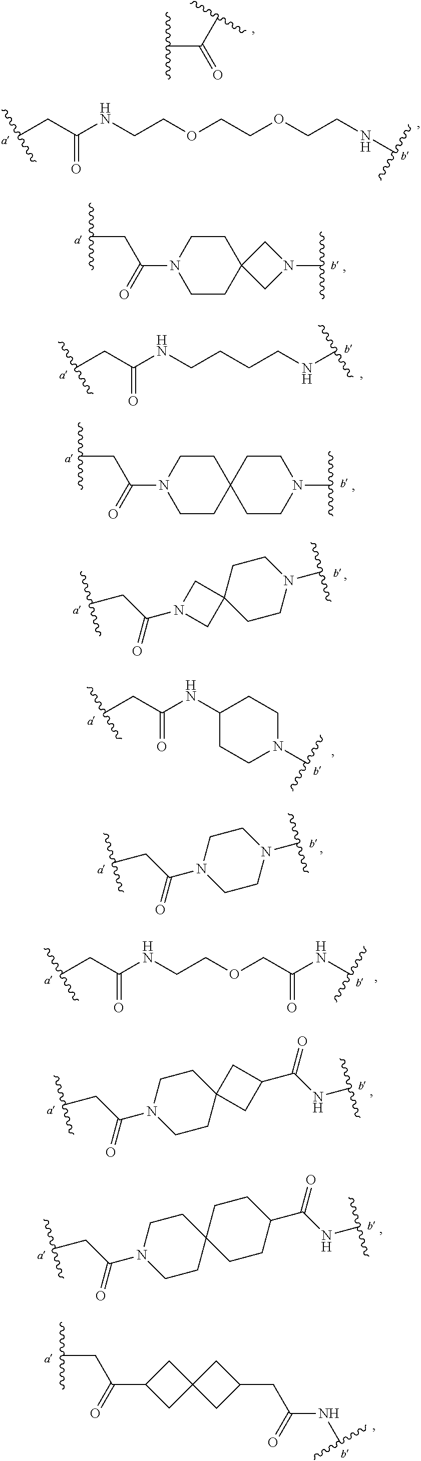

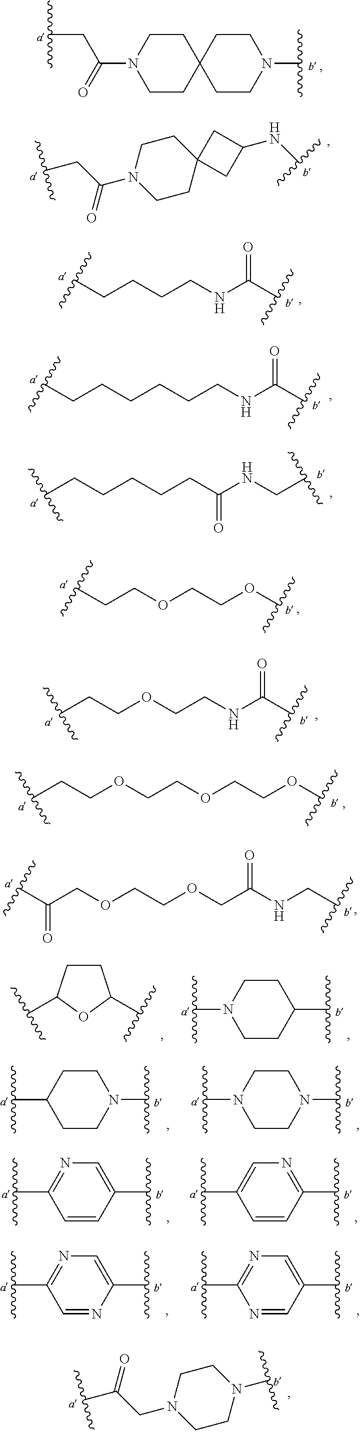

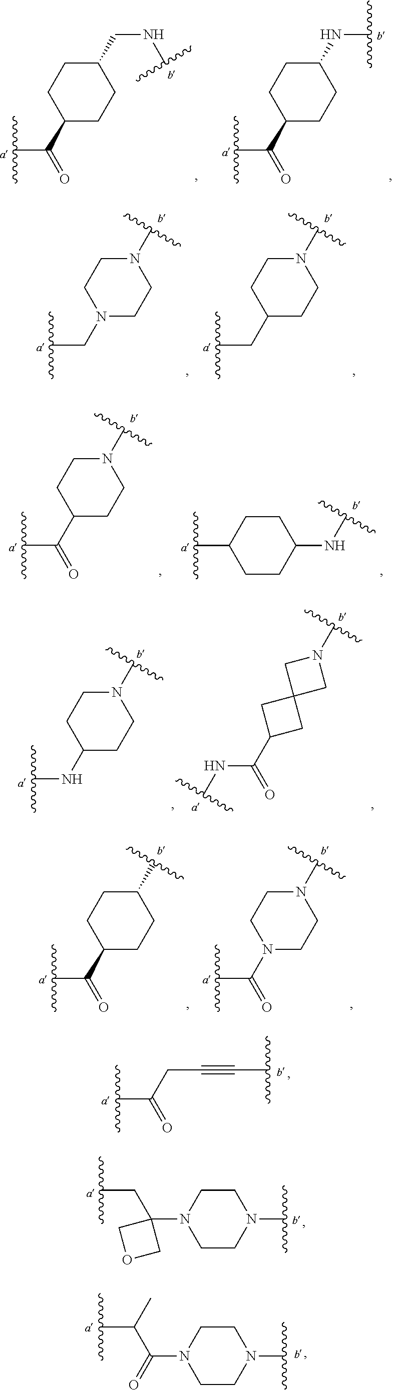

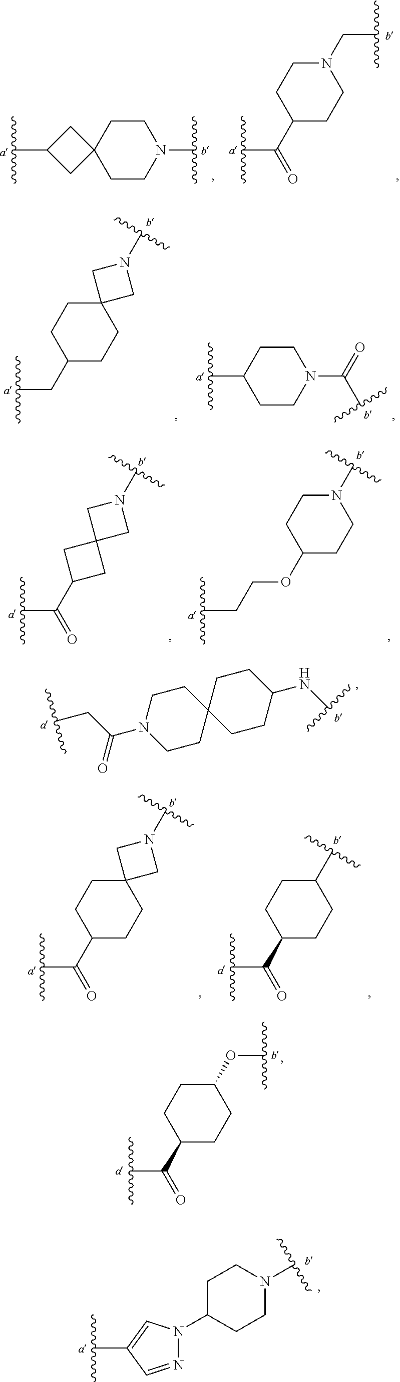

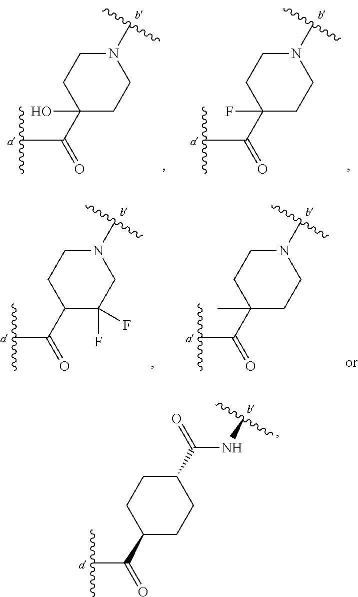

wherein the a′ end of L₂ is connected to L₀, and the b′ end of L₂ is connected to LLM.

In a preferred embodiment, when R¹ is

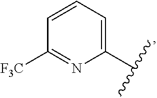 [SMILES: *c1cccc(C(F)(F)F)n1]

### L₀ is

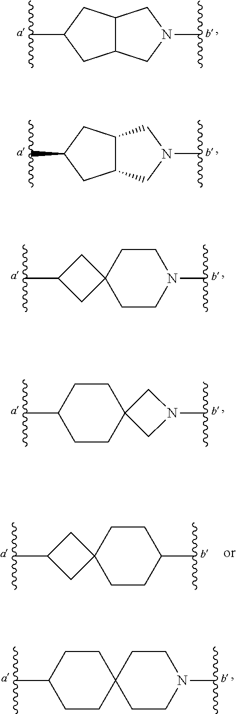 [SMILES: *C1CC2CN(*)CC2C1 ; *[C@H]1C[C@@H]2CN(*)C[C@@H]2C1 ; *C1CC2(CCN(*)CC2)C1 ; *C1CCC2(CC1)CN(*)C2 ; *C1CCC2(CC1)CC(*)C2 ; *C1CCC2(CC1)CCN(*)CC2]

wherein the a′ end is connected to ring Cy, and the b′ end is connected to L₂.

In a preferred embodiment, when R¹ is

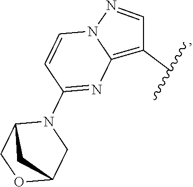 [SMILES: *c1cnn2ccc(N3C[C@H]4C[C@@H]3CO4)nc12]

### L₀ is

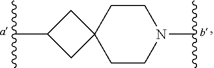 [SMILES: *C1CC2(CCN(*)CC2)C1]

wherein the a′ end is connected to ring Cy, and the b′ end is connected to L₂.

In a preferred embodiment, when R¹ is

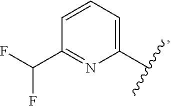 [SMILES: *c1cccc(C(F)F)n1]

### L₀ is

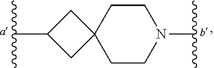 [SMILES: *C1CC2(CCN(*)CC2)C1]

wherein the a′ end is connected to ring Cy, and the b′ end is connected to L₂.

In a preferred embodiment, when R¹ is

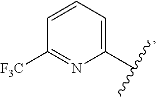 [SMILES: *c1cccc(C(F)(F)F)n1]

### L₂- is

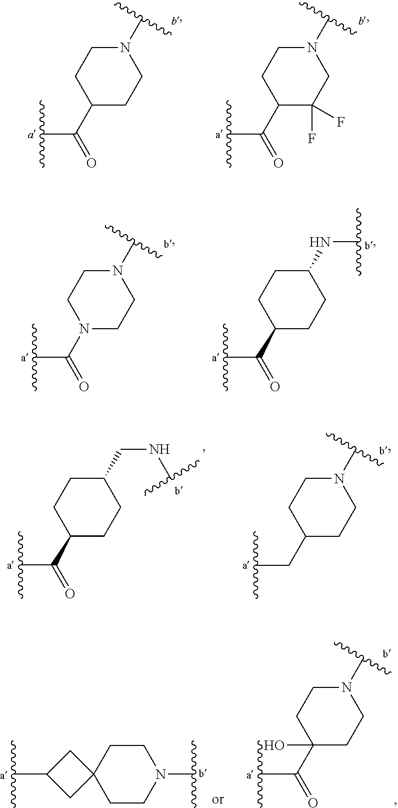

wherein the a′ end of L₂ is connected to L₀, and the b′ end of L₂ is connected to LLM.

In a preferred embodiment, when R¹ is

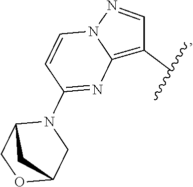 [SMILES: *c1cnn2ccc(N3C[C@H]4C[C@@H]3CO4)nc12]

### L₂- is

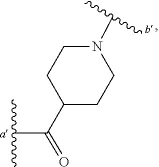 [SMILES: *C(=O)C1CCN(*)CC1]

wherein the a′ end of L₂ is connected to L₀, and the b′ end of L₂ is connected to LLM.

In a preferred embodiment, when R¹ is

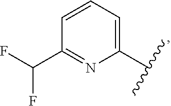 [SMILES: *c1cccc(C(F)F)n1]

### L₂- is

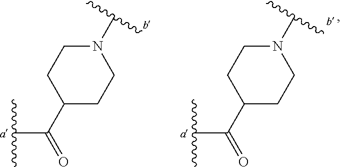 [SMILES: *C(=O)C1CCN(*)CC1 ; *C(=O)C1CCN(*)CC1]

wherein the a′ end of L₂ is connected to L₀, and the b′ end of L₂ is connected to LLM.

In a preferred embodiment, -L₀-L₂- is

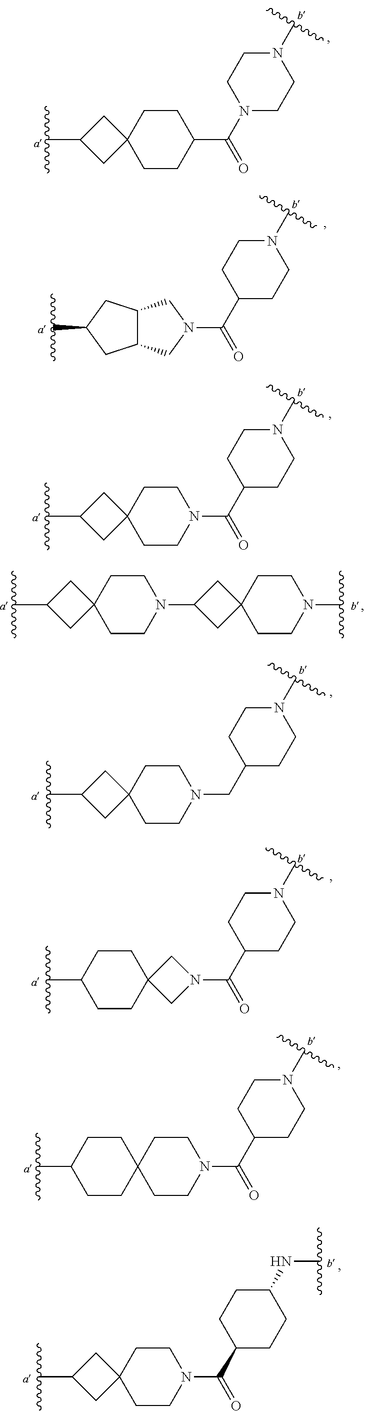

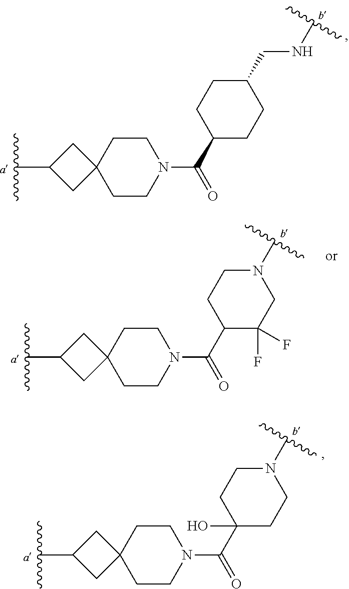 [SMILES: *NC[C@H]1CC[C@H](C(=O)N2CCC3(CC2)CC(*)C3)CC1 ; *C1CC2(CCN(C(=O)C3CCN(*)CC3(F)F)CC2)C1 ; *C1CC2(CCN(C(=O)C3(O)CCN(*)CC3)CC2)C1]

preferably

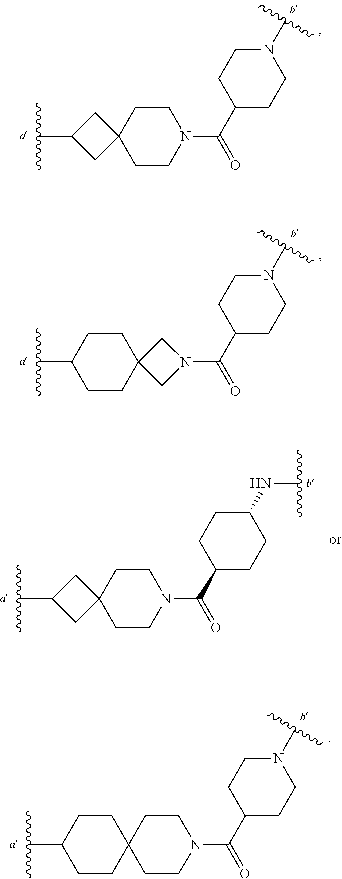 [SMILES: *C1CC2(CCN(C(=O)C3CCN(*)CC3)CC2)C1 ; *C1CCC2(CC1)CN(C(=O)C1CCN(*)CC1)C2 ; *N[C@H]1CC[C@H](C(=O)N2CCC3(CC2)CC(*)C3)CC1 ; *C1CCC2(CC1)CCN(C(=O)C1CCN(*)CC1)CC2]

In a preferred embodiment, -L₀-L₂- is

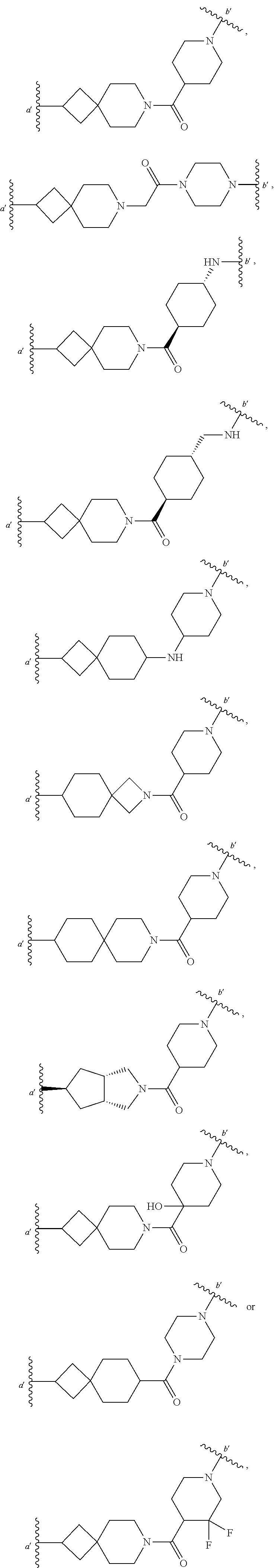

wherein the a′ end is connected to ring Cy, and the b′ end is connected to LLM.

In a preferred embodiment, when

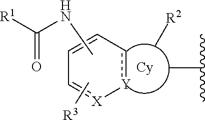

is

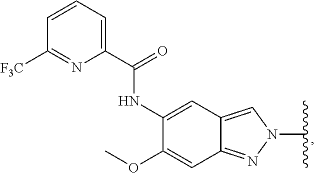 [SMILES: *n1cc2cc(NC(=O)c3cccc(C(F)(F)F)n3)c(OC)cc2n1]

L₀-L₂- is

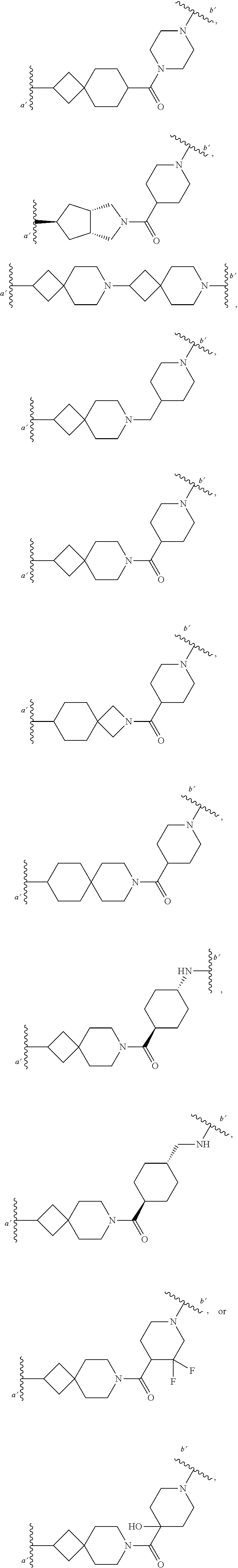

wherein the a′ end is connected to ring Cy, and the b′ end is connected to LLM; preferably

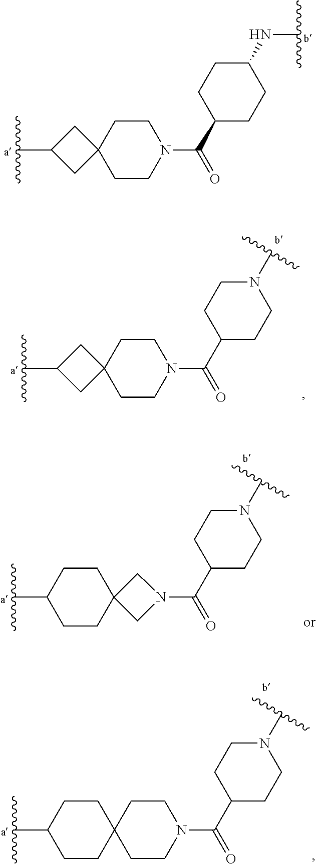 [SMILES: *N[C@H]1CC[C@H](C(=O)N2CCC3(CC2)CC(*)C3)CC1 ; *C1CC2(CCN(C(=O)C3CCN(*)CC3)CC2)C1 ; *C1CCC2(CC1)CN(C(=O)C1CCN(*)CC1)C2 ; *C1CCC2(CC1)CCN(C(=O)C1CCN(*)CC1)CC2]

wherein the a′ end is connected to ring Cy, and the b′ end is connected to LLM.

In a preferred embodiment, when

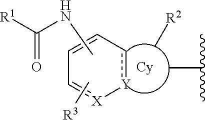

is

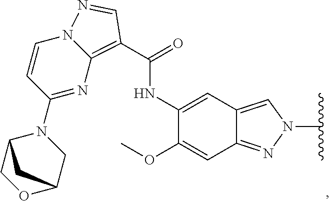 [SMILES: *n1cc2cc(NC(=O)c3cnn4ccc(N5C[C@H]6C[C@@H]5CO6)nc34)c(OC)cc2n1]

L₀-L₂- is

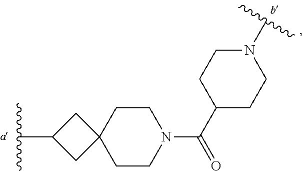 [SMILES: *C1CC2(CCN(C(=O)C3CCN(*)CC3)CC2)C1]

wherein the a′ end is connected to ring Cy, and the b′ end is connected to LLM.

In a preferred embodiment, when

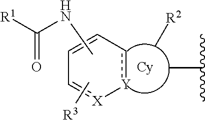

is

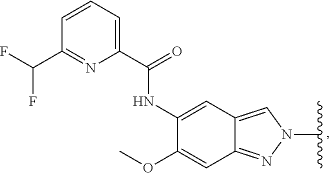 [SMILES: *n1cc2cc(NC(=O)c3cccc(C(F)F)n3)c(OC)cc2n1]

L₀-L₂- is

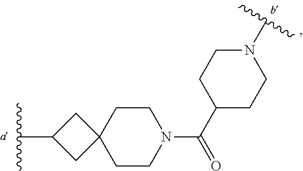 [SMILES: *C1CC2(CCN(C(=O)C3CCN(*)CC3)CC2)C1]

wherein the a′ end is connected to ring Cy, and the b′ end is connected to LLM.

In a preferred embodiment, the compound of formula I is a compound of formula I-d,

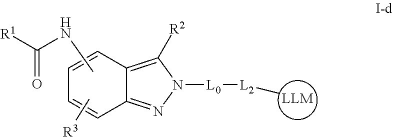

Wherein R¹ is pyridyl substituted by one or more R¹⁻¹, or

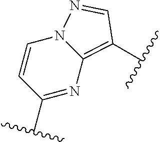 [SMILES: *c1ccn2ncc(*)c2n1]

substituted by one or more R^{1-1;}
When R¹ is pyridyl substituted by one or more R¹⁻¹, each R¹⁻¹ is independently a C₁-C₆ alkyl substituted by one or more R^{1-1-4;}
When R¹ is

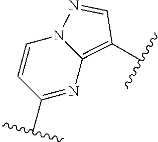 [SMILES: *c1ccn2ncc(*)c2n1]

substituted by one or more R¹⁻¹, each R¹⁻¹ is independently a 7- to 8-membered heterocycloalkyl that is unsubstituted or substituted by one or more R¹⁻¹⁻¹; the heteroatom of the 7- to 8-membered heterocycloalkyl is selected from N or O, and the number of the heteroatom is 2;
Each R¹⁻¹⁻⁴ is independently halogen; each R¹⁻¹⁻¹ is independently a C₁-C₆ alkyl substituted by one or more halogens;
R² is hydrogen;
R³ is a C₁-C₆ alkyl that is unsubstituted or substituted by one or more R³⁻⁴ or a C₁-C₆ alkoxy that is unsubstituted or substituted by one or more R^{3-7;}
Each R³⁻⁴ is independently deuterium, halogen or hydroxyl; and each R³⁻⁷ is independently deuterium;
L₀ is a 9- to 11-membered heterospirylene group; the heteroatom of the 9- to 11-membered heterospirylene group is N, and the number of heteroatoms is 1;
L₂ is -L₂⁻¹-L₂⁻²-L₂⁻³-L₂⁻⁴- or

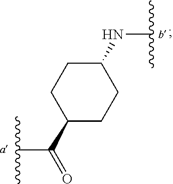 [SMILES: *N[C@H]1CC[C@H](C(*)=O)CC1]

L₂⁻¹ is

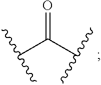 [SMILES: *C(*)=O]

L₂⁻², L₂⁻³ and L₂⁻⁴ are independently absent,

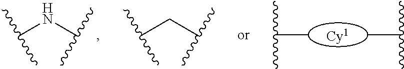

that is unsubstituted or substituted by one or more L₂¹⁻², ring Cy¹ is a 4- to 6-membered heterocycle, the heteroatom of the 4- to 6-membered heterocycle is N, and the number of heteroatoms is 1; the 4- to 6-membered heterocycle is a monocyclic ring; and each L₂¹⁻² is independently hydroxyl;
LLM is

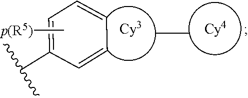

Ring Cy³ is a 5- to 6-membered heterocycle that is unsubstituted or substituted by one or more Cy³⁻¹, the heteroatoms of the 5- to 6-membered heterocycle are selected from one or more of N, S and O, and the number of heteroatoms is 1; Cy³⁻¹ is independently oxo;
Ring Cy⁴ is a 5- to 8-membered heterocycloalkyl that is unsubstituted or substituted by one or more Cy⁴⁻¹, the heteroatom of the 5- to 8-membered heterocycloalkyl is N, S or O, and the number of heteroatoms is 1; Cy⁴⁻¹ is independently oxo;
Each R⁵ is independently halogen;
p is 0, 1, 2 or 3.

In a preferred embodiment, R¹ is pyridyl substituted by one or more R¹⁻¹, or

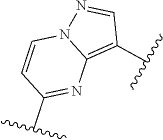 [SMILES: *c1ccn2ncc(*)c2n1]

substituted by one or more R^{1-1.}

In a preferred embodiment, when R¹ is pyridyl substituted by one or more R¹⁻¹, each R¹⁻¹ is independently a C₁-C₆ alkyl substituted by one or more R¹⁻¹⁻⁴.

In a preferred embodiment, when R¹ is

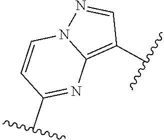 [SMILES: *c1ccn2ncc(*)c2n1]

substituted by one or more R¹⁻¹, each R¹⁻¹ is independently a 7- to 8-membered heterocycloalkyl that is unsubstituted or substituted by one or more R¹⁻¹⁻¹; the heteroatom of the 7- to 8-membered heterocycloalkyl is selected from N or O, and the number of the heteroatom is 2.

In a preferred embodiment, each R¹⁻¹⁻⁴ is independently fluorine, chlorine or bromine, preferably fluorine.

In a preferred embodiment, when R¹⁻¹ is independently a C₁-C₆ alkyl substituted by more R¹⁻¹⁻⁴; the more is 2 or 3, preferably 3.

In a preferred embodiment, each R¹⁻¹⁻¹ is independently a C₁-C₆ alkyl substituted by one or more halogens.

In a preferred embodiment, R³ is a C₁-C₆ alkyl that is unsubstituted or substituted by one or more R³ or a C₁-C₆ alkoxy that is unsubstituted or substituted by one or more R^{3-7.}

In a preferred embodiment, each R³⁻⁴ is independently deuterium, halogen or hydroxyl.

In a preferred embodiment, each R³⁻⁷ is independently deuterium.

In a preferred embodiment, L₀ is a 9- to 11-membered heterospirylene group; the heteroatom of the 9- to 11-membered heterospirylene group is N, and the number of heteroatoms is 1.

In a preferred embodiment, L₂ is -L₂⁻¹-L₂⁻²-L₂⁻³-L₂⁻⁴- or

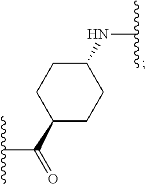 [SMILES: *N[C@H]1CC[C@H](C(*)=O)CC1]

L₂⁻¹ is

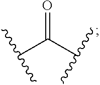 [SMILES: *C(*)=O]

L₂⁻³ and L₂⁻⁴ are independently absent,

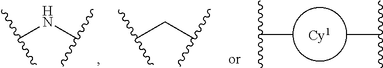

that is unsubstituted or substituted by one or more L₂¹⁻², ring Cy¹ is a 4- to 6-membered heterocycle, the heteroatom of the 4- to 6-membered heterocycle is N, and the number of heteroatoms is 1; the 4- to 6-membered heterocycle is a monocyclic ring.

In a preferred embodiment, each L₂¹⁻² is independently hydroxyl.

In a preferred embodiment, the compound of formula I is a compound of formula I-d,

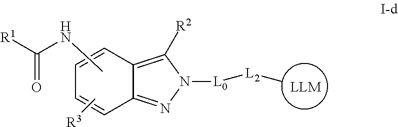

Wherein R¹ is a 5- to 9-membered heteroaryl that is unsubstituted or substituted by one or more R¹⁻¹, the heteroatom of the 5- to 9-membered heteroaryl is one or two of N and O, and the number of heteroatoms is 1, 2 or 3;
Each R¹⁻¹ is a C₁-C₆ alkyl that is unsubstituted or substituted by one or more R¹⁻¹⁻⁴, a 3- to 11-membered heterocycloalkyl that is unsubstituted or substituted by one or more R¹⁻¹⁻¹ or a 5- to 10-membered heteroaryl group that is unsubstituted or substituted by one or more R¹⁻¹⁻⁷; the heteroatoms of the 5- to 10-membered heteroaryl are selected from one or two of N, S and O, and the number of heteroatoms is 1 or 2; the heteroatoms of the 3 to 11-membered heterocycloalkyl are selected from one or two of N, S and O, and the number of heteroatoms is 1 or 2;
Each R¹⁻¹⁻¹, R¹⁻¹⁻⁴ and R¹⁻¹⁻⁷ are independently halogen or C₁-C₆ alkyl unsubstituted or substituted by one or more halogens;
R³ is a C₁-C₆ alkyl that is unsubstituted or substituted by one or more R³⁻⁴ or C₁-C₆ alkoxy that is unsubstituted or substituted by one or more R³⁻⁷ or tetrahydropyrrolyl that is unsubstituted or substituted by one or more R^{3-1;}
Each R³⁻¹ is independently deuterium, halogen or hydroxyl, each R³⁻⁴ is independently deuterium, halogen or hydroxyl, and each R³⁻⁷ is independently deuterium or hydroxyl;
L₀ is an 8- to 11-membered cycloalkylene group or an 8- to 11-membered heterocycloalkylene group; the 8- to 11-membered cycloalkylene group is a spiro ring; the 8- to 11-membered heterocycloalkylene group is a spiro ring or a fused ring; the heteroatom of the 8- to 11-membered heterocycloalkylene group is N, and the number of heteroatoms is 1.
L₂ is one of the following situations:
Situation (1): L₂ is -L₂⁻¹-L₂⁻²-L₂⁻³-L₂⁻⁴-; L₂⁻¹ and L₂⁻² are absent; L₂⁻³ is

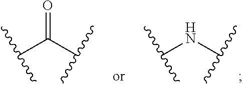

L₂⁻⁴ is ring Cy¹ that is unsubstituted or substituted by one or more L₂¹⁻², the ring Cy¹ is piperidinyl or piperazinyl; each L₂¹⁻² is independently halogen or hydroxyl;
Situation (2): L₂ is -L₂⁻¹-L₂⁻²-L₂⁻³-L₂⁻⁴-; L₂⁻¹ is absent; L₂⁻², L₂⁻³ and L₂⁻⁴ independently are

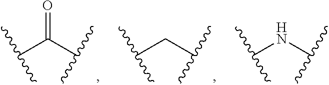

or ring Cy¹ that is unsubstituted or substituted by one or more L₂¹⁻², the ring Cy¹ is cyclohexyl, piperidinyl or piperazinyl; each L₂¹⁻² is independently halogen or hydroxyl;
Situation (3): L₂ is-L₂⁻¹-L₂⁻²-L₂⁻³-L₂⁻⁴; L₂⁻¹, L₂⁻², L₂⁻³ and L₂⁻⁴ independently are

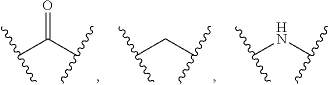

or ring Cy¹ that is unsubstituted or substituted by one or more L₂¹⁻², the ring Cy¹ is cyclohexyl, piperidinyl or piperazinyl; each L₂¹⁻² is independently halogen or hydroxyl;
LLM is

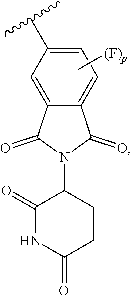

wherein p is 0 or 1.

In a preferred embodiment, the compound of Formula I is any of the following compounds:

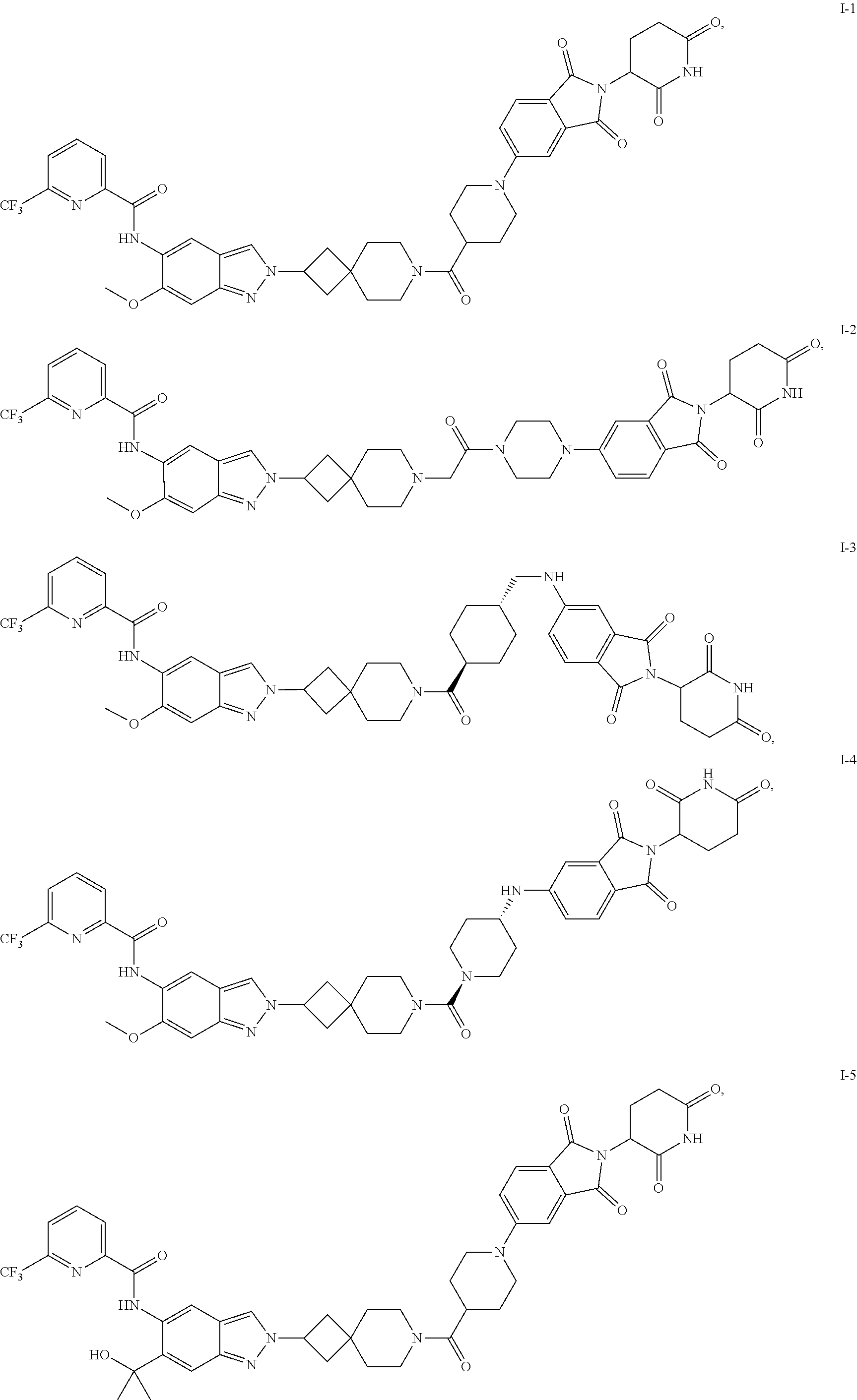

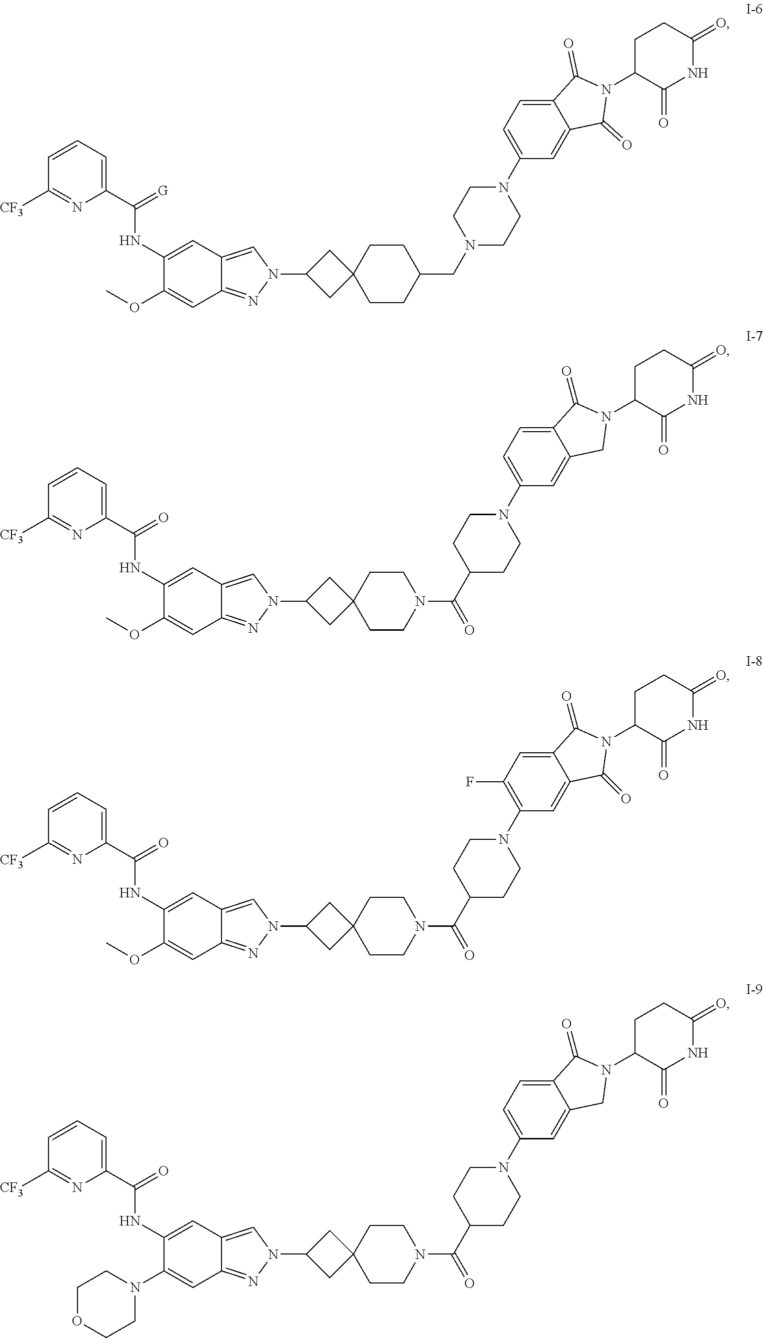

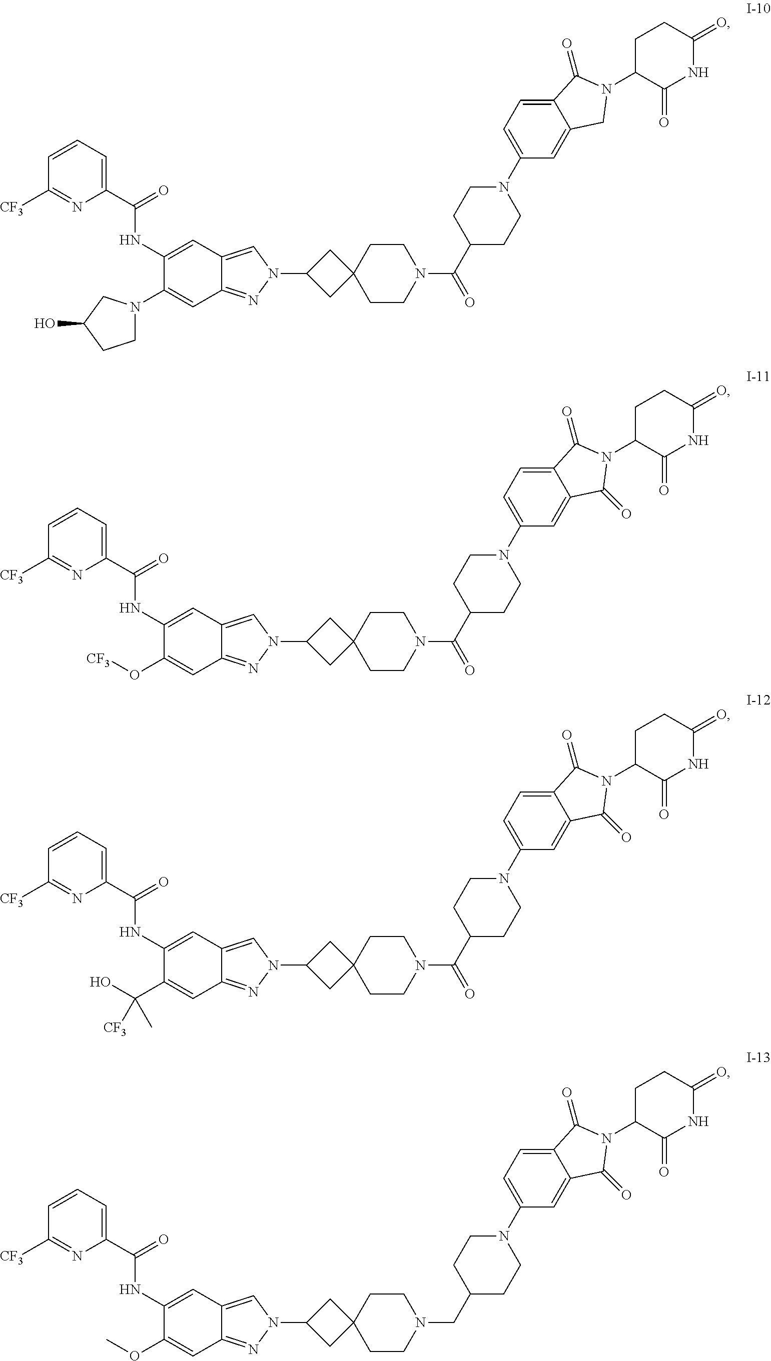 [SMILES: O=C1CCC(N2Cc3cc(N4CCC(C(=O)N5CCC6(CC5)CC(n5cc7cc(NC(=O)c8cccc(C(F)(F)F)n8)c(N8CC[C@@H](O)C8)cc7n5)C6)CC4)ccc3C2=O)C(=O)N1 ; O=C1CCC(N2C(=O)c3ccc(N4CCC(C(=O)N5CCC6(CC5)CC(n5cc7cc(NC(=O)c8cccc(C(F)(F)F)n8)c(OC(F)(F)F)cc7n5)C6)CC4)cc3C2=O)C(=O)N1 ; CC(O)(c1cc2nn(C3CC4(CCN(C(=O)C5CCN(c6ccc7c(c6)C(=O)N(C6CCC(=O)NC6=O)C7=O)CC5)CC4)C3)cc2cc1NC(=O)c1cccc(C(F)(F)F)n1)C(F)(F)F ; COc1cc2nn(C3CC4(CCN(CC5CCN(c6ccc7c(c6)C(=O)N(C6CCC(=O)NC6=O)C7=O)CC5)CC4)C3)cc2cc1NC(=O)c1cccc(C(F)(F)F)n1]

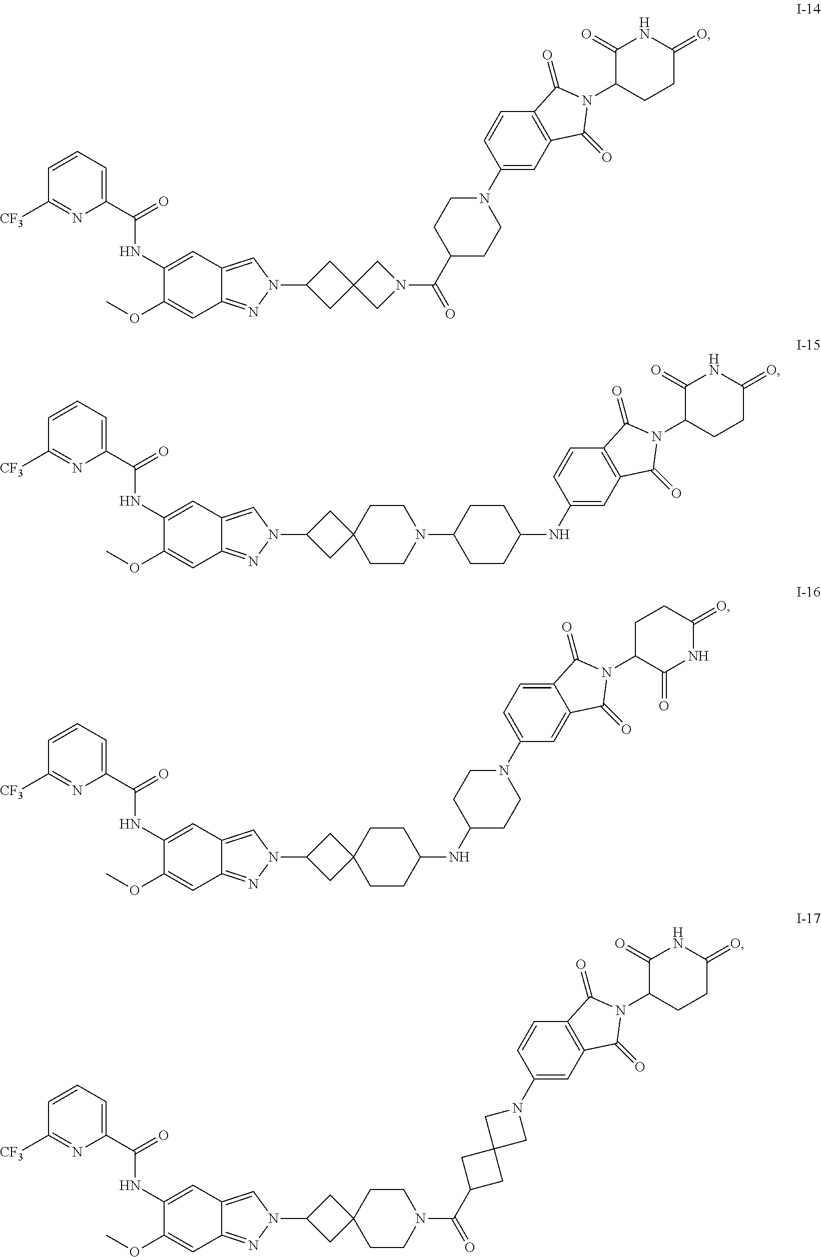 [SMILES: COc1cc2nn(C3CC4(C3)CN(C(=O)C3CCN(c5ccc6c(c5)C(=O)N(C5CCC(=O)NC5=O)C6=O)CC3)C4)cc2cc1NC(=O)c1cccc(C(F)(F)F)n1 ; COc1cc2nn(C3CC4(CCN(C5CCC(Nc6ccc7c(c6)C(=O)N(C6CCC(=O)NC6=O)C7=O)CC5)CC4)C3)cc2cc1NC(=O)c1cccc(C(F)(F)F)n1 ; COc1cc2nn(C3CC4(CCC(NC5CCN(c6ccc7c(c6)C(=O)N(C6CCC(=O)NC6=O)C7=O)CC5)CC4)C3)cc2cc1NC(=O)c1cccc(C(F)(F)F)n1 ; COc1cc2nn(C3CC4(CCN(C(=O)C5CC6(C5)CN(c5ccc7c(c5)C(=O)N(C5CCC(=O)NC5=O)C7=O)C6)CC4)C3)cc2cc1NC(=O)c1cccc(C(F)(F)F)n1]

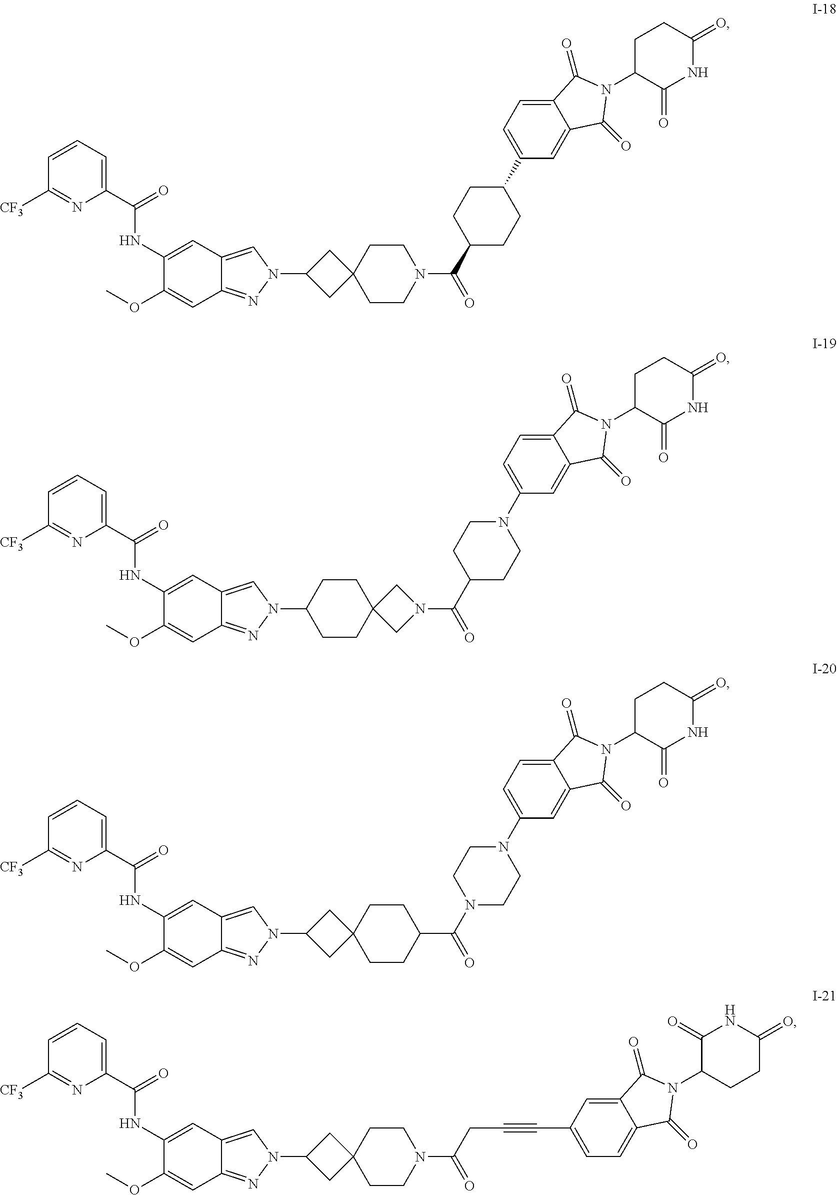 [SMILES: COc1cc2nn(C3CC4(CCN(C(=O)[C@H]5CC[C@H](c6ccc7c(c6)C(=O)N(C6CCC(=O)NC6=O)C7=O)CC5)CC4)C3)cc2cc1NC(=O)c1cccc(C(F)(F)F)n1 ; COc1cc2nn(C3CCC4(CC3)CN(C(=O)C3CCN(c5ccc6c(c5)C(=O)N(C5CCC(=O)NC5=O)C6=O)CC3)C4)cc2cc1NC(=O)c1cccc(C(F)(F)F)n1 ; COc1cc2nn(C3CC4(CCC(C(=O)N5CCN(c6ccc7c(c6)C(=O)N(C6CCC(=O)NC6=O)C7=O)CC5)CC4)C3)cc2cc1NC(=O)c1cccc(C(F)(F)F)n1 ; COc1cc2nn(C3CC4(CCN(C(=O)CC#Cc5ccc6c(c5)C(=O)N(C5CCC(=O)NC5=O)C6=O)CC4)C3)cc2cc1NC(=O)c1cccc(C(F)(F)F)n1]

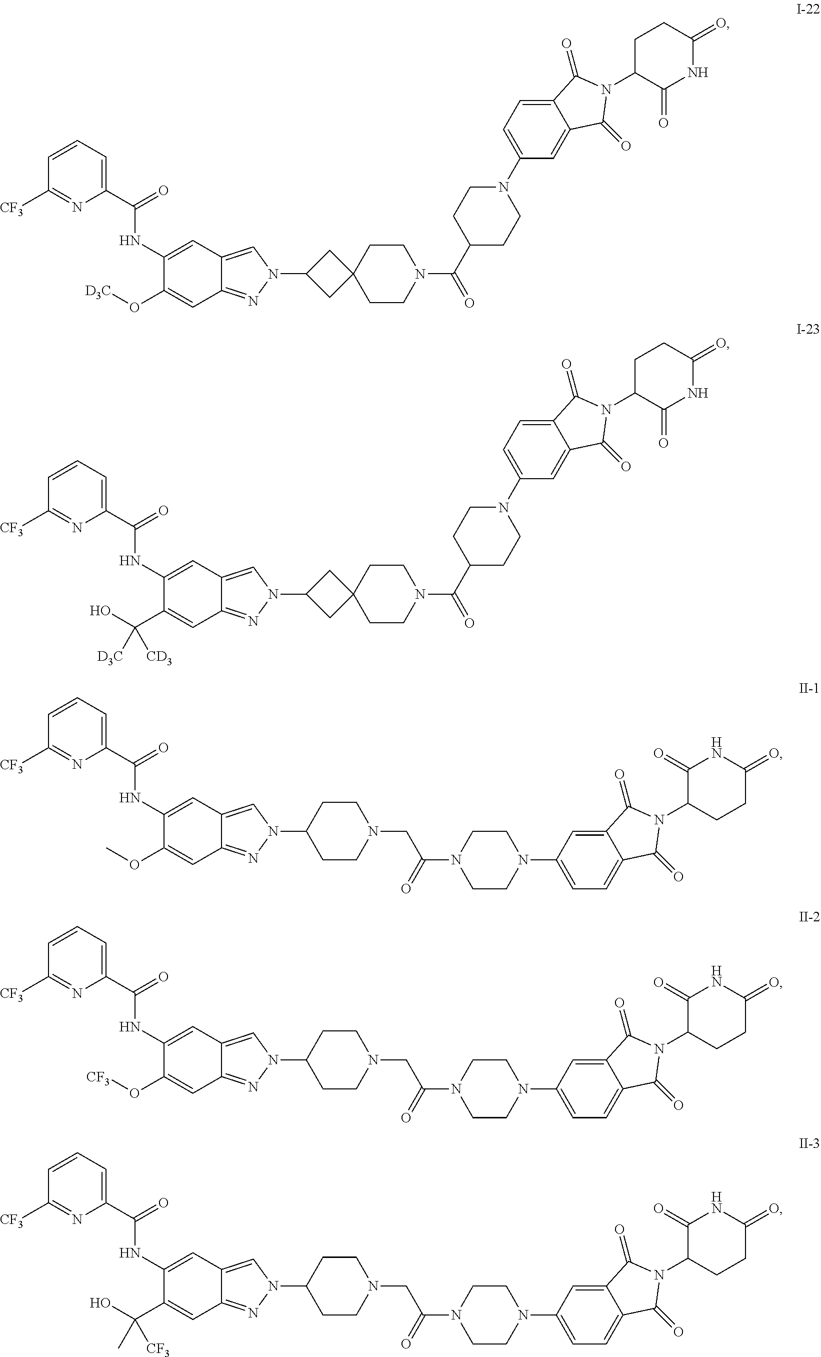

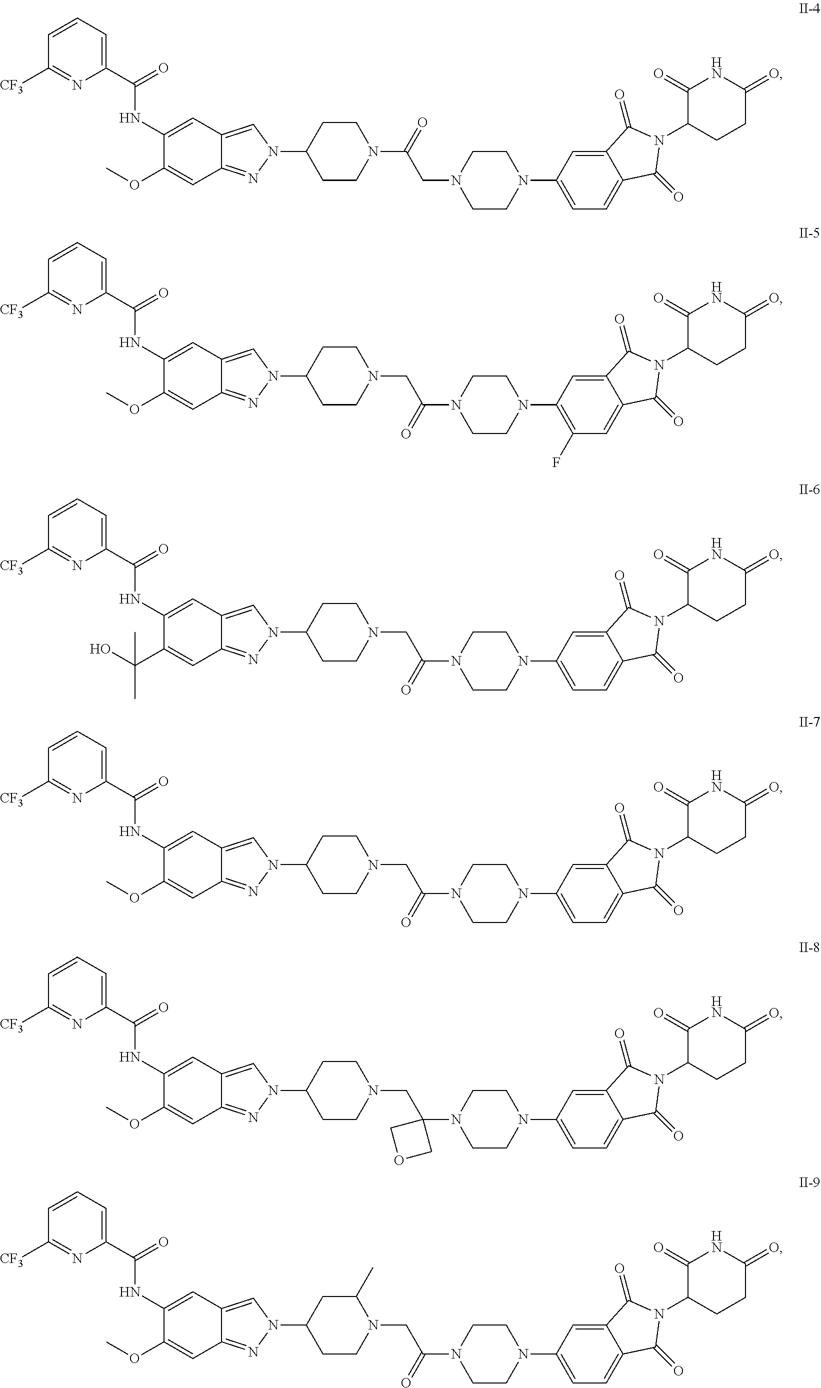

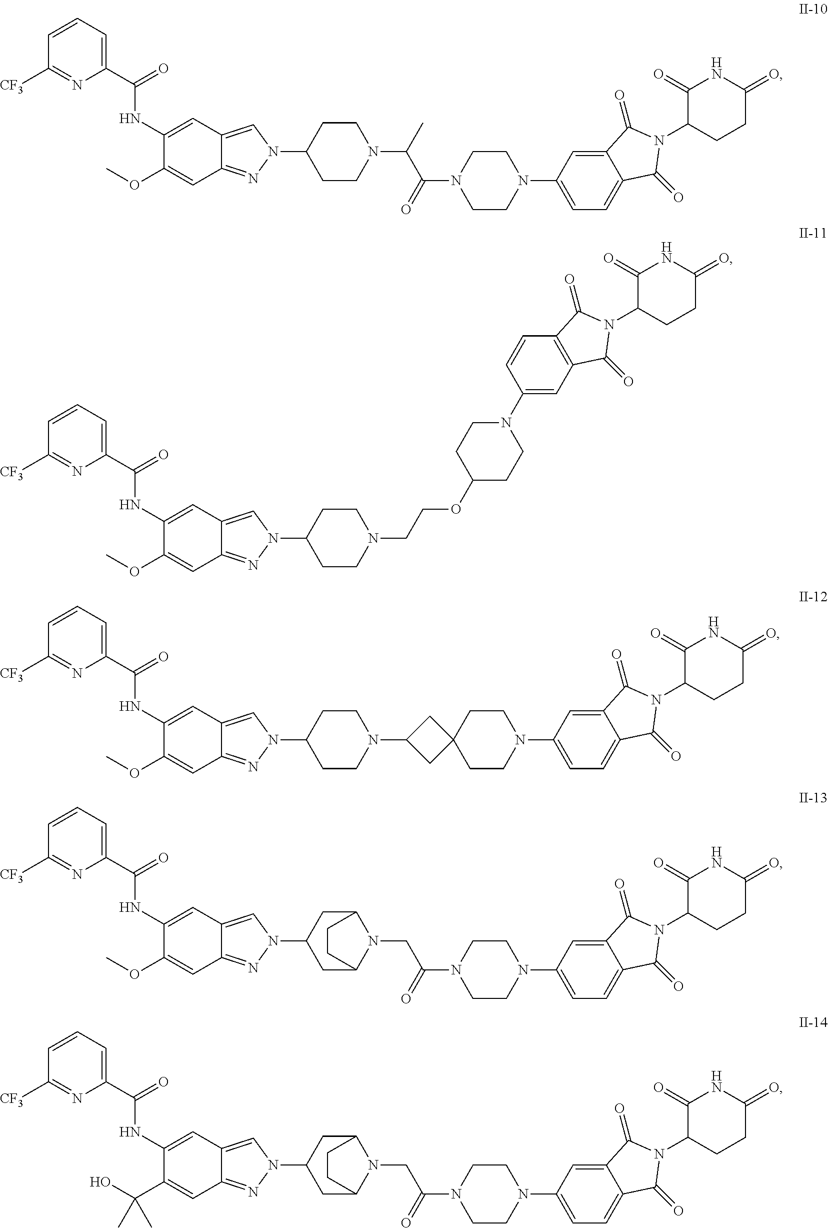

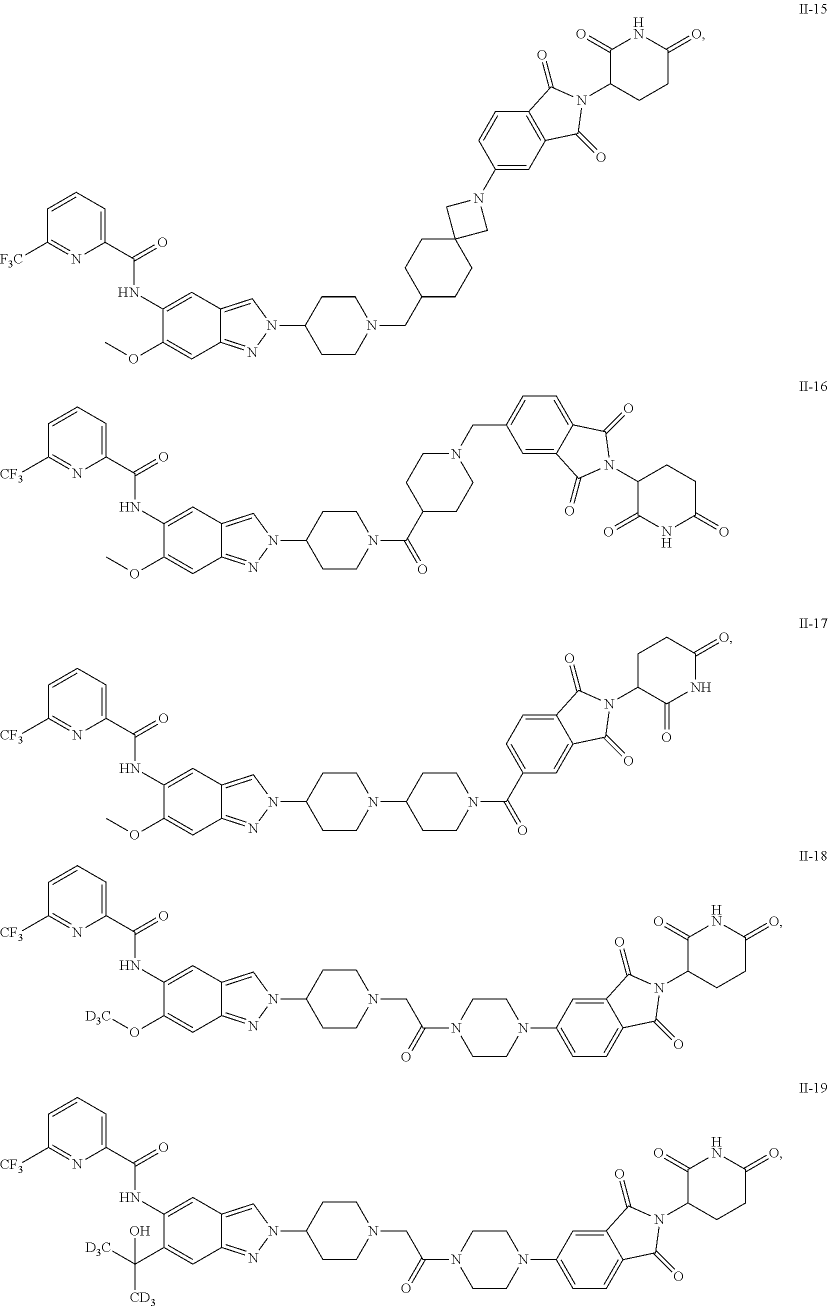

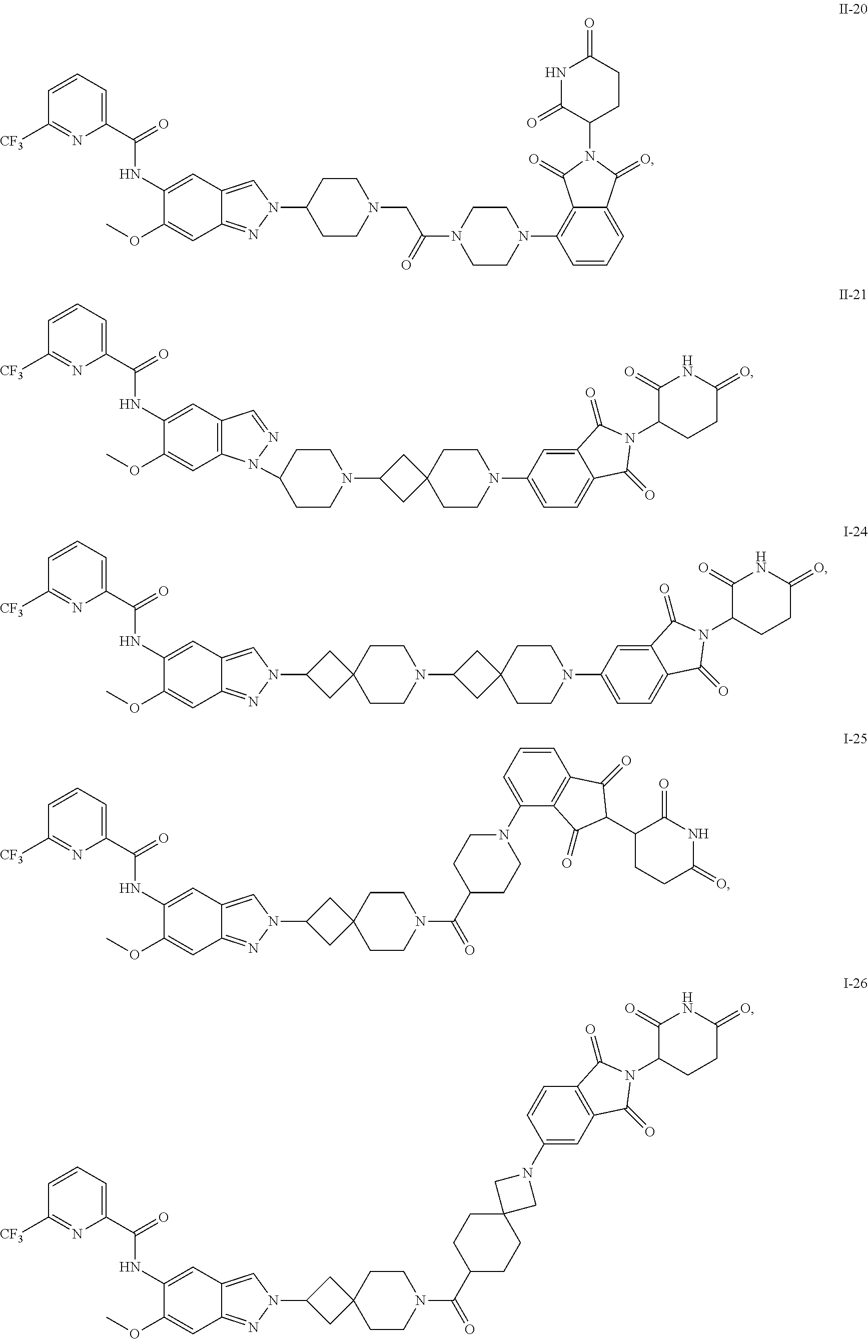

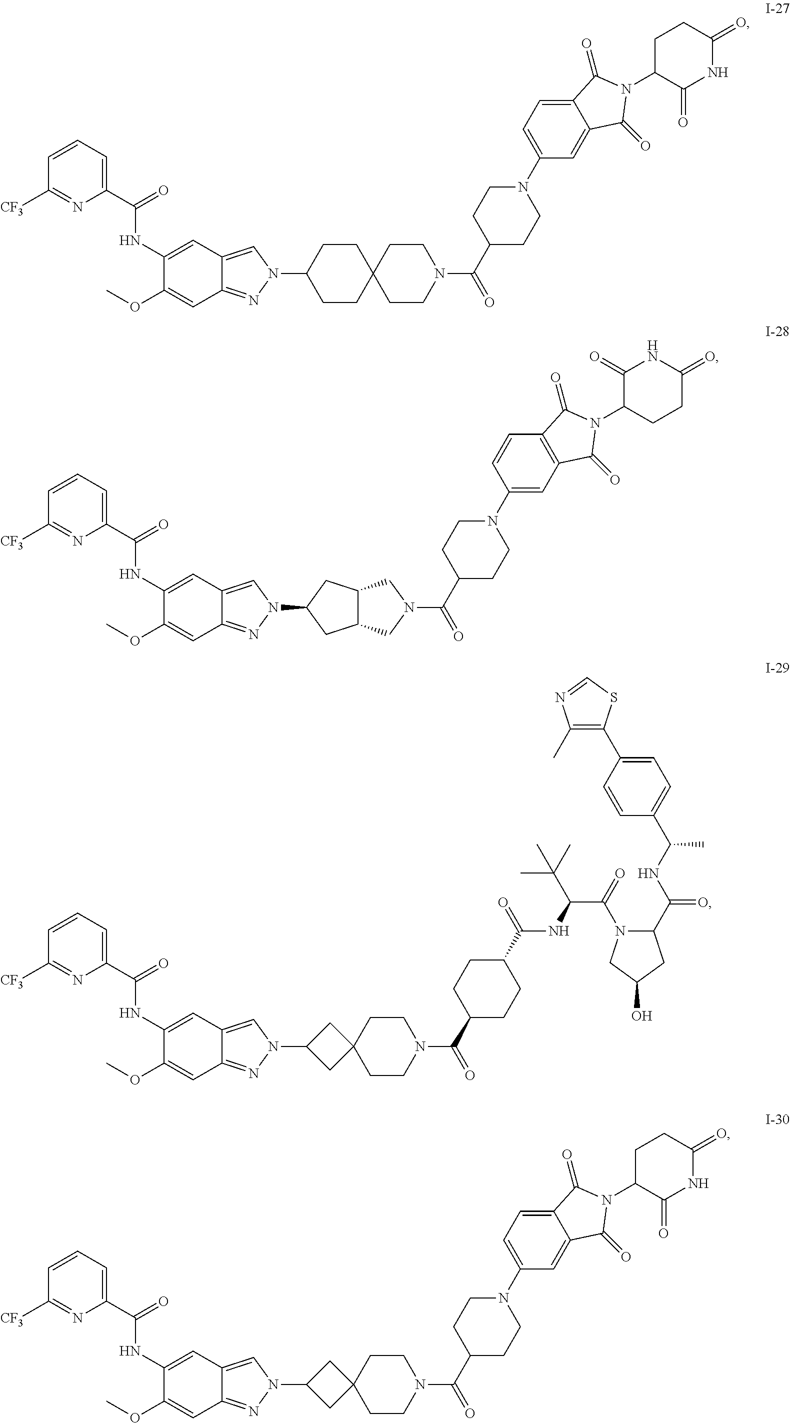

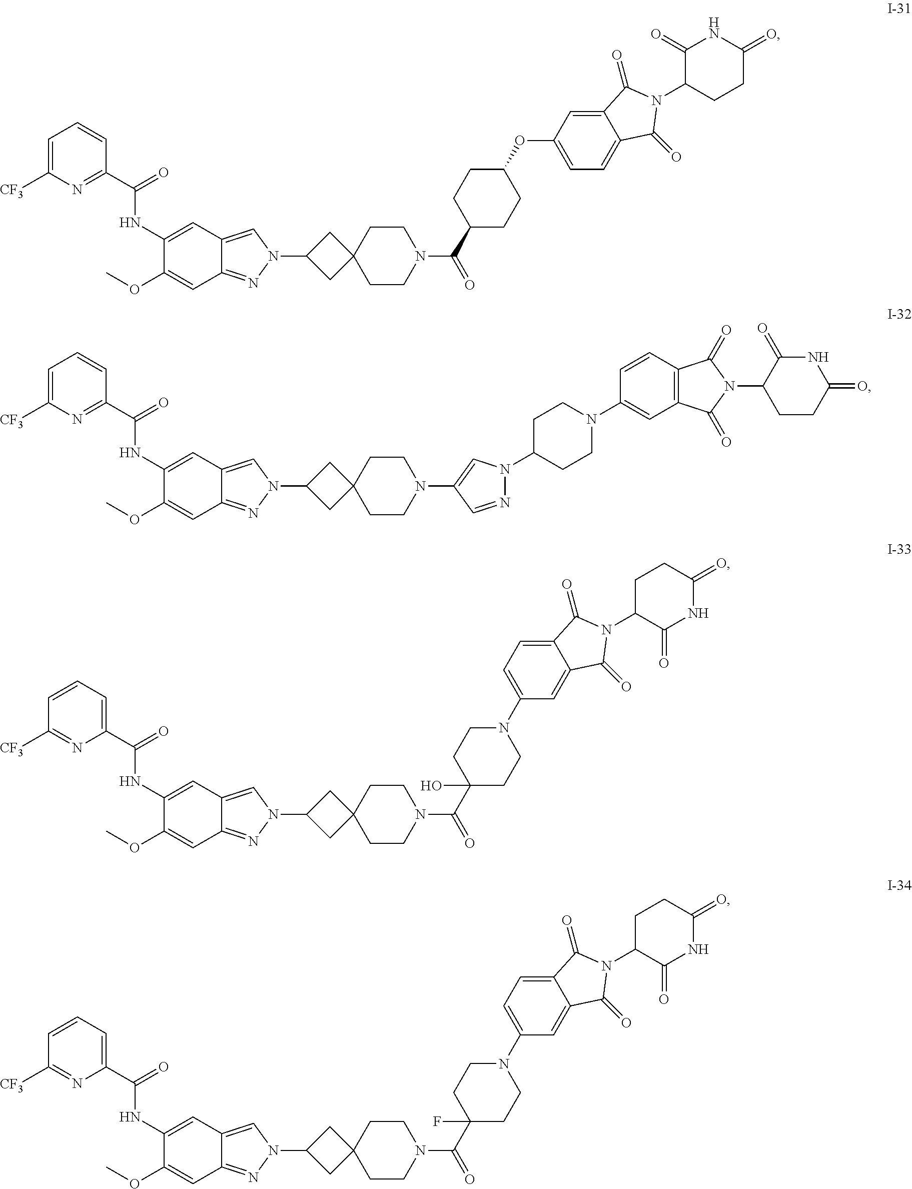 [SMILES: COc1cc2nn(C3CC4(CCN(C(=O)[C@H]5CC[C@H](Oc6ccc7c(c6)C(=O)N(C6CCC(=O)NC6=O)C7=O)CC5)CC4)C3)cc2cc1NC(=O)c1cccc(C(F)(F)F)n1 ; COc1cc2nn(C3CC4(CCN(c5cnn(C6CCN(c7ccc8c(c7)C(=O)N(C7CCC(=O)NC7=O)C8=O)CC6)c5)CC4)C3)cc2cc1NC(=O)c1cccc(C(F)(F)F)n1 ; COc1cc2nn(C3CC4(CCN(C(=O)C5(O)CCN(c6ccc7c(c6)C(=O)N(C6CCC(=O)NC6=O)C7=O)CC5)CC4)C3)cc2cc1NC(=O)c1cccc(C(F)(F)F)n1 ; COc1cc2nn(C3CC4(CCN(C(=O)C5(F)CCN(c6ccc7c(c6)C(=O)N(C6CCC(=O)NC6=O)C7=O)CC5)CC4)C3)cc2cc1NC(=O)c1cccc(C(F)(F)F)n1]

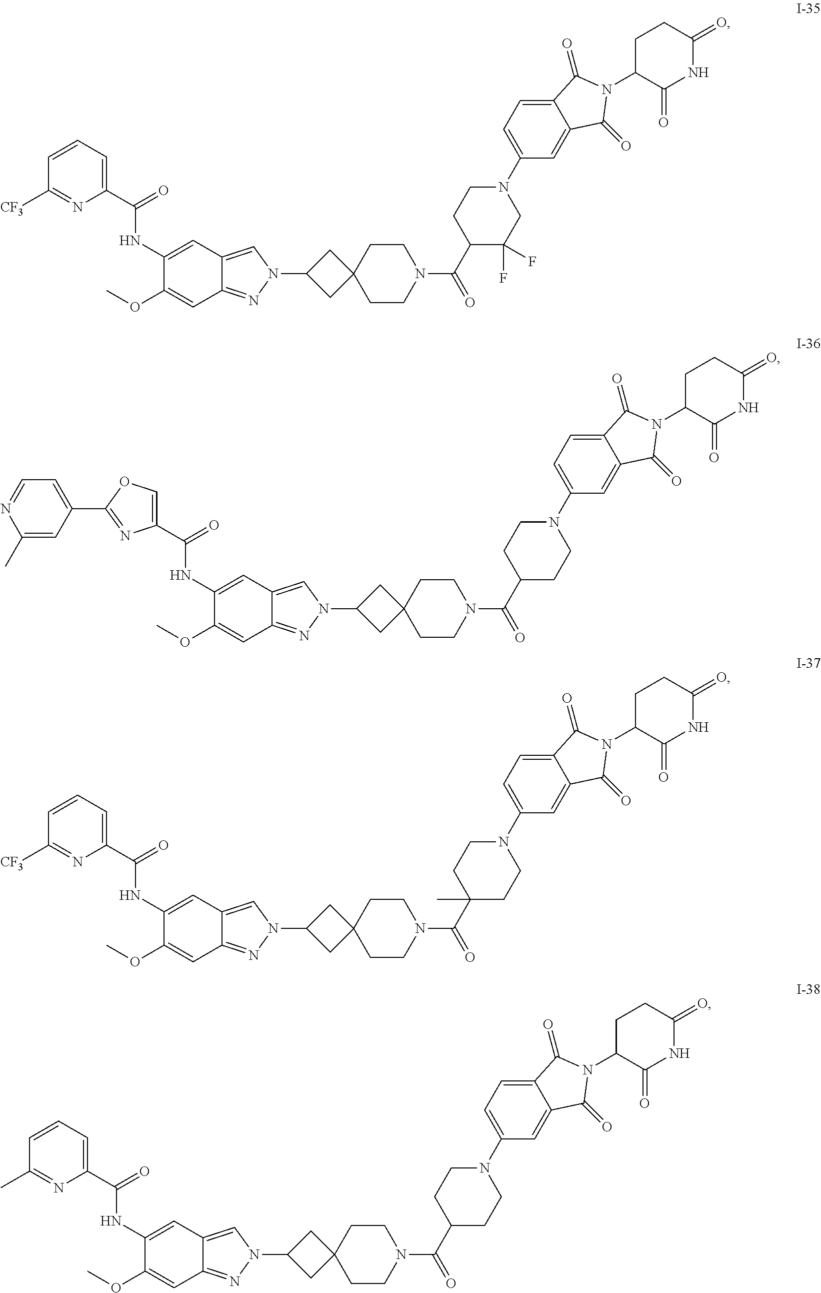 [SMILES: COc1cc2nn(C3CC4(CCN(C(=O)C5CCN(c6ccc7c(c6)C(=O)N(C6CCC(=O)NC6=O)C7=O)CC5(F)F)CC4)C3)cc2cc1NC(=O)c1cccc(C(F)(F)F)n1 ; COc1cc2nn(C3CC4(CCN(C(=O)C5CCN(c6ccc7c(c6)C(=O)N(C6CCC(=O)NC6=O)C7=O)CC5)CC4)C3)cc2cc1NC(=O)c1coc(-c2ccnc(C)c2)n1 ; COc1cc2nn(C3CC4(CCN(C(=O)C5(C)CCN(c6ccc7c(c6)C(=O)N(C6CCC(=O)NC6=O)C7=O)CC5)CC4)C3)cc2cc1NC(=O)c1cccc(C(F)(F)F)n1 ; COc1cc2nn(C3CC4(CCN(C(=O)C5CCN(c6ccc7c(c6)C(=O)N(C6CCC(=O)NC6=O)C7=O)CC5)CC4)C3)cc2cc1NC(=O)c1cccc(C)n1]

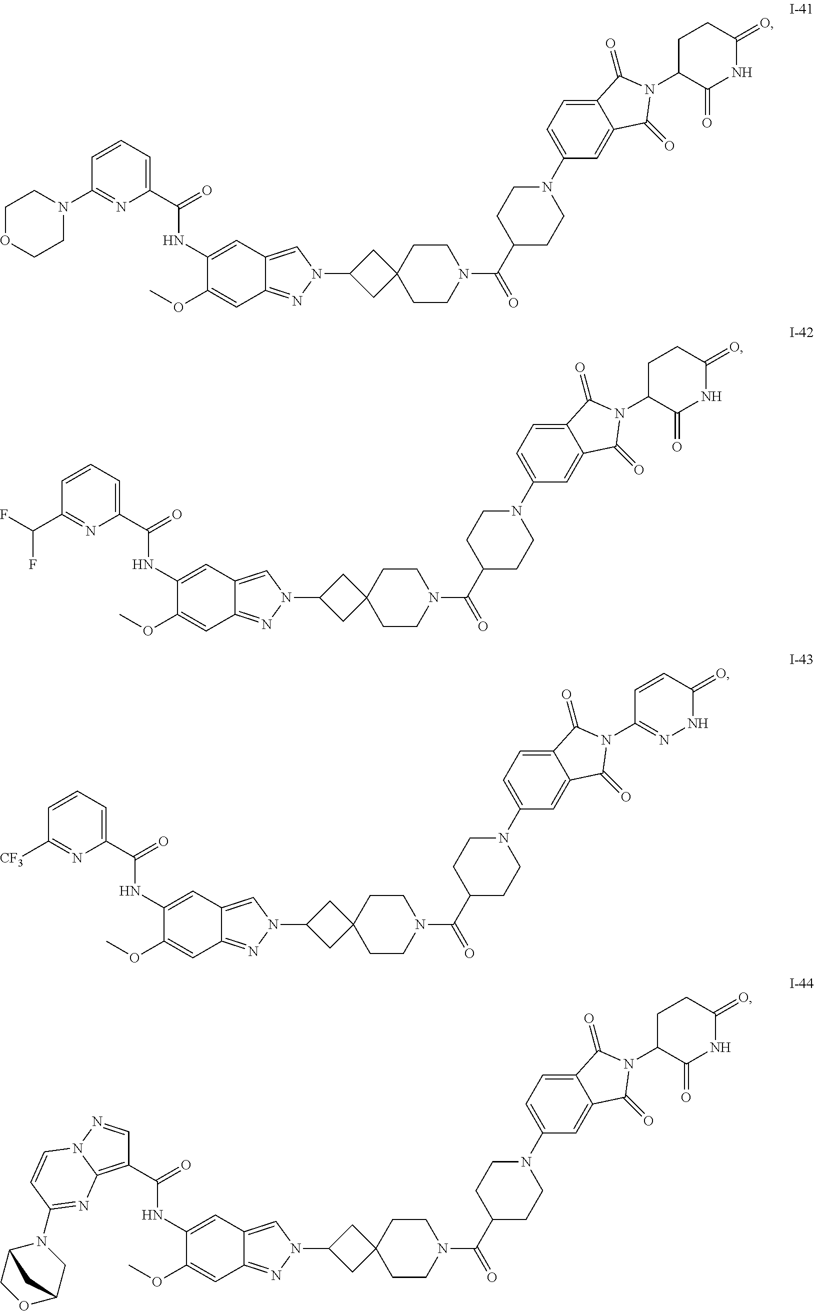 [SMILES: COc1cc2nn(C3CC4(CCN(C(=O)C5CCN(c6ccc7c(c6)C(=O)N(C6CCC(=O)NC6=O)C7=O)CC5)CC4)C3)cc2cc1NC(=O)c1cccc(N2CCOCC2)n1 ; COc1cc2nn(C3CC4(CCN(C(=O)C5CCN(c6ccc7c(c6)C(=O)N(C6CCC(=O)NC6=O)C7=O)CC5)CC4)C3)cc2cc1NC(=O)c1cccc(C(F)F)n1 ; COc1cc2nn(C3CC4(CCN(C(=O)C5CCN(c6ccc7c(c6)C(=O)N(c6ccc(=O)[nH]n6)C7=O)CC5)CC4)C3)cc2cc1NC(=O)c1cccc(C(F)(F)F)n1 ; COc1cc2nn(C3CC4(CCN(C(=O)C5CCN(c6ccc7c(c6)C(=O)N(C6CCC(=O)NC6=O)C7=O)CC5)CC4)C3)cc2cc1NC(=O)c1cnn2ccc(N3C[C@H]4C[C@@H]3CO4)nc12]

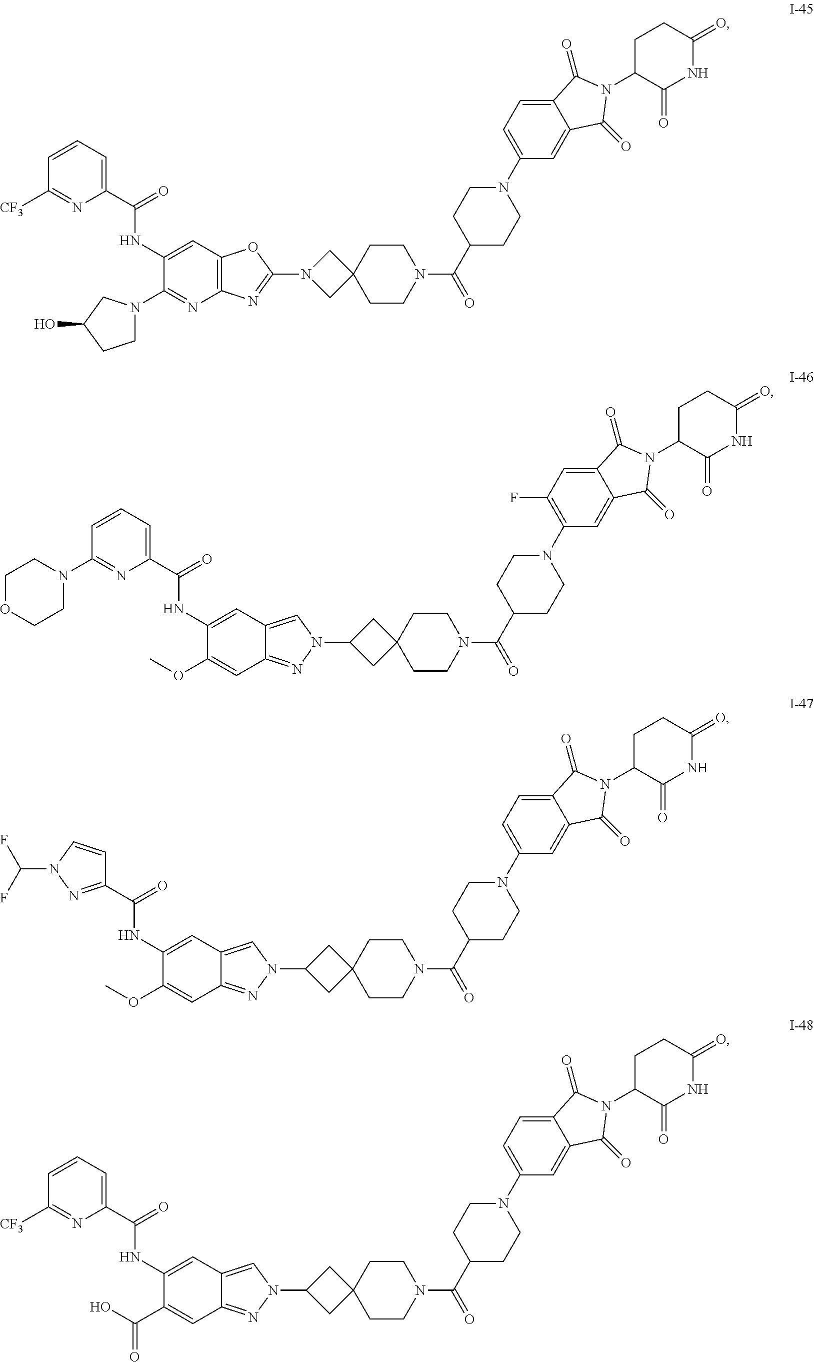 [SMILES: O=C1CCC(N2C(=O)c3ccc(N4CCC(C(=O)N5CCC6(CC5)CN(c5nc7nc(N8CC[C@@H](O)C8)c(NC(=O)c8cccc(C(F)(F)F)n8)cc7o5)C6)CC4)cc3C2=O)C(=O)N1 ; COc1cc2nn(C3CC4(CCN(C(=O)C5CCN(c6cc7c(cc6F)C(=O)N(C6CCC(=O)NC6=O)C7=O)CC5)CC4)C3)cc2cc1NC(=O)c1cccc(N2CCOCC2)n1 ; COc1cc2nn(C3CC4(CCN(C(=O)C5CCN(c6ccc7c(c6)C(=O)N(C6CCC(=O)NC6=O)C7=O)CC5)CC4)C3)cc2cc1NC(=O)c1ccn(C(F)F)n1 ; O=C1CCC(N2C(=O)c3ccc(N4CCC(C(=O)N5CCC6(CC5)CC(n5cc7cc(NC(=O)c8cccc(C(F)(F)F)n8)c(C(=O)O)cc7n5)C6)CC4)cc3C2=O)C(=O)N1]

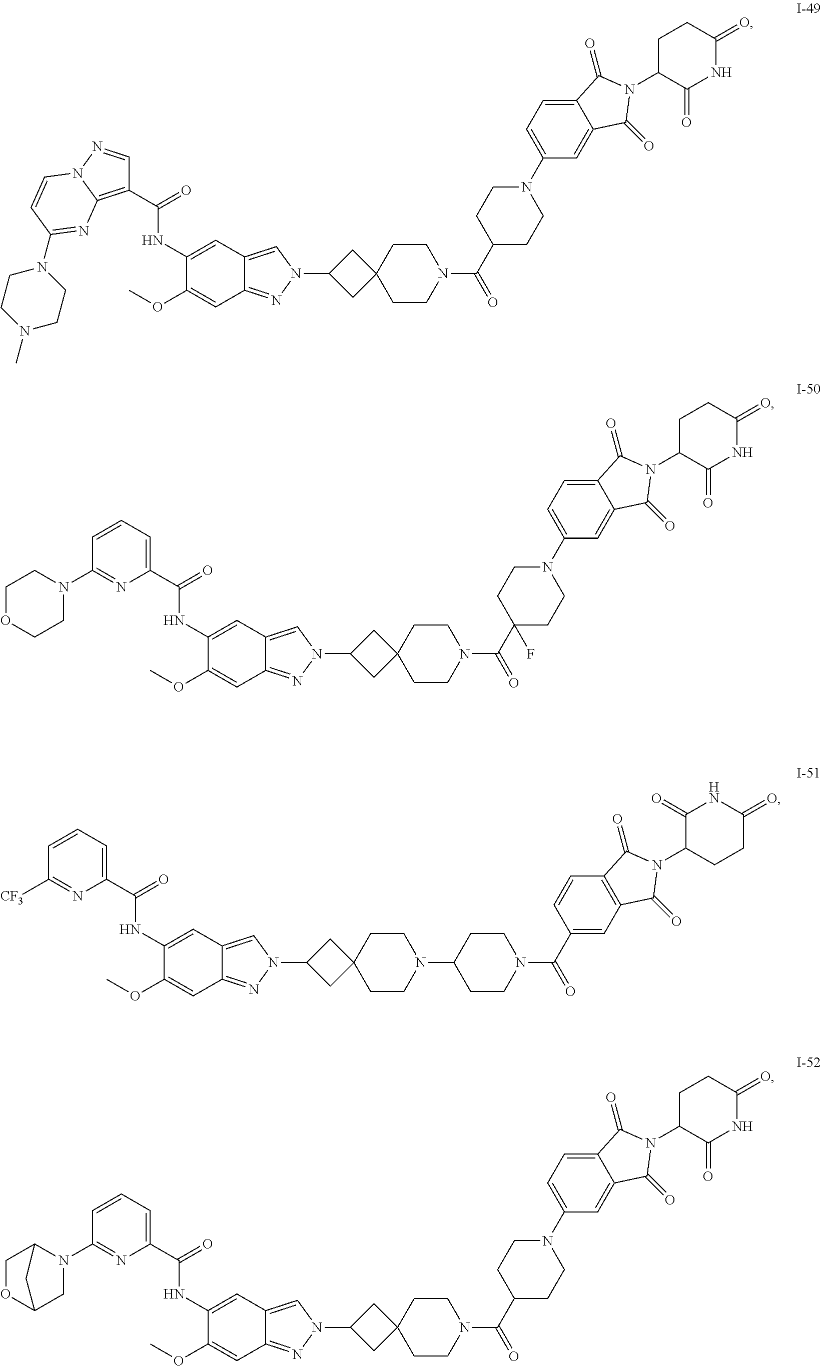 [SMILES: COc1cc2nn(C3CC4(CCN(C(=O)C5CCN(c6ccc7c(c6)C(=O)N(C6CCC(=O)NC6=O)C7=O)CC5)CC4)C3)cc2cc1NC(=O)c1cnn2ccc(N3CCN(C)CC3)nc12 ; COc1cc2nn(C3CC4(CCN(C(=O)C5(F)CCN(c6ccc7c(c6)C(=O)N(C6CCC(=O)NC6=O)C7=O)CC5)CC4)C3)cc2cc1NC(=O)c1cccc(N2CCOCC2)n1 ; COc1cc2nn(C3CC4(CCN(C5CCN(C(=O)c6ccc7c(c6)C(=O)N(C6CCC(=O)NC6=O)C7=O)CC5)CC4)C3)cc2cc1NC(=O)c1cccc(C(F)(F)F)n1 ; COc1cc2nn(C3CC4(CCN(C(=O)C5CCN(c6ccc7c(c6)C(=O)N(C6CCC(=O)NC6=O)C7=O)CC5)CC4)C3)cc2cc1NC(=O)c1cccc(N2CC3CC2CO3)n1]

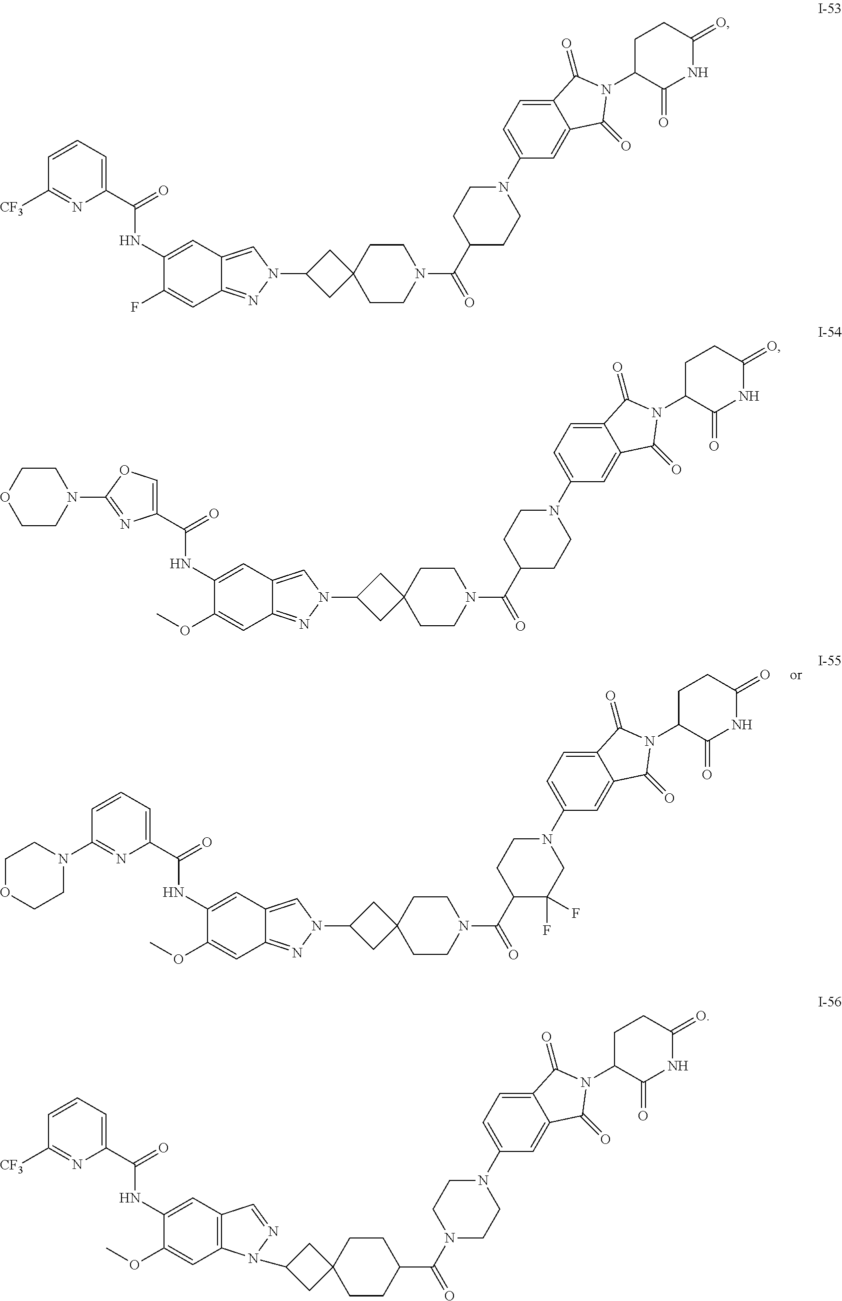 [SMILES: O=C1CCC(N2C(=O)c3ccc(N4CCC(C(=O)N5CCC6(CC5)CC(n5cc7cc(NC(=O)c8cccc(C(F)(F)F)n8)c(F)cc7n5)C6)CC4)cc3C2=O)C(=O)N1 ; COc1cc2nn(C3CC4(CCN(C(=O)C5CCN(c6ccc7c(c6)C(=O)N(C6CCC(=O)NC6=O)C7=O)CC5)CC4)C3)cc2cc1NC(=O)c1coc(N2CCOCC2)n1 ; COc1cc2nn(C3CC4(CCN(C(=O)C5CCN(c6ccc7c(c6)C(=O)N(C6CCC(=O)NC6=O)C7=O)CC5(F)F)CC4)C3)cc2cc1NC(=O)c1cccc(N2CCOCC2)n1 ; COc1cc2c(cnn2C2CC3(CCC(C(=O)N4CCN(c5ccc6c(c5)C(=O)N(C5CCC(=O)NC5=O)C6=O)CC4)CC3)C2)cc1NC(=O)c1cccc(C(F)(F)F)n1]

In a preferred embodiment,

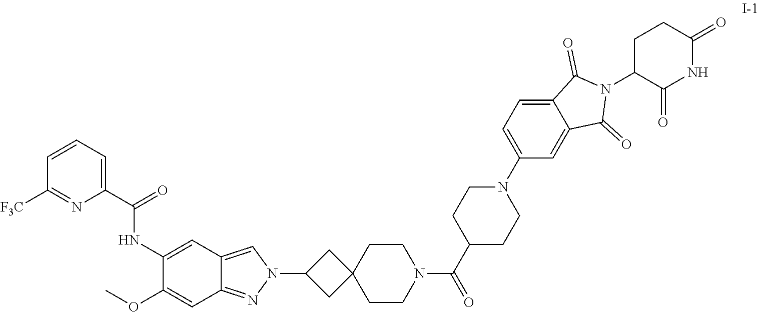 [SMILES: COc1cc2nn(C3CC4(CCN(C(=O)C5CCN(c6ccc7c(c6)C(=O)N(C6CCC(=O)NC6=O)C7=O)CC5)CC4)C3)cc2cc1NC(=O)c1cccc(C(F)(F)F)n1]

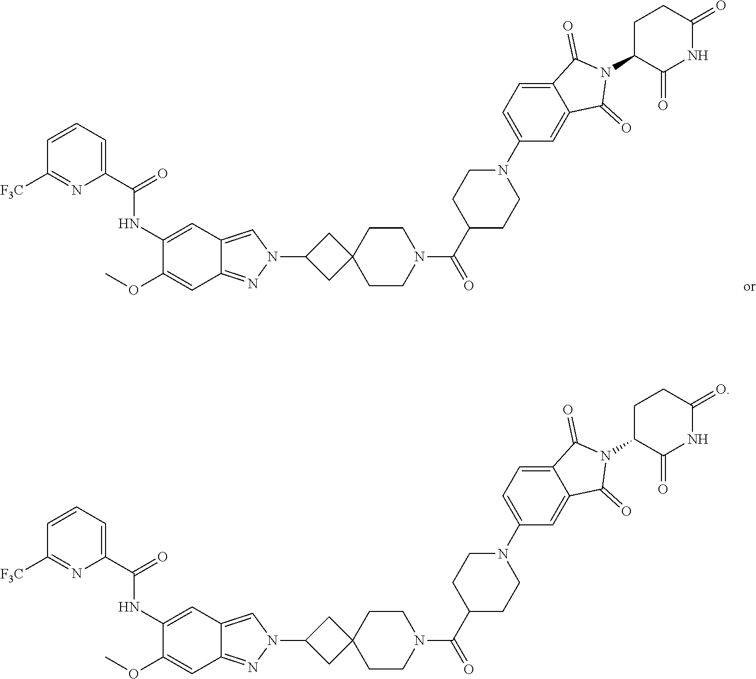 [SMILES: COc1cc2nn(C3CC4(CCN(C(=O)C5CCN(c6ccc7c(c6)C(=O)N([C@H]6CCC(=O)NC6=O)C7=O)CC5)CC4)C3)cc2cc1NC(=O)c1cccc(C(F)(F)F)n1 ; COc1cc2nn(C3CC4(CCN(C(=O)C5CCN(c6ccc7c(c6)C(=O)N([C@@H]6CCC(=O)NC6=O)C7=O)CC5)CC4)C3)cc2cc1NC(=O)c1cccc(C(F)(F)F)n1]

In a preferred embodiment,

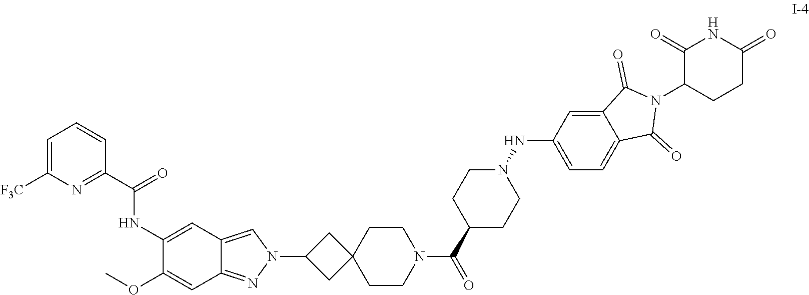

is

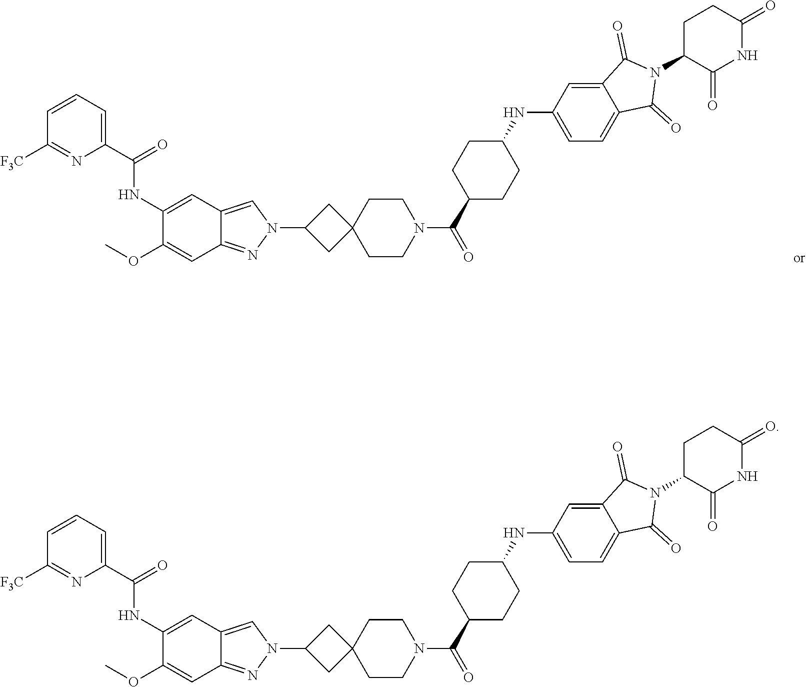 [SMILES: COc1cc2nn(C3CC4(CCN(C(=O)[C@H]5CC[C@H](Nc6ccc7c(c6)C(=O)N([C@H]6CCC(=O)NC6=O)C7=O)CC5)CC4)C3)cc2cc1NC(=O)c1cccc(C(F)(F)F)n1 ; COc1cc2nn(C3CC4(CCN(C(=O)[C@H]5CC[C@H](Nc6ccc7c(c6)C(=O)N([C@@H]6CCC(=O)NC6=O)C7=O)CC5)CC4)C3)cc2cc1NC(=O)c1cccc(C(F)(F)F)n1]

In a preferred embodiment,

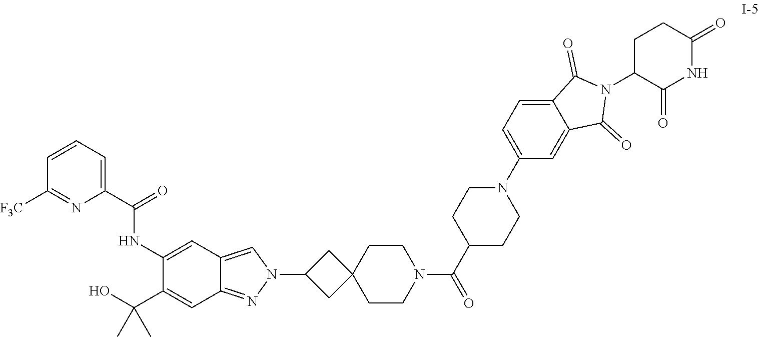 [SMILES: CC(C)(O)c1cc2nn(C3CC4(CCN(C(=O)C5CCN(c6ccc7c(c6)C(=O)N(C6CCC(=O)NC6=O)C7=O)CC5)CC4)C3)cc2cc1NC(=O)c1cccc(C(F)(F)F)n1]

is

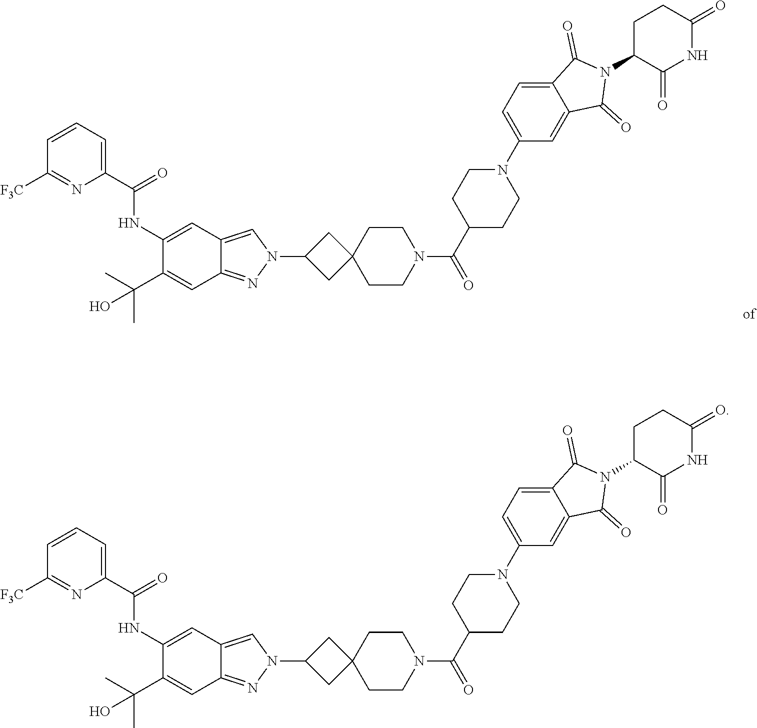 [SMILES: CC(C)(O)c1cc2nn(C3CC4(CCN(C(=O)C5CCN(c6ccc7c(c6)C(=O)N([C@H]6CCC(=O)NC6=O)C7=O)CC5)CC4)C3)cc2cc1NC(=O)c1cccc(C(F)(F)F)n1 ; CC(C)(O)c1cc2nn(C3CC4(CCN(C(=O)C5CCN(c6ccc7c(c6)C(=O)N([C@@H]6CCC(=O)NC6=O)C7=O)CC5)CC4)C3)cc2cc1NC(=O)c1cccc(C(F)(F)F)n1]

In a preferred embodiment,

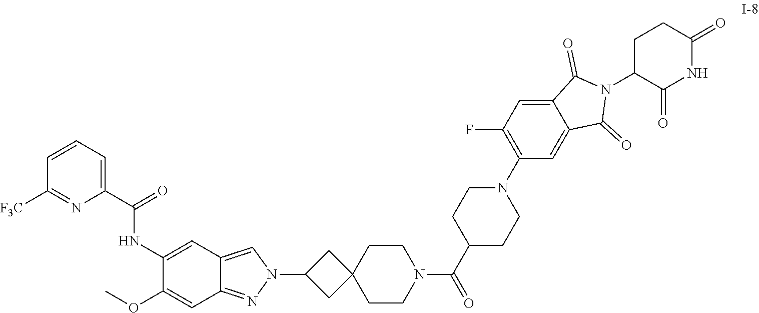 [SMILES: COc1cc2nn(C3CC4(CCN(C(=O)C5CCN(c6cc7c(cc6F)C(=O)N(C6CCC(=O)NC6=O)C7=O)CC5)CC4)C3)cc2cc1NC(=O)c1cccc(C(F)(F)F)n1]

is

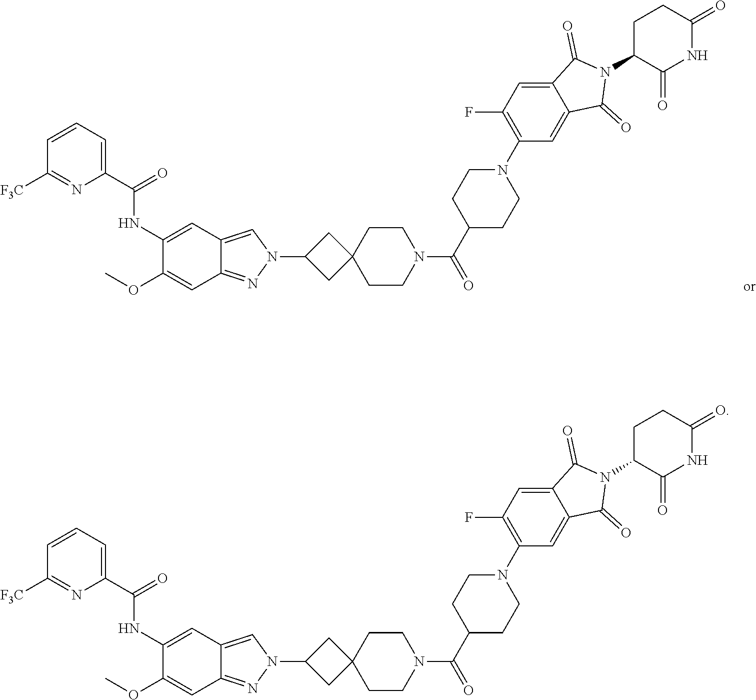 [SMILES: COc1cc2nn(C3CC4(CCN(C(=O)C5CCN(c6cc7c(cc6F)C(=O)N([C@H]6CCC(=O)NC6=O)C7=O)CC5)CC4)C3)cc2cc1NC(=O)c1cccc(C(F)(F)F)n1 ; COc1cc2nn(C3CC4(CCN(C(=O)C5CCN(c6cc7c(cc6F)C(=O)N([C@@H]6CCC(=O)NC6=O)C7=O)CC5)CC4)C3)cc2cc1NC(=O)c1cccc(C(F)(F)F)n1]

In a preferred embodiment,

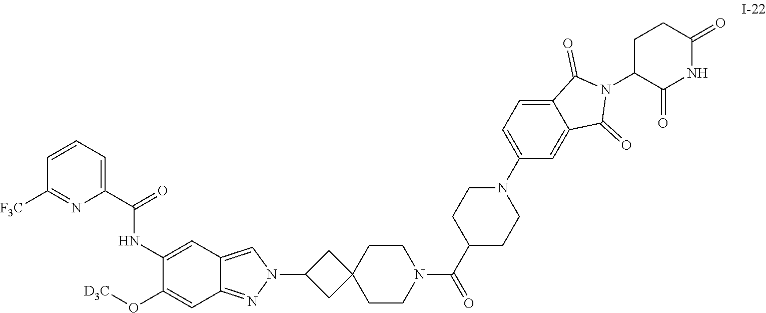 [SMILES: [2H]C([2H])([2H])Oc1cc2nn(C3CC4(CCN(C(=O)C5CCN(c6ccc7c(c6)C(=O)N(C6CCC(=O)NC6=O)C7=O)CC5)CC4)C3)cc2cc1NC(=O)c1cccc(C(F)(F)F)n1]

is

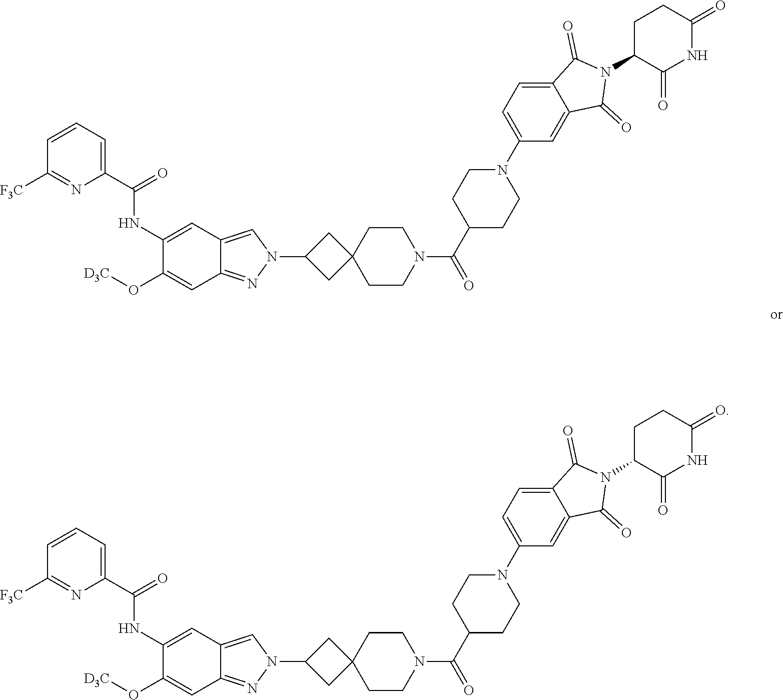 [SMILES: [2H]C([2H])([2H])Oc1cc2nn(C3CC4(CCN(C(=O)C5CCN(c6ccc7c(c6)C(=O)N([C@H]6CCC(=O)NC6=O)C7=O)CC5)CC4)C3)cc2cc1NC(=O)c1cccc(C(F)(F)F)n1 ; [2H]C([2H])([2H])Oc1cc2nn(C3CC4(CCN(C(=O)C5CCN(c6ccc7c(c6)C(=O)N([C@@H]6CCC(=O)NC6=O)C7=O)CC5)CC4)C3)cc2cc1NC(=O)c1cccc(C(F)(F)F)n1]

In a preferred embodiment,

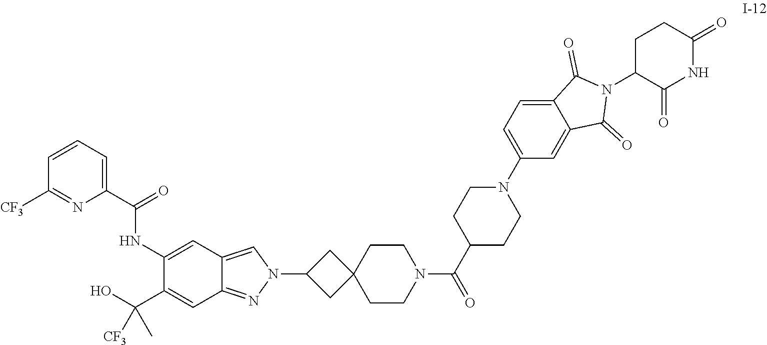 [SMILES: CC(O)(c1cc2nn(C3CC4(CCN(C(=O)C5CCN(c6ccc7c(c6)C(=O)N(C6CCC(=O)NC6=O)C7=O)CC5)CC4)C3)cc2cc1NC(=O)c1cccc(C(F)(F)F)n1)C(F)(F)F]

is

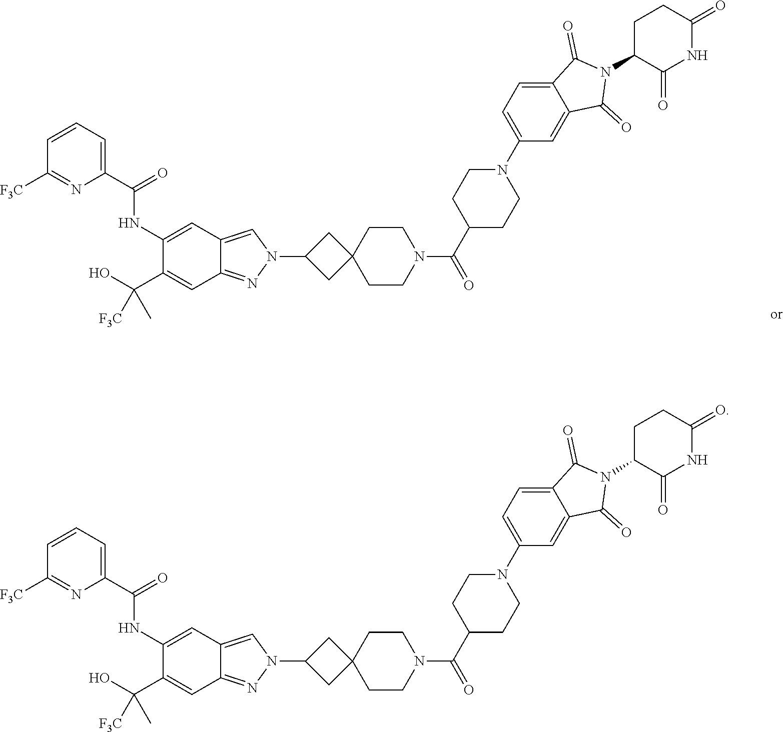 [SMILES: CC(O)(c1cc2nn(C3CC4(CCN(C(=O)C5CCN(c6ccc7c(c6)C(=O)N([C@H]6CCC(=O)NC6=O)C7=O)CC5)CC4)C3)cc2cc1NC(=O)c1cccc(C(F)(F)F)n1)C(F)(F)F ; CC(O)(c1cc2nn(C3CC4(CCN(C(=O)C5CCN(c6ccc7c(c6)C(=O)N([C@@H]6CCC(=O)NC6=O)C7=O)CC5)CC4)C3)cc2cc1NC(=O)c1cccc(C(F)(F)F)n1)C(F)(F)F]

In a preferred embodiment,

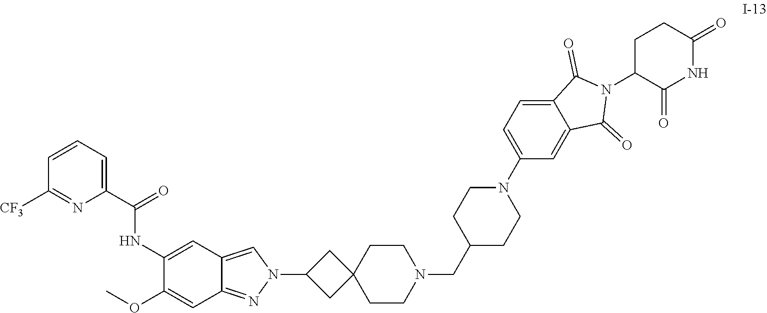 [SMILES: COc1cc2nn(C3CC4(CCN(CC5CCN(c6ccc7c(c6)C(=O)N(C6CCC(=O)NC6=O)C7=O)CC5)CC4)C3)cc2cc1NC(=O)c1cccc(C(F)(F)F)n1]

is

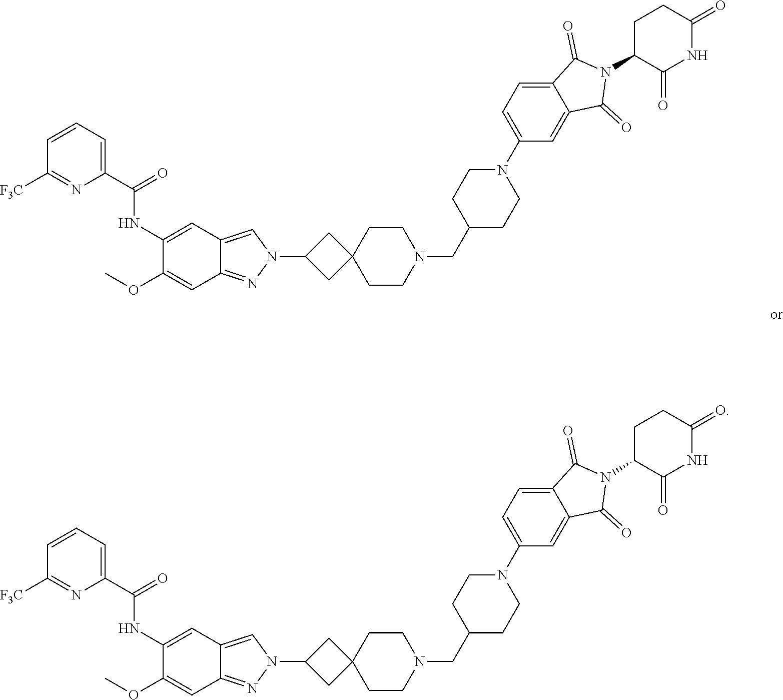 [SMILES: COc1cc2nn(C3CC4(CCN(CC5CCN(c6ccc7c(c6)C(=O)N([C@H]6CCC(=O)NC6=O)C7=O)CC5)CC4)C3)cc2cc1NC(=O)c1cccc(C(F)(F)F)n1 ; COc1cc2nn(C3CC4(CCN(CC5CCN(c6ccc7c(c6)C(=O)N([C@@H]6CCC(=O)NC6=O)C7=O)CC5)CC4)C3)cc2cc1NC(=O)c1cccc(C(F)(F)F)n1]

In a preferred embodiment,

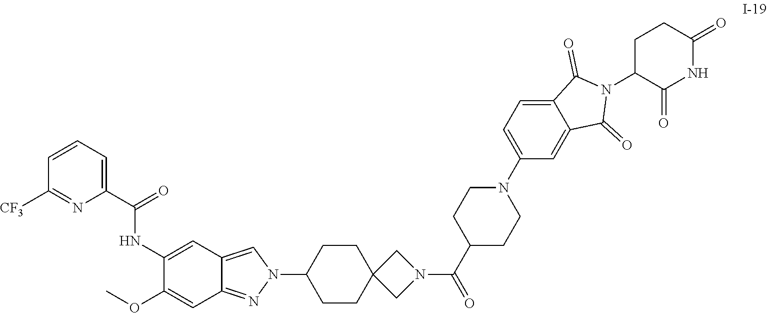 [SMILES: COc1cc2nn(C3CCC4(CC3)CN(C(=O)C3CCN(c5ccc6c(c5)C(=O)N(C5CCC(=O)NC5=O)C6=O)CC3)C4)cc2cc1NC(=O)c1cccc(C(F)(F)F)n1]

is

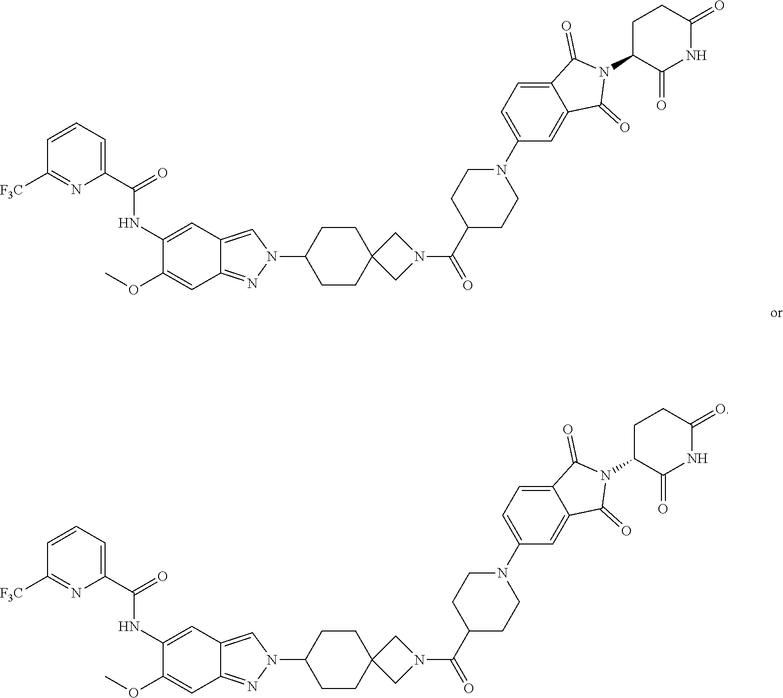 [SMILES: COc1cc2nn(C3CCC4(CC3)CN(C(=O)C3CCN(c5ccc6c(c5)C(=O)N([C@H]5CCC(=O)NC5=O)C6=O)CC3)C4)cc2cc1NC(=O)c1cccc(C(F)(F)F)n1 ; COc1cc2nn(C3CCC4(CC3)CN(C(=O)C3CCN(c5ccc6c(c5)C(=O)N([C@@H]5CCC(=O)NC5=O)C6=O)CC3)C4)cc2cc1NC(=O)c1cccc(C(F)(F)F)n1]

In a preferred embodiment,

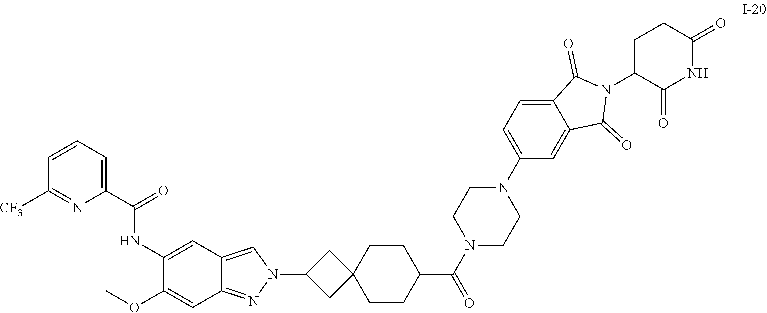 [SMILES: COc1cc2nn(C3CC4(CCC(C(=O)N5CCN(c6ccc7c(c6)C(=O)N(C6CCC(=O)NC6=O)C7=O)CC5)CC4)C3)cc2cc1NC(=O)c1cccc(C(F)(F)F)n1]

is

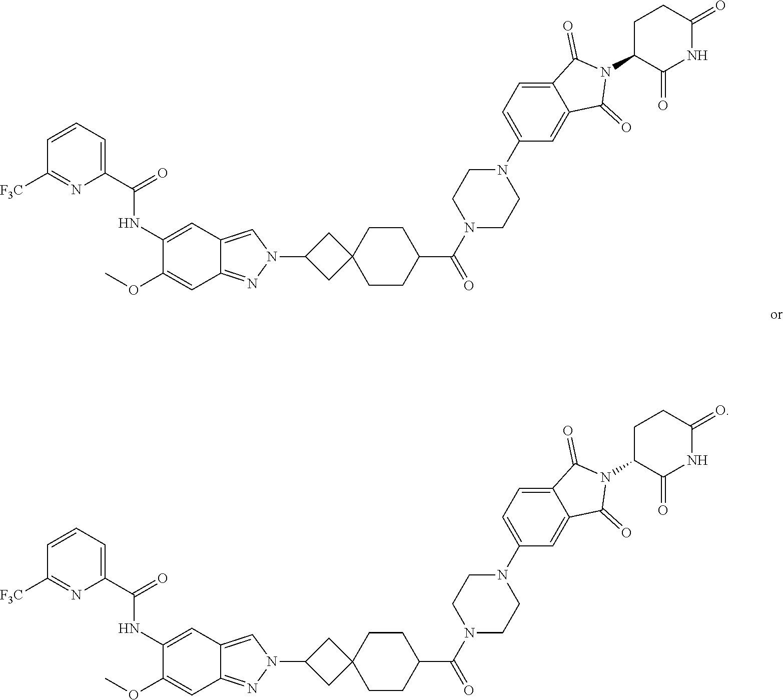 [SMILES: COc1cc2nn(C3CC4(CCC(C(=O)N5CCN(c6ccc7c(c6)C(=O)N([C@H]6CCC(=O)NC6=O)C7=O)CC5)CC4)C3)cc2cc1NC(=O)c1cccc(C(F)(F)F)n1 ; COc1cc2nn(C3CC4(CCC(C(=O)N5CCN(c6ccc7c(c6)C(=O)N([C@@H]6CCC(=O)NC6=O)C7=O)CC5)CC4)C3)cc2cc1NC(=O)c1cccc(C(F)(F)F)n1]

In a preferred embodiment,

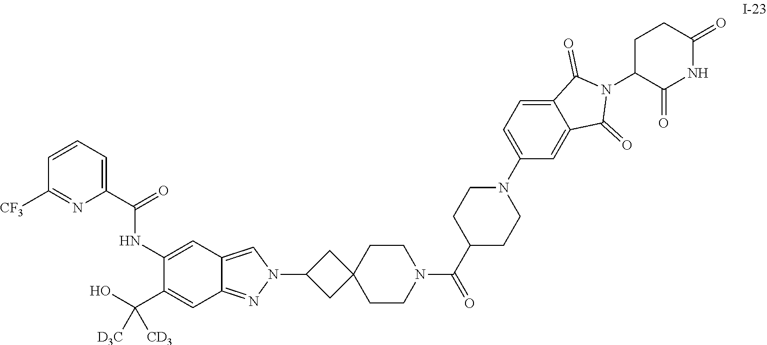 [SMILES: [2H]C([2H])([2H])C(O)(c1cc2nn(C3CC4(CCN(C(=O)C5CCN(c6ccc7c(c6)C(=O)N(C6CCC(=O)NC6=O)C7=O)CC5)CC4)C3)cc2cc1NC(=O)c1cccc(C(F)(F)F)n1)C([2H])([2H])[2H]]

is

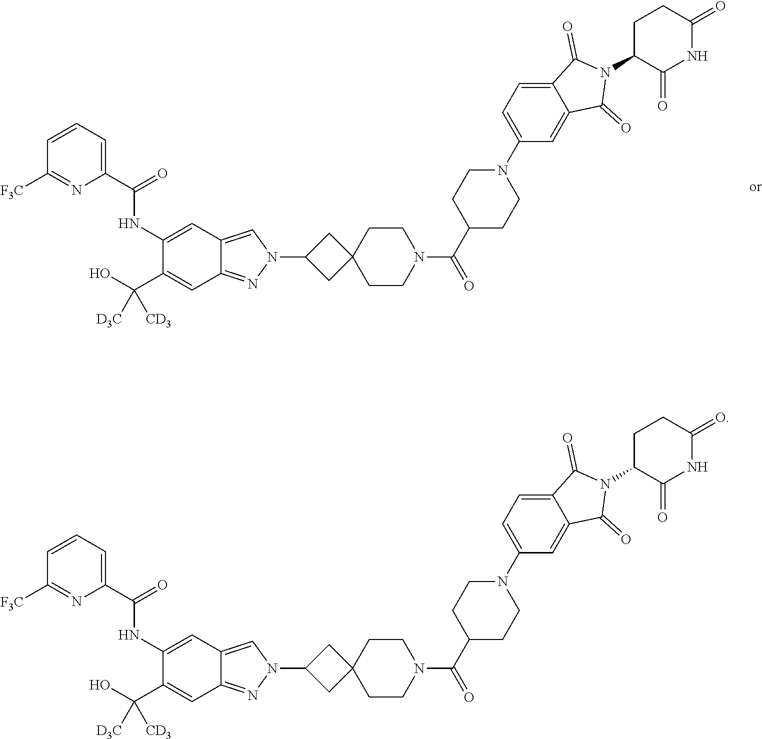 [SMILES: [2H]C([2H])([2H])C(O)(c1cc2nn(C3CC4(CCN(C(=O)C5CCN(c6ccc7c(c6)C(=O)N([C@H]6CCC(=O)NC6=O)C7=O)CC5)CC4)C3)cc2cc1NC(=O)c1cccc(C(F)(F)F)n1)C([2H])([2H])[2H] ; [2H]C([2H])([2H])C(O)(c1cc2nn(C3CC4(CCN(C(=O)C5CCN(c6ccc7c(c6)C(=O)N([C@@H]6CCC(=O)NC6=O)C7=O)CC5)CC4)C3)cc2cc1NC(=O)c1cccc(C(F)(F)F)n1)C([2H])([2H])[2H]]

In a preferred embodiment,

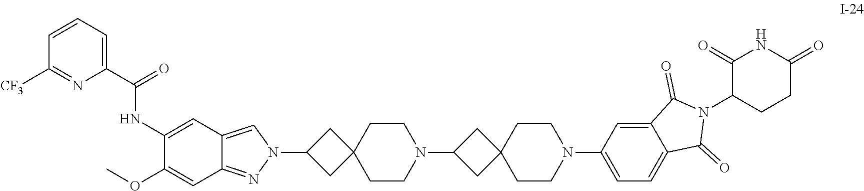 [SMILES: COc1cc2nn(C3CC4(CCN(C5CC6(CCN(c7ccc8c(c7)C(=O)N(C7CCC(=O)NC7=O)C8=O)CC6)C5)CC4)C3)cc2cc1NC(=O)c1cccc(C(F)(F)F)n1]

is

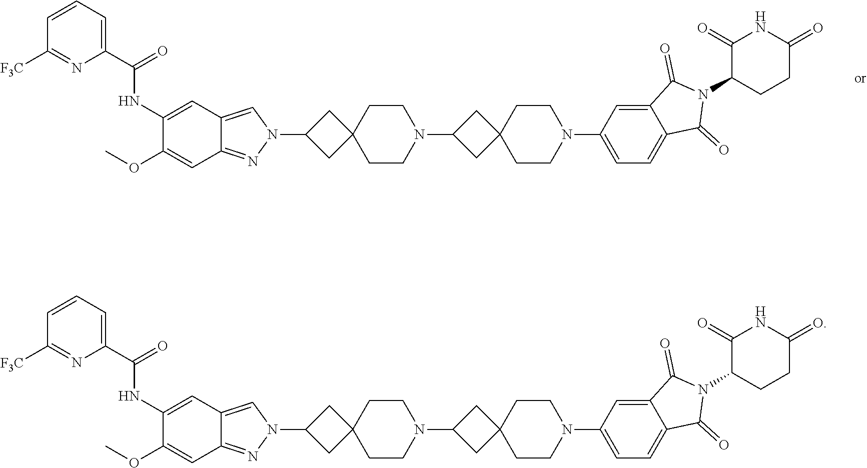 [SMILES: COc1cc2nn(C3CC4(CCN(C5CC6(CCN(c7ccc8c(c7)C(=O)N([C@@H]7CCC(=O)NC7=O)C8=O)CC6)C5)CC4)C3)cc2cc1NC(=O)c1cccc(C(F)(F)F)n1 ; COc1cc2nn(C3CC4(CCN(C5CC6(CCN(c7ccc8c(c7)C(=O)N([C@H]7CCC(=O)NC7=O)C8=O)CC6)C5)CC4)C3)cc2cc1NC(=O)c1cccc(C(F)(F)F)n1]

In a preferred embodiment,

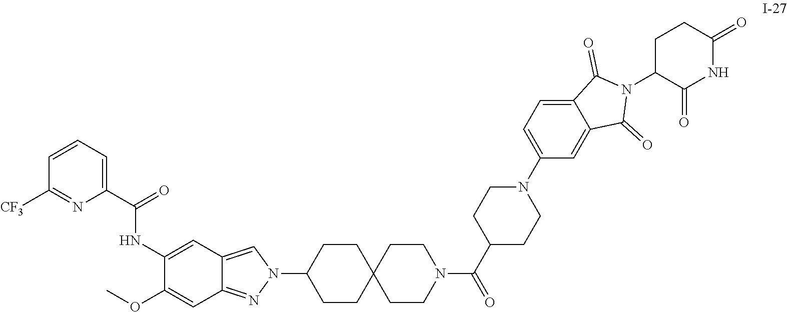 [SMILES: COc1cc2nn(C3CCC4(CC3)CCN(C(=O)C3CCN(c5ccc6c(c5)C(=O)N(C5CCC(=O)NC5=O)C6=O)CC3)CC4)cc2cc1NC(=O)c1cccc(C(F)(F)F)n1]

is

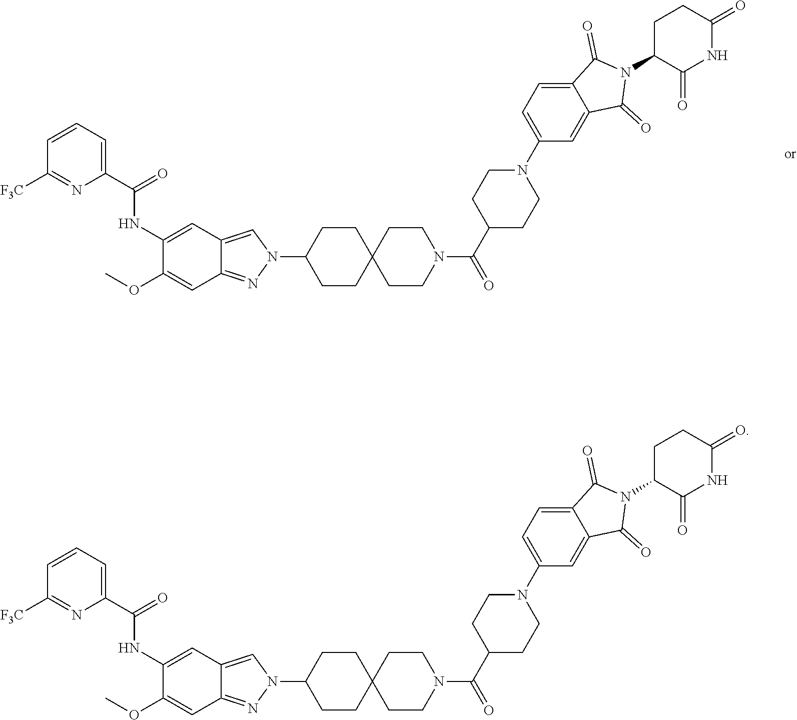 [SMILES: COc1cc2nn(C3CCC4(CC3)CCN(C(=O)C3CCN(c5ccc6c(c5)C(=O)N([C@H]5CCC(=O)NC5=O)C6=O)CC3)CC4)cc2cc1NC(=O)c1cccc(C(F)(F)F)n1 ; COc1cc2nn(C3CCC4(CC3)CCN(C(=O)C3CCN(c5ccc6c(c5)C(=O)N([C@@H]5CCC(=O)NC5=O)C6=O)CC3)CC4)cc2cc1NC(=O)c1cccc(C(F)(F)F)n1]

In a preferred embodiment,

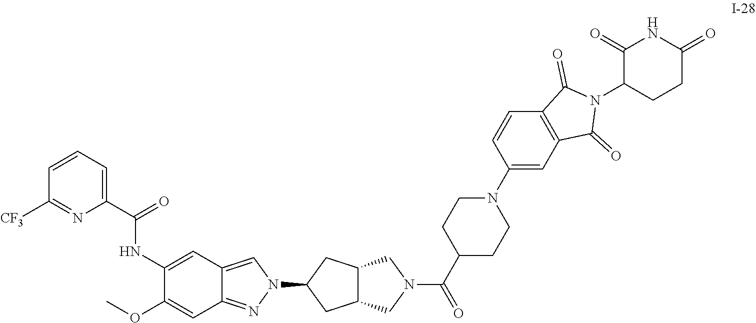 [SMILES: COc1cc2nn([C@H]3C[C@@H]4CN(C(=O)C5CCN(c6ccc7c(c6)C(=O)N(C6CCC(=O)NC6=O)C7=O)CC5)C[C@@H]4C3)cc2cc1NC(=O)c1cccc(C(F)(F)F)n1]

is

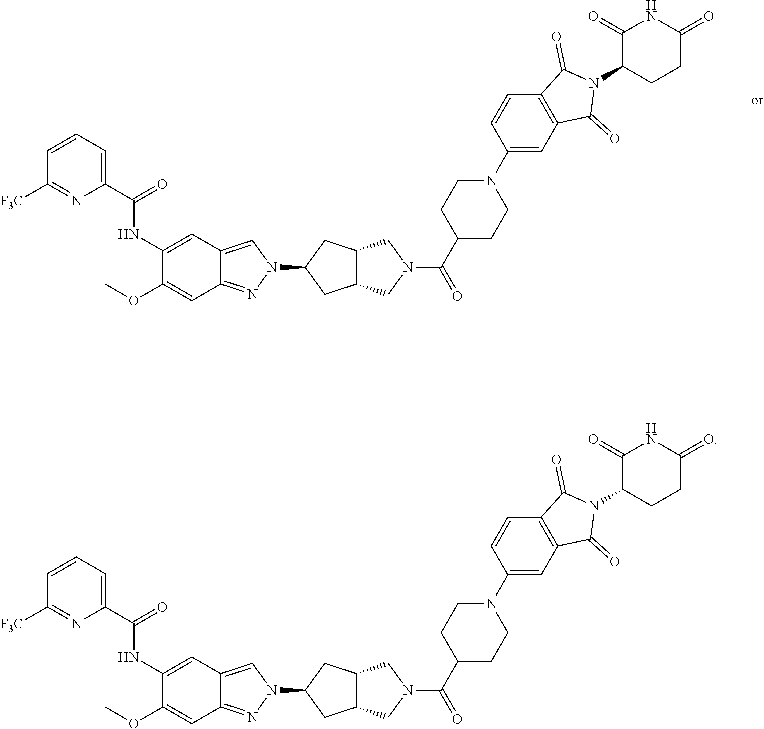 [SMILES: COc1cc2nn([C@H]3C[C@@H]4CN(C(=O)C5CCN(c6ccc7c(c6)C(=O)N([C@@H]6CCC(=O)NC6=O)C7=O)CC5)C[C@@H]4C3)cc2cc1NC(=O)c1cccc(C(F)(F)F)n1 ; COc1cc2nn([C@H]3C[C@@H]4CN(C(=O)C5CCN(c6ccc7c(c6)C(=O)N([C@H]6CCC(=O)NC6=O)C7=O)CC5)C[C@@H]4C3)cc2cc1NC(=O)c1cccc(C(F)(F)F)n1]

In a preferred embodiment,

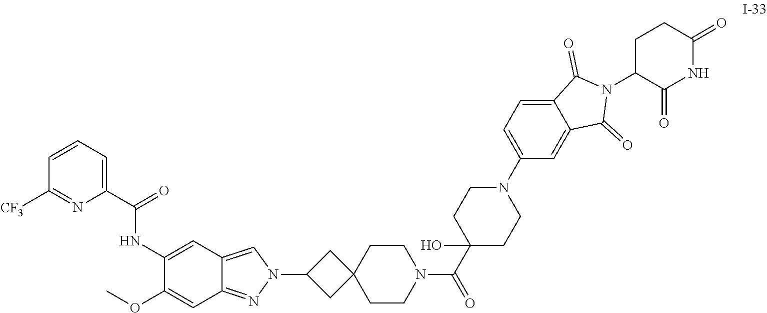 [SMILES: COc1cc2nn(C3CC4(CCN(C(=O)C5(O)CCN(c6ccc7c(c6)C(=O)N(C6CCC(=O)NC6=O)C7=O)CC5)CC4)C3)cc2cc1NC(=O)c1cccc(C(F)(F)F)n1]

is

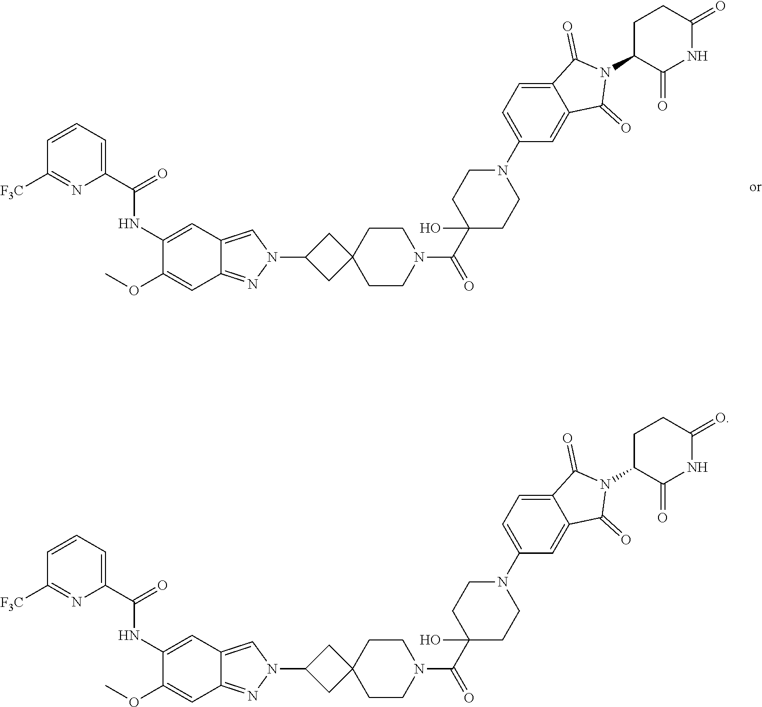 [SMILES: COc1cc2nn(C3CC4(CCN(C(=O)C5(O)CCN(c6ccc7c(c6)C(=O)N([C@H]6CCC(=O)NC6=O)C7=O)CC5)CC4)C3)cc2cc1NC(=O)c1cccc(C(F)(F)F)n1 ; COc1cc2nn(C3CC4(CCN(C(=O)C5(O)CCN(c6ccc7c(c6)C(=O)N([C@@H]6CCC(=O)NC6=O)C7=O)CC5)CC4)C3)cc2cc1NC(=O)c1cccc(C(F)(F)F)n1]

In a preferred embodiment,

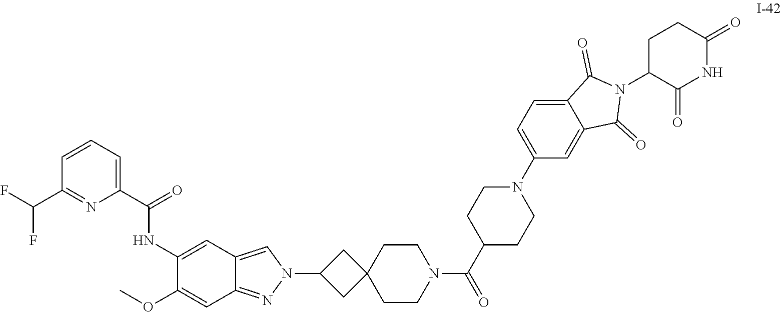 [SMILES: COc1cc2nn(C3CC4(CCN(C(=O)C5CCN(c6ccc7c(c6)C(=O)N(C6CCC(=O)NC6=O)C7=O)CC5)CC4)C3)cc2cc1NC(=O)c1cccc(C(F)F)n1]

is

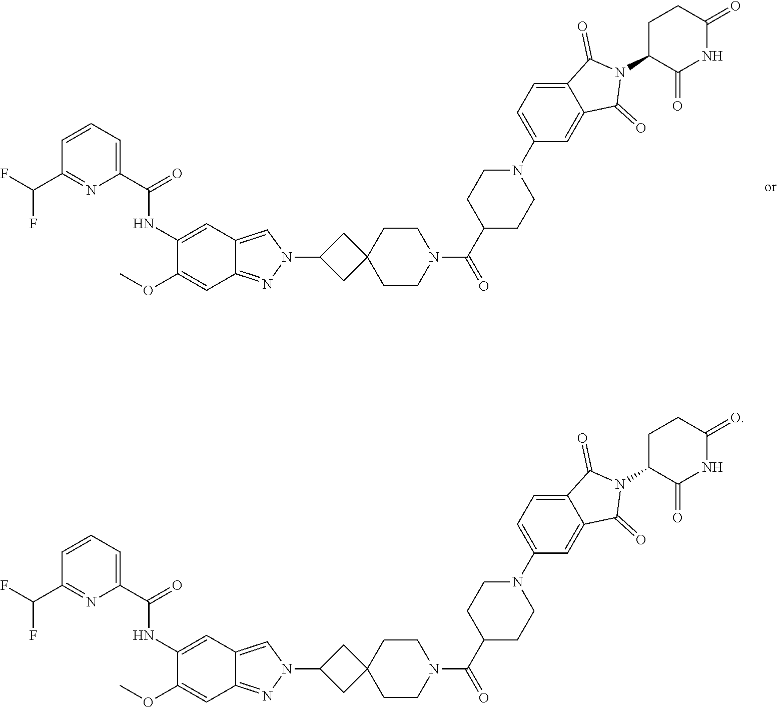 [SMILES: COc1cc2nn(C3CC4(CCN(C(=O)C5CCN(c6ccc7c(c6)C(=O)N([C@H]6CCC(=O)NC6=O)C7=O)CC5)CC4)C3)cc2cc1NC(=O)c1cccc(C(F)F)n1 ; COc1cc2nn(C3CC4(CCN(C(=O)C5CCN(c6ccc7c(c6)C(=O)N([C@@H]6CCC(=O)NC6=O)C7=O)CC5)CC4)C3)cc2cc1NC(=O)c1cccc(C(F)F)n1]

In a preferred embodiment,

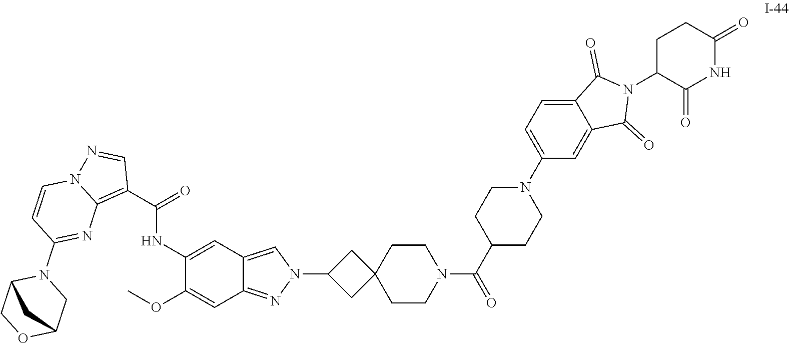 [SMILES: COc1cc2nn(C3CC4(CCN(C(=O)C5CCN(c6ccc7c(c6)C(=O)N(C6CCC(=O)NC6=O)C7=O)CC5)CC4)C3)cc2cc1NC(=O)c1cnn2ccc(N3C[C@H]4C[C@@H]3CO4)nc12]

is

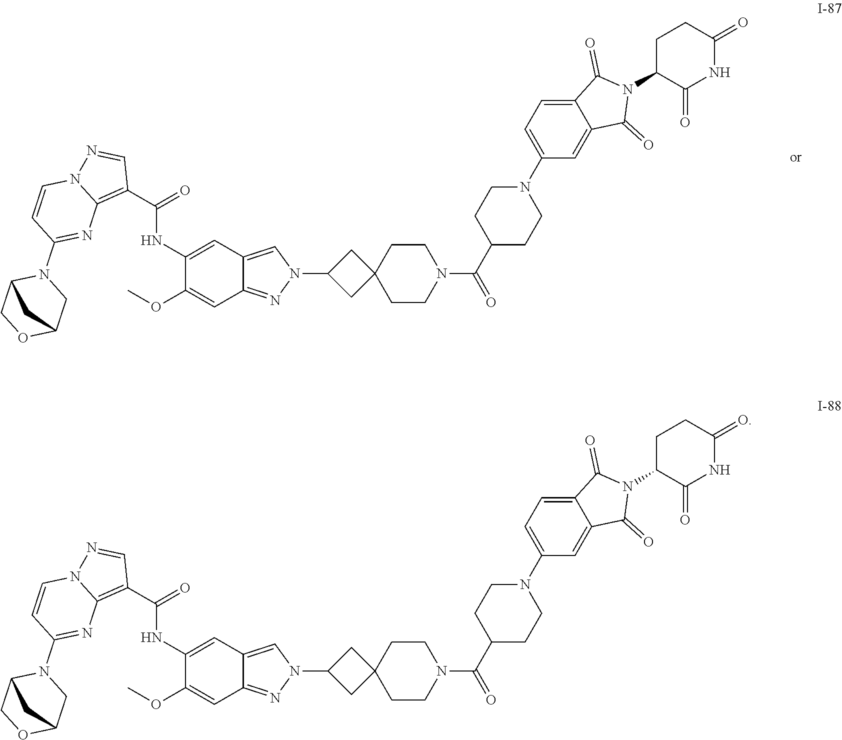 [SMILES: COc1cc2nn(C3CC4(CCN(C(=O)C5CCN(c6ccc7c(c6)C(=O)N([C@H]6CCC(=O)NC6=O)C7=O)CC5)CC4)C3)cc2cc1NC(=O)c1cnn2ccc(N3C[C@H]4C[C@@H]3CO4)nc12 ; COc1cc2nn(C3CC4(CCN(C(=O)C5CCN(c6ccc7c(c6)C(=O)N([C@@H]6CCC(=O)NC6=O)C7=O)CC5)CC4)C3)cc2cc1NC(=O)c1cnn2ccc(N3C[C@H]4C[C@@H]3CO4)nc12]

In a preferred embodiment, the pharmaceutically acceptable salt of the compound of I-12 is

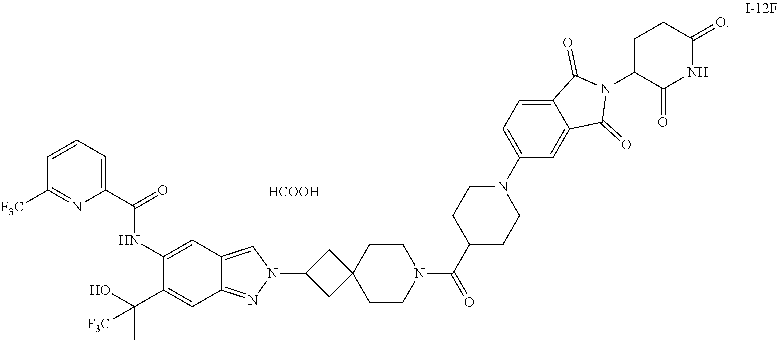 [SMILES: CC(O)(c1cc2nn(C3CC4(CCN(C(=O)C5CCN(c6ccc7c(c6)C(=O)N(C6CCC(=O)NC6=O)C7=O)CC5)CC4)C3)cc2cc1NC(=O)c1cccc(C(F)(F)F)n1)C(F)(F)F ; "HCOOH"]

In a preferred embodiment,

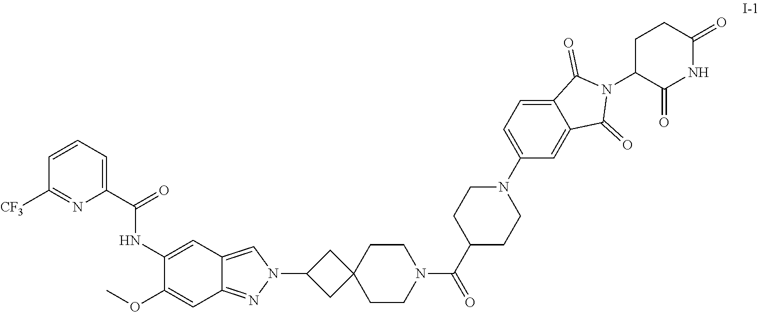 [SMILES: COc1cc2nn(C3CC4(CCN(C(=O)C5CCN(c6ccc7c(c6)C(=O)N(C6CCC(=O)NC6=O)C7=O)CC5)CC4)C3)cc2cc1NC(=O)c1cccc(C(F)(F)F)n1]

is a compound with a retention time of 2.262 min or 3.319 min under the following conditions: chromatographic column: IH 25×250 mm, 10 um (Daicel); mobile phase: CO₂/(MeOH:MeCN=1:1)=50/50; flow rate: 120 mL/min.

In a preferred embodiment,

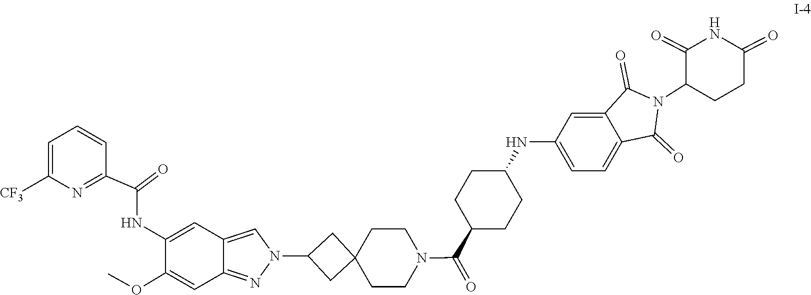 [SMILES: COc1cc2nn(C3CC4(CCN(C(=O)[C@H]5CC[C@H](Nc6ccc7c(c6)C(=O)N(C6CCC(=O)NC6=O)C7=O)CC5)CC4)C3)cc2cc1NC(=O)c1cccc(C(F)(F)F)n1]

is a compound with a retention time of 12.068 mi or 14.283 mi under the following conditions: chromatographic column: IC 25×250 mm, 10 um (Daicel); mobile phase: HEX (0.1% DEA):EtOH (0.1% DEA):=50:50; flow rate:50 mL/min.

In a preferred embodiment,

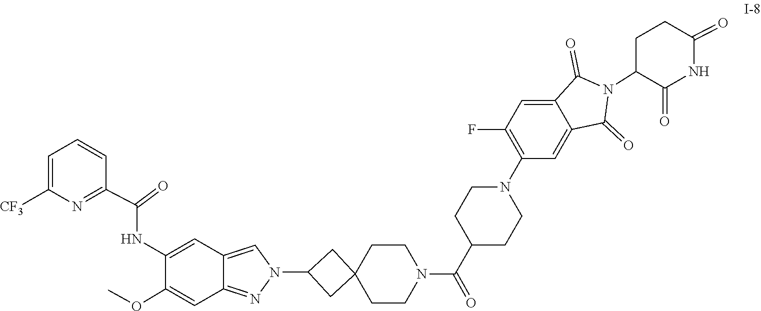 [SMILES: COc1cc2nn(C3CC4(CCN(C(=O)C5CCN(c6cc7c(cc6F)C(=O)N(C6CCC(=O)NC6=O)C7=O)CC5)CC4)C3)cc2cc1NC(=O)c1cccc(C(F)(F)F)n1]

is a compound with a retention time of 1.759 min or 2.520 min under the following conditions: chromatographic column: IH 25×250 mm, 10 um (Daicel); mobile phase: CO₂/[MeOH:MeCN=1:1]=50/50; flow rate:120 mL/min.

In a preferred embodiment,

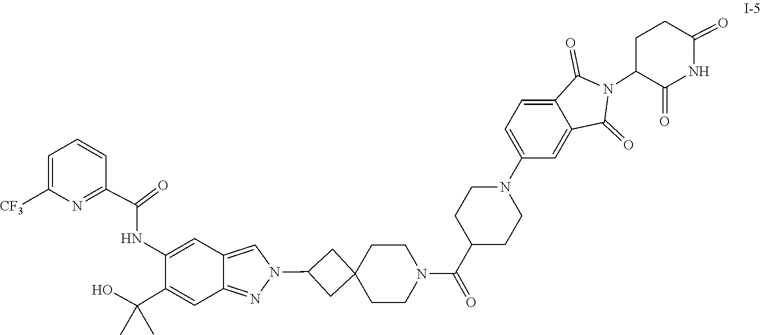 [SMILES: CC(C)(O)c1cc2nn(C3CC4(CCN(C(=O)C5CCN(c6ccc7c(c6)C(=O)N(C6CCC(=O)NC6=O)C7=O)CC5)CC4)C3)cc2cc1NC(=O)c1cccc(C(F)(F)F)n1]

is a compound with a retention time of 1.299 min or 1.936 min under the following conditions: chromatographic column: AS 25×250 mm, 10 um (Daicel); mobile phase: CO₂/[MeOH (0.2% NH₃(7M in MOH):MeCN=1:1]=40/60; flow rate:100 mL/min.

In a preferred embodiment,

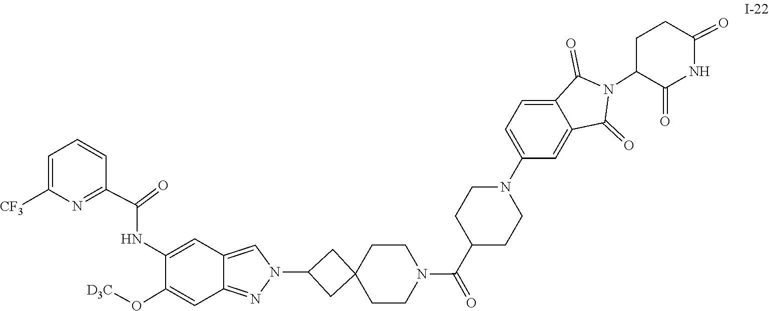 [SMILES: [2H]C([2H])([2H])Oc1cc2nn(C3CC4(CCN(C(=O)C5CCN(c6ccc7c(c6)C(=O)N(C6CCC(=O)NC6=O)C7=O)CC5)CC4)C3)cc2cc1NC(=O)c1cccc(C(F)(F)F)n1]

is a compound with a retention time of 2.309 min or 3.384 min under the following conditions: chromatographic column: IH 25×250 mm, 10 μm (Daicel); mobile phase: CO₂/[MeOH:MeCN=1:1]=50/50; flow rate: 120 mL/min.

The present disclosure also provides a pharmaceutical composition comprising substance Z and pharmaceutical excipients, wherein the substance Z is a compound of formula I, a pharmaceutically acceptable salt or an isotope compound thereof.

In a preferred embodiment, in the pharmaceutical composition, the substance Z is an effective amount of substance Z.

In a preferred embodiment, the pharmaceutical composition is used to treat and/or prevent Myd88 and/or IRAK4-related diseases.

The present disclosure also provides a use of substance Z in the preparation of an IRAK4 degrading agent and a medicament for the treatment and/or prevention of Myd88 and/or IRAK4 related diseases, wherein the substance Z is a compound of formula I, and a pharmaceutically acceptable salt or an isotopic compound thereof.

The present disclosure also provides a method for treating and/or preventing Myd88 and/or IRAK4-related diseases, which includes administering an effective amount of substance Z to the patient, wherein the substance Z is a compound of formula I, and a pharmaceutically acceptable salt or an isotopic compound thereof.

In a preferred embodiment, the IRAK4-related diseases include one or more of autoimmune diseases, inflammatory diseases, tumors, cardiovascular and cerebrovascular diseases and central nervous system diseases.

In a preferred embodiment, the IRAK4-related diseases include one or more of chronic lung diseases, autoimmune diseases, inflammatory diseases, tumors, cardiovascular and cerebrovascular diseases and central nervous system diseases.

In a preferred embodiment, the autoimmune diseases include psoriasis and rheumatoid arthritis.

In a preferred embodiment, the autoimmune diseases include psoriasis, systemic lupus erythematosus and rheumatoid arthritis.

In a preferred embodiment, the inflammatory disease includes ulcerative colitis.

In a preferred embodiment, the inflammatory disease includes inflammatory bowel disease, such as ulcerative colitis.

In a preferred embodiment, the tumors can be hematological tumors and solid tumors.

In a preferred embodiment, the hematological tumors include large B-cell lymphoma and acute and chronic lymphocytic leukemia; In a preferred embodiment, the solid tumors include intestinal cancer and skin cancer caused by MYD88 mutations.

In a preferred embodiment, the cardiovascular and cerebrovascular diseases include stroke and atherosclerosis.

In a preferred embodiment, the central nervous system disease includes primary central nervous system lymphoma.

The present disclosure also provides a use of substance Z or the above-mentioned pharmaceutical composition in the preparation of a medicament, wherein the substance Z is a compound of formula I, a pharmaceutically acceptable salt or an isotope compound thereof; and the medicament is used for treatment one or more of autoimmune diseases, inflammatory diseases, tumors, cardiovascular and cerebrovascular diseases and central nervous system diseases.

In a preferred embodiment, the autoimmune diseases, inflammatory diseases, tumors, cardiovascular and cerebrovascular diseases and central nervous system diseases are as described in any embodiment of the present disclosure.

The present disclosure also provides a compound of formula II or III, a salt, a Boc-protected compound or an isotope compound thereof,

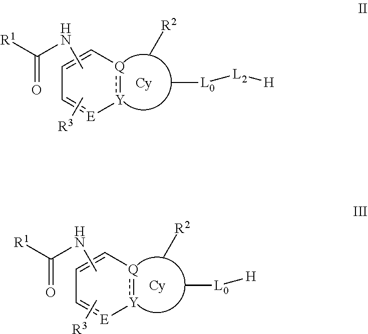

R¹, R², R³, Q, Y, ring Cy, L₀ and L₂ are as described above;

Preferably, the compound of formula II or III is an of the following compounds:

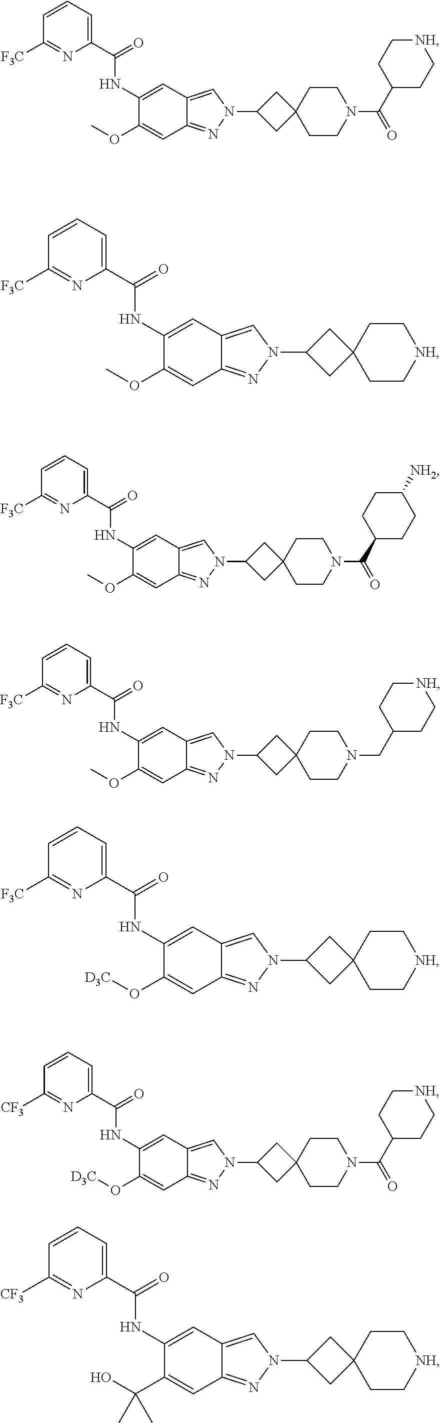

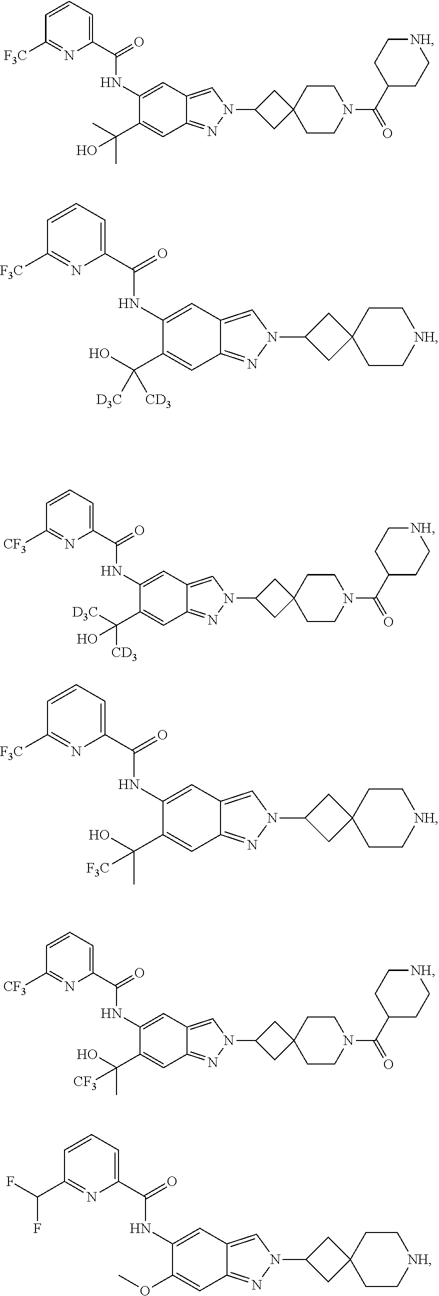

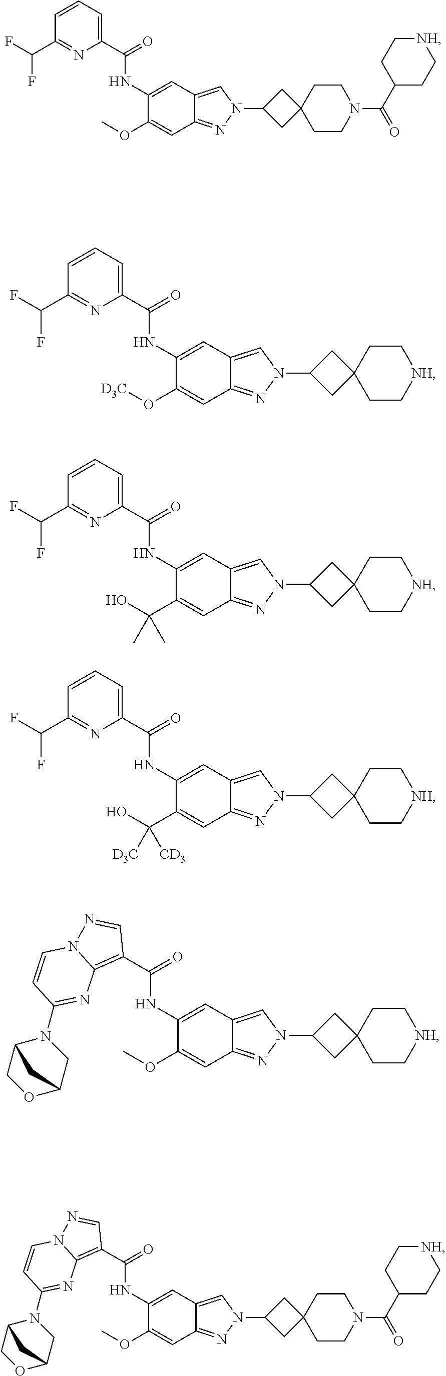 [SMILES: COc1cc2nn(C3CC4(CCN(C(=O)C5CCNCC5)CC4)C3)cc2cc1NC(=O)c1cccc(C(F)F)n1 ; [2H]C([2H])([2H])Oc1cc2nn(C3CC4(CCNCC4)C3)cc2cc1NC(=O)c1cccc(C(F)F)n1 ; CC(C)(O)c1cc2nn(C3CC4(CCNCC4)C3)cc2cc1NC(=O)c1cccc(C(F)F)n1 ; [2H]C([2H])([2H])C(O)(c1cc2nn(C3CC4(CCNCC4)C3)cc2cc1NC(=O)c1cccc(C(F)F)n1)C([2H])([2H])[2H] ; COc1cc2nn(C3CC4(CCNCC4)C3)cc2cc1NC(=O)c1cnn2ccc(N3C[C@H]4C[C@@H]3CO4)nc12 ; COc1cc2nn(C3CC4(CCN(C(=O)C5CCNCC5)CC4)C3)cc2cc1NC(=O)c1cnn2ccc(N3C[C@H]4C[C@@H]3CO4)nc12]

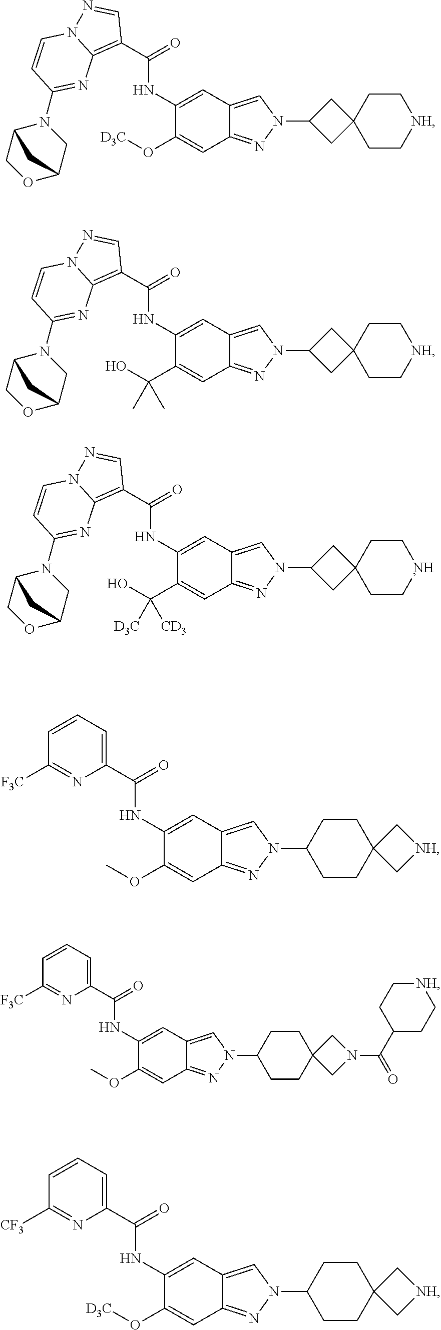 [SMILES: [2H]C([2H])([2H])Oc1cc2nn(C3CC4(CCNCC4)C3)cc2cc1NC(=O)c1cnn2ccc(N3C[C@H]4C[C@@H]3CO4)nc12 ; CC(C)(O)c1cc2nn(C3CC4(CCNCC4)C3)cc2cc1NC(=O)c1cnn2ccc(N3C[C@H]4C[C@@H]3CO4)nc12 ; [2H]C([2H])([2H])C(O)(c1cc2nn(C3CC4(CCNCC4)C3)cc2cc1NC(=O)c1cnn2ccc(N3C[C@H]4C[C@@H]3CO4)nc12)C([2H])([2H])[2H] ; COc1cc2nn(C3CCC4(CC3)CNC4)cc2cc1NC(=O)c1cccc(C(F)(F)F)n1 ; COc1cc2nn(C3CCC4(CC3)CN(C(=O)C3CCNCC3)C4)cc2cc1NC(=O)c1cccc(C(F)(F)F)n1 ; [2H]C([2H])([2H])Oc1cc2nn(C3CCC4(CC3)CNC4)cc2cc1NC(=O)c1cccc(C(F)(F)F)n1]

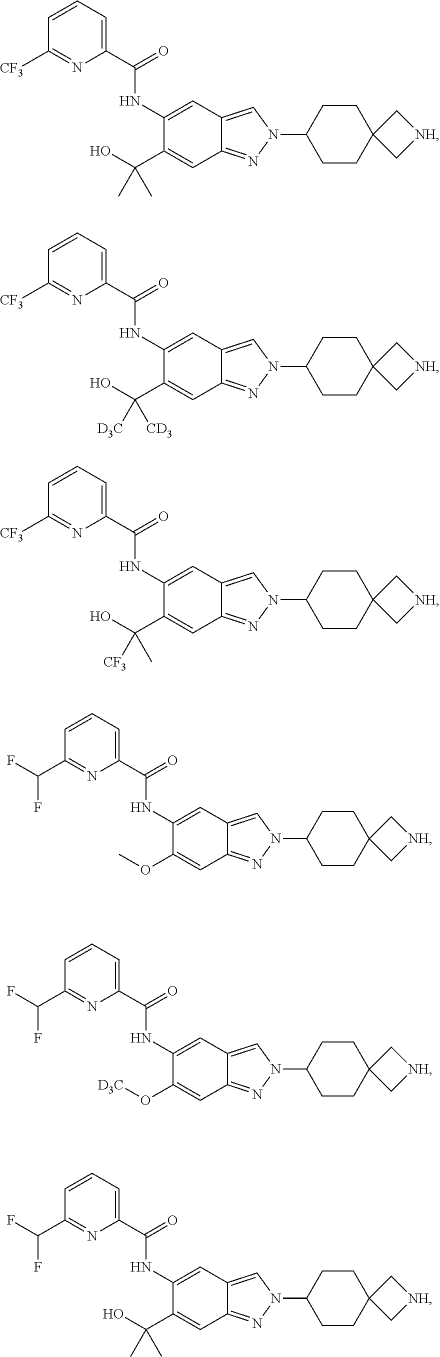 [SMILES: CC(C)(O)c1cc2nn(C3CCC4(CC3)CNC4)cc2cc1NC(=O)c1cccc(C(F)(F)F)n1 ; [2H]C([2H])([2H])C(O)(c1cc2nn(C3CCC4(CC3)CNC4)cc2cc1NC(=O)c1cccc(C(F)(F)F)n1)C([2H])([2H])[2H] ; CC(O)(c1cc2nn(C3CCC4(CC3)CNC4)cc2cc1NC(=O)c1cccc(C(F)(F)F)n1)C(F)(F)F ; COc1cc2nn(C3CCC4(CC3)CNC4)cc2cc1NC(=O)c1cccc(C(F)F)n1 ; [2H]C([2H])([2H])Oc1cc2nn(C3CCC4(CC3)CNC4)cc2cc1NC(=O)c1cccc(C(F)F)n1 ; CC(C)(O)c1cc2nn(C3CCC4(CC3)CNC4)cc2cc1NC(=O)c1cccc(C(F)F)n1]

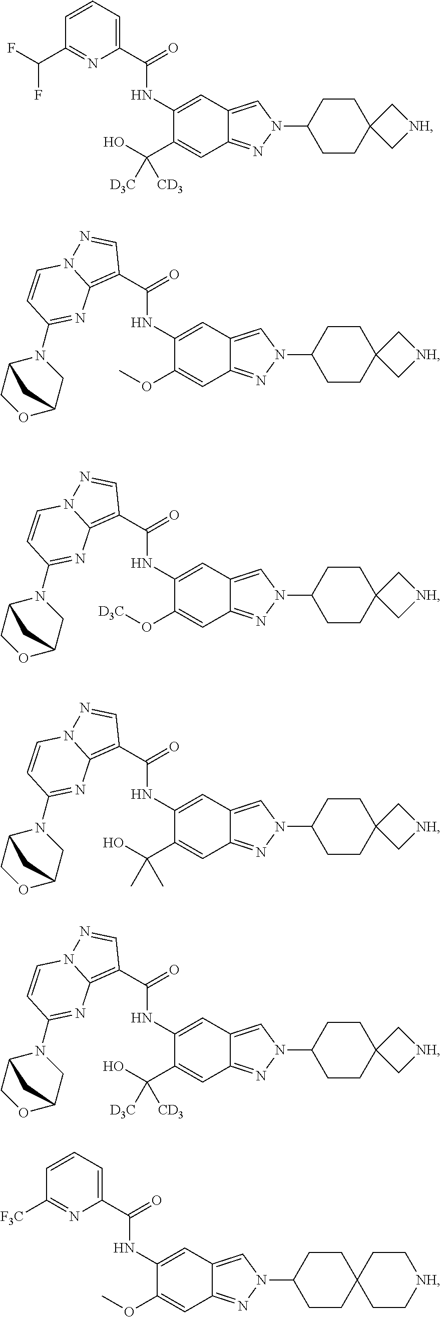

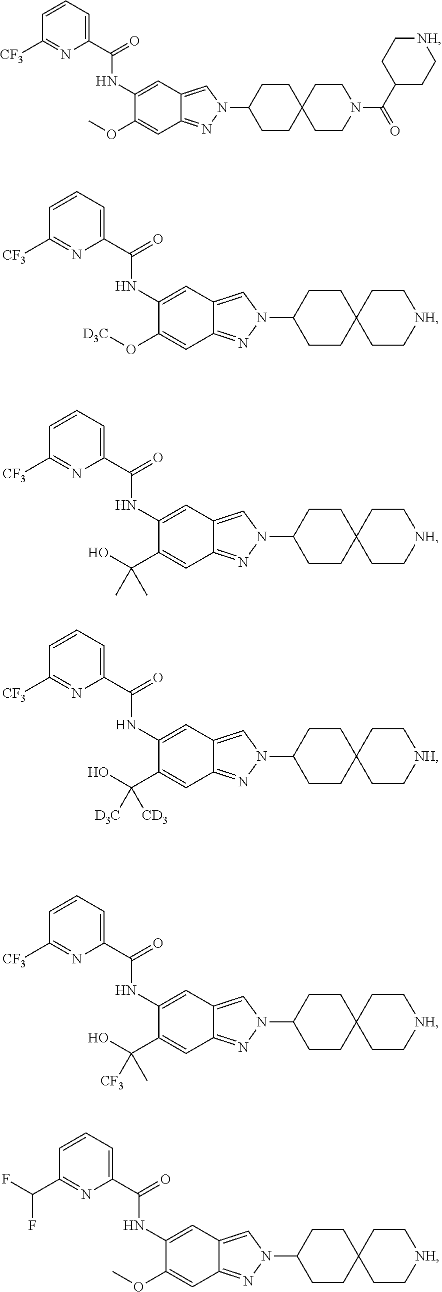 [SMILES: COc1cc2nn(C3CCC4(CC3)CCN(C(=O)C3CCNCC3)CC4)cc2cc1NC(=O)c1cccc(C(F)(F)F)n1 ; [2H]C([2H])([2H])Oc1cc2nn(C3CCC4(CCNCC4)CC3)cc2cc1NC(=O)c1cccc(C(F)(F)F)n1 ; CC(C)(O)c1cc2nn(C3CCC4(CCNCC4)CC3)cc2cc1NC(=O)c1cccc(C(F)(F)F)n1 ; [2H]C([2H])([2H])C(O)(c1cc2nn(C3CCC4(CCNCC4)CC3)cc2cc1NC(=O)c1cccc(C(F)(F)F)n1)C([2H])([2H])[2H] ; CC(O)(c1cc2nn(C3CCC4(CCNCC4)CC3)cc2cc1NC(=O)c1cccc(C(F)(F)F)n1)C(F)(F)F ; COc1cc2nn(C3CCC4(CCNCC4)CC3)cc2cc1NC(=O)c1cccc(C(F)F)n1]

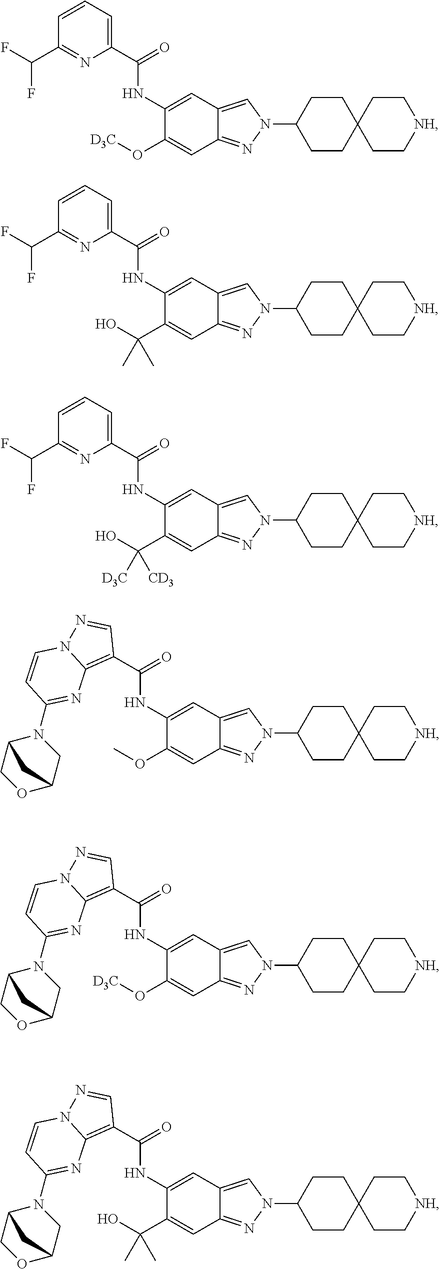 [SMILES: [2H]C([2H])([2H])Oc1cc2nn(C3CCC4(CCNCC4)CC3)cc2cc1NC(=O)c1cccc(C(F)F)n1 ; CC(C)(O)c1cc2nn(C3CCC4(CCNCC4)CC3)cc2cc1NC(=O)c1cccc(C(F)F)n1 ; [2H]C([2H])([2H])C(O)(c1cc2nn(C3CCC4(CCNCC4)CC3)cc2cc1NC(=O)c1cccc(C(F)F)n1)C([2H])([2H])[2H] ; COc1cc2nn(C3CCC4(CCNCC4)CC3)cc2cc1NC(=O)c1cnn2ccc(N3C[C@H]4C[C@@H]3CO4)nc12 ; [2H]C([2H])([2H])Oc1cc2nn(C3CCC4(CCNCC4)CC3)cc2cc1NC(=O)c1cnn2ccc(N3C[C@H]4C[C@@H]3CO4)nc12 ; CC(C)(O)c1cc2nn(C3CCC4(CCNCC4)CC3)cc2cc1NC(=O)c1cnn2ccc(N3C[C@H]4C[C@@H]3CO4)nc12]

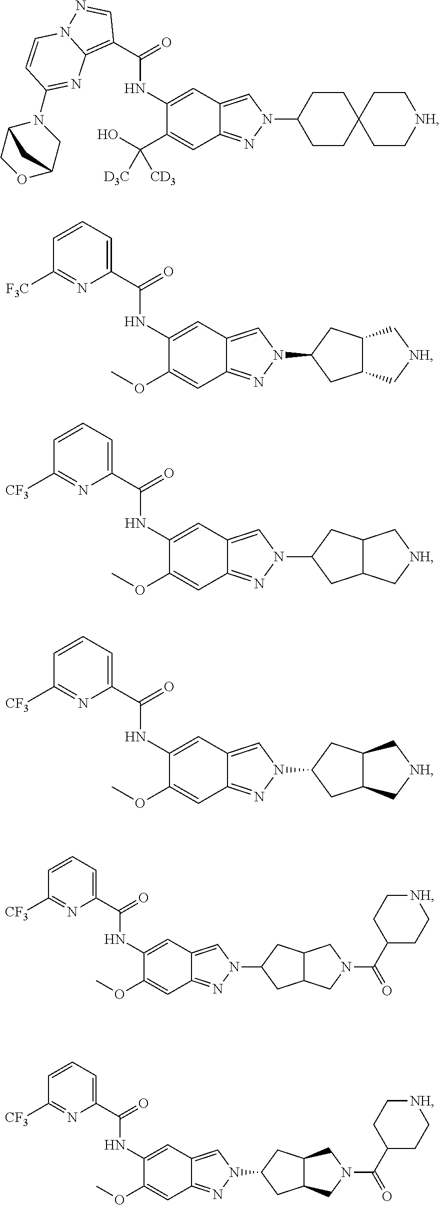 [SMILES: [2H]C([2H])([2H])C(O)(c1cc2nn(C3CCC4(CCNCC4)CC3)cc2cc1NC(=O)c1cnn2ccc(N3C[C@H]4C[C@@H]3CO4)nc12)C([2H])([2H])[2H] ; COc1cc2nn([C@@H]3C[C@H]4CNC[C@H]4C3)cc2cc1NC(=O)c1cccc(C(F)(F)F)n1 ; COc1cc2nn(C3CC4CNCC4C3)cc2cc1NC(=O)c1cccc(C(F)(F)F)n1 ; COc1cc2nn([C@@H]3C[C@H]4CNC[C@H]4C3)cc2cc1NC(=O)c1cccc(C(F)(F)F)n1 ; COc1cc2nn(C3CC4CN(C(=O)C5CCNCC5)CC4C3)cc2cc1NC(=O)c1cccc(C(F)(F)F)n1 ; COc1cc2nn([C@H]3C[C@@H]4CN(C(=O)C5CCNCC5)C[C@@H]4C3)cc2cc1NC(=O)c1cccc(C(F)(F)F)n1]

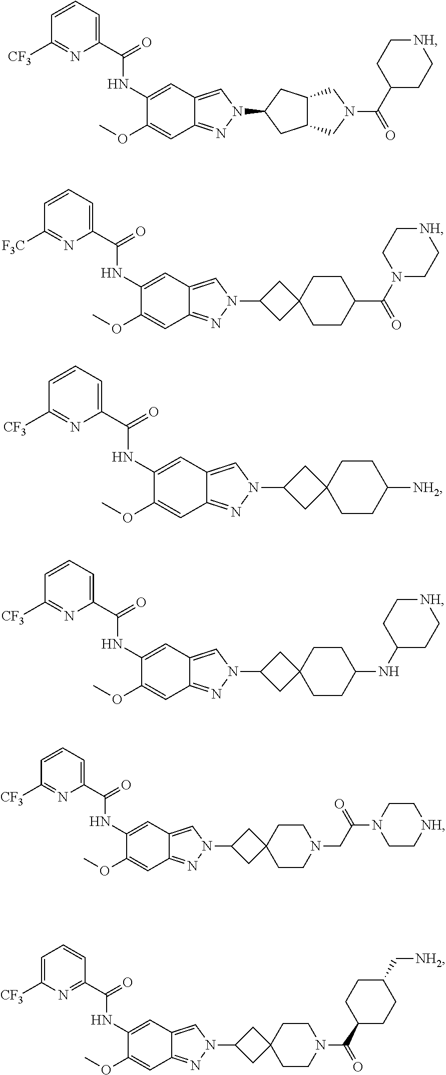 [SMILES: COc1cc2nn([C@H]3C[C@@H]4CN(C(=O)C5CCNCC5)C[C@@H]4C3)cc2cc1NC(=O)c1cccc(C(F)(F)F)n1 ; COc1cc2nn(C3CC4(CCC(C(=O)N5CCNCC5)CC4)C3)cc2cc1NC(=O)c1cccc(C(F)(F)F)n1 ; COc1cc2nn(C3CC4(CCC(N)CC4)C3)cc2cc1NC(=O)c1cccc(C(F)(F)F)n1 ; COc1cc2nn(C3CC4(CCC(NC5CCNCC5)CC4)C3)cc2cc1NC(=O)c1cccc(C(F)(F)F)n1 ; COc1cc2nn(C3CC4(CCN(CC(=O)N5CCNCC5)CC4)C3)cc2cc1NC(=O)c1cccc(C(F)(F)F)n1 ; COc1cc2nn(C3CC4(CCN(C(=O)[C@H]5CC[C@H](CN)CC5)CC4)C3)cc2cc1NC(=O)c1cccc(C(F)(F)F)n1]

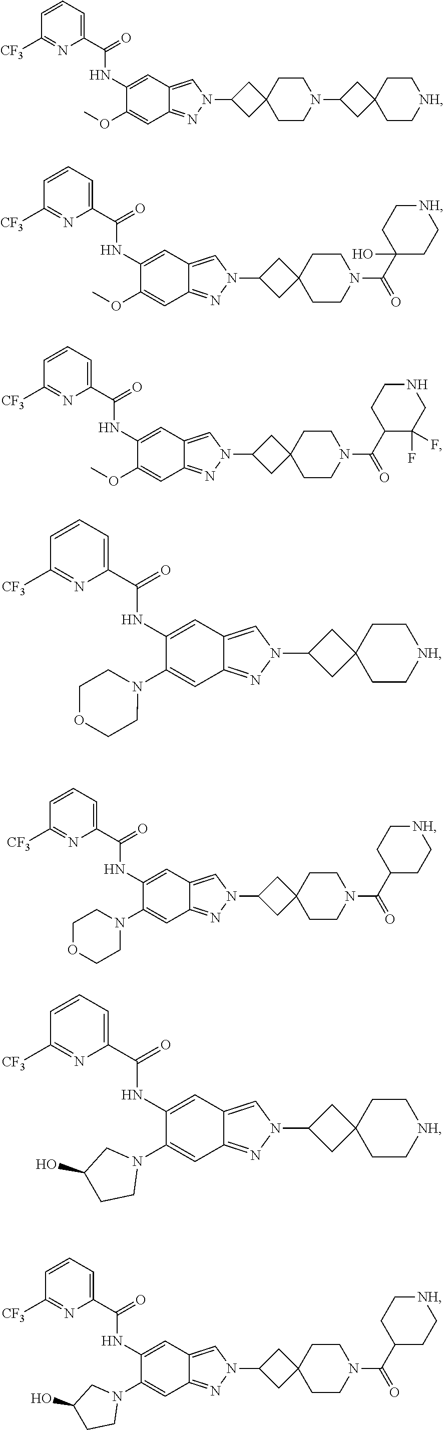

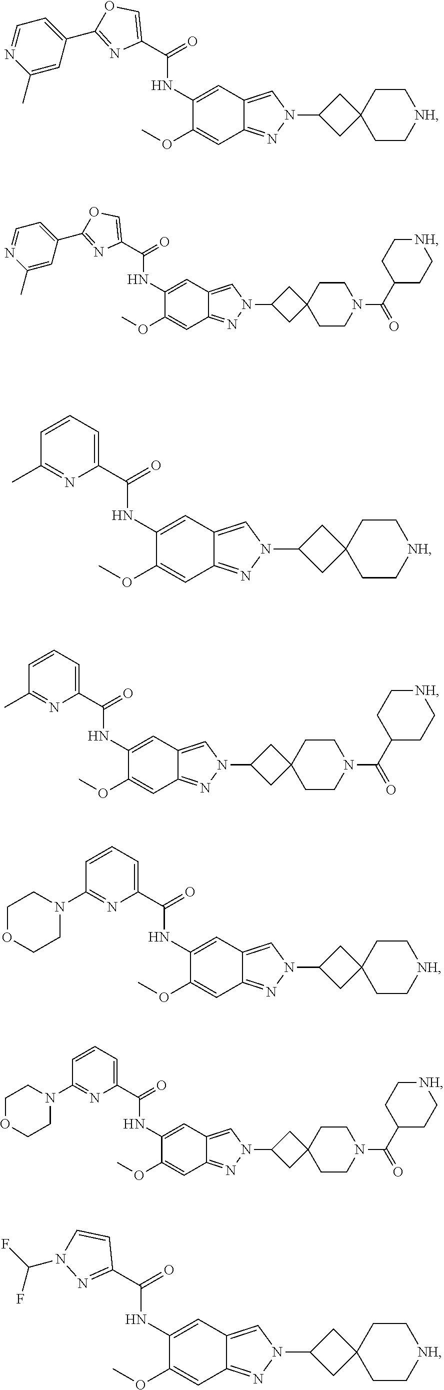

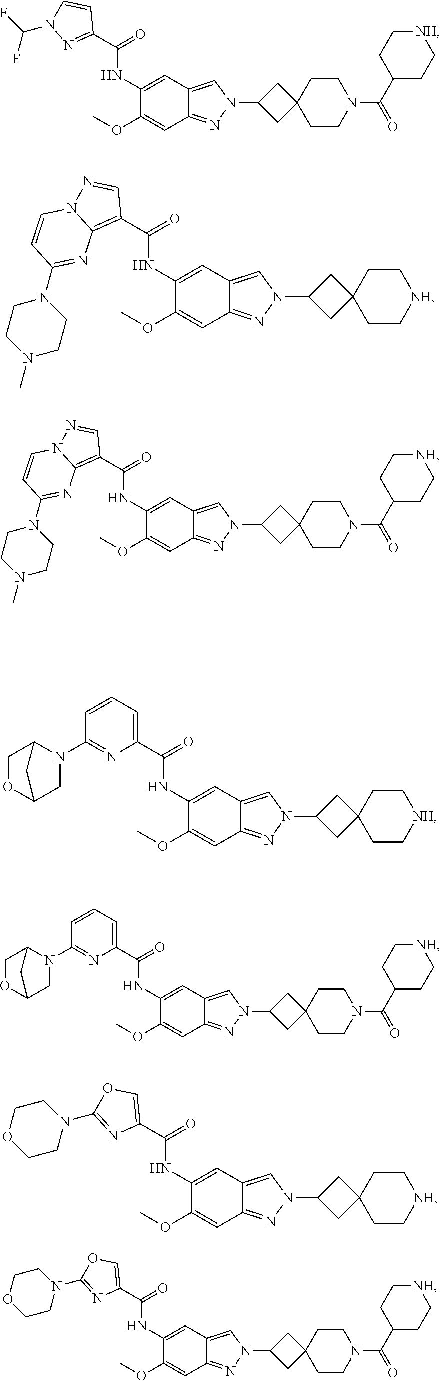

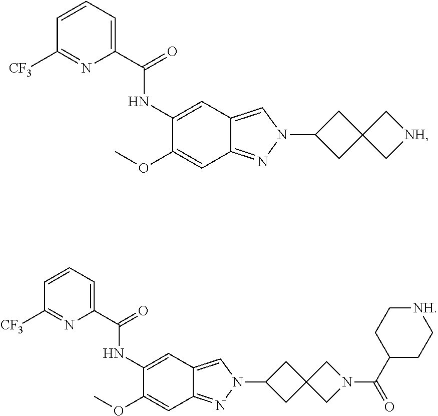 [SMILES: COc1cc2nn(C3CC4(CNC4)C3)cc2cc1NC(=O)c1cccc(C(F)(F)F)n1 ; COc1cc2nn(C3CC4(C3)CN(C(=O)C3CCNCC3)C4)cc2cc1NC(=O)c1cccc(C(F)(F)F)n1]

The present disclosure also provides a use of a compound of formula II or III, a salt, a Boc-protected compound or an isotope compound thereof as described above in the preparation of a compound of formula I, a pharmaceutically acceptable salt or an isotopic compound thereof as described above.

Unless otherwise specified, the terms used in the present disclosure have the following meanings:

The terms “compound” and “pharmaceutically acceptable salt” may exist in the form of a single tautomer or a mixture thereof, preferably in the form of a more stable tautomer, if tautomers are present.

If a linker is expressed as “absent”, the structures on both sides of the linker are directly connected by a single bond, for example -A-B-C-. When B is not present, -A-B-C- is -A-C-.

The term “ [symbol] ” refers to presence or absence.

The term “oxo” means that the hydrogen or lone pair of electrons on the non-oxygen atom is replaced by oxygen. For example,

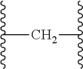 [SMILES: *C*]

is

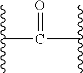 [SMILES: *C(*)=O]

after being replaced by oxygen and

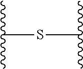 [SMILES: *S*]

is

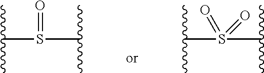

after being replaced by oxygen

The term “halogen” refers to fluorine, chlorine, bromine or iodine.

The term “cycloalkyl” refers to a saturated monocyclic cyclic group having a specified number of carbon atoms (e.g., C₃-C₁₀), which consists only of carbon atoms. The monocyclic alkyl groups include, but are not limited to, cyclopropyl, cyclobutyl, cyclopentyl, cyclohexyl, and the like.

The term “alkyl” refers to a straight or branched alkyl group having a specified number of carbon atoms (e.g., C₁-C₆). Alkyl groups include, but are not limited to, methyl, ethyl, n-propyl, isopropyl, n-butyl, tert-butyl, isobutyl, sec-butyl, n-pentyl, n-hexyl, and the like.

The term “heterocycloalkyl” refers to a cyclic group having a specified number of ring atoms (e.g., 3 to 11 members, also e.g., 3 to 8 members), a specified number of heteroatoms (such as 1, 2 or 3), and a specified heteroatom types (one or more of N, O and S), in which heteroatoms can be connected to other groups as connecting groups or not be connected to other groups (for example, the piperidinyl can be

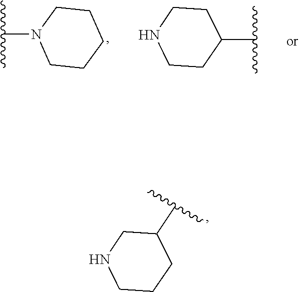 [SMILES: *N1CCCCC1 ; *C1CCNCC1 ; *C1CCCNC1]

and the like). It is a monocyclic ring, a fused ring, a bridged ring or a spiro ring, and each ring is saturated. Heterocycloalkyl includes, but are not limited to, azetidinyl, tetrahydropyrrolyl, tetrahydrofuryl, morpholinyl, piperidinyl, and the like.

The term “heterocycle” refers to a cyclic group having a specified number of ring atoms (e.g., 3 to 12 members), a specified number of heteroatoms (e.g., 1, 2, or 3), and a specified type of heteroatoms (one or more of N, O, and S), in which heteroatoms can be connected to other groups as connecting groups, or not be connected to other groups (for example, the piperidine ring can be

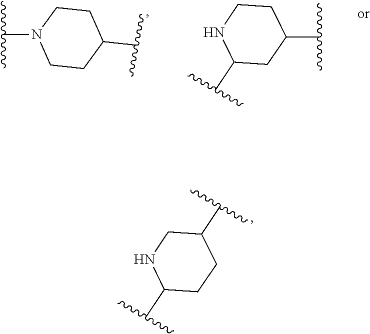 [SMILES: *C1CCN(*)CC1 ; *C1CCNC(*)C1 ; *C1CCC(*)NC1]

and the like). It is a monocyclic ring, a fused ring, a bridged ring or a spiro ring, and each ring is saturated. Heterocycloalkyl includes, but are not limited to, azetidine ring, tetrahydropyrrole ring, tetrahydrofuran ring, morpholine ring, and piperidyl ring.

The term “heteroaryl” refers to a cyclic group having a specified number of ring atoms (e.g., 5 to 9 members), a specified number of heteroatoms (e.g., 1, 2, or 3), and a specified type of heteroatom (one or more of N, O, and S), which is monocyclic or polycyclic, and at least one ring is aromatic (in compliance with Huckel's rule). The heteroaryl group is connected to other segments in the molecule through an aromatic ring or a non-aromatic ring. Heteroaryl groups include, but are not limited to, furyl, pyrrolyl, thienyl, pyrazolyl, imidazolyl, oxazolyl, thiazolyl, pyridyl, pyrimidinyl, indolyl, and the like.

The term “heteroaromatic ring” refers to a cyclic group having a specified number of ring atoms (e.g., 5 to 9 members), a specified number of heteroatoms (e.g., 1, 2, or 3), and a specified type of heteroatom (one or more of N, O, and S), which is monocyclic or polycyclic, and at least one ring is aromatic (in compliance with Huckel's rule). The heteroaromatic ring is connected to other segments in the molecule through an aromatic ring or a non-aromatic ring. Heteroaromatic rings include, but are not limited to, furan ring, pyrrole ring, thiophene ring, pyrazole ring, imidazole ring, oxazole ring, thiazole ring, pyridine ring, pyrimidine ring, indole ring, and the like.

The “-” at the end of a group means that the group is connected to other fragments in the molecule by this site. For example, CH₃—C(═O)— refers to an acetyl group.

The “ [symbol] ” in a structural fragment means that the structural fragment is connected to other fragments in the molecule by this site. For example,

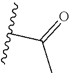 [SMILES: *C(C)=O]

refers to an acetyl group.

The term “cycloalkylene group” is a divalent group connected to the rest of the molecule by two single bonds. Other definitions are the same as the term “cycloalkyl”.

The term “heterocycloalkylene group” is a divalent group connected to the rest of the molecule by two single bonds. Other definitions are the same as the term “heterocycloalkyl”.

The term “isotopic compound” refers to a compound in which one or more atoms have an isotopic abundance that differs from its natural abundance. For example, one or more atoms in a compound are replaced by atoms that have a lower mass number in nature—a hydrogen atom in the compound is replaced by deuterium, or a C is replaced by ¹³C.

The term “more” means 2, 3, 4 or 5.

The term “Boc-protected” generally means that N is protected by a Boc group.

Substituents such as heterocycle, aromatic ring, heteroaryl, aryl, heterocycloalkyl, alkoxy, alkyl, cycloalkyl, and the like described in the present disclosure serve as linker units to connect different components of the compound. Thus, they can also be called as heterocyclylene, arylene ring, heteroarylene group, arylene group, heterocycloalkylene group, alkyleneoxy group, alkylene group, and cycloalkylene group.

When any variable (such as the group R¹⁻¹) appears multiple times in the definition of a compound, their definitions are independent of each other and do not affect each other. For example, a C₆-C₁₀ aryl substituted by 3 R¹⁻¹ means that the C₆-C₁₀ aryl will be substituted by 3 R¹⁻¹. The definitions of the 3 R¹⁻¹ are independent of each other and do not affect each other.

The term “pharmaceutically acceptable salt” refers to a salt obtained by reacting a compound with a pharmaceutically acceptable (relatively nontoxic, safe, and suitable for use by a patient) acid or base. When the compound comprises relatively acidic functional groups, base addition salts can be obtained by contacting a free form of the compound with a sufficient amount of a pharmaceutically acceptable base in a suitable inert solvent. Pharmaceutically acceptable base addition salts include, but are not limited to, sodium salts, potassium salts, calcium salts, aluminum salts, magnesium salts, bismuth salts, ammonium salts, and the like. When the compound comprises relatively basic functional groups, acid addition salts can be obtained by contacting a free form of the compound with a sufficient amount of a pharmaceutically acceptable acid in a suitable inert solvent. Pharmaceutically acceptable acid addition salts include, but are not limited to, hydrochloride, acetate, trifluoroacetate, sulfate, methanesulfonate, and the like. For details, see Handbook of Pharmaceutical Salts: Properties, Selection, and Use (P. Heinrich Stahl, 2002).

The term “solvate of pharmaceutically acceptable salt” refers to a substance formed by combining a compound with a pharmaceutically acceptable (relatively non-toxic, safe, and suitable for use by a patient) acid or base, solvent (including but not limited to: water, methanol, ethanol, and the like), wherein the pharmaceutically acceptable salt here has the same meaning as the term “pharmaceutically acceptable salt” above, and the solvent is stoichiometric or non-stoichiometric. Pharmaceutically acceptable salt solvates include, but are not limited to, hydrochloride monohydrate.

The term “pharmaceutical excipients” refers to excipients and additives used in the preparation of a medicament and preparation of a prescription. Pharmaceutical excipients are all substances other than active ingredients included in pharmaceutical formulations. For details, see the Pharmacopoeia of the People's Republic of China (2020 Edition) or Handbook of Pharmaceutical EM cipients (Raymond C Rowe, 2009).

The term “treatment” refers to any of the following: (1) alleviating one or more biological manifestations of a disease; (2) interfering with one or more points in the biological cascade that causes the disease; and (3) slowing down the progression of one or more biological manifestations of a disease.

The term “prevention” refers to reducing the risk of developing a disease.

The term “patient” refers to any animal, preferably a mammal, most preferably a human, that has been or will be treated. Mammals include, but are not limited to, cattle, horses, sheep, pigs, cats, dogs, mice, rats, rabbits, guinea pigs, monkeys, humans, and the like.

As used in the specifications and claims, the singular form “a”, “an” and “the” includes plural references unless the context clearly dictates otherwise.

On the basis of not violating common sense in the art, the above preferred embodiments can be combined arbitrarily to obtain preferred examples of the present disclosure.

The reagents and raw materials used in the present invention are all commercially available.

The positive and progressive effect of the present disclosure is that the compound of the present disclosure has an inhibitory or/and degradative effect on IRAK4, and can nearly completely degrade the IRAK4 protein at a lower drug concentration. The compounds of the present disclosure can also inhibit the scaffolding function of the Myddosome complex. The compounds of the present disclosure have potential clinical application value and are expected to be applied to various IRAK4-related immune diseases and various hematological diseases and solid tumors caused by Myd88 mutations.

### DETAILED DESCRIPTION OF THE PREFERRED EMBODIMENT

The present disclosure is further described below by way of examples. However, the present disclosure is not limited to the scope of the described examples. Experimental methods that do not indicate specific conditions in the following examples should be selected according to conventional methods and conditions, or according to product specifications.

### Example 1: Synthesis of Compound I-1

### Synthesis of Compound I-1

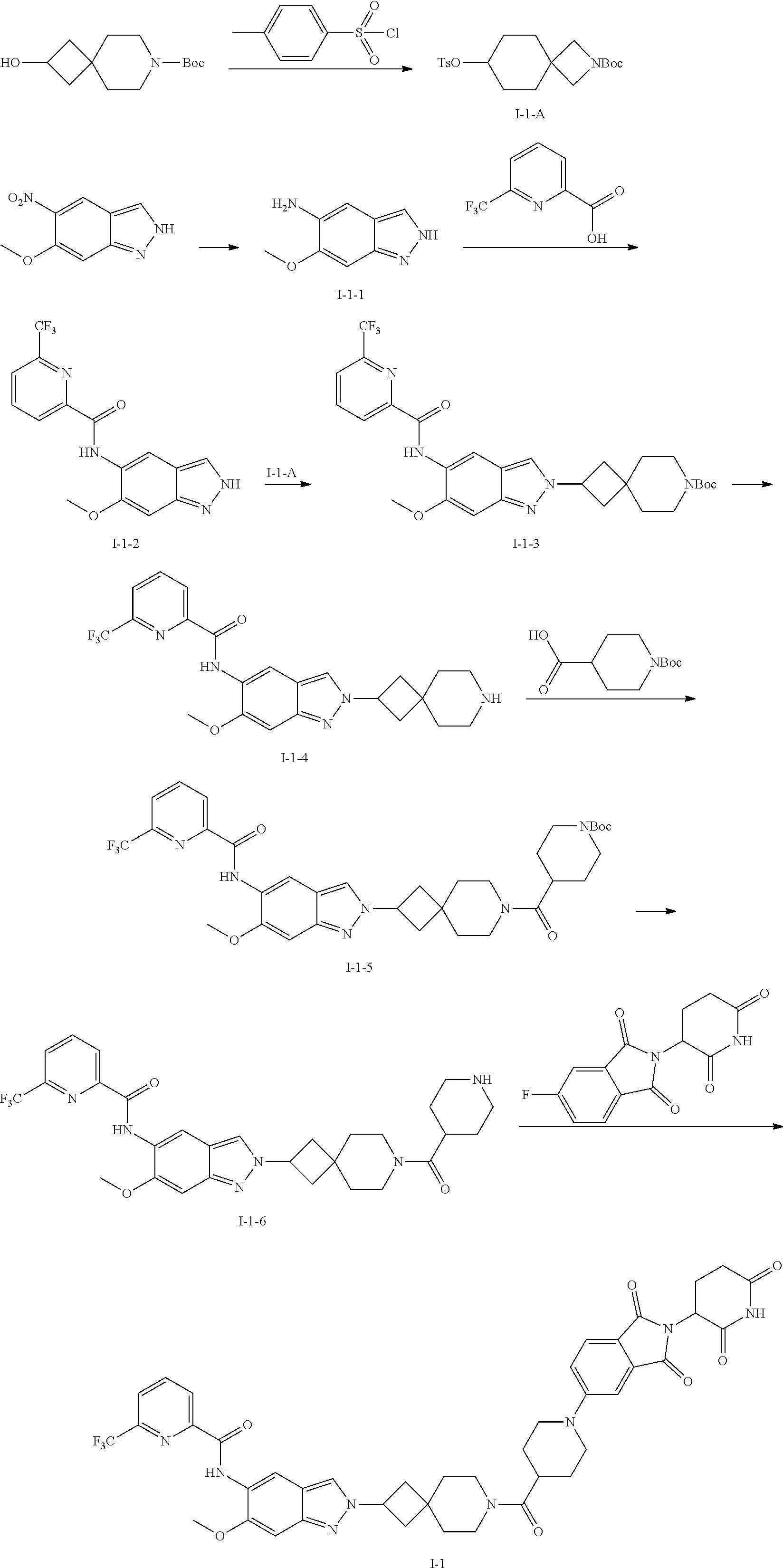

### Step 1: Synthesis of I-1-A

A mixed solution of 7-tert-butoxycarbonyl-7-azaspiro[3.5]-2-nonanol (600 mg, 2.49 mmol), 4-dimethylaminopyridine (60.75 mg, 497.25 μmol, 83.68 μL), triethylamine (503.17 mg, 4.97 mmol, 693.07 μL) and methylene chloride (1.35 mL) was stirred to dissolve, and p-toluenesulfonyl chloride (521.40 mg, 2.73 mmol) was added. The reaction was raised to 40° C. and stirred for 18 hours until the reaction was complete. The reaction system was added with water (30 mL) to dilute, and stirred and extracted with dichloromethane (20 mL×3). The combined organic phase was washed with brine (20 mL), dried over anhydrous sodium sulfate, filtered and concentrated in vacuum to obtain I-1-A (1 g, crude product), MS (ESI) m/z: 396.2 [M+H]⁺.

### Step 2: Synthesis of I-1-1

A mixed solution of 6-methoxy-5-nitro-2H-indazole (2 g, 10.35 mmol), Pd/C (0.3 g, 10% purity) and methanol (30 mL) was stirred and replaced with hydrogen three times. The reaction was continued under a hydrogen environment at 25° C. for 18 hours until the reaction was complete. The mixed solution was filtered through diatomaceous earth to remove the catalyst and the filter cake was washed with methanol (10 mL×2). The filtrate was concentrated under reduced pressure to obtain product I-1-1 as a brown solid (1.59 g, crude product), MS (ESI) m/z: 164.1 [M+H]⁺, which was used directly in the next step.

### Step 3: Synthesis of I-1-2

To a mixed solution of I-1-1(1.59 g, 9.74 mmol), 6-(trifluoromethyl)pyridine-2-carboxylic acid (2.05 g, 10.72 mmol), diisopropylethylamine (3.78 g, 29.23 mmol, 5.09 mL) and tetrahydrofuran (25 mL), 2,4, 6-tripropyl-1,3,5,2,4,6 trioxotriphosphonane 2,4,6-trioxide (7.44 g, 11.69 mmol, 50% purity) was added dropwise with stirring at 0° C. After the dropwise addition was completed, the reaction system was stirred under nitrogen protection at 15° C. for 2 hours until the reaction was complete. The reaction mixture was added with water (50 mL), and extracted with ethyl acetate (40 mL×5). The combined organic phase was washed with water (30 mL) and saturated brine, dried over anhydrous sodium sulfate, filtered and concentrated under vacuum and reduced pressure to obtain product I-1-2 as a brown solid (3.08 g, crude product), MS (ESI) m/z: 337.1 [M+H]⁺, which was directly used in the next step.

### Step 4: Synthesis of I-1-3

A mixed solution of I-1-A (500 mg, 1.49 mmol), I-1-2 (705.71 mg, 1.78 mmol), cesium carbonate (968.93 mg, 2.97 mmol) and N,N-dimethylformamide (4 mL) was stirred at 90° C. for 5 hours until the reaction was complete. The reaction solution was cooled to room temperature, added with water (30 mL) to dilute, stirred and extracted with ethyl acetate (30 mL×3). The combined organic phase was washed with saturated brine (20 mL), dried over anhydrous sodium sulfate, filtered and concentrated in vacuum to obtain I-1-3 (250 mg, 446.76 μmol, yield 30.05%), MS (ESI) m/z: 560.2 [M+H]⁺.

### Step 5: Synthesis of I-1-4

A mixed solution of I-1-3 (250 mg, 446.76 μmol), dichloromethane (3 mL) and trifluoroacetic acid (3 mL) was stirred at room temperature for 2 hours until the reaction was complete. The reaction solution was spin-dried, and the crude product was purified by C18 column chromatography to obtain I-1-4 as a yellow solid (200 mg, 435.29 μmol, yield 97.43%), MS (ESI) m/z: 460.2 [M+H]⁺.

### Step 6: Synthesis of I-1-5

A mixed solution of I-1-4 (25.00 mg, 54.41 μmol), 1-Boc-4-piperidinecarboxylic acid (12.47 mg, 54.41 μmol), diisopropylethylamine (35.16 mg, 272.06 μmol, 47.39 μL), 2-(7-azobenzotriazole)-N,N,N′,N′-tetramethylurea hexafluorophosphate (24.83 mg, 65.29 mol) and N,N-dimethylformamide (2 mL) was added into a reaction bottle, stirred at room temperature for 12 hours until the reaction was complete. The reaction solution was diluted with water (30 mL), stirred and extracted with ethyl acetate (30 mL×3). The combined organic phase was washed with saturated brine (20 mL), dried over anhydrous sodium sulfate, filtered and concentrated in vacuum to obtain a crude product, which was purified by thin layer preparative chromatography to obtain I-1-5 (36 mg, 53.67 μmol, yield 98.64%), MS (ESI) m/z: 671.3 [M+H]⁺.

### Step 7: Synthesis of I-1-6

Trifluoroacetic acid (2 mL) was added dropwise to a mixed solution of I-1-5 (36.00 mg, 53.67 μmol) and dichloromethane (2 mL) under stirring at 0° C. After completion of the addition, the reaction was carried out at room temperature for 1 hour until complete. The reaction solution was spin-dried to obtain I-1-6 (30 mg, crude trifluoroacetate salt), MS (ESI) m/z: 571.6 [M+H]⁺, which was directly used in the next step.

### Step 8: Synthesis of I-1

A mixed solution of I-1-6 (30.00 mg, 52.58 s mol), 2-(2,6-dioxo-piperidin-3-yl)-5-fluoro-isoindole-1,3-dione (14.52 mg, 52.58 μmol), diisopropylethylamine (33.98 mg, 262.88 μmol, 45.79 μL) and N,N-dimethylformamide (2 mL) was reacted at 100° C. for 2 hours to complete the reaction. The reaction solution was diluted with water (20 mL), stirred and extracted with ethyl acetate (20 mL×3). The combined organic phase was washed with saturated brine (20 mL), dried over anhydrous sodium sulfate, filtered and concentrated in vacuum to obtain a crude product, which was purified by thin layer preparative chromatography to obtain I-1 (23 mg, 27.26 μmol, yield 51.85%), MS (ESI) m/z: 827.9 [M+H]⁺, ¹H NMR (500 MHz, DMSO-d₆) δ 11.08 (s, 1H), 10.51 (s, 1H), 8.69 (s, 1H), 8.46 (d, J=8.0 Hz, 1H), 8.41 (t, J=8.0 Hz, 1H), 8.37 (s, 1H), 8.22 (d, J=8.0 Hz, 1H), 7.67 (d, J=8.5 Hz, 1H), 7.33 (s, 1H), 7.25 (d, J=8.5 Hz, 1H), 7.21 (s, 1H), 5.24-5.12 (m, 1H), 5.07 (dd, J=13.0, 5.0 Hz, 1H), 4.07 (d, J=12.5 Hz, 2H), 3.99 (s, 3H), 3.60-3.38 (m, 4H), 3.15-3.05 (m, 2H), 3.04-2.95 (m, 1H), 2.94-2.83 (m, 1H), 2.66-2.52 (m, 2H), 2.50-2.46 (m, 2H), 2.46-2.38 (m, 2H), 2.06-1.98 (m, 1H), 1.81-1.67 (m, 4H), 1.67-1.54 (m, 4H).

Compounds I-2, I-3, I-4, I-5 and I-8 can be prepared by referring to the method of Example 1 above. The structure and characterization data are shown in the following table:

| molec- | | MS (ESI) | |
|---|---|---|---|
| ular | | m/z: | |
| ID | structure | [M + H]⁺ | ¹H NMR |
| I-2 | 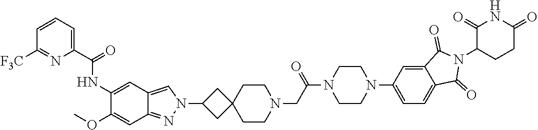 [SMILES: COc1cc2nn(C3CC4(CCN(CC(=O)N5CCN(c6ccc7c(c6)C(=O)N(C6CCC(=O)NC6=O)C7=O)CC5)CC4)C3)cc2cc1NC(=O)c1cccc(C(F)(F)F)n1] | 842.3 | (500 MHz, DMSO-d₆) δ 11.08 (s, 1H), 10.50 (s, 1H), 8.68 (s, 1H), 8.46 (d, J = 8.0 Hz, 1H), 8.41 (t, J = 8.0 Hz, 1H), 8.35 (s, 1H), 8.22 (dd, J = 8.0, 1.0 Hz, 1H), 7.71 (d, J = 8.5 Hz, 1H), 7.38 (d, J = 2.0 Hz, 1H), 7.28 (dd, J = 8.5, 2.0 Hz, 1H), 7.20 |
| | | | (s, 1H), 5.16-5.03 (m, 2H), |
| | | | 3.99 (s, 3H), 3.79-3.68 |
| | | | (m, 2H), 3.66-3.59 (m, 2H), |
| | | | 3.58-3.44 (m, 4H), 3.25-3.08 |
| | | | (m, 2H), 3.01-2.80 (m, 2H), |
| | | | 2.65-2.52 (m, 3H), 2.46-2.33 |
| | | | (m, 6H), 2.05-1.98 (m, 1H), |
| | | | 1.82-1.62 (m, 4H). |
| I-3 | 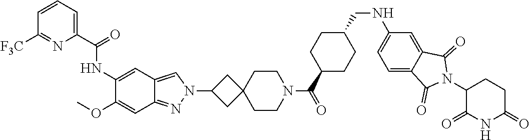 [SMILES: COc1cc2nn(C3CC4(CCN(C(=O)[C@H]5CC[C@H](CNc6ccc7c(c6)C(=O)N(C6CCC(=O)NC6=O)C7=O)CC5)CC4)C3)cc2cc1NC(=O)c1cccc(C(F)(F)F)n1] | 855.3 | (500 MHz, CDCl₃) δ 10.72 (s, 1H), 8.82 (s, 1H), 8.49 (d, J = 8.0 Hz, 1H), 8.12 (t, J = 8.0 Hz, 1H), 7.99 (s, 1H), 7.90-7.84 (m, 2H), 7.62 (d, J = 8.5 Hz, 1H), 7.11 (d, J = 8.5 Hz, 1H), 6.97 (s, 1H), 6.75 (d, J = 8.0 Hz, 1H), 5.08-5.00 (m, 1H), 4.93 (dd, J = 12.5, 5.0 Hz, 1H), |
| | | | 4.04 (s, 3H), 3.65-3.61 |
| | | | (m, 1H), 3.58-3.47 (m, 2H), |
| | | | 3.45-3.38 (m, 1H), 3.11 (d, |
| | | | J = 6.0 Hz, 2H), 2.92-2.86 |
| | | | (m, 1H), 2.85-2.78 (m, 1H), |
| | | | 2.77-2.69 (m, 1H), 2.63-2.53 |
| | | | (m, 4H), 2.16-2.10 (m, 1H), |
| | | | 1.99-1.92 (m, 2H), 1.87-1.71 |
| | | | (m, 8H), 1.68-1.59 (m, 5H). |
| I-4 | 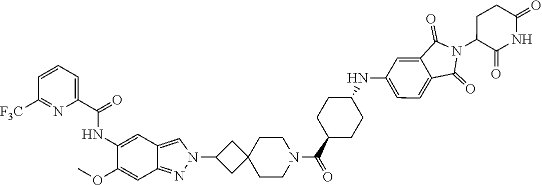 [SMILES: COc1cc2nn(C3CC4(CCN(C(=O)[C@H]5CC[C@H](Nc6ccc7c(c6)C(=O)N(C6CCC(=O)NC6=O)C7=O)CC5)CC4)C3)cc2cc1NC(=O)c1cccc(C(F)(F)F)n1] | 841.3 | (500 MHz, DMSO-d₆) δ 11.05 (s, 1H), 10.51 (s, 1H), 8.69 (s, 1H), 8.46 (d, J = 8.0 Hz, 1H), 8.41 (t, J = 8.0 Hz, 1H), 8.37 (s, 1H), 8.22 (d, J = 8.0 Hz, 1H), 7.55 (d, J = 8.0 Hz, 1H), 7.21 (s, 1H), 7.00 (d, J = 7.0 Hz, 2H), 6.88 (d, J = 8.5 Hz, 1H), 5.21-5.13 (m, 1H), 5.03 (dd, J = 13.0, 5.5 Hz, 1H), 3.99 (s, 3H), 3.57-3.47 (m, 2H), 3.47-3.37 |
| | | | (m, 3H), 2.93-2.79 (m, 1H), |
| | | | 2.71-2.54 (m, 2H), 2.54-2.51 |
| | | | (m, 1H), 2.47-2.35 (m, 3H), |
| | | | 2.05-1.95 (m, 3H), 1.79-1.52 |
| | | | (m, 8H), 1.38-1.22 (m, 3H). |
| I-5 | 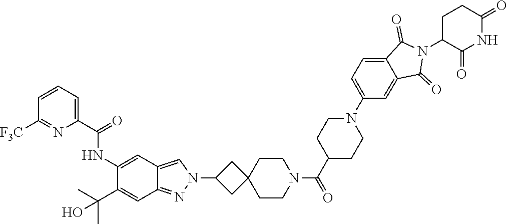 [SMILES: CC(C)(O)c1cc2nn(C3CC4(CCN(C(=O)C5CCN(c6ccc7c(c6)C(=O)N(C6CCC(=O)NC6=O)C7=O)CC5)CC4)C3)cc2cc1NC(=O)c1cccc(C(F)(F)F)n1] | 855.2 | (500 MHz, DMSO-d₆) δ 12.42-12.36 (m, 1H), 11.08 (s, 1H), 8.73 (s, 1H), 8.45 (d, J = 8.0 Hz, 1H), 8.41 (s, 1H), 8.37 (t, J = 8.0 Hz, 1H), 8.16 (d, J = 8.0 Hz, 1H), 7.67 (d, J = 8.5 Hz, 1H), 7.62 (s, 1H), 7.33 (s, 1H), 7.25 (d, J = 8.5 Hz, 1H), 5.97 (s, 1H), 5.28-5.18 (m, 1H), 5.07 (dd, J = 13.0, 5.0 Hz, 1H), 4.07 (d, J = 13.0 Hz, 2H), 3.66-3.38 (m, 5H), 3.15-3.05 (m, 2H), 3.05-2.94 (m, 1H), 2.94-2.84 |
| | | | (m, 1H), 2.63-2.53 (m, 2H), |
| | | | 2.47-2.39 (m, 2H), 2.05-1.98 |
| | | | (m, 1H), 1.77-1.67 (m, 4H), |
| | | | 1.65 (s, 1H), 1.63 (s, 6H), |
| | | | 1.62-1.56 (m, 2H), |
| | | | 1.27-1.22 (m, 2H). |
| I-8 | 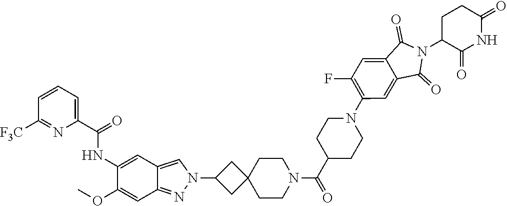 [SMILES: COc1cc2nn(C3CC4(CCN(C(=O)C5CCN(c6cc7c(cc6F)C(=O)N(C6CCC(=O)NC6=O)C7=O)CC5)CC4)C3)cc2cc1NC(=O)c1cccc(C(F)(F)F)n1] | 845.3 | (500 MHz, DMSO-d₆) δ 11.10 (s, 1H), 10.51 (s, 1H), 8.69 (s, 1H), 8.46 (d, J = 8.0 Hz, 1H), 8.41 (t, J = 8.0 Hz, 1H), 8.38 (s, 1H), 8.22 (d, J = 8.0 Hz, 1H), 7.71 (d, J = 11.0 Hz, 1H), 7.45 (d, J = 7.0 Hz, 1H), 7.21 (s, 1H), 5.23-5.14 (m, 1H), 5.11 (dd, J = 13.0, 5.5 Hz, 1H), 3.99 (s, 3H), 3.64 (d, J = 11.5 Hz, 2H), 3.58-3.39 (m, 4H), 3.09-2.97 (m, 2H), 2.96-2.83 |
| | | | (m, 2H), 2.66-2.53 (m, 2H), |
| | | | 2.49-2.34 (m, 4H), 2.10-2.01 |
| | | | (m, 1H), 1.79-1.57 (m, 8H). |

### Example 2: Synthesis of Compound II-1

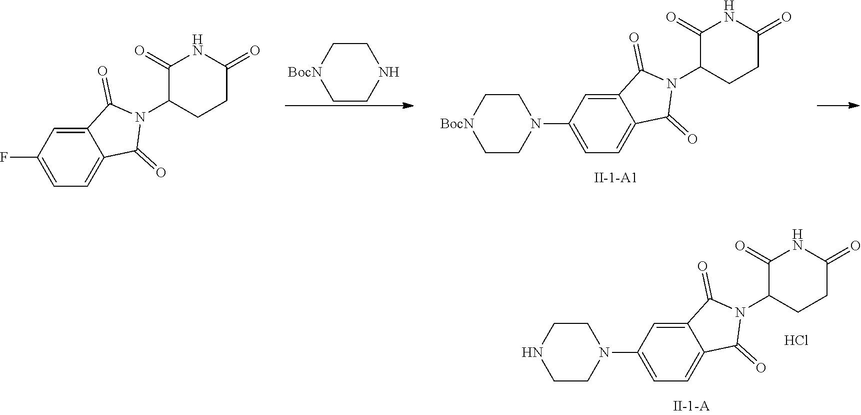 [SMILES: O=C1CCC(N2C(=O)c3ccc(F)cc3C2=O)C(=O)N1 ; CC(C)(C)OC(=O)N1CCNCC1 ; CC(C)(C)OC(=O)N1CCN(c2ccc3c(c2)C(=O)N(C2CCC(=O)NC2=O)C3=O)CC1 ; O=C1CCC(N2C(=O)c3ccc(N4CCNCC4)cc3C2=O)C(=O)N1 ; Cl]

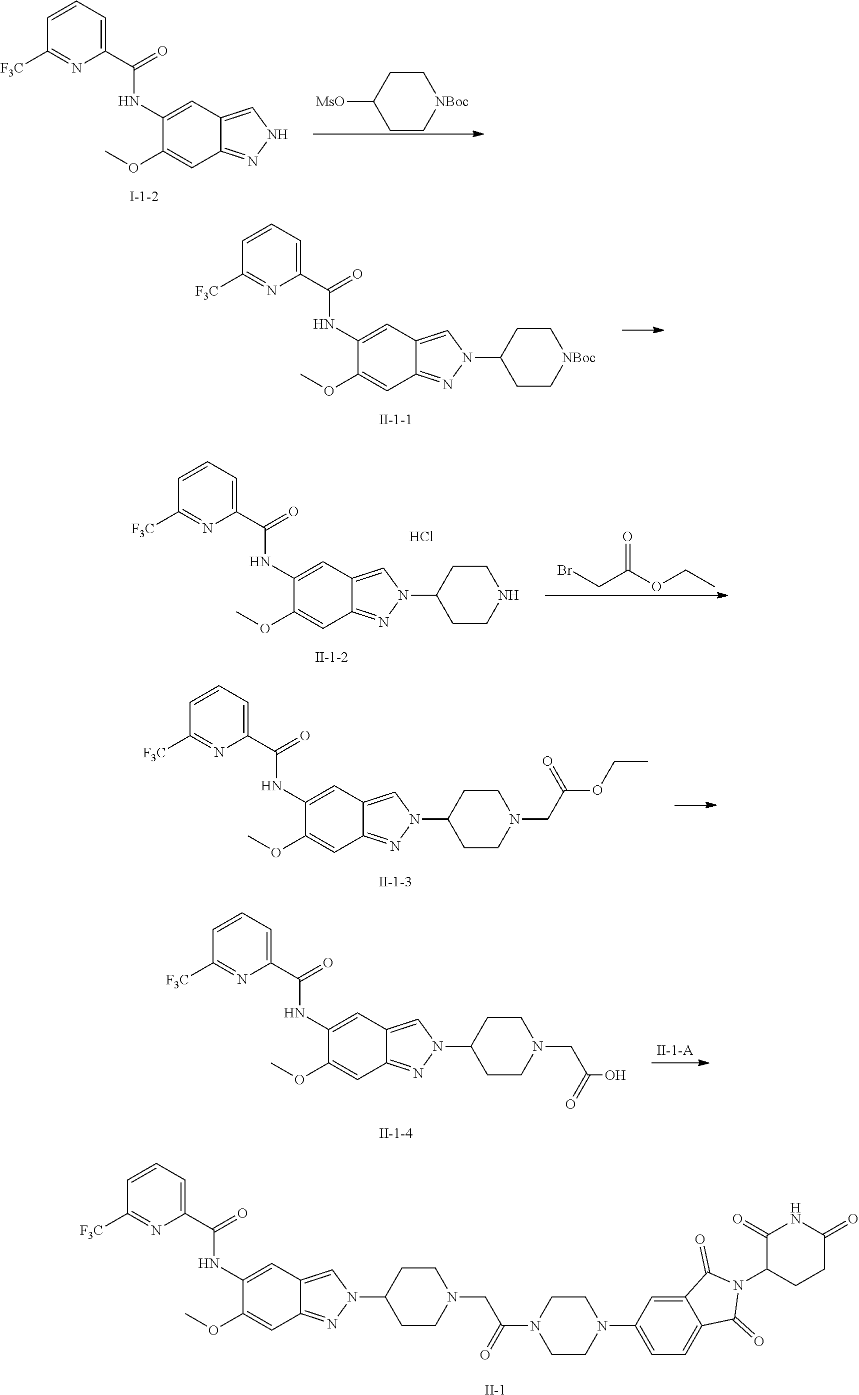

### Step 1: Synthesis of II-1-A1

2-(2,6-dioxy-3-piperidyl)-5-fluoro-isoindole-1,3-dione (247.18 mg, 894.85 μmol) and piperazine-1-carboxylic acid tert-butyl ester (200 mg, 1.07 mmol) were dissolved in dimethyl sulfoxide (3 mL), then diisopropylethylamine (346.95 mg, 2.68 mmol, 467.59 μL) was weighed and added to the reaction system. The mixed solution was stirred at 100° C. for 2 hours until the reaction was complete. The reaction system was added with water (50 mL) to dilute, stirred and extracted with ethyl acetate (50 mL×3). The combined organic phase was washed with brine (30 mL), dried over anhydrous sodium sulfate, filtered and concentrated in vacuum to obtain a crude product, which was purified by silica gel column chromatography (dichloromethane:methanol=20:1) to obtain product II-1-A 1 as a yellow solid (260 mg, 587.62 μmol, yield 65.67%), MS (ESI) m/z: 443.2 [M+H]⁺.

### Step 2: Synthesis of II-1-A

II-1-A1 (260 mg, 587.62 μmol) was dissolved in dichloromethane (3 mL), and added with hydrochloric acid-dioxane (4 M, 2 mL) dropwise under stirring at 0° C. After the dropwise addition was completed, the mixed solution was stirred at 25° C. for 2 hours until the reaction was complete. The reaction system was filtered, washed with dichloromethane (10 mL), and the filter cake was dried to obtain product II-1-A as a white solid (204 mg, 538.52 μmol, yield 91.65%), MS (ESI) m/z: 343.2 [M+H]⁺.

### Step 3: Synthesis of II-1-1

A mixed solution of I-1-2 (1.02 g, 3.03 mmol), tert-butyl 4-methylsulfonyloxypiperidine-1-carboxylate (1.10 g, 3.94 mmol), cesium carbonate (1.98 g, 6.07 mmol) and N,N-dimethylformamide (10 mL) was stirred and reacted under nitrogen protection at 70° C. for 2 hours until the reaction was complete. The reaction mixture was added with water (30 mL), and extracted with ethyl acetate (20 mL×3). The combined organic phases were washed with water (20 mL×3) and brine, dried over anhydrous sodium sulfate, filtered and concentrated under vacuum and reduced pressure to obtain a crude product, which was purified by flash chromatography column (elution with petroleum ether/ethyl acetate=5/1-1/1) to obtain product II-1-1 as an off-white solid (0.35 g, 673.70 μmol, yield 22.21%), MS (ESI) m/z: 520.2 [M+H]⁺.

### Step 4: Synthesis of II-1-2

II-1-1 (0.35 g, 673.70 μmol) was dissolved in dichloromethane (5 mL), and added with hydrochloric acid-dioxane (4 M, 7.9 mL) dropwise under stirring at 0° C. After the dropwise addition was completed, the mixed solution was stirred at 25° C. for 2 hours until the reaction was complete. The reaction system was filtered, washed with dichloromethane (10 mL), and the filter cake was dried to obtain product II-1-2 as a white solid (280 mg, 667.62 μmol, yield 99.10%), MS (ESI) m/z: 420.2 [M+H]⁺.

### Step 5: Synthesis of II-1-3

II-1-2 (120 mg, 286.12 mol) was dissolved in N,N-dimethylformamide (2 mL), and added with potassium carbonate (79.09 mg, 572.25 μmol) and ethyl 2-bromoacetate (71.67 mg, 429.19 μmol, 47.59 μL). The reaction system was stirred at 25° C. for 6 hours until the reaction was complete. The reaction system was added with water (50 mL) to dilute, stirred and extracted with ethyl acetate (50 mL×3). The combined organic phase was washed with brine (30 mL), dried over anhydrous sodium sulfate, filtered and concentrated in vacuum to obtain a crude product, which was purified by silica gel column chromatography (petroleum ether: ethyl acetate=1:1) to obtain II-1-3 as a yellow solid (80 mg, 158.26 μmol, yield 55.31%), MS (ESI) m/z: 506.2 [M+H]⁺.

### Step 6: Synthesis of II-1-4

A mixed solution of II-1-3 (100 mg, 197.83 μmol), water (1 mL), lithium hydroxide (4.74 mg, 197.83 μmol) and methanol (2 mL) was stirred at room temperature for 1 hour until the reaction was complete. Methanol was distilled off under reduced pressure, and dilute hydrochloric acid (2 M) was added to the system to adjust the pH to about 6-7. The reaction system was purified through a C18 reversed-phase chromatography column and freeze-dried to obtain II-1-4 as a yellow solid (30 mg, 62.84 μmol, collected rate 31.76%), MS (ESI) m/z: 478.2 [M+H]⁺.

### Step 7: Synthesis of II-1

A mixed solution of II-1-4 (15 mg, 31.42 μmol), II-1-A (12.91 mg, 34.07 mol), 2-(7-azobenzotriazole)-N,N,N′,N′-tetramethylurea hexafluorophosphate (17.92 mg, 47.13 μmol), diisopropylethylamine (12.18 mg, 94.25 μmol, 16.42 μL) and N,N-dimethylformamide (1 mL) was reacted under nitrogen protection at 25° C. for 2 hours until the reaction was complete. The reaction mixture was added with water (25 mL), and extracted with ethyl acetate (25 mL×3). The combined organic phases were washed with brine (30 mL), dried over anhydrous sodium sulfate, filtered and concentrated in vacuo to obtain a crude product, which was purified by C18 column chromatography to obtain product II-1 as a yellow solid (6 mg, 7.41 μmol, yield 23.58%), MS (ESI) m/z: 802.8 [M+H]⁺. ¹H NMR (500 MHz, DMSO-d₆) δ 11.08 (s, 1H), 10.50 (s, 1H), 8.69 (s, 1H), 8.46 (d, J=8.0 Hz, 1H), 8.40 (t, J=8.0 Hz, 1H), 8.37 (s, 1H), 8.22 (dd, J=8.0, 1.0 Hz, 1H), 7.71 (d, J=8.5 Hz, 1H), 7.39 (d, J=2.0 Hz, 1H), 7.29 (dd, J=8.5, 2.0 Hz, 1H), 7.17 (s, 1H), 5.08 (dd, J=13.0, 5.5 Hz, 1H), 4.46-4.36 (m, 1H), 3.98 (s, 3H), 3.83-3.72 (m, 2H), 3.70-3.61 (m, 2H), 3.61-3.53 (m, 2H), 3.52-3.43 (m, 2H), 3.29 (s, 2H), 3.04-2.94 (m, 2H), 2.93-2.85 (m, 1H), 2.62-2.52 (m, 2H), 2.33-2.24 (m, 2H), 2.19-2.07 (m, 4H), 2.05-2.00 (m, 1H).

Compounds II-5 and II-6 can be prepared by referring to the method of Example 2 above. The structure and characterization data are shown in the following table:

| | | MS | |
|---|---|---|---|
| | | (ESI) | |
| molec- | | m/z: | |
| ular | | [M + | |
| ID | structure | H]⁺ | ¹H NMR |
| II-5 | 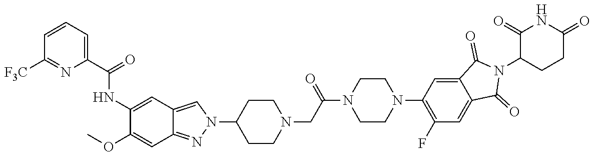 [SMILES: COc1cc2nn(C3CCN(CC(=O)N4CCN(c5cc6c(cc5F)C(=O)N(C5CCC(=O)NC5=O)C6=O)CC4)CC3)cc2cc1NC(=O)c1cccc(C(F)(F)F)n1] | 820.3 | (500 MHz, DMSO-d₆) δ 11.12 (s, 1H), 10.50 (s, 1H), 8.69 (s, 1H), 8.46 (d, J = 8.0 Hz, 1H), 8.40 (t, J = 8.0 Hz, 1H), 8.37 (s, 1H), 8.21 (d, J = 8.0 Hz, 1H), 7.77 (d, J = 11.0 Hz, 1H), 7.52 (d, J = 7.5 Hz, 1H), 7.17 (s, 1H), 5.12 (dd, J = 13.0, 5.0 Hz, 1H), 4.49-4.36 (m, 1H), 3.85-3.75 (m, 2H), 3.70-3.61 (m, |
| | | | 2H), 3.36-3.31 (m, 5H), 3.28 (s, |
| | | | 2H), 3.26-3.22 (m, 2H), 2.99 (d, |
| | | | J = 11.0 Hz, 2H), 2.94-2.84 (m, |
| | | | 1H), 2.66-2.52 (m, 2H), 2.34-2.24 |
| | | | (m, 2H), 2.19-2.01 (m, 5H). |
| II-6 | 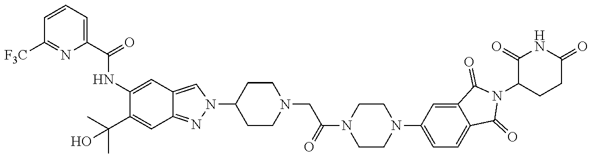 [SMILES: CC(C)(O)c1cc2nn(C3CCN(CC(=O)N4CCN(c5ccc6c(c5)C(=O)N(C5CCC(=O)NC5=O)C6=O)CC4)CC3)cc2cc1NC(=O)c1cccc(C(F)(F)F)n1] | 830.2 | (500 MHz, DMSO-d₆) δ 12.37 (s, 1H), 11.09 (s, 1H), 8.71 (s, 1H), 8.45 (d, J = 8.0 Hz, 1H), 8.40 (s, 1H), 8.37 (t, J = 8.0 Hz, 1H), 8.16 (d, J = 8.0 Hz, 1H), 7.72 (d, J = 8.5 Hz, 1H), 7.58 (s, 1H), 7.39 (d, J = 2.0 Hz, 1H), 7.29 (dd, J = 8.5, 2.0 Hz, 1H), 5.95 (s, 1H), 5.08 (dd, J = 13.0, 5.0 Hz, 1H), 4.51-4.38 (m, 1H), 3.82-3.75 (m, |
| | | | 2H), 3.66-3.54 (m, 4H), 3.52-3.46 |
| | | | (m, 2H), 3.29 (s, 2H), 3.00 (d, J = |
| | | | 11.0 Hz, 2H), 2.93-2.83 (m, 1H), |
| | | | 2.66-2.54 (m, 2H), 2.34-2.26 (m, |
| | | | 2H), 2.18-2.08 (m, 4H), 2.06-1.99 |
| | | | (m, 1H), 1.62 (s, 6H). |

### Example 3: Synthesis of Compound of Compound I-9

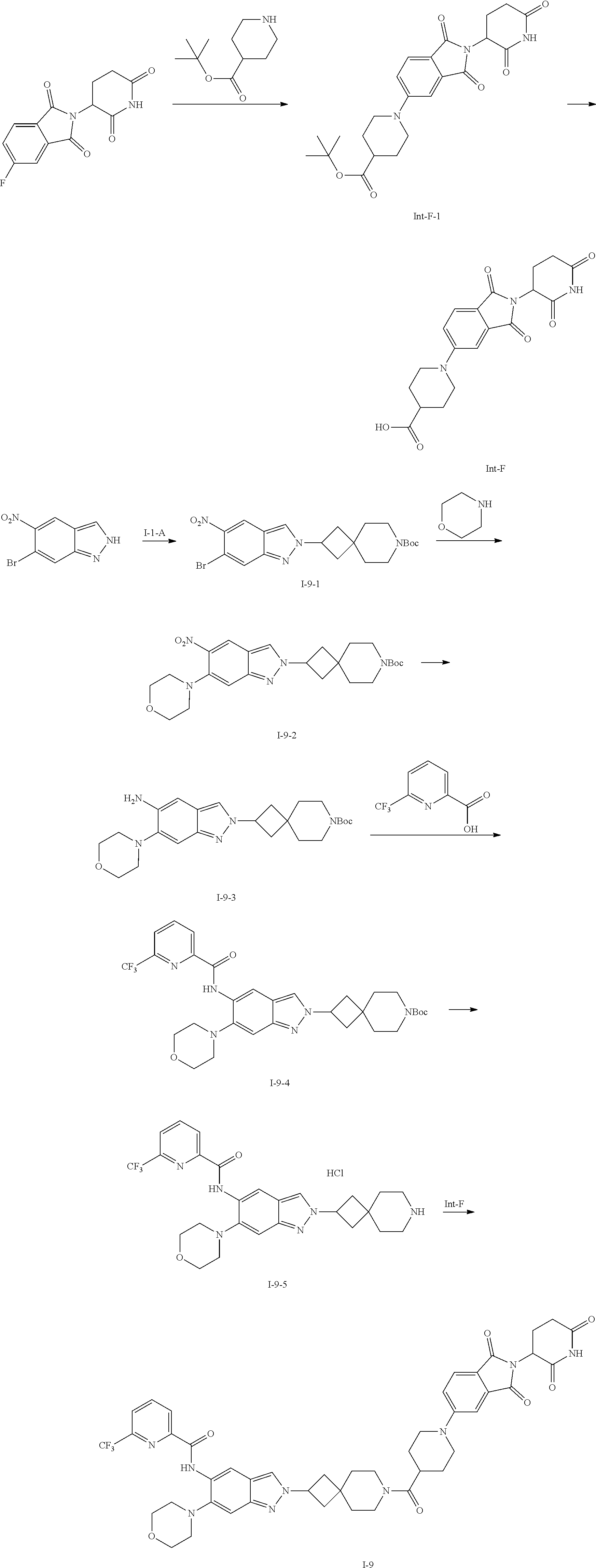

### Step 1: Synthesis of (Int-F-1)

To a solution of 2-(2,6-dioxopiperidin-3-yl)-5-fluoroisoindole-1,3-dione (5.0 g, 18.0 mmol) in anhydrous DMSO (20.0 mL) was added DIPEA (4.7 g, 36.2 mmol) and piperidine-4-carboxylic acid tert-butyl ester (4.0 g, 21.6 mmol), and the reaction mixture was stirred at 90° C. for 2 hours. After cooling to room temperature, the reaction mixture was diluted with water (50 mL) and extracted with ethyl acetate (50 mL×3). The combined organic phases were washed with saturated brine (50 mL), dried over magnesium sulfate, filtered, and concentrated under reduced pressure. The crude product was purified by silica gel column chromatography (PE/EA=5/1) to obtain product int-F-1as a yellow solid (7.5 g, yield 94.2%), MS (ESI) m/z: 442.2 [M+H]⁺.

### Step 2: Synthesis of (Int-F)

TFA (20 mL) was added dropwise to a solution of int-F-1 (5 g, 11.3 mmol) in DCM (50 mL) at 0° C. After the dropwise addition was completed, the mixture was stirred at 25° C. for 16 hours. Diethyl ether was added to the reaction solution under stirring, the solid was filtered after precipitating, and the filter cake was washed with diethyl ether to obtain product int-F as a yellow solid (3.7 g, yield 84.9%), MS (ESI) m/z: 386.1 [M+H]⁺.

### Step 3: Synthesis of (I-9-1)

6-bromo-5-nitro-2H-indazole (1 g, 4.13 mmol) and I-1-A (2.45 g, 6.20 mmol) were dissolved in DMF (20 mL), and added with Cs₂CO₃ (1.35 g) and KI (685.87 mg, 4.13 mmol) with stirring. The reaction solution was stirred at 100° C. for 2 hours, added with water (50 mL), and extracted with ethyl acetate (50 mL×3. The combined organic phases were washed with saturated brine, dried over anhydrous sodium sulfate, filtered, and concentrated under reduced pressure. The crude product was purified by silica gel column chromatography (PE:EA=1:1) to obtain solid I-9-1 as a light yellow (500 mg, yield 26.01%), MS (ESI) m/z: 465.1 [M+H]⁺.

### Step 4: Synthesis of (I-9-2)

I-9-1 (250 mg, 537.24 μmol) and morpholine (936.09 mg, 10.74 mmol) were dissolved in 1,4-dioxane (2 mL), and added with CuI (51.16 mg, 268.62 μmol) and K₃PO₄ (456.15 mg, 2.15 mmol) with stirring. The mixture was reacted at 90° C. for 6 hours under nitrogen protection, added with saturated ammonium chloride solution, and extracted with ethyl acetate (50 mL×3). The combined organic phases were washed with saturated brine, dried over anhydrous sodium sulfate, filtered, and concentrated under reduced pressure. The crude product was purified by silica gel column chromatography to obtain I-9-2 as a light yellow solid (70 mg, yield 27.63%), MS (ESI) m/z: 472.2 [M+H]⁺.

### Step 5: Synthesis of (I-9-3)

I-9-2 (70 mg, 148.45 μmol) was dissolved in MeOH (2 mL), added with wet Pd/C (70 mg, 10% w/w) and replaced with hydrogen three times. The mixture was reacted at 25° C. under a hydrogen atmosphere for 6 hours, and filtered through diatomaceous earth. The filter cake was rinsed three times with ethyl acetate (30 mL), and the filtrate was collected. The solvent was distilled off under reduced pressure to obtain product I-9-3 as a white solid(60 mg, yield 91.53%), MS (ESI) m/z: 442.3 [M+H]⁺.

### Step 6: Synthesis of (I-9-4)

6-(trifluoromethyl)pyridine-2-carboxylic acid (25 mg, 130.82 μmol) and I-9-3 (57.76 mg, 130.82 μmol) were dissolved in DMF (1 mL), added with DIPEA (50.72 mg, 392.45 μmol) and HATU (74.61 mg, 196.22 mol) under stirring. mg, 392.45 mol), and reacted at 25° C. for 2 hours. The reaction system was added with water (30 mL), and extracted with ethyl acetate (30 mL×3). The combined organic phases were washed with saturated brine, dried over anhydrous sodium sulfate, filtered, and concentrated under reduced pressure. The crude product was purified by silica gel column chromatography to obtain the product I-9-4 as a light yellow solid (50 mg, yield 62.18%), MS (ESI) m/z: 615.3 [M+H]⁺.

### Step 7: Synthesis of (I-9-5)

I-9-4 (70 mg, 113.88 μmol) was dissolved in DCM (3 mL), added with HCl-dioxane solution (4 M, 284.71 μL) dropwise under stirring. After the dropwise addition was completed, the reaction system was reacted at 25° C. for 2 hours. The reaction solution was filtered, and the filter cake was washed three times with DCM (30 mL) and dried to obtain the product I-9-5 as a white solid (50 mg, yield 79.68%, hydrochloride), MS (ESI) m/z: 515.2 [M+H]⁺.

### Step 8: Synthesis of (I-9)

To a solution of I-9-5 (40 mg, 72.59 μmol, hydrochloride), int-F (33.57 mg, 87.11 mol) and DMF (1.5 mL) was added DIPEA (28.15 mg, 217.78 μmol) and HATU (41.40 mg, 108.89 μmol), and the resulting mixture was stirred at 125° C. for 1 hour. The reaction system was added with water(20 mL), and extracted with ethyl acetate (20 mL×3). Th combined organic phases were washed with saturated brine, dried over anhydrous sodium sulfate, filtered, and concentrated under reduced pressure. The crude product was purified by preparative thin layer chromatography (DCM:MeOH=20:1) to obtain product I-9 as a yellow solid (23 mg, yield 35.93%), MS (ESI) m/z: 8882.7 [M+H]+. 1H NMR(500 MHz, DMSO-d6) δ 11.15 (s, 1H), 11.14 (s, 1H), 8.85 (s, 1H), 8.56 (d, J=7.5 Hz, 1H), 8.49-8.46 (m, 2H), 8.29 (d, J=8.5 Hz, 1H), 7.73 (d, J=d,) 78.5 Hz, 1H), 7.39 (s, 1H), 7.31 (d, J=8.5 Hz, 1H), 5.29-5.25 (m, 1H), 5.13 (dd, J=12.5, 5.5 Hz, 1H), 4.15-4.10 (m, 2H), 3.96-3.92 (i, 4H), 3.63-3.47 (4, 4H), 3.19-3.13 (m, 2H), 3.09-3.04 (5, 1H), 3.01-2.98 (i, 4H), 2.96-2.91 (n, 1H), 2.71-2.65 (m, 1H), 2.65-2.60 (m, 1H), 2.53-2.45 (i, 3H), 2.11-2.03 (i, 2H), 1.82-31.74 (i, 4H), 1.72-1.64 (, 4H).

Compound I-10 can be prepared by referring to the method of Example 3 above. The structure and characterization data are shown in the following table:

| | | MS | |
|---|---|---|---|
| | | (ESI) | |
| Mole- | | m/z: | |
| cular | | [M + | |
| ID | structure | H]⁺ | ¹H NMR |
| I-10 | 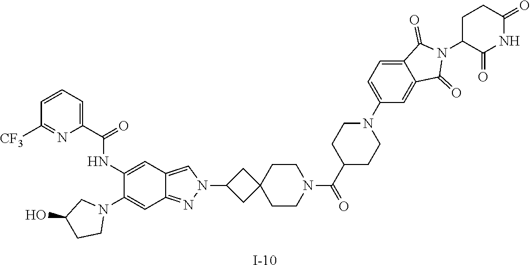 | 882.6 | (500 MHz, DMSO-d₆) δ 11.08 (s, 1H), 10.74 (s, 1H), 8.65 (s, 1H), 8.48 (d, J = 8.0 Hz, 1H), 8.40 (t, J = 8.0 Hz, 1H), 8.38 (s, 1H), 8.21 (d, J = 8.0 Hz, 1H), 7.67 (d, J = 8.5 Hz, 1H), 7.42 (s, 1H), 7.33 (s, 1H), 7.25 (d, J = 8.5 Hz, 1H), 5.21-5.15 (m, 1H), 5.07 (dd, J = 13.0, 5.5 Hz, 1H), 5.01 (d, J = 3.9 Hz, 1H), 4.48-4.44 (m, 1H), 4.07 (d, J = 13.5 Hz, 2H), 3.58-3.52 (m, 2H), 3.51- 3.46 (m, 2H), 3.32-3.28 (m, 2H), 3.27- 3.18 (m, 2H), 3.15-3.08 (m, 3H), 3.04-2.99 (m, 1H), 2.90-2.84 (m, 1H), 2.81 (dd, J = 9.5, 4.5 Hz, 1H), 2.65-2.53 (m, 2H), 2.45-2.38 (m, 2H), 2.22-2.16 (m, 1H), 2.03-1.99 |
| | | | (m, 1H), 1.88-1.83 (m, 1H), 1.76- |
| | | | 1.68 (m, 4H), 1.66-1.58 (m, 4H). |

### Example 4: Synthesis of I-12F

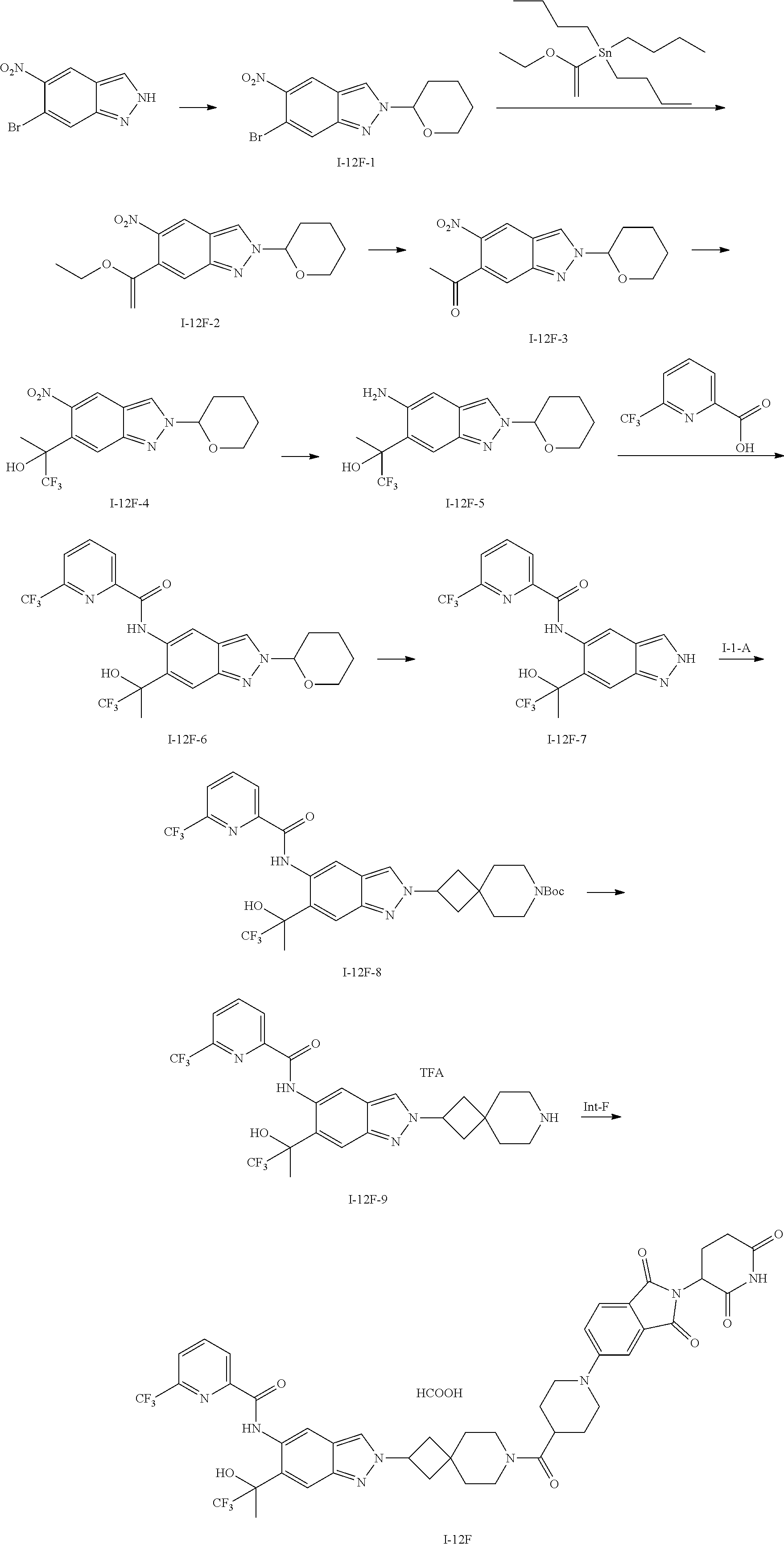

### Step 1: Synthesis of (I-12F-1)

To a solution of 6-bromo- 5-nitro- 1H -indazole (4 g, 16.7 mmol) and 3,4-dihydro-2H-pyran (DHP, 2.8 g, 33.3 mmol) in DCM (50 ml) was added TsOH (621 mg, 3.3 mmol), and the mixture was stirred at 0° C. for 0.5 h. The reaction mixture was diluted with H₂O (120 mL) and extracted with EA (30 mL×3). The combined organic layers were dried over anhydrous Na₂SO₄, filtered and concentrated to obtain a crude product, which was purified by silica gel column chromatography to obtain product I-12F-1 as a yellow solid (4.8 g, yield 88%), MS (ESI) m/z: 326.0 [M+H]⁺.

### Step 2: Synthesis of (I-12F-2)

To a solution of I-12F-1 (4.1 g, 15.6 mmol) in 1,4-dioxane (15 mL) were added tributyl(1-ethoxyvinyl)stannane (5.45 g, 15.1 mmol) and TEA (3.2 g, 31.5 mmol), and the mixture was stirred at 25° C. for 15 minutes under a nitrogen atmosphere. Then Pd(PPh₃)₂Cl₂ (0.9 g, 1.3 mmol) was added, and the resulting mixture was stirred at 100° C. for 16 hours. The reaction solution was cooled to room temperature and diluted with saturated aqueous potassium fluoride solution (50 mL). The resulting mixture was filtered and the filtrate was extracted with EtOAc (60 mL×3). The combined organic layers were dried over anhydrous Na₂SO₄ and concentrated. The crude product was purified by silica gel column chromatography (PE:EtOAc=3:1) to obtain the product II-12F-2 as a yellow oil (2.9 g, yield 12.7%), MS(ESI) m/z:318.3 [M+H]⁺.

### Step 3: Synthesis of (I-12F-3)

To a solution of I-12F-2 (2.5 g, 7.9 mmol), acetone (15 mL) and water (15 mL) was added TsOH (0.15 g, 0.8 mmol), and the mixture was stirred at 50° C. for 2 h, diluted with water (30 mL) and extracted with ethyl acetate (50 mL×3). The combined organic layers were washed with saturated brine, dried over anhydrous sodium sulfate, filtered and concentrated to obtain the product I-12F-3 as a white solid (1.8 g, yield 78.8%), MS (ESI) m/z: 290.1[M+H]⁺.

### Step 4: Synthesis of (I-12F-4)

To a solution of I-12F-3 (2.0 g, 6.92 mmol) in THF (20 mL) was added a solution of trifluoromethyltrimethylsilane (2.04 mL, 13.84 mmol) in THF (27.6 mL) at 25° C. After stirring for 15 minutes, TBA F (6.92 mL, 6.92 mmol) was added, and then the mixture was microwaved at room temperature for 16 hours. The reaction system was poured into a saturated NH₄Cl solution, and then extracted with ethyl acetate (50 mL×2). The combined organic layers were washed with saturated brine, dried over anhydrous sodium sulfate, filtered and concentrated under reduced pressure. The crude product was purified by flash silica gel column chromatography (petroleum ether: ethyl acetate=2:1) to obtain the product I-12F-4 as a yellow solid (1.4 g, yield 22.5%), MS (ESI) m/z: 360.1 [M+H]⁺.

### Step 5: Synthesis of (I-12F-5)

To a solution of I-12F-4 (0.6 g, 1.7 mmol) and MeOH (10 mL) was added wet Pd/C (0.3 g, 10% purity) at 25° C., and the mixture was replaced with H₂ three times under stirring and continued to stir under H₂ environment for 16 hours. The resulting mixture was filtered and concentrated to obtain the product I-12F-5 as a brown solid (0.5 g, yield 91%), MS (ESI) m/z: 330.2 [M+H]⁺.

### Step 6: Synthesis of (I-12F-6)

A mixture of I-12F-5 (1 g, 3.04 mmol), 6-(trifluoromethyl)pyridine-2-carboxylic acid (696.38 mg, 3.64 mmol), HATU (1.39 g, 3.64 mol), DIPEA (784.90 mg, 6.07 mmol, 1.06 mL) and DMF (10 mL) was stirred at 25° C. for 3 hours until the reaction was complete. The reaction was quenched with water and the aqueous layer was extracted with EA (50 mL×3). The combined organic layers were washed with saturated brine, dried over anhydrous sodium sulfate, filtered and concentrated under reduced pressure. The crude product was purified by silica gel column chromatography and eluted with PE/EA (0-45%) to obtain the product I-12F-6 as a white solid (1.4 g, yield 91.77%), MS (ESI) m/z: 503.3 [M+H]⁺.

### Step 7: Synthesis of (I-12F-7)

A mixture of I-12F-6 (0.7 g, 1.39 mmol) and HCl-dioxane solution (4.0 M, 1.39 mmol, 2 mL) was stirred at 25° C. for 16 h. The resulting mixture was concentrated to dryness under reduced pressure to obtain product I-12F-7 as a white solid (0.4 g, yield 63.13%, hydrochloride), MS (ESI) m/z: 419.3 [M+H]⁺.

### Step 8: Synthesis of (I-12F-8)

A mixture of I-12F-7 (1 g, 2.20 mmol), I-1-A (1.30 g, 3.30 mmol), Cs₂CO₃ (3.58 g, 10.99 mmol) and KI (365.03 mg, 2.20 mmol) in DMF (10 mL) was stirred at 90° C. under nitrogen protection for 4 hours. The reaction was quenched with water (50 mL) and extracted with EA (30 mL×3). The combined organic layers were washed with saturated brine, dried over anhydrous sodium sulfate, filtered and concentrated in vacuo. The crude product was purified by reverse-phase C18 column chromatography (mobile phase: MeCN and water containing 0.1% HCOOH, gradient from 1% to 70% over 60 minutes; detector: UV 254 nm) to obtain the product I-12F-8 as a white solid (0.21 g, yield 14.88%), MS (ESI) m/z: 642.5 [M+H]⁺.

### Step 9: Synthesis of (I-12F-9)

I-12F-8 (0.1 g, 155.86 μmol) was dissolved in DCM (2 mL), then TFA (1.49 g, 13.07 mmol, 1 mL) was added, and the mixture was stirred at 25° C. for 2 h. The resulting mixture was concentrated under reduced pressure to obtain the product I-12F-9 as a white solid (0.1 g, crude product, TFA salt), MS (ESI) m/z: 542.3 [M+H]⁺.

### Step 10: Synthesis of (I-12F)

I-12F-9 (0.084 g, 128.14 mol, A), int-F (74.07 mg, 192.22 μmol), HATU (63.34 mg, 166.59 μmol) and DIPEA (33.12 mg, 256.29 mol, 44.64 μL) were dissolved in DMF (1.21 mL), and the mixture was stirred at 25° C. for 2 h. The reaction solution was purified by reverse-phase C18 column chromatography (mobile phase: MeCN and water containing 0.1% HCOOH, gradient from 1% to 60% over 10 minutes; detector: UV 254 nm) to obtain the product I-12F as a yellow solid (0.032 g, yield 25.37%, formate), MS (ESI) m/z: 909.6 [M+H]⁺. ¹H NMR (500 MHz, DMSO-d₆) δ 12.28 (s, 1H), 11.08 (s, 1H), 8.83 (s, 1H), 8.48 (s, 1H), 8.44 (d, J=7.5 Hz, 1H), 8.37 (t, J=7.5 Hz, 1H), 8.17 (d, J=7.5 Hz, 1H), 7.87 (s, 1H), 7.76 (s, 1H), 7.67 (d, J=8.5 Hz, 1H), 7.33 (s, 81H), 7.25 (d, J=9.0 Hz, 1H), 5.35-5.20 (m, 2H), 5.07 (dd, J=12.5, 5.5 Hz, 1H), 4.07 (d, J=812.5 Hz, 2H), 3.58-3.52 (m, 4H), 3.12-3.01 (n, 3H), 2.94-2.83 (m, 2H), 2.65-2.54 (d, 3H), 2.36 (s, 2H), 2.04-1.99 (m, 1H), 1.97 (s, 3H), 1.69-71.57 (m, 10H).

Compounds I-7, 1-13, 1-14, 1-16, 1-17, 1-19, and I-20 can be prepared by referring to the method of Example 1 and Example 4 above. The structure and characterization data are shown in the following table:

| Molecular | | MS (ESI) | |
|---|---|---|---|
| ID | structure | m/z: [M + H]⁺ | ¹H NMR |
| I-7 | 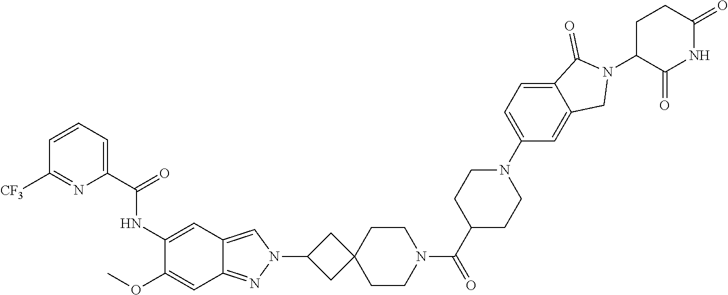 [SMILES: COc1cc2nn(C3CC4(CCN(C(=O)C5CCN(c6ccc7c(c6)CN(C6CCC(=O)NC6=O)C7=O)CC5)CC4)C3)cc2cc1NC(=O)c1cccc(C(F)(F)F)n1] | 813.5 | (500 MHz, DMSO-d₆) δ 10.95 (s, 1H), 10.51 (s, 1H), 8.69 (s, 1H), 8.47 (d, J = 8.0 Hz, 1H), 8.41 (t, J = 8.0 Hz, 1H), 8.38 (s, 1H), 8.25-8.20 (m, 1H), 7.51 (d, J = 8.5 Hz, 1H), 7.21 (s, 1H), 7.07 (s, 1H), 7.06-7.01 (m, 1H), 5.22- 5.12 (m, 1H), 5.05 (dd, J = 13.5, 5.0 Hz, 1H), 4.33 (d, J = 16.9 Hz, 1H), 4.21 (d, J = 16.9 Hz, 1H), 3.99 (s, 3H), 3.94-3.86 (m, 2H), 3.60-3.40 (m, 4H), 3.00-2.86 (m, 4H), 2.67-2.56 (m, 1H), 2.53 (m, 1H), 2.49-2.46 (m, 1H), 2.45-2.34 (m, 4H), 2.00-1.93 (m, 1H), 1.77-1.72 (m, 1H), 1.72- 1.62 (m, 6H), 1.62-1.56 (m, 2H). |
| | I-7 | | |
| I-13 | 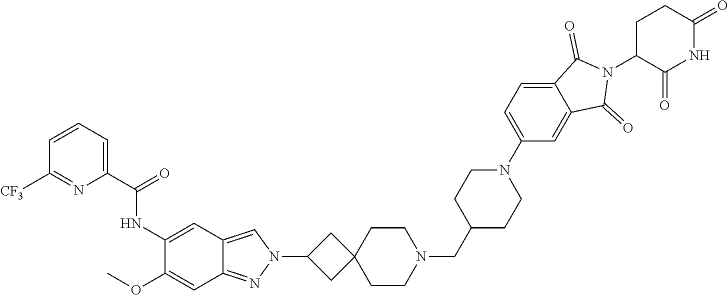 [SMILES: COc1cc2nn(C3CC4(CCN(CC5CCN(c6ccc7c(c6)C(=O)N(C6CCC(=O)NC6=O)C7=O)CC5)CC4)C3)cc2cc1NC(=O)c1cccc(C(F)(F)F)n1] | 813.7 | (500 MHz, DMSO-d₆) δ 11.07 (s, 1H), 10.51 (s, 1H), 8.68 (s, 1H), 8.46 (d, J = 7.5 Hz, 1H), 8.41 (t, J = 7.5 Hz, 1H), 8.35 (s, 1H), 8.22 (d, J = 7.5 Hz, 1H), 7.65 (d, J = 8.5 Hz, 1H), 7.31 (d, J = 2.0 Hz, 1H), 7.23 (dd, J = 8.8, 2.1 Hz, 1H), 7.21 (s, 1H), 5.14-5.04 (m, 2H), 4.07-4.01 (m, 2H), 3.99 (s, 3H), 3.00- 2.93 (m, 2H), 2.92-2.84 (m, 1H), 2.65-2.54 (m, 2H), 2.44-2.23 (m, 8H), 2.12 (d, J = 6.5 Hz, 2H), 2.05- 1.98 (m, 1H), 1.88-1.76 (m, 3H), 1.73- 1.62 (m, 4H), 1.19-1.09 (m, 2H). |
| | I-13 | | |
| I-14 | 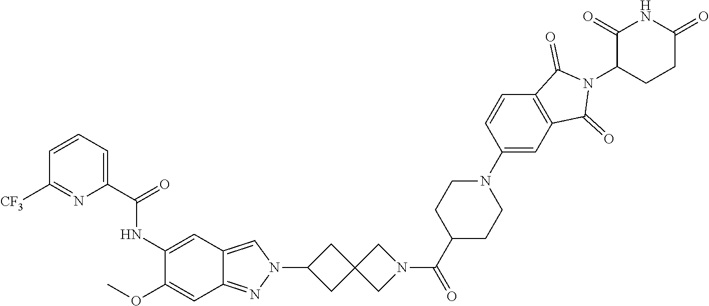 [SMILES: COc1cc2nn(C3CC4(C3)CN(C(=O)C3CCN(c5ccc6c(c5)C(=O)N(C5CCC(=O)NC5=O)C6=O)CC3)C4)cc2cc1NC(=O)c1cccc(C(F)(F)F)n1] | 799.5 | (500 MHz, CDCl₃) δ 10.72 (s, 1H), 8.82 (s, 1H), 8.49 (d, J = 8.0 Hz, 1H), 8.13 (t, J = 8.0 Hz, 1H), 7.97 (s, 1H), 7.87-7.85 (m, 2H), 7.69 (dd, J = 8.5, 1.5 Hz, 1H), 7.28 (d, J = 2.5 Hz, 1H), 7.10 (s, 1H), 7.06 (dd, J = 8.5, 2.5 Hz, 1H), 4.96-4.92 (m, 2H), 4.36 (s, 1H, 4.32 (s, 1H), 4.16 (s, 1H), 4.12 (s, 1H), 4.04 (s, 3H), 3.99-3.96 (m, 2H), 3.09- 3.04 (m, 4H), 2.91-2.84 (m, 4H), 2.74-2.69 (m, 1H), 2.49-2.44 (m, 1H), 2.16-2.11 (m, 1H), 1.91-1.82 (m, 4H). |
| | I-14 | | |
| I-16 | 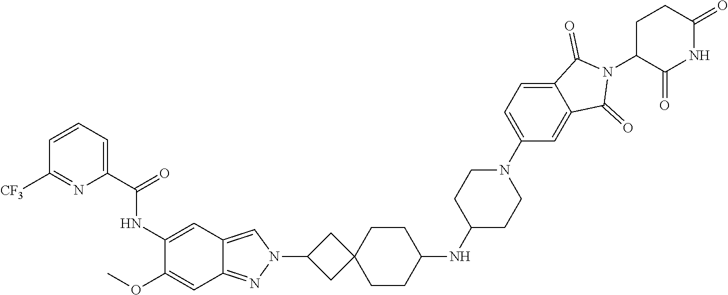 [SMILES: COc1cc2nn(C3CC4(CCC(NC5CCN(c6ccc7c(c6)C(=O)N(C6CCC(=O)NC6=O)C7=O)CC5)CC4)C3)cc2cc1NC(=O)c1cccc(C(F)(F)F)n1] | 813.5 | (500 MHz, DMSO-d₆) δ 11.08 (s, 1H), 10.51 (s, 1H), 8.68 (s, 1H), 8.46 (d, J = 7.5 Hz, 1H), 8.41 (t, J = 7.5 Hz, 1H), 8.36 (s, 1H), 8.22 (d, J = 8.0 Hz, 2H), 7.68 (d, J = 8.5 Hz, 1H), 7.35 (s, 1H), 7.27 (d, J = 8.5 Hz, 1H), 7.20 (s, 1H), 5.12-5.05 (m, 2H), 4.06 (d, J = 12.5 Hz, 2H), 3.99 (s, 3H), 3.09-3.01 (m, 3H), 2.87-2.82 (m, 3H), 2.65-2.57 (m, 2H), 2.42-2.35 (m, 2H), 2.34-2.28 (m, 2H), 2.05-1.99 (m, 1H), 1.96- 1.91 (m, 3H), 1.86-1.80 (m, 4H), 1.50- 1.42 (m, 2H), 1.39-1.34 (m, 2H). |
| | I-16 | | |
| I-17 | 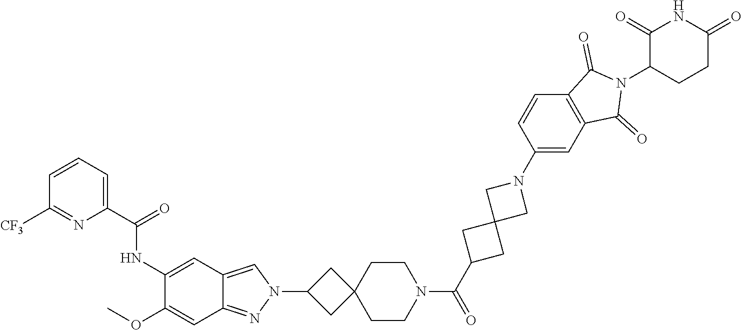 [SMILES: COc1cc2nn(C3CC4(CCN(C(=O)C5CC6(C5)CN(c5ccc7c(c5)C(=O)N(C5CCC(=O)NC5=O)C7=O)C6)CC4)C3)cc2cc1NC(=O)c1cccc(C(F)(F)F)n1] | 839.6 | (500 MHz, DMSO-d₆) δ 1H NMR (500 MHz, DMSO) δ 11.07 (s, 1H), 10.51 (s, 1H), 8.69 (s, 1H), 8.46 (d, J = 7.5 Hz, 1H), 8.41 (t, J = 8.0 Hz, 1H), 8.37 (s, 1H), 8.22 (dd, J = 8.0, 1.0 Hz, 1H), 7.63 (d, J = 8.0 Hz, 1H), 7.21 (s, 1H), 6.79 (d, J = 2.0 Hz, 1H), 6.65 (dd, J = 8.5, 2.0 Hz, 1H), 5.19-5.13 (m, 1H), 5.05 (dd, J = 12.8, 5.4 Hz, 1H), 3.99 (s, 3H), 3.78 (s, 2H), 3.70 (s, 2H), 3.50 (br, 2H), 3.41 (br, 2H), 2.64-2.56 (m, 2H), 2.53 (s, 2H), 2.44-2.39 (m, 2H), 2.04-1.98 (m, 1H), 1.94 (d, J = 10.5 Hz, 2H), 1.74-1.66 (m, 2H), 1.65- 1.55 (m, 6H). |
| | I-17 | | |
| I-19 | 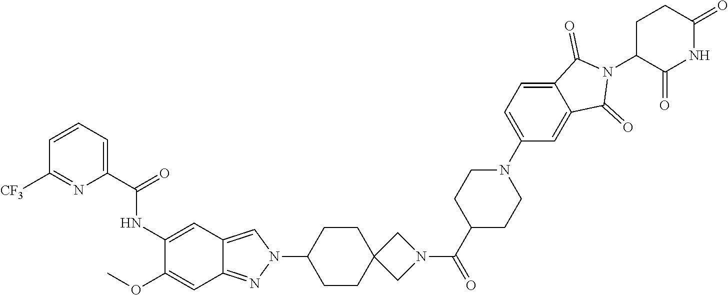 [SMILES: COc1cc2nn(C3CCC4(CC3)CN(C(=O)C3CCN(c5ccc6c(c5)C(=O)N(C5CCC(=O)NC5=O)C6=O)CC3)C4)cc2cc1NC(=O)c1cccc(C(F)(F)F)n1] | 827.5 | (500 MHz, DMSO-d₆) δ 11.08 (s, 1H), 10.50 (d, J = 2.0 Hz, 1H), 8.69 (d, J = 3.5 Hz, 1H), 8.46 (d, J = 7.5 Hz, 1H), 8.41 (t, J = 7.5 Hz, 1H), 8.34 (d, J = 2.5 Hz, 1H), 8.22 (d, J = 7.5 Hz, 1H), 7.67 (d, J = 8.5 Hz, 1H), 7.35 (d, J = 1.3 Hz, 1H), 7.27 (dd, J = 8.7, 1.7 Hz, 1H), 7.16 (d, J = 14.0 Hz, 1H), 5.07 (dd, J = 12.5, 5.5 Hz, 1H), 4.47-4.37 (m, 1H), 4.12-4.05 (m, 2H), 4.04 (s, 1H), 3.98 (d, J = 3.0 Hz, 3H), 3.91 (s, 1H), 3.66 (s, 1H), 3.56 (s, 1H), 3.10- 3.00 (m, 2H), 2.94-2.83 (m, 2H), 2.66- 2.53 (m, 3H), 2.10-1.99 (m, 4H), 1.98-1.88 (m, 2H), 1.78-1.66 (m, 4H), 1.63-1.53 (m, 2H). |
| | I-19 | | |
| I-20 | 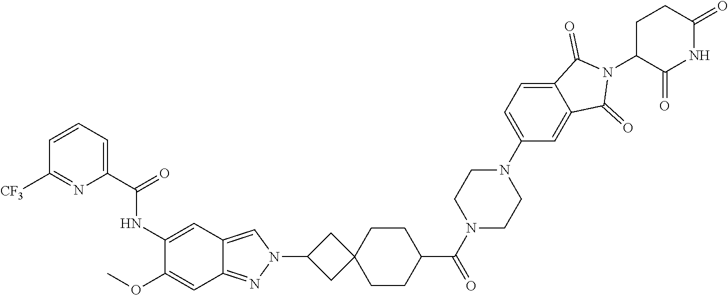 [SMILES: COc1cc2nn(C3CC4(CCC(C(=O)N5CCN(c6ccc7c(c6)C(=O)N(C6CCC(=O)NC6=O)C7=O)CC5)CC4)C3)cc2cc1NC(=O)c1cccc(C(F)(F)F)n1] | 827.5 | (400 MHz, DMSO-d₆) δ 11.09 (s, 1H), 10.51 (s, 1H), 8.68 (s, 1H), 8.46 (d, J = 8.0 Hz, 1H), 8.40 (t, J =7.5 Hz, 1H), 8.36 (s, 1H), 8.21 (d, J = 7.5 Hz, 1H), 7.71 (d, J = 8.5 Hz, 1H), 7.36 (s, 1H), 7.26 (d, J = 8.5 Hz, 1H), 7.21 (s, 1H), 5.19-5.00 (m, 2H), 3.99 (s, 3H), 3.69 (br, 2H), 3.61 (br, 2H), 3.55-3.46 (m, 6H), 2.95-2.82 (m, 1H), 2.68-2.58 (m, 2H), 2.42-2.35 (m, 1H), 2.35- 2.25 (m, 2H), 2.07-1.92 (m, 2H), 1.87- 1.78 (m, 1H), 1.67-1.39 (m, 6H). |
| | I-20 | | |

### Step 1: Synthesis of (I-22-1)

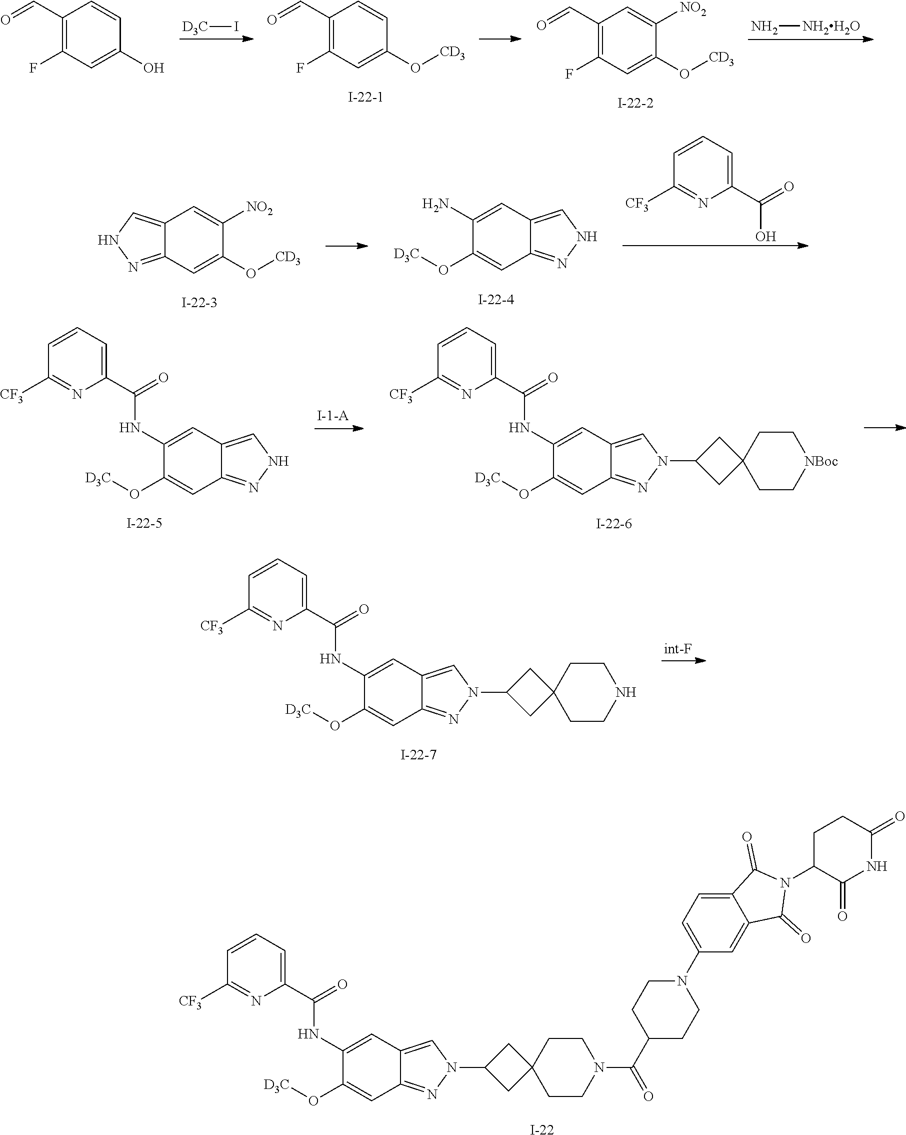

To a solution of 2-fluoro-4-hydroxybenzaldehyde (25 g, 178.4 mmol), K₂CO₃ (49.3 g, 356.9 mmol) in DMF (400 mL) was added CD₃-I (31 g, 214.2 mmol) under nitrogen at 0° C., and the mixture was stirred at 25° C. for 16 hours. The mixture was quenched with NH₄Cl solution (saturated, 400 mL) and extracted with ethyl acetate (500 mL×3). The combined organic phases were washed with saturated brine, dried over anhydrous sodium sulfate, filtered, and concentrated under reduced pressure to obtain product I-22-1 as a white solid (27 g, yield 95.7%), MS (ESI) m/z: 158.2 [M+H]⁺. ¹H NMR (400 MHz, CDCl₃) δ 10.21 (s, 1H), 7.82 (t, J=8.4 Hz, 1H), 6.78 (dd, J=8.8, 2.4 Hz, 1H), 6.64 (dd, J=12.4, 2.4 Hz, 1H).

### Step 2: Synthesis of (I-22-2)

To a solution of K NO₃ (18 g, 179 mmol) and concentrated H₂SO₄ (130 mL) was added I-22-1 (25 g, 163 mmol) in batches at 0° C. After 0.5 hours, the dropwise addition was completed, and the mixture was stirred at 25° C. for 2 hours. The reaction mixture was quenched with ice water, filtered, washed with H₂O and dried under vacuum to obtain product I-22-2 as a yellow solid (30 g, yield 93.2%), MS (ESI) m/z: 203.2 [M+H]⁺. ¹H NMR (400 MHz, CDCl₃) δ 10.22 (s, 1H), 8.46 (d, J=7.2 Hz, 1H), 6.87 (d, J=11.6 Hz, 1H).

### Step 3: Synthesis of (I-22-3)

A solution of I-22-2 (27 g, 133.5 mmol), hydrazine hydrate (13.4 g, 267.1 mmol) and DMF (60 mL) was stirred at 140° C. for 2 hours. After cooling to room temperature, the reaction mixture was washed with ice water to obtain product I-22-3 as a yellow solid (30 g, yield 97%), MS (ESI) m/z: 197.2 [M+H]⁺. ¹H NMR (400 MHz, DMSO-d₆) δ 13.36 (s, 1H), 8.41 (s, 1H), 8.20 (s, 1H), 7.21 (s, 1H).

### Step 4: Synthesis of (I-22-4)

A mixed solution of I-22-3 (16 g, 81.6 mmol), a solution of wet Pd/C (3 g, 10% purity) in THF (1 L) and DMF (100 mL) was stirred under a hydrogen atmosphere at 25° C. for 16 hours. The reaction system was filtered through diatomaceous earth and washed with THF (1 L). The filtrate was distilled under reduced pressure to obtain product I-22-4 as a brown solid (11.5 g, yield 84.9%), MS (ESI) m/z: 167.2 [M+H]⁺.

### Step 5: Synthesis of (I-22-5)

A mixed solution of I-22-4 (23 g, 138 mmol), HATU (56 g, 162 mmol), DIEA (32 g, 277 mmol) and DMF (100 mL) was stirred at 25° C. for 10 min, and then a solution of 6-(trifluoromethyl)picolinic acid (23 g, 135 mmol) in DMF (20 mL) was added at 0° C. The resulting mixture was stirred at 25° C. for 16 h, diluted with H₂O (300 mL) and extracted with ethyl acetate (300 mL×3). The combined organic phases were concentrated under reduced pressure to obtain product I-22-5 as a brown solid (24 g, yield 51.1%), MS (ESI) m/z: 340.1[M+H]⁺.

### Step 6: Synthesis of (I-22-6)

A solution of I-22-5 (24 g, 70.8 mmol), I-1-A (42 g, 106.2 mmol), Cs₂CO₃ (69 g, 212.4 mmol), KI (11.8 g, 70.8 mmol) and DMF (120 mL) was stirred at 90° C. for 16 hours under a nitrogen atmosphere. The mixture was diluted with H₂O (200 mL) and extracted with EtOAc (300 mL×3). The combined organic phases were washed with saturated brine, dried over anhydrous sodium sulfate, filtered and concentrated. The crude product was purified by flash silica gel column chromatography (PE/EtOAc=1/1) to obtain product I-22-6 as a yellow solid (5 g, yield 13%), MS (ESI) m/z: 563.2 [M+H]⁺.

### Step 7: Synthesis of (I-22-7)

A solution of I-22-6 (5 g, 8.9 mmol) and HCl-dioxane (1 M, 100 mL) was stirred at 25° C. for 1 hour. Diethyl ether was added to the reaction mixture under stirring. The supernatant was removed after standing and repeated three times to remove the residual HCl in the reaction mixture to obtain the product I-22-7 as a white solid (4 g, yield 97%), MS (ESI) m/z: 463.2 [M+H]⁺.

### Step 8: Synthesis of (I-22)

A solution of int-F (2.9 g, 7.5 mmol), DIE A (3.9 g, 30.0 mmol), H A TU (3.4 g, 9.0 mmol) and DMF (20 mL) was stirred at 25° C. for 10 min, and then a solution of I-22-7 (3.7 g, 7.5 mmol) in DMF (10 mL) was added at 0° C. The resulting mixture was stirred for 2 hours until the reaction was complete. The mixture was diluted with H₂O (100 mL) and extracted with ethyl acetate (100 mL×3). The combined organic phases were washed with saturated brine (200 mL), dried over anhydrous sodium sulfate, filtered and concentrated under reduced pressure to obtain a crude product, which was purified by reversed phase C18 column chromatography to obtain product I-22 as a yellow solid (2.1 g, yield 32%), MS (ESI)m/z:830.2[M+H]⁺. ¹H NMR (400 MHz, DMSO-d₆) δ 11.08 (s, 1H), 10.51 (s, 1H), 8.69 (s, 1H), 8.52-8.33 (m, 3H), 8.22 (d, J=7.6 Hz, 1H), 7.67 (d, J=8.4 Hz, 1H), 7.33 (s, 1H), 7.29-7.17 (m, 2H), 5.231-5.12 (m, 1H), 5.07 (dd, J=12.8, 5.2 Hz, 1H), 4.07 (d, J=13.2 Hz, 2H), 3.58-3.31 (m, 4H), 3.10-3.05 (m, 3H), 2.92-2.84 (m, 1H), 2.61-2.54 (m, 2H), 2.48-2.35 (m, 4H), 2.07-1.96 (m, 1H), 1.77-1.53 (m, 8H).

### Example 6: Synthesis of I-23

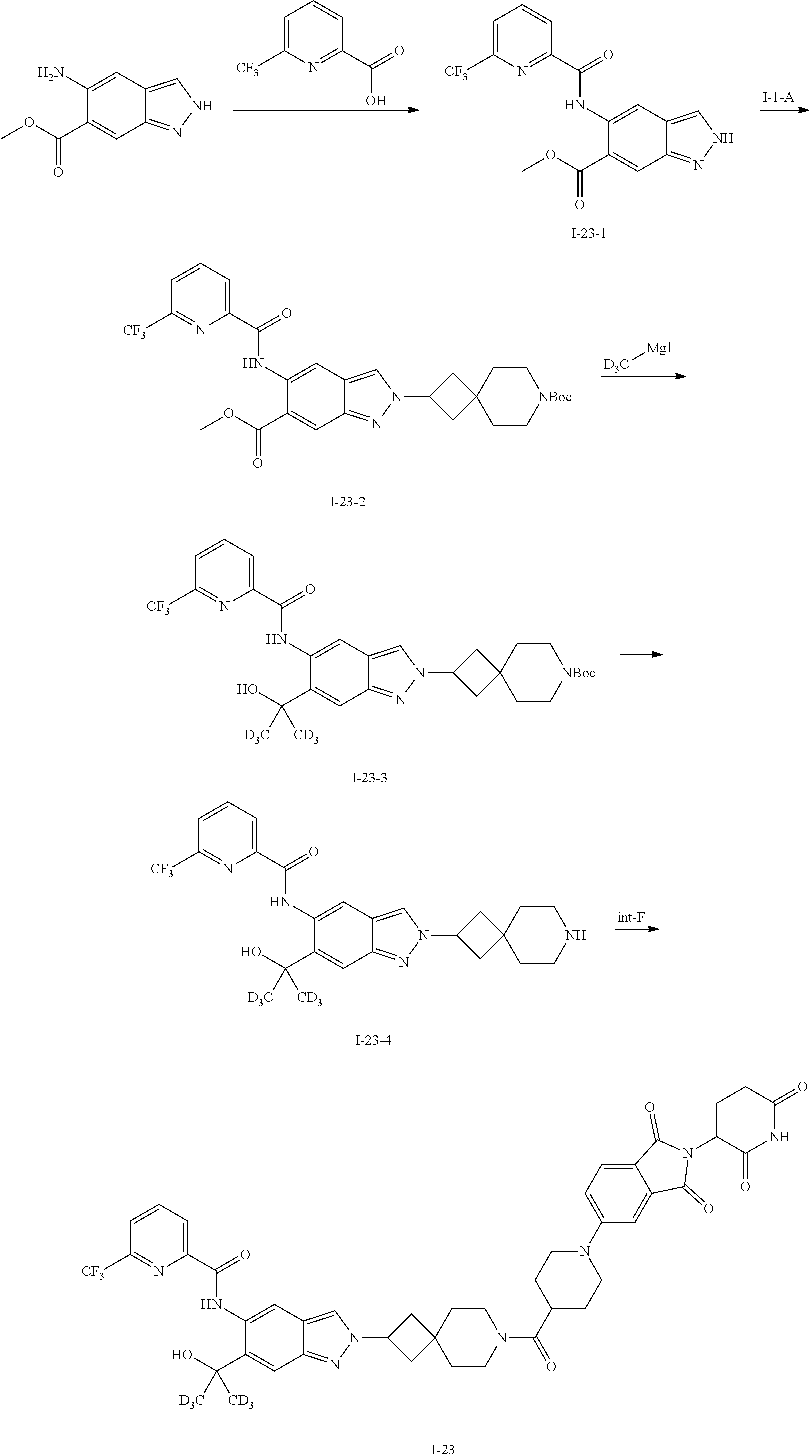

### Step 1: Synthesis of (I-23-1)

5-amino-2H-indazole-6-carboxylic acid methyl ester (5 g, 26 mmol) was dissolved in tetrahydrofuran (50 mL), and the reaction solution was slowly added with DIPEA (27 g, 209 mmol), and then added with T₃P (propyl phosphoric anhydride, 25 g, 78.6 mmol), and then added with 6-(trifluoromethyl)picolinic acid (7.5 g, 39 mmol). After the dropwise dropwise addition was completed, the reaction solution was stirred at 25° C. for 16 hours. Water was added to the reaction system to precipitate the solid, which was filtered, and dried, and the filter cake was filtered under vacuum to obtain the product I-23-1 as a light yellow solid (9.3 g, yield 98%).

### Step 2: Synthesis of (I-23-2)

A solution of I-23-1(5 g, 13.72 mmol), I-1-A (10.86 g, 27.45 mmol), Cs₂CO₃ (13.4 g, 41.2 mmol), KI (2.28 g, 13.72 mmol) and DMF (50 mL) was stirred at 50° C. for 16 hours. The mixture was diluted with H₂O (50 mL) and extracted with EtOAc (150 mL×3). The combined organic phases were washed with saturated brine, dried over anhydrous sodium sulfate, filtered and concentrated. The crude product was purified by flash silica gel column chromatography (PE/EtOAc=1/1) to obtain product I-23-2 as a brown solid (1.0 g, yield 12.3%), MS (ESI) m/z: 588.2 [M+H]⁺.

### Step 3: Synthesis of (I-23-3)

To a solution of I-23-2 (850 mg, 1.45 mmol) in THF (40 mL) was added dropwise (methyl-D₃) magnesium iodide (14.5 mL, 14.5 mmol) at 0° C., and the mixture was stirred at 25° C. for 16 hours. The reaction solution was poured into water and extracted with EtOAc (50 mL×3). The combined organic phases were washed with saturated brine, dried over anhydrous sodium sulfate, filtered, and concentrated under reduced pressure. The crude product was purified by silica gel column chromatography (PE/EtOAc=2/1) to obtain product I-23-3 as a yellow solid (100 mg, yield 11.6%), MS (ESI) m/z: 594.4 [M+H]⁺.

### Step 4: Synthesis of (I-23-4)

To a solution of I-23-3 (100 mg, 0.17 mmol) in DCM (5 mL) was added dropwise TFA (2 mL) at 0° C., and the mixture was stirred at 25° C. for 2 h. Diethyl ether was added to the reaction mixture under stirring. The supernatant was removed after standing and repeated three times to remove the residual TFA in the reaction solution, and dried under vacuum to obtain product I-23-4 as a yellow solid (80 mg, yield 95.2%), MS (ESI) m/z: 494.3 [M+H]⁺.

### Step 5: Synthesis of (I-23)

To a solution of int-F (80 mg, 0.21 mmol), HATU (92 mg, 0.24 mmol) and DMF (5 mL) were added DIEA (53 mg, 0.42 mmol) and I-23-4 (100 mg, 0.21 mmol), and the mixture was stirred at 25° C. for 16 hours. The mixture was added with water (10 mL) and extracted with EtOAc (30 mL×3). The combined organic phases were washed with saturated brine, dried over anhydrous sodium sulfate, filtered, and concentrated under reduced pressure. The crude product was purified by preparative H PLC to obtain product I-23 as a white solid (24.05 mg, yield 13.9%), MS (ESI) m/z: 861.3 [M+H]⁺. ¹H NMR (400 MHz, DMSO-d₆) δ 12.42 (s, 1H), 11.11 (s, 1H), 8.77 (s, 1H), 8.50 (d, J=7.6 Hz, 1H), 8.46-8.37 (m, 2H), 8.20 (d, J=7.6 Hz, 1H), 7.71 (d, J=8.4 Hz, 1H), 7.65 (s, 1H), 7.37 (s, 1H), 7.29 (d, J=8.4 Hz, 1H), 5.98 (s, 1H), 5.35-5.21 (m, 1H), 5.11 (m, 1H), 4.11 (d, J=11.6 Hz, 2H), 3.61-3.46 (m, 6H), 3.16-3.04 (m, 2H), 3.04-2.96 (m, 1H), 2.95-2.81 (m, 1H), 2.69-2.55 (m, 2H), 2.47-2.36 (m, 2H), 2.10-1.96 (m, 1H). 1.84-1.57 (m, 8H).

Compounds I-24 to I-29, I-31˜I-38, I-48, I-51, I-53, I-56 can be prepared by referring to the method of Examples 1, 3, 5 and 6 above. The structure and characterization data are shown in the following table:

| Mole- | | | |
|---|---|---|---|
| cular | | MS (ESI) | |
| ID | structure | m/z: [M + H]⁺ | ¹H NMR |
| I-24 | 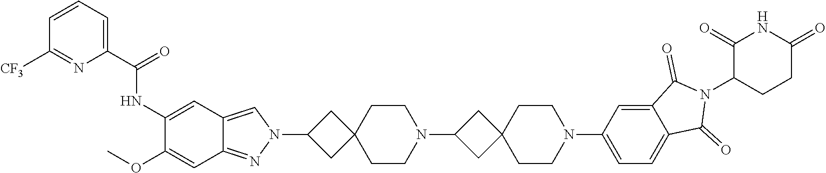 [SMILES: COc1cc2nn(C3CC4(CCN(C5CC6(CCN(c7ccc8c(c7)C(=O)N(C7CCC(=O)NC7=O)C8=O)CC6)C5)CC4)C3)cc2cc1NC(=O)c1cccc(C(F)(F)F)n1] I-24 | 839.7 | (500 MHz, DMSO-d₆) δ 11.08 (s, 1H), 10.51 (s, 1H), 8.68 (s, 1H), 8.46 (d, J = 8.0 Hz, 1H), 8.40 (t, J = 8.0 Hz, 1H), 8.36 (s, 1H), 8.22 (d, J = 8.0 Hz, 1H), 7.65 (d, J = 8.5 Hz, 1H), 7.32 (s, 1H), 7.24 (d, J = 8.5 Hz, 1H), 7.21 (s, 1H), 5.16-5.09 (m, 1H), 5.06 (dd, J = 12.5, 5.5 Hz, 1H), 3.99 (s, 3H), 3.47 (br, 2H), 3.39 (br, 2H), 2.92-2.84 (m, 1H), 2.65-2.53 (m, 3H), 2.53- 2.51 (m, 1H), 2.47-2.29 (m, 4H), 2.07-1.95 (m, 1H), 1.78- |
| | | | 1.42 (m, 9H), 1.36-1.24 (m, |
| | | | 2H), 1.24-1.20 (m, 2H). |
| I-25 | 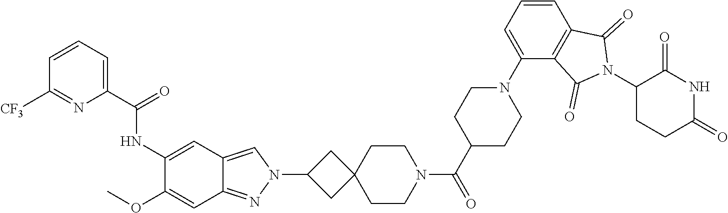 [SMILES: COc1cc2nn(C3CC4(CCN(C(=O)C5CCN(c6cccc7c6C(=O)N(C6CCC(=O)NC6=O)C7=O)CC5)CC4)C3)cc2cc1NC(=O)c1cccc(C(F)(F)F)n1] I-25 | 827.6 | (500 MHz, DMSO-d₆) δ 11.09 (s, 1H), 10.51 (s, 1H), 8.69 (s, 1H), 8.47 (d, J = 7.5 Hz, 1H), 8.41 (t, J = 8.0 Hz, 1H), 8.38 (s, 1H), 8.22 (dd, J = 7.5, 0.5 Hz, 1H), 7.69 (t, J = 8.0 Hz, 1H+), 7.34 (t, J = 8.0 Hz, 2H), 7.22 (s, 1H), 5.22-5.13 (m, 1H), 5.10 (dd, J = 12.5, 5.4 Hz, 1H), 3.99 (s, 3H), 3.78-3.69 (m, 2H), 3.60-3.49 (m, 2H), 3.50-3.40 (m, 2H), 3.04-2.95 (m, 2H), 2.93-2.83 (m, 2H), 2.63-2.54 (m, 2H), 2.54-2.52 (m, 1H), 2.49-2.46 (m, 1H), 2.46-2.38 (m, 2H), 2.08-1.98 (m, 1H), |
| | | | 1.87-1.78 (m, 2H), 1.76-1.68 |
| | | | (m, 4H), 1.68-1.57 (m, 2H). |
| I-26 | 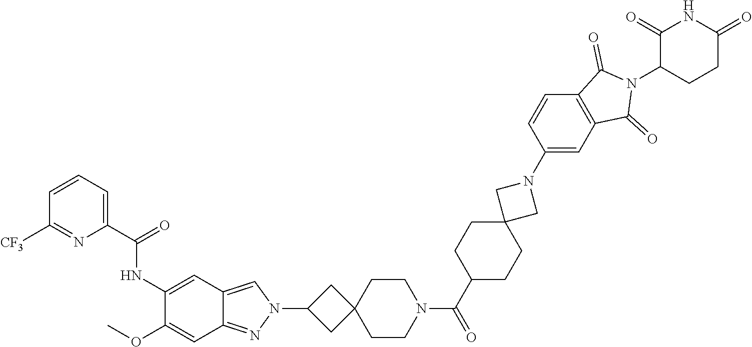 [SMILES: COc1cc2nn(C3CC4(CCN(C(=O)C5CCC6(CC5)CN(c5ccc7c(c5)C(=O)N(C5CCC(=O)NC5=O)C7=O)C6)CC4)C3)cc2cc1NC(=O)c1cccc(C(F)(F)F)n1] | 867.6 | (500 MHz, DMSO-d₆) δ 11.07 (s, 1H), 10.51 (s, 1H), 8.69 (s, 1H), 8.46 (d, J = 7.5 Hz, 1H), 8.41 (t, J = 8.0 Hz, 1H), 8.37 (s, 1H), 8.22 (dd, J = 7.5, 0.8 Hz, 1H), 7.63 (d, J = 8.0 Hz, 1H), 7.21 (s, 1H), 6.79 (d, J = 1.5 Hz, 1H), 6.65 (dd, J = 8.5, 2.0 Hz, 1H), 5.20-5.12 (m, 1H), 5.05 (dd, J = 13.0, 5.5 Hz, 1H), 3.99 (s, 3H), 3.78 (s, 2H), 3.70 (s, 2H), 3.50 (br, 2H), 3.42 (br, 2H), 2.93-2.83 (m, 1H), 2.65- 2.55 (m, 2H), 2.54 (s, 2H), 2.49- 2.47 (m, 1H), 2.44-2.38 (m, 2H), 2.03-1.98 (m, 1H), 1.96- 1.91 (m, 2H), 1.74-1.66 (m, 2H), 1.65-1.55 (m, 6H), 1.47- 1.39 (m, 2H). |
| | I-26 | | |
| I-27 | 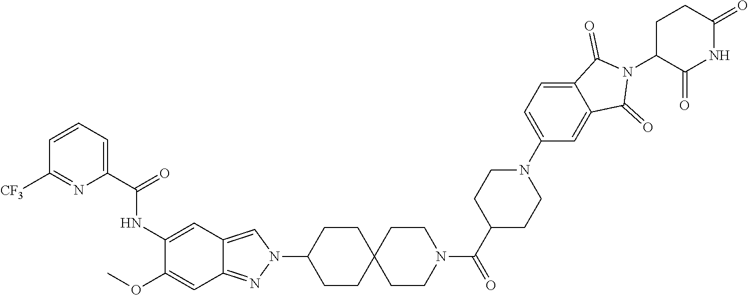 [SMILES: COc1cc2nn(C3CCC4(CC3)CCN(C(=O)C3CCN(c5ccc6c(c5)C(=O)N(C5CCC(=O)NC5=O)C6=O)CC3)CC4)cc2cc1NC(=O)c1cccc(C(F)(F)F)n1] I-27 | 855.6 | (500 MHz, DMSO-d₆) δ 11.08 (s, 1H), 10.51 (s, 1H), 8.69 (d, J = 4.5 Hz, 1H), 8.46 (d, J = 7.5 Hz, 1H), 8.41 (t, J = 8.0 Hz, 1H), 8.38 (s, 1H), 8.22 (d, J = 8.0 Hz, 1H), 7.67 (d, J = 8.5 Hz, 1H), 7.33 (s, 1H), 7.25 (dd, J = 8.5, 2.0 Hz, 1H), 7.17 (s, 1H), 5.07 (dd, J = 13.0, 5.5 Hz, 1H), 4.45-4.34 (m, 1H), 4.12-4.02 (m, 2H), 3.99 (d, J = 1.0 Hz, 3H), 3.66-3.45 (m, 4H), 3.15- 3.04 (m, 2H), 3.03-2.94 (m, 1H), 2.94-2.84 (m, 1H), 2.63- 2.52 (m, 2H), 2.13-2.00 (m, 3H), 1.99-1.91 (m, 2H), 1.91- 1.81 (m, 2H), 1.77-1.68 (m, 2H), 1.68-1.51 (m, 4H), 1.46- 1.27 (m, 4H). |
| I-28 | 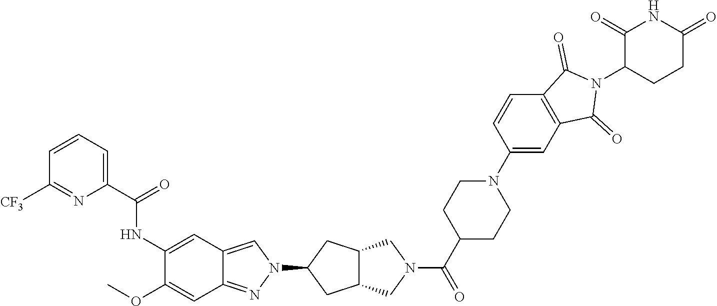 [SMILES: COc1cc2nn([C@H]3C[C@@H]4CN(C(=O)C5CCN(c6ccc7c(c6)C(=O)N(C6CCC(=O)NC6=O)C7=O)CC5)C[C@@H]4C3)cc2cc1NC(=O)c1cccc(C(F)(F)F)n1] I-28 | 813.5 | (500 MHz, DMSO-d₆) δ 11.08 (s, 1H), 10.51 (s, 1H), 8.69 (s, 1H), 8.46 (d, J = 7.5 Hz, 1H), 8.42 (d, J = 7.5 Hz, 1H), 8.36 (s, 1H), 8.24-8.19 (m, 1H), 7.67 (d, J = 8.5 Hz, 1H), 7.34 (d, J = 2.0 Hz, 1H), 7.26 (d, J = 8.5 Hz, 1H), 7.18 (s, 1H), 5.20-5.14 (m, 1H), 5.07 (dd, J = 12.5, 5.0 Hz, 1H), 4.14-4.06 (m, 2H), 3.99 (s, 3H), 3.84-3.78 (m, 1H), 3.58 (dd, J = 12.4, 8.5 Hz, 1H), 3.50 (dd, J = 10.9, 4.5 Hz, 1H), 3.30-3.26 (m, 1H), 3.11- 3.03 (m, 3H), 2.97-2.92 (m, 1H), 2.90-2.78 (m, 2H), 2.65- 2.53 (m, 2H), 2.41-2.33 (m, 2H), 2.17-2.08 (m, 2H), 2.05- 1.99 (m, 1H), 1.83-1.73 (m, 2H), 1.68-1.59 (m, 2H). |
| I-29 | 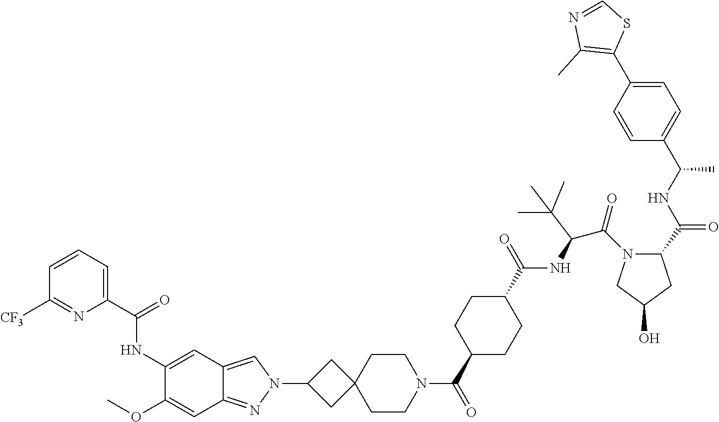 [SMILES: COc1cc2nn(C3CC4(CCN(C(=O)[C@H]5CC[C@H](C(=O)N[C@H](C(=O)N6C[C@H](O)C[C@H]6C(=O)N[C@@H](C)c6ccc(-c7scnc7C)cc6)C(C)(C)C)CC5)CC4)C3)cc2cc1NC(=O)c1cccc(C(F)(F)F)n1] | 1040.6 | (500 MHz, DMSO-d₆) δ 10.53- 10.49 (m, 1H), 8.99 (s, 1H), 8.69 (s, 1H), 8.47 (d, J = 7.5 Hz, 1H), 8.41 (d, J = 7.5 Hz, 1H), 8.40-8.38 (m, 1H), 8.37 (s, 1H), 8.22 (d, J = 7.5 Hz, 1H), 7.68 (d, J = 8.0 Hz, 1H), 7.44 (d, J = 8.0 Hz, 2H), 7.38 (d, J = 8.0 Hz, 2H), 7.21 (s, 1H), 5.19- 5.13 (m, 1H), 5.13-5.10 (m, 1H), 4.95-4.90 (m, 1H), 4.53- 4.49 (m, 1H), 4.46-4.41 (m, 1H), 4.31-4.27 (m, 1H), 3.99 (s, 3H), 3.64-3.55 (m, 2H), 3.52-3.45 (m, 2H), 3.43-3.39 (m, 2H), 2.63-2.57 (m, 1H), 2.49-2.44 (m, 5H), 2.44-2.35 (m, 3H), 2.04-1.99 (m, 1H), 1.82-1.76 (m, 2H), 1.73-1.64 (m, 5H), 1.63-1.55 (m, 2H), 1.51-1.43 (m, 2H), 1.40-1.33 (m, 5H), 0.97-0.90 (m, 9H). |
| | I-29 | | |
| I-31 | 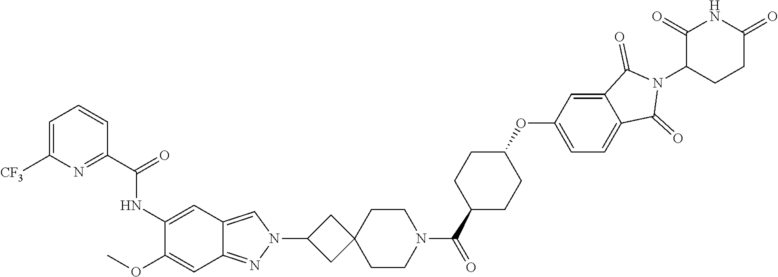 [SMILES: COc1cc2nn(C3CC4(CCN(C(=O)[C@H]5CC[C@H](Oc6ccc7c(c6)C(=O)N(C6CCC(=O)NC6=O)C7=O)CC5)CC4)C3)cc2cc1NC(=O)c1cccc(C(F)(F)F)n1] I-31 | 842.6 | (500 MHz, DMSO-d₆) δ 11.11 (s, 1H), 10.51 (s, 1H), 8.69 (s, 1H), 8.46 (d, J = 7.5 Hz, 1H), 8.41 (t, J = 7.5 Hz, 1H), 8.37 (s, 1H), 8.22 (dd, J = 7.5, 1.0 Hz, 1H), 7.81 (d, J = 8.5 Hz, 1H), 7.48 (d, J = 1.5 Hz, 1H), 7.37 (dd, J = 8.1, 1.0 Hz, 1H), 7.21 (s, 1H), 5.25-5.14 (m, 1H, 5.12 (dd, J = 12.5, 5.4 Hz, 1H), 4.72-4.58 (m, 1H), 3.99 (s, 3H), 3.60-3.47 (m, 2H), 3.47- 3.38 (m, 2H), 2.96-2.85 (m, 1H), 2.77-2.66 (m, 1H), 2.66- 2.53 (m, 2H), 2.49-2.45 (m, 2H), 2.45-2.37 (m, 2H), 2.17- 2.08 (m, 2H), 2.08-2.02 (m, 1H), 1.82-1.70 (m, 3H), 1.70- 1.56 (m, 5H), 1.56-1.43 (m, 2H). |
| I-32 | 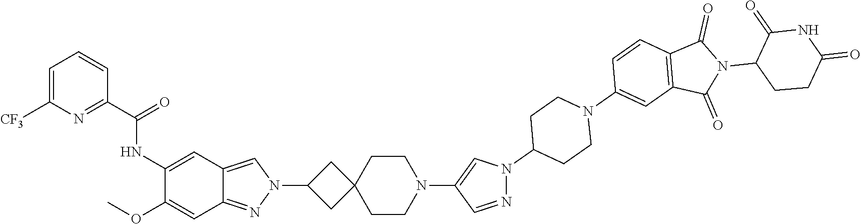 [SMILES: COc1cc2nn(C3CC4(CCN(c5cnn(C6CCN(c7ccc8c(c7)C(=O)N(C7CCC(=O)NC7=O)C8=O)CC6)c5)CC4)C3)cc2cc1NC(=O)c1cccc(C(F)(F)F)n1] I-32 | 865.6 | (500 MHz, DMSO-d) δ 11.08 (s, 1H), 10.51 (s, 1H), 8.68 (s, 1H), 8.46 (d, J = 7.5 Hz, 1H), 8.41 (t, J = 8.0 Hz, 1H), 8.36 (s, 1H), 8.22 (dd, J = 7.5, 1.0 Hz, 1H), 7.69 (d, J = 8.5 Hz, 1H), 7.39 (d, J = 2.0 Hz, 1H), 7.37 (s, 1H), 7.31 (dd, J = 8.5, 2.0 Hz, 1H), 7.21 (s, 1H), 7.17 (s, 1H), 5.17-5.13 (m, 1H), 5.08 (dd, J = 13.0, 5.5 Hz, 1H), 4.38-4.33 (m, 1H), 4.19-4.14 (m, 2H), 3.99 (s, 3H), 3.19-3.14 (m, 2H), 2.88-2.85 (m, 2H), 2.81- 2.76 (m, 2H), 2.65-2.60 (m, 1H), 2.59-2.54 (m, 1H), 2.46- |
| | | | 2.42 (m, 2H), 2.41-2.37 (m, |
| | | | 2H), 2.05-2.01 (m, 3H), 1.99- |
| | | | 1.96 (m, 1H), 1.94-1.89 (m, |
| | | | 2H), 1.81-1.77 (m, 2H), 1.77- |
| | | | 1.73 (m, 2H). |
| I-33 | 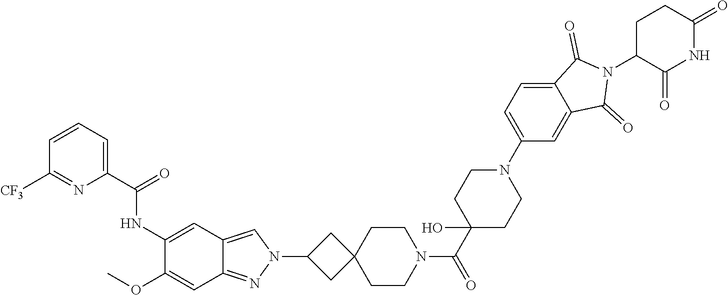 [SMILES: COc1cc2nn(C3CC4(CCN(C(=O)C5(O)CCN(c6ccc7c(c6)C(=O)N(C6CCC(=O)NC6=O)C7=O)CC5)CC4)C3)cc2cc1NC(=O)c1cccc(C(F)(F)F)n1] | 843.5 | (500 MHz, DMSO-d₆) δ 11.08 (s, 1H), 10.51 (s, 1H), 8.70- 8.67 (m, 1H), 8.46 (d, J = 7.5 Hz, 1H), 8.40 (t, J = 7.5 Hz, 1H), 8.37 (s, 1H), 8.22 (dd, J = 7.5, 1.0 Hz, 1H), 7.66 (d, J = 8.5 Hz, 1H), 7.34 (d, J = 2.0 Hz, 1H), 7.25 (dd, J = 8.5, 2.0 Hz, 1H), 7.21 (s, 1H), 5.71 (s, 1H), 5.18-5.13 (m, 1H), 5.07 (dd, J = 12.5, 5.5 Hz, 1H), 3.99 (s, 3H), 3.81-3.76 (m, 2H), 3.43- 3.36 (m, 3H), 3.32-3.28 (m, 1H), 2.92-2.84 (m, 1H), 2.65- 2.51 (m, 3H), 2.49-2.45 (m, 2H), 2.44-2.39 (m, 2H), 2.03- 1.96 (m, 3H), 1.83-1.72 (m, 3H), 1.72-1.60 (m, 4H). |
| | I-33 | | |
| I-34 | 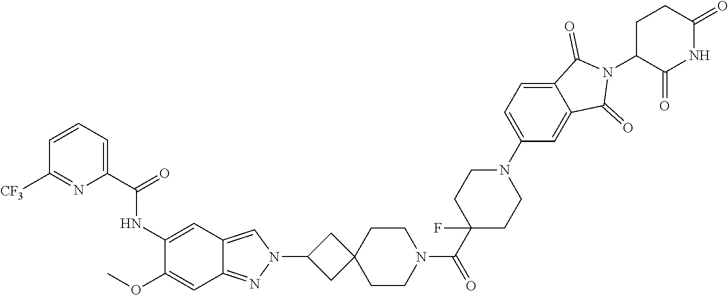 [SMILES: COc1cc2nn(C3CC4(CCN(C(=O)C5(F)CCN(c6ccc7c(c6)C(=O)N(C6CCC(=O)NC6=O)C7=O)CC5)CC4)C3)cc2cc1NC(=O)c1cccc(C(F)(F)F)n1] | 845.5 | (500 MHz, DMSO-d₆) δ 11.09 (s, 1H), 10.51 (s, 1H), 8.69 (s, 1H), 8.46 (d, J = 7.5 Hz, 1H), 8.40 (t, J = 7.5 Hz, 1H), 8.37 (s, 1H), 8.22 (dd, J = 7.5, 1.0 Hz, 1H), 7.70 (d, J = 8.5 Hz, 1H), 7.41 (d, J = 2.0 Hz, 1H), 7.31 (dd, J = 8.5, 2.0 Hz, 1H), 7.22 (s, 1H), 5.20-5.13 (m, 1H), 5.08 (dd, J = 12.5, 5.0 Hz, 1H), 3.99 (s, 3H), 3.98-3.91 (m, 2H), 3.77-3.61 (m, 2H), 3.57- 3.41 (m, 2H), 3.33-3.28 (m, 1H), 2.94-2.85 (m, 1H), 2.65- 2.51 (m, 3H), 2.50-2.46 (m, 2H), 2.46-2.39 (m, 2H), 2.24- 2.10 (m, 2H), 2.07-1.99 (m, 3H), 1.79-1.63 (m, 4H). |
| | I-34 | | |
| I-35 | 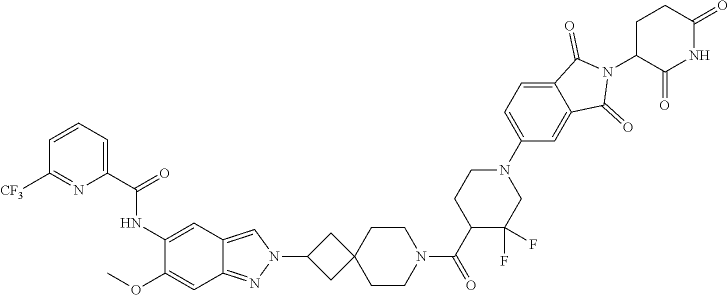 [SMILES: COc1cc2nn(C3CC4(CCN(C(=O)C5CCN(c6ccc7c(c6)C(=O)N(C6CCC(=O)NC6=O)C7=O)CC5(F)F)CC4)C3)cc2cc1NC(=O)c1cccc(C(F)(F)F)n1] | 863.6 | (500 MHz, DMSO-d₆) δ 11.09 (s, 1H), 10.51 (s, 1H), 8.69 (s, 1H), 8.47 (d, J = 7.5 Hz, 1H), 8.41 (t, J = 7.5 Hz, 1H), 8.38 (s, 1H), 8.22 (dd, J = 7.8, 0.8 Hz, 1H), 7.69 (d, J = 8.0 Hz, 1H), 7.45 (s, 1H), 7.38-7.33 (m, 1H), 7.21 (d, J = 2.0 Hz, 1H), 5.20-5.15 (m, 1H), 5.08 (dd, J = 12.8, 5.4 Hz, 1H), 4.38-4.31 (m, 1H), 3.99 (s, 3H), 3.98- 3.92 (m, 1H), 3.89-3.67 (m, 3H), 3.61-3.46 (m, 4H), 2.92- 2.85 (m, 1H), 2.65-2.59 (m, 1H), 2.58-2.54 (m, 1H), 2.46- 2.41 (m, 2H), 2.10-1.96 (m, 3H), 1.87-1.82 (m, 1H), 1.79- 1.59 (m, 5H). |
| | I-35 | | |
| I-36 | 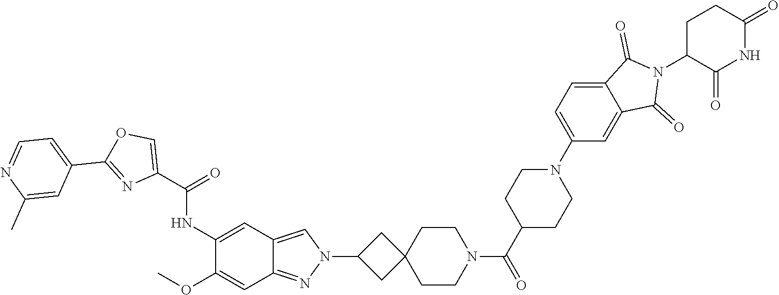 [SMILES: COc1cc2nn(C3CC4(CCN(C(=O)C5CCN(c6ccc7c(c6)C(=O)N(C6CCC(=O)NC6=O)C7=O)CC5)CC4)C3)cc2cc1NC(=O)c1coc(-c2ccnc(C)c2)n1] | 840.3 | (500 MHz, CDCl₃) δ 9.57 (s, 1H), 8.76 (s, 1H), 8.70 (d, J = 4.0 Hz, 1H), 8.41 (s, 1H), 8.04 (s, 1H), 7.88 (s, 1H), 7.86 (s, 1H), 7.82 (s, 1H), 7.69 (d, J = 8.0 Hz, 1H), 7.29 (s, 1H), 7.11 (s, 1H), 7.06 (d, J = 8.5 Hz, 1H), 5.06-4.99 (m, 1H), 4.97-4.91 (m, 1H), 4.07 (s, 3H), 3.98 (d, J = 13.0 Hz, 2H), 3.67-3.45 (m, 4H), 3.07 (t, J = 11.5 Hz, 2H), 2.92-2.78 (m, 3H), 2.73 (s, 3H), 2.58 (d, J = 8.0 Hz, 3H), 2.37-2.32 (m, 1H), 2.17-2.10 (m, 1H), 2.07-2.02 (m, 1H), 1.98-1.92 (m, 2H), 1.88-1.79 (m, 4H), 1.77-1.73 (m, 2H). |
| | I-36 | | |
| I-37 | 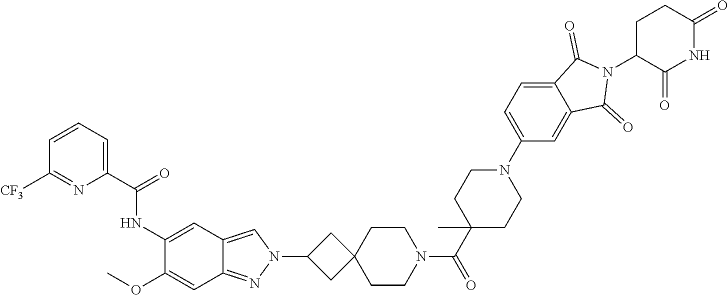 [SMILES: COc1cc2nn(C3CC4(CCN(C(=O)C5(C)CCN(c6ccc7c(c6)C(=O)N(C6CCC(=O)NC6=O)C7=O)CC5)CC4)C3)cc2cc1NC(=O)c1cccc(C(F)(F)F)n1] | 841.5 | (500 MHz, DMSO-d₆) δ 11.07 (s, 1H), 10.51 (s, 1H), 8.69 (s, 1H), 8.47 (d, J = 7.5 Hz, 1H), 8.41 (t, J = 7.5 Hz, 1H), 8.37 (s, 1H), 8.22 (d, J = 7.5 Hz, 1H), 7.65 (d, J = 8.5 Hz, 1H), 7.29 (d, J = 1.5 Hz, 1H), 7.24-7.17 (m, 2H), 5.22-5.12 (m, 1H), 5.06 (dd, J = 12.5, 5.5 Hz, 1H), 3.99 (s, 3H), 3.73-3.45 (m, 6H), 2.93-2.83 (m, 1H), 2.66-2.52 (m, 3H), 2.48-2.35 (m, 4H), 2.23-2.13 (m, 2H), 2.08-1.94 (m, 2H), 1.75-1.54 (m, 6H), 1.29 (s, 3H). |
| | I-37 | | |
| I-38 | 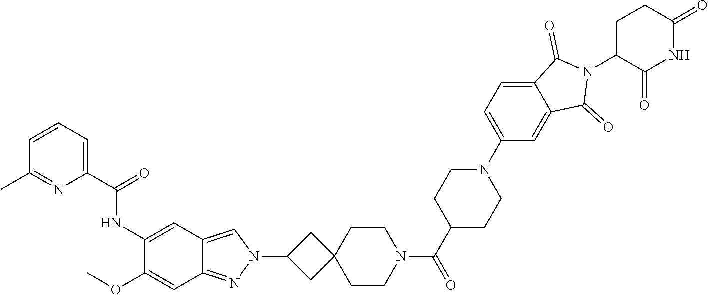 [SMILES: COc1cc2nn(C3CC4(CCN(C(=O)C5CCN(c6ccc7c(c6)C(=O)N(C6CCC(=O)NC6=O)C7=O)CC5)CC4)C3)cc2cc1NC(=O)c1cccc(C)n1] | 773.5 | (500 MHz, DMSO-d₆) δ 11.07 (s, 1H), 10.,71 (s, 1H), 8.70 (s, 1H), 8.35 (s, 1H), 8.02-7.93 (m, 2H), 7.67 (d, J = 8.5 Hz, 1H), 7.56 (dd, J = 7.5, 2.0 Hz, 1H), 7.33 (s, 1H), 7.25 (d, J = 9.0 Hz, 1H), 7.19 (s, 1H), 5.19- 5.12 (m, 1H), 5.07 (dd, J = 12.5, 5.5 Hz, 1H), 4.07 (d, J = 13.0 Hz, 3H), 4.01 (s, 3H), 3.58- 3.38 (m, 4H), 3.06-3.13 (m, 2H), 2.97-3.03 (m, 1H), 2.85- 2.92 (m, 1H), 2.63 (s, 3H), 2.60- 2.51 (m, 2H), 2.47-2.35 (m, 4H), 2.04-1.99 (m, 1H), 1.76- 1.56 (m, 8H). |
| | I-38 | | |
| I-48 | 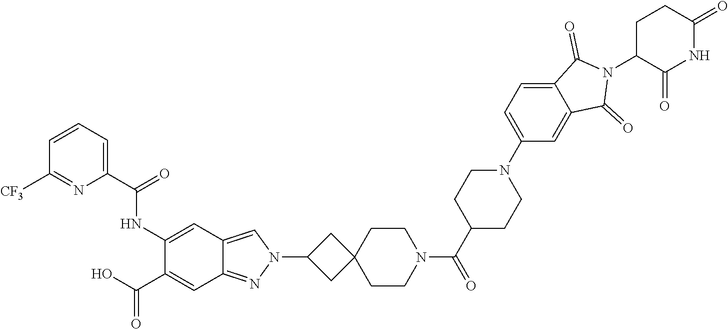 [SMILES: O=C1CCC(N2C(=O)c3ccc(N4CCC(C(=O)N5CCC6(CC5)CC(n5cc7cc(NC(=O)c8cccc(C(F)(F)F)n8)c(C(=O)O)cc7n5)C6)CC4)cc3C2=O)C(=O)N1] | 841.8 | (500 MHz, DMSO-d₆) δ 13.59 (s, 1H), 11.08 (s, 1H), 9.12 (s, 1H), 8.56 (s, 1H), 8.49 (s, 1H), 8.45 (d, J = 7.5 Hz, 1H), 8.37 (t, J = 8.0 Hz, 1H), 8.17 (d, J = 8.0 Hz, 1H), 7.67 (d, J = 8.5 Hz, 1H), 7.33 (s, 1H), 7.25 (d, J = 8.5 Hz, 1H), 5.34-5.24 (m, 1H), 5.07 (dd, J = 12.5, 5.5 Hz, 1H), 4.13-4.02 (m, 2H), 3.61- 3.54 (m, 1H), 3.54-3.47 (m, 3H), 3.13-3.06 (m, 2H), 3.04- 2.97 (m, 1H), 2.92-2.84 (m, 1H), 2.66-2.59 (m, 1H), 2.59- 2.53 (m, 2H), 2.49-2.35 (m, 3H), 2.05-1.98 (m, 1H), 1.82- 1.56 (m, 8H). |
| | I-48 | | |
| I-51 | 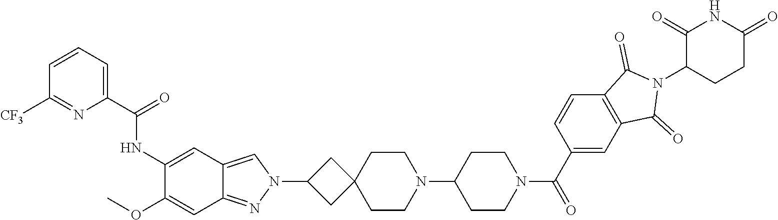 [SMILES: COc1cc2nn(C3CC4(CCN(C5CCN(C(=O)c6ccc7c(c6)C(=O)N(C6CCC(=O)NC6=O)C7=O)CC5)CC4)C3)cc2cc1NC(=O)c1cccc(C(F)(F)F)n1] I-51 | 827.7 | (500 MHz, DMSO-d₆) δ 11.15 (s, 1H), 10.51 (s, 1H), 8.68 (s, 1H), 8.46 (d, J = 7.5 Hz, 1H), 8.41 (d, J = 7.5 Hz, 1H), 8.35 (s, 1H), 8.22 (d, J = 8.0 Hz, 1H), 7.98 (d, J = 7.5 Hz, 1H), 7.92 (s, 1H), 7.90-7.84 (m, 1H), 7.21 (s, 1H), 5.18 (dd, J = 12.8, 5.4 Hz, 1H), 5.14-5.07 (m, 1H), 4.51 (d, J = 12.5 Hz, 1H), 3.99 (s, 3H), 3.48 (d, J = 9.5 Hz, 1H), 3.16-3.01 (m, 1H), 2.95-2.86 (m, 1H), 2.81 (s, 1H), 2.65-2.51 (m, 3H), 2.42-2.30 (m, 6H), 2.11- 2.04 (m, 1H), 1.83 (s, 1H), 1.67 (d, J = 20.0 Hz, 4H), 1.47 (d, J = 11.5 Hz, 2H), 1.28-1.21 |
| | | | (m, 1H). |
| I-53 | 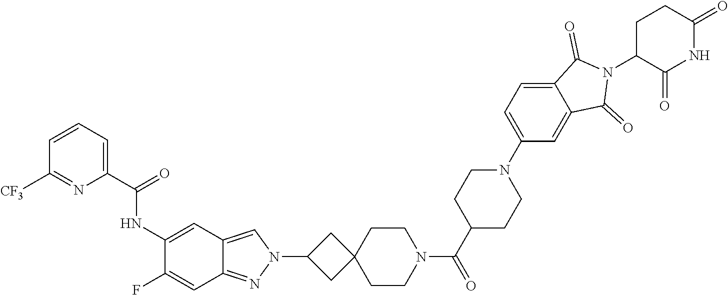 [SMILES: O=C1CCC(N2C(=O)c3ccc(N4CCC(C(=O)N5CCC6(CC5)CC(n5cc7cc(NC(=O)c8cccc(C(F)(F)F)n8)c(F)cc7n5)C6)CC4)cc3C2=O)C(=O)N1] | 815.6 | (500 MHz, DMSO-d₆) δ 11.15 (s, 1H), 10.51 (s, 1H), 8.68 (s, 1H), 8.46 (d, J = 8.0 Hz, 1H), 8.41 (t, J = 8.0 Hz, 1H), 8.35 (s, 1H), 8.22 (d, J = 8.0 Hz, 1H), 7.98 (d, J = 7.5 Hz, 1H), 7.92 (s, 1H), 7.87 (dd, J = 8.0, 1.0 Hz, 1H), 7.21 (s, 1H), 5.18 (dd, J = 13.0, 5.5 Hz, 1H), 5.14-5.07 (m, 1H), 4.55-4.47 (m, 1H), 3.99 (s, 3H), 3.51-3.43 (m, 1H), 3.10-3.01 (m, 1H), 2.95- 2.86 (m, 1H), 2.84-2.77 (m, 1H), 2.66-2.52 (m, 4H), 2.47- 2.26 (m, 7H), 2.11-2.04 (m, 1H), 1.88-1.80 (m, 1H), 1.74- 1.60 (m, 5H), 1.53-1.40 (m, 2H). |
| | I-53 | | |
| I-56 | 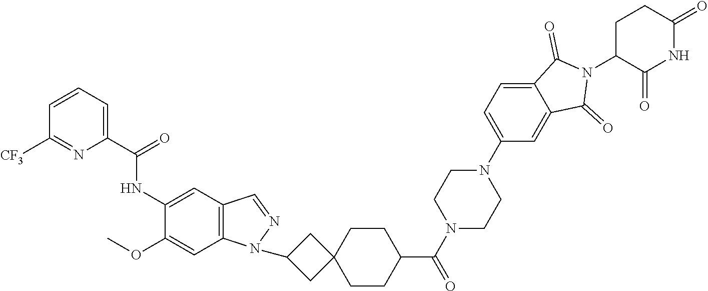 [SMILES: COc1cc2c(cnn2C2CC3(CCC(C(=O)N4CCN(c5ccc6c(c5)C(=O)N(C5CCC(=O)NC5=O)C6=O)CC4)CC3)C2)cc1NC(=O)c1cccc(C(F)(F)F)n1] | 827.5 | (400 MHz, DMSO) δ 11.09 (s, 1H), 10.43 (s, 1H), 8.72 (s, 1H), 8.47 (d, J = 7.5 Hz, 1H), 8.41 (t, J = 7.5 Hz, 1H), 8.22 (d, J = 7.5 Hz, 1H), 8.07 (s, 1H), 7.72 (d, J = 8.5 Hz, 1H), 7.42 (s, 1H), 7.36 (d, J = 2.0 Hz, 1H), 7.26 (dd, J = 8.5, 2.0 Hz, 1H), 5.36-5.26 (m, 1H), 5.08 (dd, J = 12.5, 5.5 Hz, 1H), 4.04 (s, 3H), 3.71-3.49 (m, 10H), 2.92-2.83 (m, 1H), 2.69-2.60 (m, 2H), 2.59-2.54 (m, 1H), 2.43-2.37 (m, 1H), 2.32-2.21 (m, 2H), 2.09-1.97 (m, 2H), 1.85-1.76 (m, 1H), 1.71-1.65 (m, 1H), 1.58-1.49 (m, 4H). |
| | I-56 | | |

### Example 7: Synthesis of I-30

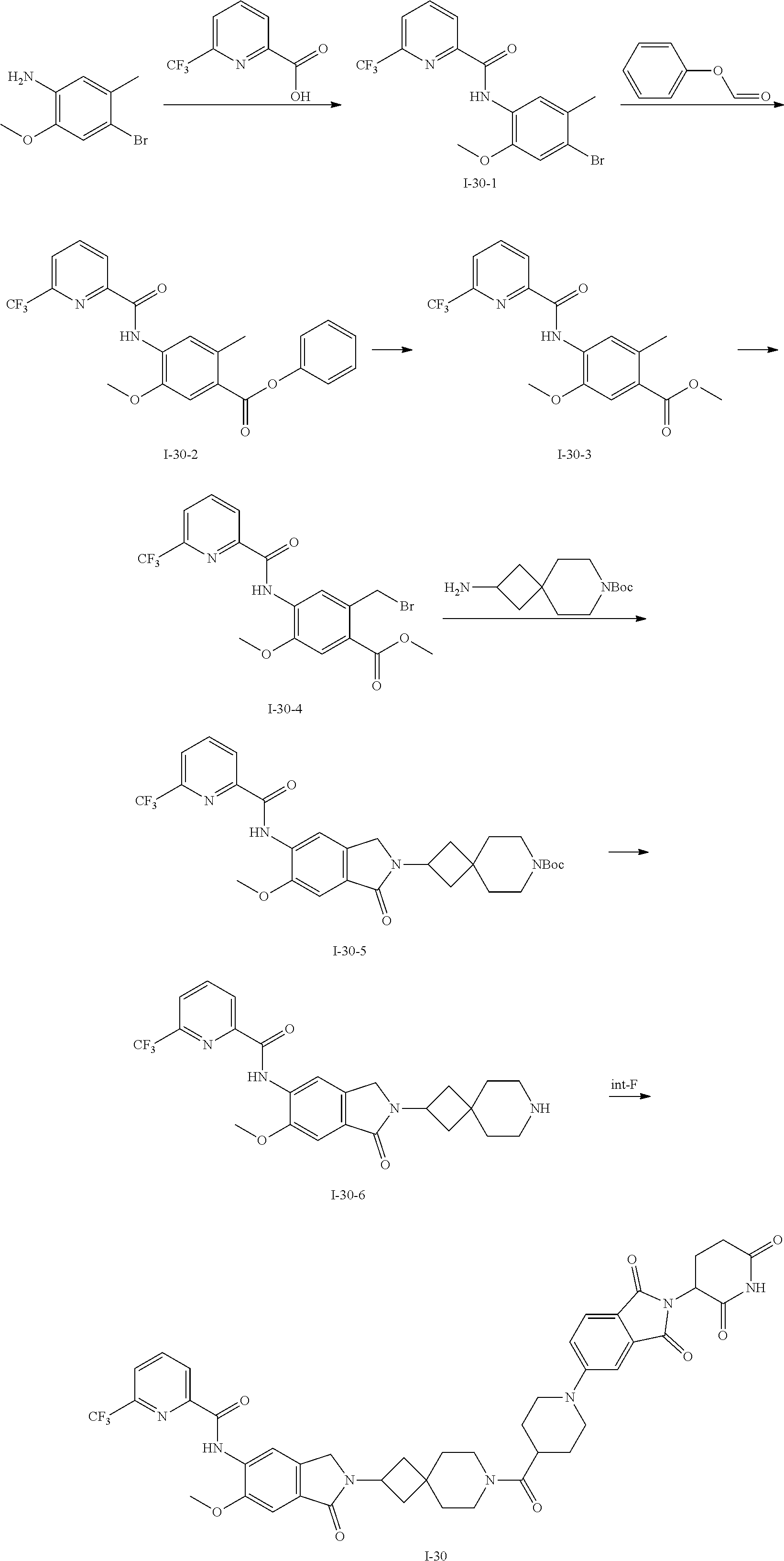

Under nitrogen protection, 4-bromo-2-methoxy-5-methyl-aniline (1 g, 4.63 mmol), 6-(trifluoromethyl)pyridine-2-carboxylic acid (1.06 g, 5.55 mmol), DIPEA (1.20 g, 9.26 mmol, 1.61 mL) and anhydrous THF (15 mL) were added to the reaction flask and cooled to 0° C. T₃P (2.95 g, 9.26 mmol) was slowly added dropwise, and the reaction was stirred at 25° C. for 1 hour. H₂O (60 mL) was added to the reaction system. The product was precipitated, filtered, the filter cake was washed with water, and dried under vacuum to obtain the product I-30-1 as white solid (1.77 g, yield 98.16%), MS (ESI) m/z:389.2 [M+H]⁺.

### Step 2: Synthesis of (I-30-2)

Under nitrogen protection, I-30-1 (1.77 g, 4.54 mmol), phenyl formate (1.1 g, 9.08 mmol), Pd(OAc)₂ (palladium acetate, 101 mg, 0.45 mmol), P(t-Bu)₃·HBF₄ (tri-tert-butylphosphine tetrafluoroborate, 516.4 mg, 1.78 mmol), Et₃N (triethylamine, 917.08 mg, 9.08 mmol), and acetonitrile (3 mL) were added into a reaction bottle, and warmed to 80° C. for reaction overnight. The mixture was returned to room temperature, filtered, and the filtrate was concentrated under reduced pressure, added with ethyl acetate to dilute, washed twice with saturated brine, dried over anhydrous sodium sulfate, filtered, and concentrated under reduced pressure to obtain a crude product, which was purified by reverse-phase C18 column chromatography (mobile phase 0-60% water/acetonitrile) to obtain product I-30-2 as a white solid (1.3 g, yield 70%), MS (ESI) m/z: 431.1 [M+H]⁺.

### Step 3: Synthesis of (I-30-3)

I-30-2 (1.3 g, 3.2 mmol), potassium carbonate (1.77 g, 12.8 mmol) and methanol (4 mL) were added to a reaction bottle and reacted at room temperature for 2 hours. Water was added to precipitate a product, which was filtered, and the filter cake was washed with water and vacuum-dried to obtain a crude product. The crude product was purified by reversed-phase C18 column chromatography (mobile phase 0-60% water/acetonitrile) to obtain product I-30-3 as a white solid (900 mg, yield 75%), MS (ESI) m/z: 369.1[M+H]⁺.

### Step 4: Synthesis of (I-30-4)

Under nitrogen protection, I-30-3 (900 mg, 2.4 mmol), NBS (512.6 mg, 2.88 mmol), AIBN (39 mg, 0.24 mmol) and CCl₄ (9 mL) were added into a reaction bottle, warmed to 80° C., and reacted for 5 hours. The reaction solution was returned to room temperature and filtered, and the filtrate was added with H₂O (30 mL), and extracted with EA (25 mL×3). The organic phases were combined, dried over anhydrous Na₂SO₄, filtered, and concentrated under reduced pressure to obtain a crude product, which was purified by reverse-phase C18 column chromatography, eluting with (MeCN/H₂O+1% o HCOOH) to obtain the desired product I-30-4 as a white solid (802 mg, yield 76%), MS (ESI) m/z 447.1 [M+H]⁺.

### Step 5: Synthesis of (I-30-5)

A mixed solution of I-30-4 (300 mg, 670.84 μmol), 2-amino-7-azaspiro[3.5]non-7-carboxylic acid tert-butyl ester (177.35 mg, 737.92 μmol), DIPEA (260.10 mg, 2.01 mmol, 350.53 μL) and ACN (4 mL) was stirred at 25° C. for 3 hours. The reaction solution was added with H₂O (20 mL), and extracted with EA (15 mL×3). The combined organic phases were washed with saturated brine (20 mL), dry over anhydrous Na₂SO₄, filtered and concentrated under reduced pressure. The crude product was purified by flash silica gel column chromatography (DCM:MeOH=30:1) to obtain product I-30-5 as a brown solid (135 mg, yield 35.02%), MS (ESI) m/z: 575.3 [M+H]⁺.

### Step 6: Synthesis of (I-30-6)

I-30-5 (135 mg, 234.95 μmol) was dissolved in DCM (3 mL), added with TFA (1.5 mL), and stirred at 25° C. for 2 hours. The resulting mixture was concentrated under reduced pressure to obtain product I-30-6 as a white solid (86 mg, yield 70.32%, TFA salt), MS (ESI) m/z: 475.2 [M+H]⁺.

### Step 7: Synthesis of (I-30)

A mixed solution of I-30-6 (40 mg, 76.85 μmol), int-F (32.58 mg, 84.53 mol), HATU (35.06 mg, 92.22 μmol), DIPEA (39.73 mg, 307.40 mol, 53.54 μL) and DMF (2 mL) was stirred at 25° C. for 1 hour. The reaction solution was added with H₂O (25 mL), and extracted with EA (20 mL×3). The combined organic phases were washed with saturated brine (20 mL), dried over anhydrous Na₂SO₄, filtered and concentrated under reduced pressure. The crude product was purified by preparative thin layer chromatography (DCM:MeOH=20:1) to obtain product I-30 as a yellow solid (27 mg, yield 40.90%). ¹H NMR (500 MHz, DMSO-d₆) δ 11.08 (s, 1H), 10.58 (s, 1H), 8.67 (s, 1H), 8.48 (d, J=7.5 Hz, 1H), 8.42 (t, J=8.0 Hz, 1H), 8.25 (d, J=7.5 Hz, 1H), 7.66 (d, J=8.5 Hz, 1H), 7.36 (s, 1H), 7.33 (d, J=1.5 Hz, 1H), 7.25 (dd, J=8.5, 2.0 Hz, 1H), 5.07 (dd, J=13.0, 5.5 Hz, 1H), 4.85-4.77 (m, 1H), 4.59 (s, 2H), 4.10-4.03 (m, 2H), 4.02 (s, 3H), 3.57-3.50 (m, 1H), 3.50-3.41 (m, 2H), 3.40-3.36 (m, 1H), 3.13-3.05 (m, 2H), 3.04-2.97 (m, 1H), 2.93-2.84 (m, 1H), 2.66-2.52 (m, 3H), 2.23-2.09 (m, 4H), 2.05-1.98 (m, 1H), 1.74-1.66 (m, 3H), 1.66-1.56 (m, 4H), 1.54-1.49 (m, 1H).

### Example 8: Synthesis of I-41

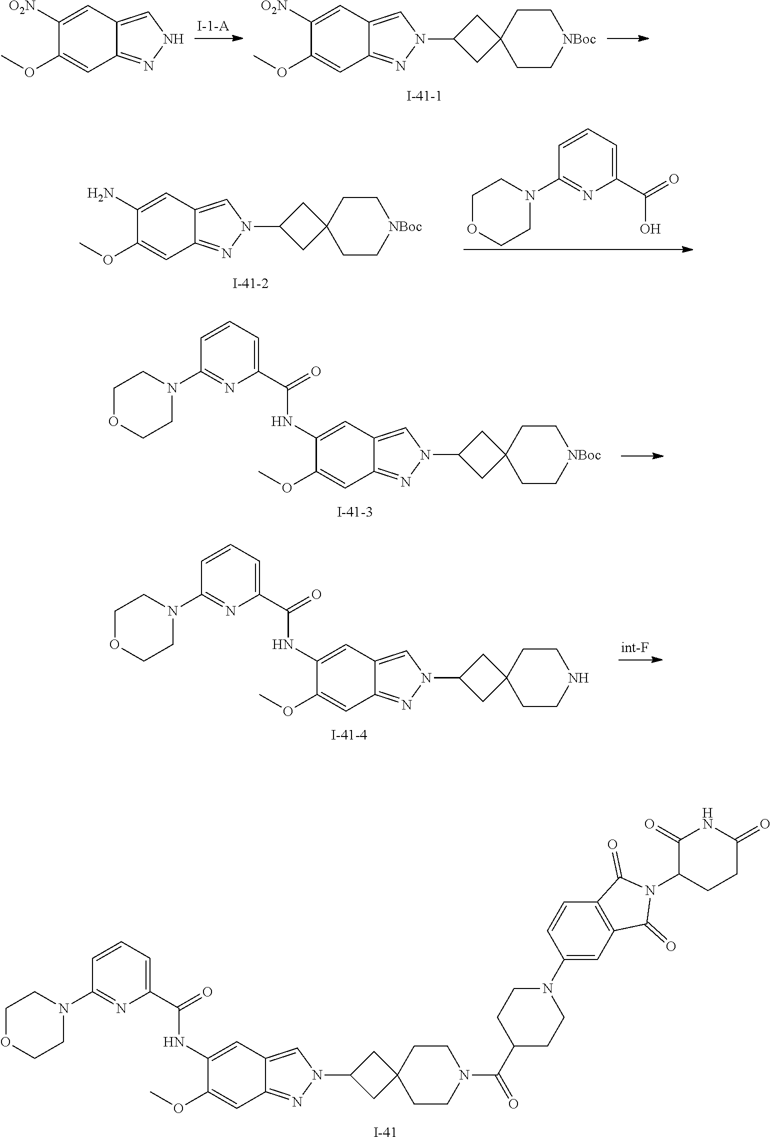

### Step 1: Synthesis of (I-41-1)

Under nitrogen protection, a mixture of 6-methoxy-5-nitro-2H-indazole (1.17 g, 6.07 mmol), DMF (12 mL), cesium carbonate (4.94 g, 15.17 mmol) and I-1-A (3 g, 7.59 mmol) was stirred at 90° C. for 3 hours. The reaction mixture was returned to room temperature, added with H₂O (60 mL), and extracted with DCM (35 mL×3). The combined organic phases were washed with saturated brine (50 mL), dried over anhydrous Na₂SO₄, filtered and concentrated under reduced pressure. The crude product was purified by flash silica gel column chromatography (DCM/MeOH=98/2) to obtain product I-41-1 as a white solid (770 mg, yield 24.38%), MS (ESI) m/z: 417.5 [M+H]⁺.

### Step 2: Synthesis of (I-41-2)

Under a hydrogen atmosphere, I-41-1 (770 mg, 1.85 mmol) was dissolved in MeOH (7 mL), added with catalyst wet Pd/C (80 mg, 10% purity), and reacted at 25° C. for 4 hours. The reaction solution was filtered through diatomaceous earth to remove the catalyst, and the filtrate was concentrated under reduced pressure to obtain product II-41-2 as a yellow solid (500 mg, yield 69.97%), MS (ESI) m/z: 387.2 [M+H]⁺.

### Step 3: Synthesis of (I-41-3)

A mixed solution of II-41-2 (50 mg, 129.37 μmol), 6-morpholinepyridine-2-carboxylic acid (26.94 mg, 129.37 mol), HATU (49.19 mg, 129.37 μmol), DIPEA (33.44 mg, 258.74 μmol, 45.07 μL) and DMF (2 mL) was stirred at 25° C. for 2 hours. The reaction solution was added with H 20 (300 mL), and extracted with EA (20 mL×3). The combined organic phases were washed with saturated brine (20 mL), dried over anhydrous Na₂SO₄, filtered and concentrated under reduced pressure. The crude product was purified by preparative thin layer chromatography to obtain product II-41-3 as a white solid (64 mg, yield 85.78%), MS (ESI)m/z: 577.5 [M+H]⁺.

### Step 4: Synthesis of (I-41-4)

A mixed solution of I-41-3 (63 mg, 109.25 μmol), TFA (2 mL) and DCM (2 mL) was stirred at 25° C. for 1 hour. The reaction solution was concentrated under reduced pressure to obtain product I-41-4 as a yellow oil (64 mg, crude product, TFA salt), MS (ESI) m/z: 477.3 [M+H]⁺.

### Step 5: Synthesis of (I-41)

A mixed solution of I-41-4 (52 mg, 109.11 μmol), int-F (42.05 mg, 109.11 μmol), HATU (41.49 mg, 109.11 μmol), DIPEA (28.20 mg, 218.23 mol, 38.01 μL) and DMF (2 mL) was stirred at 25° C. for 2 hours. The reaction solution was added with H₂O (300 mL), and extracted with EA (20 mL×3). The combined organic phases were washed with saturated brine, dried over anhydrous sodium sulfate, and purified by preparative thin layer chromatography to obtain the product I-41 as a yellow solid (28 mg, yield 29.80%), MS (ESI) m/z: 844.6 [M+H]⁺. ¹H NMR (500 MHz, DMSO-d₆) δ 11.08 (s, 1H), 10.79 (s, 1H), 8.64 (s, 1H), 8.33 (s, 1H), 7.84-7.78 (m, 1H), 7.67 (d, J=8.5 Hz, 1H), 7.46 (d, J=7.0 Hz, 1H), 7.33 (s, 1H), 7.25 (d, J=8.5 Hz, 1H), 7.18 (s, 1H), 7.16 (d, J=8.5 Hz, 1H), 5.22-5.12 (m, 1H), 5.07 (dd, J=12.5, 5.5 Hz, 1H), 4.11-4.03 (m, 2H), 3.97 (s, 3H), 3.84-3.78 (m, 4H), 3.69-3.59 (m, 4H), 3.56-3.39 (m, 4H), 3.15-3.05 (m, 2H), 3.04-2.95 (m, 1H), 2.93-2.84 (m, 1H), 2.68-2.53 (m, 2H), 2.48-2.32 (m, 4H), 2.05-1.99 (m, 1H), 1.75-1.57 (m, 8H).

Compounds I-42, I-44, I-46, I-47, I-49, 1-50, I-52, I-54, and I-55 can be prepared by referring to the method of Example 8 above. The structure and characterization data are shown in the following table:

| Molecular | | MS (ESI) | |
|---|---|---|---|
| ID | structure | m/z: [M + H]⁺ | ¹H NMR |
| I-42 | 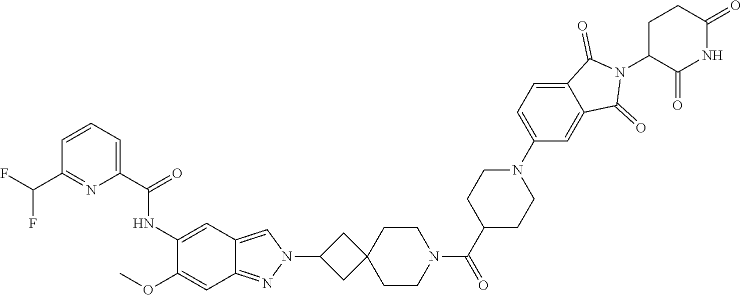 [SMILES: COc1cc2nn(C3CC4(CCN(C(=O)C5CCN(c6ccc7c(c6)C(=O)N(C6CCC(=O)NC6=O)C7=O)CC5)CC4)C3)cc2cc1NC(=O)c1cccc(C(F)F)n1] | 809.5 | (500 MHz, DMSO-d₆) δ 11.07 (s, 1H), 10.56 (s, 1H), 8.69 (s, 1H), 8.37 (s, 1H), 8.36-8.29 (m, 2H), 8.00 (dd, J = 7.5, 1.0 Hz, 1H), 7.67 (d, J = 8.5 Hz, 1H), 7.33 (s, 1H), 7.27-7.04 (m, 3H), 5.21-5.13 (m, 1H), 5.07 (dd, J = 12.5, 5.5 Hz, 1H), 4.11- 4.04 (m, 2H), 4.01 (s, 3H), 3.58- 3.39 (m, 4H), 3.14-3.05 (m, 2H), 3.04-2.96 (m, 1H), 2.93-2.84 (m, 1H), 2.65-2.54 (m, 2H), 2.47-2.35 (m, 4H), 2.05-1.98 (m, 1H), 1.77- 1.67 (m, 4H), 1.66-1.57 (m, 4H). |
| | I-42 | | |
| I-44 | 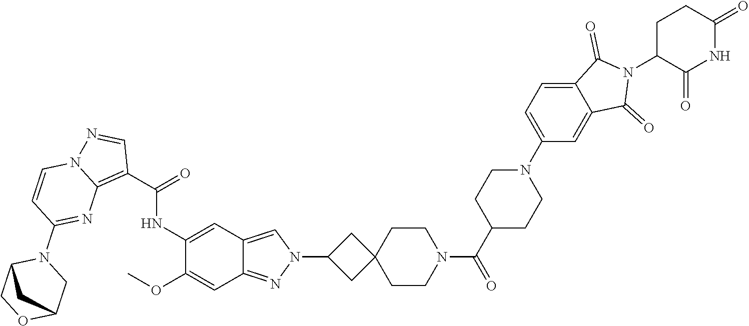 [SMILES: COc1cc2nn(C3CC4(CCN(C(=O)C5CCN(c6ccc7c(c6)C(=O)N(C6CCC(=O)NC6=O)C7=O)CC5)CC4)C3)cc2cc1NC(=O)c1cnn2ccc(N3C[C@H]4C[C@@H]3CO4)nc12] | 896.6 | (500 MHz, DMSO-d₆) δ 11.08 (s, 1H), 9.86 (d, J = 21.5 Hz, 1H), 8.79 (d, J = 7.5 Hz, 1H), 8.69 (s, 1H), 8.29 (s, 1H), 8.28 (s, 1H), 7.66 (d, J = 8.5 Hz, 1H), 7.33 (s, 1H), 7.24 (d, J = 7.5 Hz, 1H), 7.17 (d, J = 8.5 Hz, 1H), 6.88-6.46 (m, 1H), 5.46-5.04 (m, 3H), 4.89-4.81 (m, 1H), 4.12-4.02 (m, 2H), 3.94 (s, 3H), 3.87-3.82 (m, 1H), 3.78-3.60 (m, 2H), 3.58-3.53 (m, 1H), 3.52-3.44 (m, 2H), 3.44- 3.38 (m, 1H), 3.31-3.27 (m, 1H), 3.14-3.05 (m, 2H), 3.05-2.95 (m, 1H), 2.93-2.83 (m, 1H), 2.65-2.53 (m, 2H), 2.48-2.35 (m, 4H), 2.11- 1.98 (m, 3H), 1.80-1.67 (m, 4H), 1.67-1.56 (m, 4H). |
| | I-44 | | |
| I-46 | 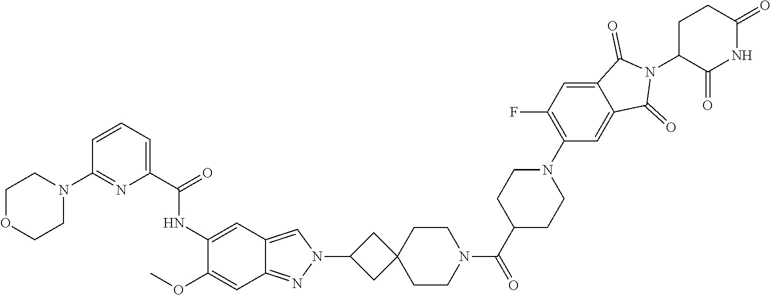 [SMILES: COc1cc2nn(C3CC4(CCN(C(=O)C5CCN(c6cc7c(cc6F)C(=O)N(C6CCC(=O)NC6=O)C7=O)CC5)CC4)C3)cc2cc1NC(=O)c1cccc(N2CCOCC2)n1] | 862.5 | (500 MHz, DMSO-d₆) δ 11.11 (s, 1H), 10.79 (s, 1H), 8.64 (s, 1H), 8.33 (s, 1H), 7.81 (dd, J = 8.0, 7.0 Hz, 1H), 7.71 (d, J = 11.0 Hz, 1H), 7.46 (d, J = 7.0 Hz, 1H), 7.44 (d, J = 7.5 Hz, 1H), 7.18 (s, 1H), 7.15 (d, J = 8.5 Hz, 1H), 5.20-5.13 (m, 1H), 5.11 (dd, J = 13.0, 5.5 Hz, 1H), 3.97 (s, 3H), 3.83-3.78 (m, 4H), 3.66-3.62 (m, 2H), 3.62-3.58 (m, 4H), 3.55- 3.49 (m, 2H), 3.47-3.41 (m, 2H), 3.03-2.97 (m, 2H), 2.93-2.86 (m, 2H), 2.65-2.55 (m, 2H), 2.49-2.45 (m, 2H), 2.44-2.39 (m, 2H), 2.06- 2.02 (m, 1H), 1.78-1.71 (m, 5H), 1.69-1.59 (m, 3H). |
| | I-46 | | |
| I-47 | 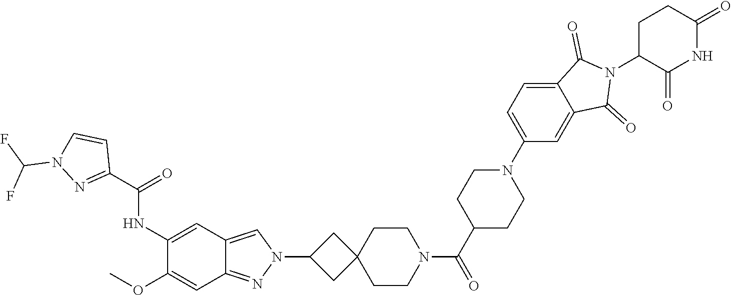 [SMILES: COc1cc2nn(C3CC4(CCN(C(=O)C5CCN(c6ccc7c(c6)C(=O)N(C6CCC(=O)NC6=O)C7=O)CC5)CC4)C3)cc2cc1NC(=O)c1ccn(C(F)F)n1] | 798.6 | (500 MHz, DMSO-d₆) δ 11.08 (s, 1H), 9.40 (s, 1H), 8.51 (s, 1H), 8.45 (d, J = 3.0 Hz, 1H), 8.35 (s, 1H), 8.09 (s, 1H), 7.98 (s, 1H), 7.86 (s, 1H), 7.67 (d, J = 8.5 Hz, 1H), 7.33 (s, 1H), 7.24 (d, J = 8.5 Hz, 1H), 7.19 (s, 1H), 7.03 (d, J = 2.5 Hz, 1H), 5.20-5.13 (m, 1H), 5.07 (dd, J = 13.0, 5.5 Hz, 1H), 4.07 (d, J = 12.5 Hz, 2H), 3.97 (s, 3H), 3.59-3.39 (m, 4H), 3.15- 3.06 (m, 2H), 3.04-2.97 (m, 1H), 2.92-2.84 (m, 1H), 2.62-2.52 (m, 2H), 2.49-2.44 (m, 2H), 2.44-2.38 (m, 2H), 2.04-1.99 (m, 1H), 1.77- 1.67 (m, 4H), 1.67-1.57 (m, 4H). |
| | I-47 | | |
| I-49 | 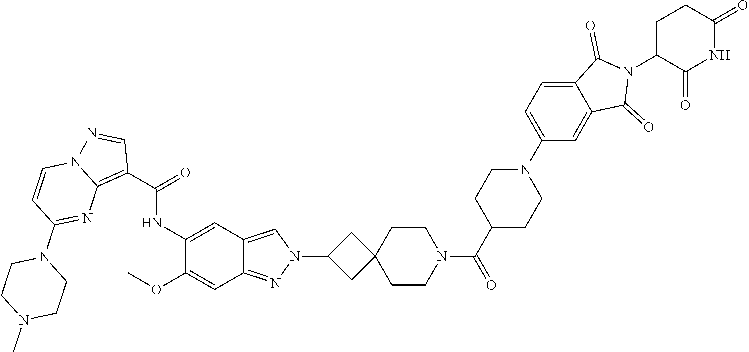 [SMILES: COc1cc2nn(C3CC4(CCN(C(=O)C5CCN(c6ccc7c(c6)C(=O)N(C6CCC(=O)NC6=O)C7=O)CC5)CC4)C3)cc2cc1NC(=O)c1cnn2ccc(N3CCN(C)CC3)nc12] | 897.6 | (500 MHz, DMSO-d₆) δ 11.08 (s, 1H), 9.76 (s, 1H), 8.80 (d, J = 7.0 Hz, 1H), 8.67 (s, 1H), 8.29 (s, 2H), 7.66 (d, J = 8.5 Hz, 1H), 7.33 (s, 1H), 7.25 (d, J = 8.5 Hz, 1H), 7.16 (s, 1H), 6.93 (d, J = 8.0 Hz, 1H), 5.21-5.11 (m, 1H), 5.07 (dd, J = 12.5, 5.0 Hz, 1H), 4.10-4.02 (m, 2H), 3.93 (s, 3H), 3.91-3.82 (m, 3H), 3.58-3.39 (m, 4H), 3.16-3.05 (m, 2H), 3.05-2.83 (m, 3H), 2.67-2.52 (m, 4H), 2.46- 2.33 (m, 5H), 2.27 (s, 3H), 2.07- 1.94 (m, 2H), 1.76-1.56 (m, 8H). |
| | I-49 | | |
| I-50 | 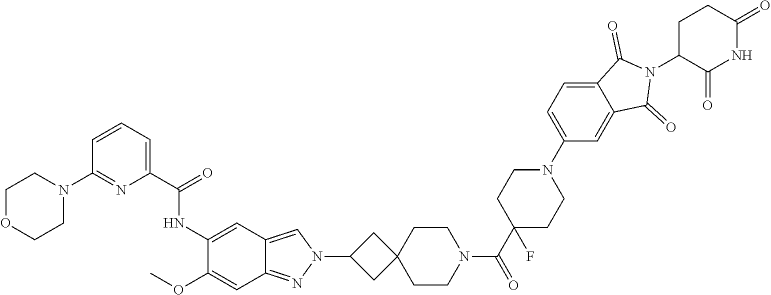 [SMILES: COc1cc2nn(C3CC4(CCN(C(=O)C5(F)CCN(c6ccc7c(c6)C(=O)N(C6CCC(=O)NC6=O)C7=O)CC5)CC4)C3)cc2cc1NC(=O)c1cccc(N2CCOCC2)n1] | 862.7 | (500 MHz, DMSO-d₆) δ 11.08 (s, 1H), 10.79 (s, 1H), 8.63 (s, 1H), 8.33 (s, 1H), 7.82 (dd, J = 8.5, 7.3 Hz, 1H), 7.70 (d, J = 8.5 Hz, 1H), 7.46 (d, J = 7.0 Hz, 1H), 7.41 (d, J = 2.0 Hz, 1H), 7.31 (dd, J = 8.7, 2.1 Hz, 1H), 7.18 (s, 1H), 7.16 (d, J = 8.5 Hz, 1H), 5.34-5.30 (m, 1H), 5.17-5.13 (m, 1H), 5.08 (dd, J = 13.0, 5.5 Hz, 1H), 3.97 (s, 3H), 3.95-3.92 (m, 1H), 3.82-3.79 (m, 4H), 3.62-3.58 (m, 4H), 2.64-2.56 (m, 2H), 2.45-2.39 (m, 3H), 2.38-2.35 (m, 1H), 2.05- 1.96 (m, 6H), 1.78-1.64 (m, 6H), 1.55-1.42 (m, 4H). |
| | I-50 | | |
| I-52 | 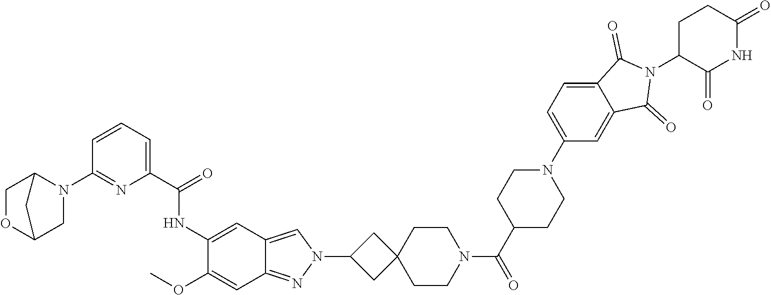 [SMILES: COc1cc2nn(C3CC4(CCN(C(=O)C5CCN(c6ccc7c(c6)C(=O)N(C6CCC(=O)NC6=O)C7=O)CC5)CC4)C3)cc2cc1NC(=O)c1cccc(N2CC3CC2CO3)n1] | 856.7 | (500 MHz, DMSO-d₆) δ 11.08 (s, 1H), 10.82 (s, 1H), 8.64 (s, 1H), 8.33 (s, 1H), 7.75 (dd, J = 8.0, 7.5 Hz, 1H), 7.67 (d, J = 8.5 Hz, 1H), 7.38 (d, J = 7.0 Hz, 1H), 7.33 (s, 1H) 7.25 (d, J = 8.5 Hz, 1H), 7.17 (s, 1H), 6.86 (d, J = 8.0 Hz, 1H), 5.19-5.11 (m, 1H), 5.07 (dd, J = 12.5, 5.5 Hz, 1H), 4.95 (s, 1H), 4.76 (s, 1H), 4.07 (d, J = 12.0 Hz, 2H), 3.98 (s, 3H), 3.89 (d, J = 7.0 Hz, 1H), 3.74 (d, J = 7.5 Hz, 1H), 3.60 (d, J = 10.0 Hz, 1H), 3.57-3.39 (m, 4H), 3.38-3.35 (m, 1H), 3.14- 3.06 (m, 2H), 3.03-2.96 (m, 1H), 2.92-2.85 (m, 1H), 2.65-2.53 (m, 2H), 2.47-2.33 (m, 4H), 2.05-1.94 (m, 3H, 1.78-1.55 (m, 8H). |
| | I-52 | | |
| I-54 | 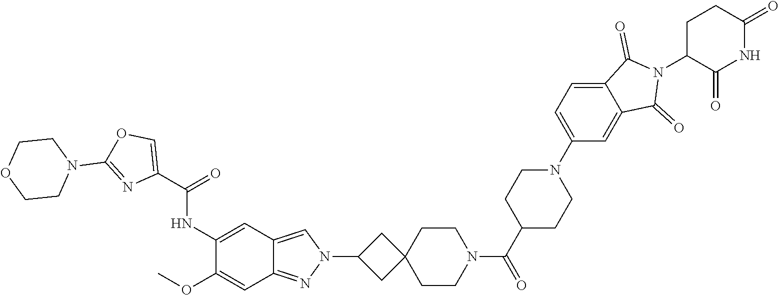 [SMILES: COc1cc2nn(C3CC4(CCN(C(=O)C5CCN(c6ccc7c(c6)C(=O)N(C6CCC(=O)NC6=O)C7=O)CC5)CC4)C3)cc2cc1NC(=O)c1coc(N2CCOCC2)n1] | 834.7 | (500 MHz, DMSO-d₆) δ 11.08 (s, 1H), 9.34 (s, 1H), 8.55 (s, 1H), 8.33 (s, 1H), 8.28 (s, 1H), 7.67 (d, J = 8.5 Hz, 1H), 7.33 (s, 1H), 7.25 (d, J = 8.5 Hz, 1H), 7.16 (s, 1H), 5.17-5.12 (m, 1H), 5.07 (dd, J = 13.0, 5.5 Hz, 1H), 4.09-4.05 (m, 2H), 3.96 (s, 3H), 3.75-3.69 (m, 4H), 3.49-3.45 (m, 5H), 3.43-3.38 (m, 2H), 3.15-3.05 (m, 3H), 3.03-2.98 (m, 1H), 2.94- 2.83 (m, 2H), 2.44-2.36 (m, 3H), 2.08-1.95 (m, 3H), 1.74-1.67 (m, 4H), 1.65-1.57 (m, 4H). |
| | I-54 | | |
| I-55 | 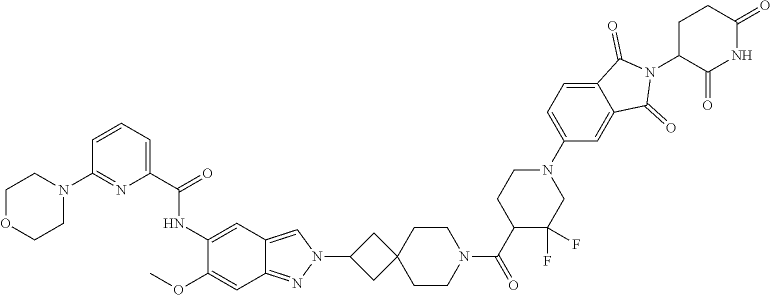 [SMILES: COc1cc2nn(C3CC4(CCN(C(=O)C5CCN(c6ccc7c(c6)C(=O)N(C6CCC(=O)NC6=O)C7=O)CC5(F)F)CC4)C3)cc2cc1NC(=O)c1cccc(N2CCOCC2)n1] | 880.3 | (500 MHz, DMSO-d₆) δ 11.08 (s, 1H), 10.79 (s, 1H), 8.63 (s, 1H), 8.34 (d, J = 2.0 Hz, 1H), 7.82 (dd, J = 8.5, 7.0 Hz, 1H), 7.69 (d, J = 8.5 Hz, 1H), 7.48-7.44 (m, 2H), 7.36-7.34 (m, 1H), 7.18 (d, J = 2.0 Hz, 1H), 7.16 (d, J = 8.5 Hz, 1H), 5.34-5.30 (m, 1H), 5.18-5.14 (m, 1H), 55.08 (dd, J = 13.0, 5.5 Hz, 2H), 4.40-4.30 (m, 2H), 3.97 (s, 3H), 3.95-3.92 (m, 1H), 3.82-3.79 (m, 4H), 3.61-3.58 (m, 4H), 3.54-3.48 (m, 2H), 2.93- 2.85 (m, 2H), 2.65-2.62 (m, 1H), 2.45-2.40 (m, 3H), 2.37-2.35 (m, 1H), 2.06-1.96 (m, 5H), 1.75-1.64 (m, 5H). |
| | I-55 | | |

### Example 9: Synthesis of I-43

### Step 1: Synthesis of (I-43-1)

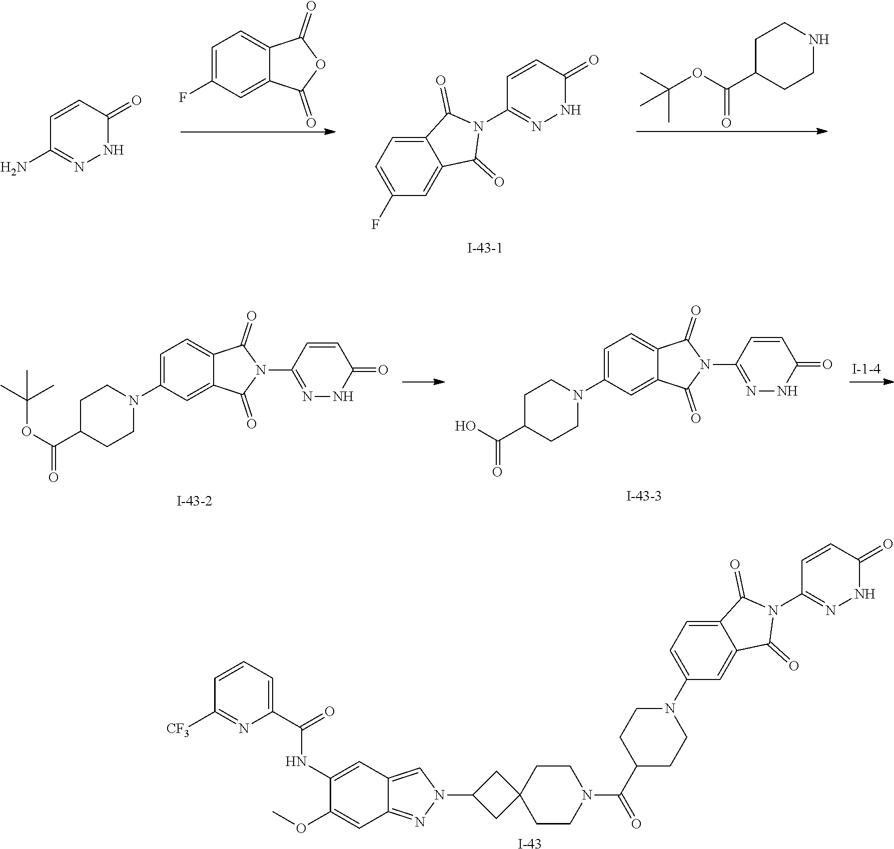

A mixture of 5-fluoroisobenzofuran-1,3-di one (0.9 g, 8.6 mmol), 6-aminopyridazine-3(2H)-one (1.3 g, 7.8 mmol), NaOAc (1.3 g, 15.5 mmol)) and HOAc (7 mL) was stirred at 80° C. for 16 hours. The mixture was returned to room temperature and concentrated under reduced pressure. The residue was poured into water and stirred. The solid was precipitated, filtered, washed with diethyl ether, and the filter cake was dried under vacuum to obtain the product I-43-1 as a white solid (1.2 g, yield 55%), MS (ESI) m/z: 260.2 [M+H]⁺.

### Step 2: Synthesis of (I-43-2)

To a solution of I-43-1(1.1 g, 4.4 mmol) in anhydrous DMSO (10.0 mL) was added DIEA (1.14 g, 8.8 mmol) and piperidine-4-carboxylic acid tert-butyl ester (0.82 g, 4.4 mmol), and the mixture was reacted at 90° C. for 2 hours. The mixture was returned to room temperature, diluted with water (100 mL), and extracted with ethyl acetate (80 mL×3). The combined organic phases were washed with saturated brine (50 mL), dried over anhydrous magnesium sulfate, filtered, and concentrated under reduced pressure. The crude product was purified by reverse C18 column chromatography to obtain product I-43-2 as a yellow solid (200 mg, yield 11%), MS (ESI) m/z: 425.3 [M+H]⁺.

### Step 3: Synthesis of (I-43-3)

TFA (1 mL) was added dropwise to a solution of I-43-2 (200 mg, 0.47 mmol) and DCM (5 mL) at 0° C., and the mixture was stirred at 25° C. for 16 hours. Diethyl ether was added to precipitate a solid, the supernatant was removed after standing and repeated three times, and dried under vacuum to obtain product I-43-3 as a yellow solid (180 mg, yield 99%), MS (ESI)m/z: 369.1 [M+H]⁺.

### Step 4: Synthesis of (I-43)

A mixed solution of I-43-3 (60 mg, 0.14 mmol), I-1-4 (52 mg, 0.14 mmol), HCTU (67 mg, 0.16 mmol), TEA (142 mg, 1.4 mmol) and DMF (3 mL) was stirred at 25° C. for 1 hour. The reaction solution was purified by preparative HPLC to obtain the product I-43 as a yellow solid (11.2 mg, yield 10%), MS (ESI) m/z: 810.2 [M+H]⁺. ¹H NMR (400 MHz, DMSO-d₆) δ 10.51 (s, 1H), 8.69 (s, 1H), 8.47 (d, J=7.5 Hz, 1H), 8.44-8.39 (m, 1H), 8.38 (d, J=1.0 Hz, 1H), 8.22 (dd, J=7.5, 1.0 Hz, 1H), 7.74 (d, J=8.5 Hz, 1H), 7.56 (d, J=9.5 Hz, 1H), 7.40 (d, J=1.5 Hz, 1H), 7.33-7.28 (m, 1H), 7.21 (s, 1H), 7.06 (d, J=9.5 Hz, 1H), 5.21-5.14 (m, 1H), 4.20-4.07 (m, 3H), 3.99 (s, 3H),3.59-3.38 (m, 4H), 3.13-3.02 (m, 3H), 2.70-2.65 (m, 1H) 2.49-2.36 (m, 4H), 1.84-1.49 (m, 8H).

### Example 10: Synthesis of I-45

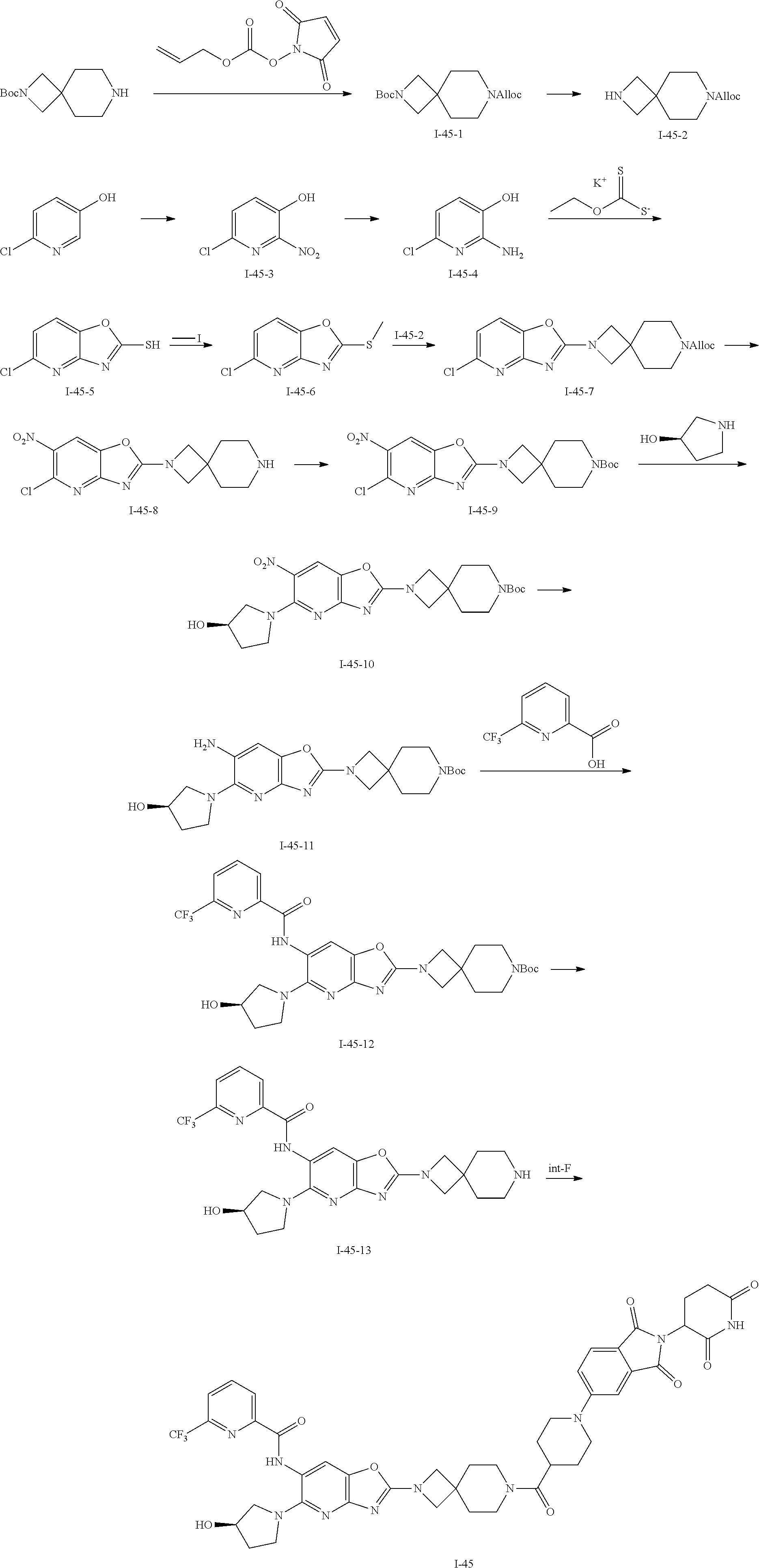

### Step 1: Synthesis of (I-45-1)

To a solution of K₂CO₃ (18.35 g, 132.8 mmol) and 2,7-diazaspiro[3.5]nonane-2-carboxylic acid tert-butyl ester (10 g, 44.25 mmol) in acetone/H₂O (120/40 mL) was add (2,5-dioxy-2,5-dihydro-1H-pyrrol-1-yl) allyl carbonate (10.6 g, 53.1 mmol) under a nitrogen atmosphere at 0° C., and the solution was stirred at 25° C. for 16 hours. The reaction system was diluted with ethyl acetate (500 mL), washed with 3N HCl, dried over anhydrous sodium sulfate, and concentrated under reduced pressure to obtain a crude product, which was purified by flash silica gel column chromatography to obtain product I-45-1 as a yellow solid (13.0 g, yield 94%), ¹H NMR (400 MHz, CDCl₃) δ 6.00-5.87 (m, 1H), 5.31-5.20 (m, 2H), 4.59-4.57 (m, 2H), 3.65 (s, 4H), 3.46-3.38 (m, 4H), 1.74-1.70 (m, 4H), 1.43 (s, 9H).

### Step 2: Synthesis of (I-45-2)

TFA (24 mL) was added to a solution of I-45-1 (12.2 g, 39.0 mmol) in DCM (240 mL) at 0° C., and the reaction was carried out at 25° C. for 2 hours. Diethyl ether was added to precipitate a solid, the supernatant was removed after standing and repeated three times, and dried under vacuum to obtain product I-45-2 as a brown solid (10 g, yield 100%), ¹H NMR (400 MHz, DMSO-d₆) δ 5.97-5.87 (m, 1H), 5.29-5.17 (m, 2H), 4.53-4.51 (m, 2H), 3.73 (t, J=6.4 Hz, 4H), 1.78-1.66 (m, 4H).

### Step 3: Synthesis of (I-45-3)

To a solution of KNO₃ (31 g, 308 mmol) and concentrated H₂SO₄ (250 mL) was added 6-chloropyridin-3-ol (22 g, 170 mmol) in batches at 0° C. After 0.5 hours, the dropwise addition was completed, and the mixture was stirred at 25° C. for 2 hours. The reaction mixture was quenched with ice water, the solid was filtered, washed with H₂O, and the filter cake was dried under vacuum to obtain product I-45-3 as a yellow solid (20 g, yield 69%), MS (ESI) m/z: 175.1 [M+H]⁺. ¹H NMR (400 MHz, DMSO-d₆) δ 11.92 (s, 1H), 7.73 (q, J=8.8 Hz, 2H).

### Step 4: Synthesis of (I-45-4)

Under a nitrogen atmosphere, Fe (25.4 g, 453 mmol) was added to a solution of I-45-3 (15 g, 90 mmol) and N H₄Cl (24 g, 453 mmol) in MeOH/H 20 (150 mL/50 mL), and the solution was reacted at 70° C. for 4 hours. The reaction solution was returned to room temperature, added with water (300 mL) to dilute, and extracted with ethyl acetate (200 mL×3). The combined organic phases were washed with saturated brine (300 mL), dried over anhydrous sodium sulfate, filtered, and concentrated under reduced pressure to obtain a crude product, which was purified by silica gel column chromatography (PE/EtOAc=5/1) to obtain product I-45-4 as a yellow solid (5 g, yield 24%), MS (ESI) m/z: 145.0 [M+H]⁺.

### Step 5: Synthesis of (I-45-5)

A mixture of I-45-4 (7 g, 48 mmol) and potassium O-ethyldithiocarbonate (10.8 g, 68 mmol) in pyridine (80 mL) was stirred at 110° C. for 16 hours under a nitrogen atmosphere. The reaction system was returned to room temperature and diluted with ice water (100 mL), acidified with dilute hydrochloric acid (4 M) to pH=3-4 under stirring, and then extracted with ethyl acetate (100 mL×3). The organic phases were combined, washed with saturated brine (300 mL), dried over anhydrous sodium sulfate, filtered, and concentrated under reduced pressure to obtain product I-45-5 as a yellow solid (4 g, yield 45%), MS (ESI) m/z: 186.1 [M+H]⁺.

### Step 6: Synthesis of (I-45-6)

To a solution of I-45-5 (6 g, 32 mmol) and EtOAc (60 mL) were added methyl iodide (9 g, 64 mmol) and K₂CO₃ (8.8 g, 64 mmol), and the resulting mixture was sealed and stirred at 25° C. under nitrogen protection for 2 hours. The reaction solution was diluted with H 20 (100 mL) and extracted with EtOAc (100 mL×3). The combined organic layers were washed with saturated brine, dried over anhydrous sodium sulfate, filtered and concentrated under reduced pressure. The crude product was purified by silica gel column chromatography (PE/EtOAc=10/1) to obtain product I-45-6 as a yellow solid (4 g, yield 62.5%), MS (ESI) m/z: 200.9 [M+H]⁺. ¹H NMR (400 MHz, CDCl₃) δ 7.58 (d, J=8.4 Hz, 1H), 7.14 (d, J=8.4 Hz, 1H), 2.73 (s, 3H).

### Step 7: Synthesis of (I-45-7)

Under a nitrogen atmosphere, I-45-6 (4.2 g, 21 mmol), I-45-2 (22 g, 105 mmol), DIEA (13.6 g, 105 mol) and 1,4-dioxane (40 mL) solution was stirred at 110° C. for 16 hours. The reaction solution was diluted with H₂O (200 mL) and extracted with EtOAc (300 mL×3). The combined organic layers were washed with saturated brine, dried over anhydrous sodium sulfate, filtered and concentrated. The crude product was purified by silica gel column chromatography (PE/EtOAc=3/1) to obtain product I-45-7 as a yellow solid (5 g, yield 65%), MS (ESI) m/z: 363.1 [M+H]⁺.

### Step 8: Synthesis of (I-45-8)

To a solution of I-45-7 (5 g, 13.7 mmol) and concentrated H₂SO₄ (40 mL) were slowly added concentrated H₂SO₄ (10 mL) and concentrated HNO₄ (10 mL) dropwise under stirring at −30° C. to −20° C. The dropwise addition was complete after 2 hours. The reaction solution was stirred for 20 minutes and then ice water was added to quench the reaction. The solid was filtered, washed with H₂O, and the filter cake was dried under vacuum to obtain the product I-45-8 as a yellow solid (2.2 g, yield 50%), MS (ESI) m/z: 324.1 [M+H]⁺.

### Step 9: Synthesis of (I-45-9)

To a solution of I-45-8 (2.2 g, 6.8 mmol), D M A P (0.08 g, 0.6 mmol), TEA (2.06 g, 20.4 mmol) and THF (20 mL) was added di-tert-butyl dicarbonate (2.22 g, 10 mmol) at 0° C. The resulting mixture was stirred for 1 hour until the reaction was complete. The mixture was diluted with H₂O (200 mL) and extracted with EtOAc (200 mL×3). The combined organic layers were washed with saturated brine, dried over anhydrous sodium sulfate, filtered and concentrated under reduced pressure. The crude product was purified by preparative thin layer chromatography (PE/EtOAc=1/1) to obtain product I-45-9 as a yellow solid (600 mg, yield 21%), MS (ESI) m/z: 424.0 [M+H]⁺.

### Step 10: Synthesis of (I-45-10)

A mixture of I-45-9 (600 mg, 1.4 mmol), (R)-pyrrolidin-3-ol (699 mg, 5.66 mmol), DIEA (362 mg, 2.8 mmol) and toluene (10 mL) was sealed and stirred under nitrogen protection at 110° C. for 16 hours. The reaction mixture was returned to room temperature, diluted with H 20 (100 mL) and extracted with EtOAc (100 mL×3). The combined organic layers were washed with saturated brine, dried over anhydrous sodium sulfate, filtered and concentrated under reduced pressure. The crude product was purified by preparative thin layer chromatography (PE/EtOAc=1/1) to obtain product I-45-10 as a yellow solid (350 mg, yield 52.6%), MS (ESI) m/z: 475.3 [M+H]⁺.

### Step 11: Synthesis of (I-45-11)

To a solution of I-45-10 (350 mg, 0.73 mmol) in MeOH (2 mL) was added wet Pd/C (35 mg, 10% purity) and the solution was stirred under a hydrogen atmosphere at 25° C. for 4 hours. The reaction system was filtered through diatomaceous earth, and the filter cake was washed with EtOAc (30 mL). The filtrate was concentrated under reduced pressure to obtain the product I-45-11 as a green solid (200 mg, yield 61.5%), MS (ESI) m/z: 445.2 [M+H]⁺.

### Step 12: Synthesis of (I-45-12)

A mixture of 6-(trifluoromethyl)picolinic acid (51.5 mg, 0.27 mmol), I-45-11 (120 mg, 0.27 mmol), HOBT (54.6 mg, 0.4 mmol), EDCI (52 mg, 0.4 mol), DMAP (6.6 mg, 0.05 mmol) and DMF (2 mL) was stirred at 25° C. for 16 h. The reaction solution was diluted with H₂O (50 mL) and extracted with EtOAc (50 mL×3). The combined organic layers were washed with saturated brine, dried over anhydrous sodium sulfate, filtered and concentrated. The crude product was purified by preparative thin layer chromatography (PE/EtOAc=3/1) to obtain product I-45-12 as a yellow solid (50 mg, yield 30%), MS (ESI) m/z: 618.1 [M+H]⁺.

### Step 13: Synthesis of (I-45-13)

I-45-12 (50 mg, 0.08 mmol) was added to H CI-dioxane (1 M, 3 mL) at 0° C., and the reaction mixture was stirred at 25° C. for 1 hour. Diethyl ether was added to the reaction mixture under stirring, and the supernatant was removed after standing and repeated three times to obtain the product I-45-13 as a yellow solid (35 mg, yield 85.3%), MS (ESI) m/z 518.2 [M+H]⁺.

### Step 14: Synthesis of (I-45)

A mixed solution of int-F (28.3 mg, 0.073 mmol), TEA (30 mg, 0.294 mmol), I-45-13 (38 mg, 0.073 mmol), HCTU (33.4 mg, 0.080 mmol) and DMF (20 mL) was stirred at 25° C. for 16 hours until the reaction was complete. The reaction mixture was diluted with H₂O (50 mL) and extracted with ethyl acetate (50 mL×3). The combined organic phases were washed with saturated brine (50 mL), dried over anhydrous sodium sulfate, filtered, and concentrated under reduced pressure. The crude product was purified by preparative H PLC to obtain the product I-45 as a yellow solid (23 mg, yield 35%). ¹H NMR (400 MHz, DMSO-d₆) δ 11.07 (s, 1H), 10.17 (s, 1H), 8.51-8.32 (m, 2H), 8.19 (dd, J=7.5, 1.0 Hz, 1H), 8.01 (s, 1H), 7.66 (d, J=8.5 Hz, 1H), 7.33 (d, J=1.5 Hz, 1H), 7.24 (dd, J=8.5, 2.0 Hz, 1H), 5.06 (dd, J=13.0, 5.5 Hz, 1H), 4.88-4.81 (m, 1H), 4.29 (s, 1H), 4.08-4.01 (m, 6H), 3.71-3.50 (m, 4H), 3.48-3.33 (m, 4H), 3.08-3.01 (m, 4H), 2.67-2.56 (m, 1H), 2.70-2.53 (m, 2H), 2.07-1.89 (m, 2H), 1.86-1.58 (m, 8H).

### Example 11: Synthesis of Compound II-2

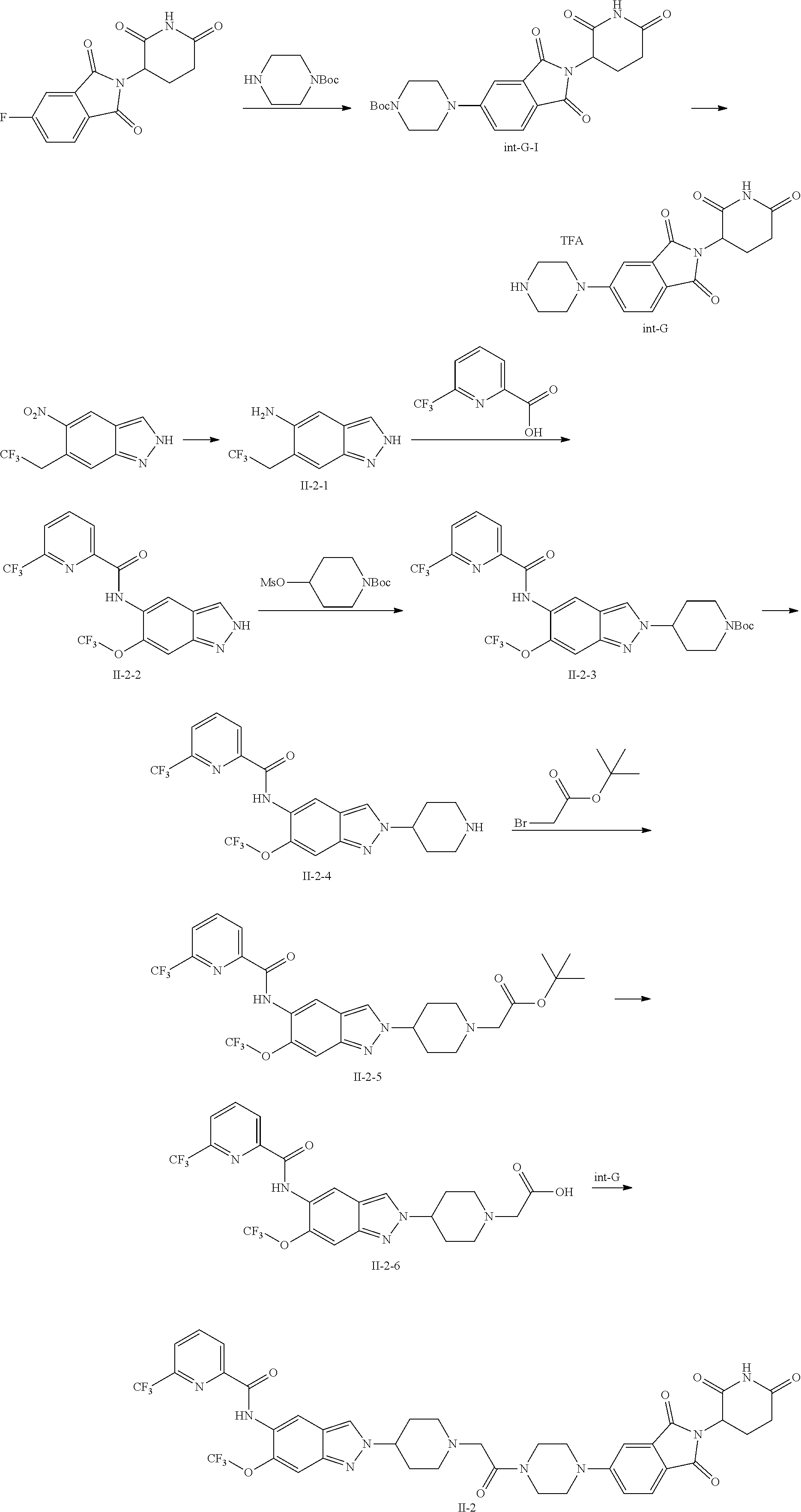

### Step 1: Synthesis of (Int-G-1)

A mixed solution of 2-(2,6-dioxo-3-piperidinyl)-5-fluoro-isoindole-1,3-dione (1 g, 3.62 mmol), piperazine-1-carboxylic acid tert-butyl ester (809.14 mg, 4.34 mmol), DIPEA (935.80 mg, 7.24 mmol, 1.26 mL) and DMSO (10 mL) was stirred at 100° C. for 2 hours. The reaction solution was returned to room temperature, added with water (60 mL) with stirring to precipitate the solid, filtered and dried to obtain the product int-G-1 as a white solid (1.2 g, crude product), MS (ESI) m/z: 443.3 [M+H]⁺.

### Step 2: Synthesis of (int-G)

A mixed solution of int-G-1 (35 mg, 79.10 μmol), TFA (1 mL) and DCM (1 mL) was stirred at 25° C. for 1 hour. The mixture was concentrated under reduced pressure to obtain the product int-G as a yellow solid (25 mg, yield 69.25%, TFA salt), which is directly used in the next step of reaction, MS (ESI) m/z: 343.2 [M+H]⁺.

### Step 3: Synthesis of (II-2-1)

5-nitro-6-(trifluoromethoxy)-2H-indazole (250 mg, 1.01 mmol) was dissolved in MeOH (3 mL), and catalyst wet Pd/C (26.14 mg, 10% purity) was added. The mixed solution was replaced with hydrogen three times under stirring, and kept at the hydrogen environment for 2 hours. The reaction solution was filtered through diatomaceous earth to remove the catalyst, and the filtrate was spin-dried to obtain product II-2-1 as a brown solid (211 mg, yield 96.05%), MS (ESI) m/z: 218.1 [M+H]⁺.

### Step 4: Synthesis of (II-2-2)

A mixed solution of II-2-1 (320 mg, 1.47 mmol), 6-(trifluoromethyl)pyridine-2-carboxylic acid (309.79 mg, 1.62 mmol), DMF (5 mL), HATU (840.49 mg, 2.21 mmol) and DIPEA (380.91 mg, 2.95 mmol, 513.35 μL) was stirred at 25° C. for 6 hours until the reaction was complete. The reaction solution was added with water (30 mL), and extracted with EA (50 mL×3). The combined organic layers were washed with saturated brine, dried over anhydrous sodium sulfate, filtered and concentrated in vacuo. The crude product was purified by silica gel column chromatography (DCM:MeOH=50:1) to obtain product 11-2-2 as a brown solid (295 mg, yield 51.30%), MS (ESI) m/z: 391.2 [M+H]⁺.

### Step 5: Synthesis of (II-2-3)

A mixed solution of II-2-2 (100 mg, 256.25 μmol), tert-butyl 4-methylsulfonyloxypiperidine-1-carboxylate (143.17 mg, 512.51 μmol), NaOH (20.50 mg, 512.51 μmol) and DMF (3 mL) was stirred at 110° C. for 6 hours. The reaction solution was added with water (30 mL), and extracted with EA (50 mL×3). The combined organic layers were washed with brine, dried over sodium sulfate, filtered and concentrated in vacuo. The crude product was purified by silica gel column chromatography (PE:EA=3:1) to obtain product II-2-3 as brown solid (71 mg, yield 48.31%), MS (ESI) m/z: 574.5 [M+H]⁺.

### Step 6: Synthesis of (II-2-4)

II-2-3 (71 mg, 123.80 mol) was dissolved in DCM (5 mL), and HCl-dioxane solution (4 M, 309.51 μL) was added dropwise to the mixture at 25° C. After the dropwise addition was completed, the reaction system was stirred for 2 hours. The reaction solution was added with water (30 mL), adjusted the pH to neutral with saturated NaHCO₃, and then extracted with DCM (20 mL×3). The combined organic layers were washed with saturated brine, dried over anhydrous sodium sulfate, filtered and concentrated in vacuum to obtain product II-2-4 as a white solid (48 mg, yield 81.90%), MS (ESI) m/z: 474.1 [M+H]⁺.

### Step 7: Synthesis of (II-2-5)

A mixture of II-2-4 (48 mg, 101.40 μmol), tert-butyl 2-bromoacetate (23.73 mg, 121.68 μmol), DIPEA (26.21 mg, 202.80 mol, 35.32 μL) and DMF (2 mL) was stirred at 25° C. for 2 hours. The reaction solution was added with water (20 mL), and extracted with EA (20 mL×3). The combined organic layers were washed with saturated brine, dried over anhydrous sodium sulfate, filtered and concentrated in vacuum. The crude product was purified by silica gel column chromatography (DCM:MeOH=20:1) to obtain product II-2-5 as a white solid (45 mg, yield 75.54%), MS (ESI) m/z: 588.5 [M+H]⁺.

### Step 8: Synthesis of (II-2-6)

A mixed solution of II-2-5 (45 mg, 76.59 μmol), DCM (1.5 mL) and TFA (1.5 mL) was stirred at 25° C. for 2 hours. The mixture was repeatedly concentrated under reduced pressure to remove the solvent to obtain product II-2-6 as a white solid (24 mg, yield 58.96%), MS (ESI) m/z: 532.1[M+H]⁺.

### Step 9: Synthesis of (II-2)

A mixed solution of II-2-6 (24 mg, 45.16 μmol), int-G (22.67 mg, 49.68 μmol, TFA salt), DMF (2 mL), DIPEA (17.51 mg, 135.49 μmol, 23.60 μL) and HATU (25.76 mg, 67.74 μmol) was stirred at 25° C. for 2 hours. The reaction solution was added with water (20 mL), and extracted with EA (20 mL×3). The combined organic layers were washed with saturated brine, dried over anhydrous sodium sulfate, filtered and concentrated in vacuo. The crude product was purified by preparative thin layer chromatography to obtain product II-2 as a yellow solid (23 mg, yield 58.32%), MS (ESI) m/z: 856.3 [M+H]⁺. ¹H NMR (500 MHz, DMSO-d₆) δ 11.08 (s, 1H), 10.39 (s, 1H), 8.69 (s, 1H), 8.62 (s, 1H), 8.47 (d, J=7.5 Hz, 1H), 8.42 (t, J=8.0 Hz, 1H), 8.24 (dd, J=7.5, 1.0 Hz, 1H), 7.82 (s, 1H), 7.72 (d, J=8.5 Hz, 1H), 7.39 (d, J=2.0 Hz, 1H), 7.29 (dd, J=8.5, 2.0 Hz, 1H), 5.08 (dd, J=13.0, 5.5 Hz, 1H), 4.57-4.50 (m, 1H), 3.80-3.75 (m, 2H), 3.65-3.60 (s, 2H), 3.60-3.55 (m, 2H), 3.52-3.47 (m, 2H), 3.01 (d, J=11.0 Hz, 2H), 2.93-2.84 (d, 1H), 2.66-2.59 (m, 1H), 2.59-2.53 (m, 1H), 2.37-2.24 (m, 3H), 2.17-2.09 (, 4H), 2.06-1.94 (m, 2H).

Compounds II-4, II-7, II-10, II-12, II-13, II-16, II-17, II-20, and II-21 can be prepared by referring to the method of Examples 2 and 11 above. The structure and characterization data are shown in the following table:

| Molecular | | MS (ESI) | |
|---|---|---|---|
| ID | structure | m/z: [M + H]⁺ | ¹H NMR |
| II-4 | 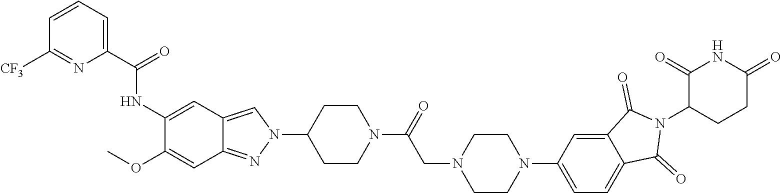 [SMILES: COc1cc2nn(C3CCN(C(=O)CN4CCN(c5ccc6c(c5)C(=O)N(C5CCC(=O)NC5=O)C6=O)CC4)CC3)cc2cc1NC(=O)c1cccc(C(F)(F)F)n1] II-4 | 802.6 | (500 MHz, DMSO-d₆) δ 11.14 (s, 1H), 10.56 (s, 1H), 8.75 (s, 1H), 8.51 (d, J = 7.5 Hz, 1H), 8.48-8.41 (m, 2H), 8.27 (d, J = 7.5, 1.0 Hz, 1H), 7.74 (d, J = 8.5 Hz, 1H), 7.42 (s, 1H), 7.33 (d, J = 8.5, 2.0 Hz, 1H), 7.21 (s, 1H), 5.13 (dd, J = 12.8, 5.4 Hz, 1H), 4.82-4.73 (m, 1H), 4.60-4.53 (m, 1H), 4.34-4.26 (m, 1H), 4.03 (s, 3H), 3.57-3.51 (m, 4H), 3.48-3.46 (m, 1H), 3.37-3.20 (m, 3H), 2.97- 2.86 (m, 2H), 2.69-2.65 (m, 4H), 2.64-2.59 (m, 1H), 2.26-2.12 (m, 3H), 2.11-2.05 (m, 1H), 1.99-1.90 (m, 1H). |
| II-7 | 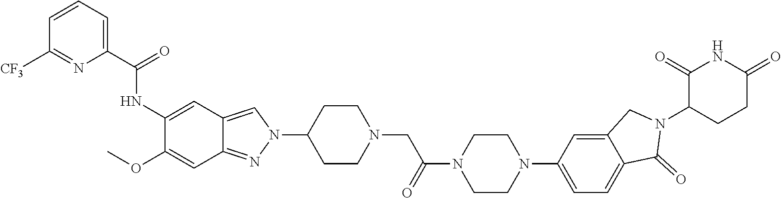 [SMILES: COc1cc2nn(C3CCN(CC(=O)N4CCN(c5ccc6c(c5)CN(C5CCC(=O)NC5=O)C6=O)CC4)CC3)cc2cc1NC(=O)c1cccc(C(F)(F)F)n1] II-7 | 788.6 | (500 MHz, DMSO-d₆) δ 10.95 (s, 1H), 10.50 (s, 1H), 8.69 (s, 1H), 8.46 (d, J = 7.5 Hz, 1H), 8.41 (t, J = 7.5 Hz, 1H), 8.37 (s, 1H), 8.22 (d, J = 7.5 Hz, 1H), 7.56 (d, J = 8.0 Hz, 1H), 7.17 (s, 1H), 7.12 (d, J = 8.5 Hz, 2H), 5.05 (dd, J = 13.0, 5.0 Hz, 1H), 4.44- 4.38 (m, 1H), 4.35 (d, J = 16.9 Hz, 1H), 4.23 (d, J = 17.0 Hz, 1H), 3.98 (s, 3H), 3.81-3.72 (m, 2H), 3.66- 3.60 (m, 2H), 3.32-3.31 (m, 1H), 3.04-2.97 (m, 2H), 2.95-2.82 (m, 2H), 2.67-2.55 (m, 3H), 2.44- 2.35 (m, 2H), 2.32-2.25 (m, 2H), 2.16-2.0 (m, 4H), 2.03-1.92 (m, |
| | | | 2H). |
| II-10 | 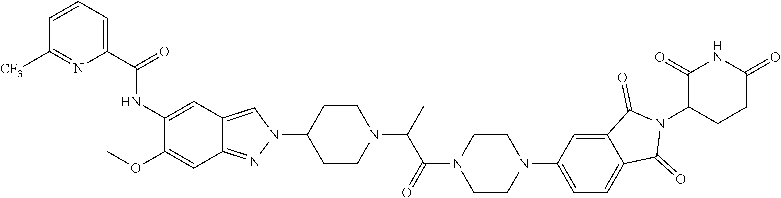 [SMILES: COc1cc2nn(C3CCN(C(C)C(=O)N4CCN(c5ccc6c(c5)C(=O)N(C5CCC(=O)NC5=O)C6=O)CC4)CC3)cc2cc1NC(=O)c1cccc(C(F)(F)F)n1] II-10 | 816.6 | (500 MHz, DMSO-d₆) δ 11.08 (s, 1H), 10.49 (s, 1H), 8.68 (s, 1H), 8.46 (d, J = 8.0 Hz, 1H), 8.40 (t, J = 8.0 Hz, 1H), 8.35 (d, J = 1.5 Hz, 1H), 8.21 (d, J = 7.5 Hz, 1H), 7.72 (d, J = 8.5 Hz, 1H), 7.40 (d, J = 2.0 Hz, 1H), 7.30 (dd, J = 8.5, 2.0 Hz, 1H), 7.15 (s, 1H), 5.09 (dd, J = 13.0, 5.5 Hz, 1H), 4.42-4.29 (m, 1H), 3.97 (d, J = 2.0 Hz, 3H), 3.89-3.79 (m, 2H), 3.78-3.59 (m, 3H), 3.55-3.41 (m, 2H), 3.01-2.83 (m, 2H), 2.72 (d, J = 11.0 Hz, 1H), 2.67-2.54 (m, 3H), 2.54-2.51 (m, 1H), 2.42-2.32 (m, 1H), 2.21-1.93 (m, 5H), 1.23- |
| | | | 1.2 (m, 1H), 1.10 (d, J = 6.0 Hz, 3H). |
| II-12 | 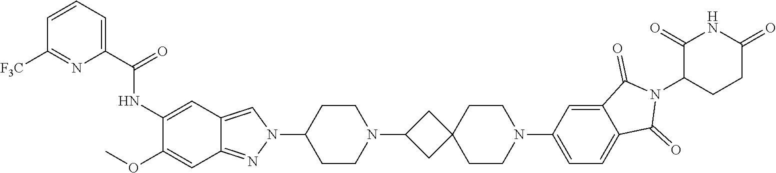 [SMILES: COc1cc2nn(C3CCN(C4CC5(CCN(c6ccc7c(c6)C(=O)N(C6CCC(=O)NC6=O)C7=O)CC5)C4)CC3)cc2cc1NC(=O)c1cccc(C(F)(F)F)n1] II-12 | 799.6 | (500 MHz, DMSO-d₆) δ 11.07 (s, 1H), 10.51 (s, 1H), 8.69 (s, 1H), 8.47 (d, J = 7.5 Hz, 1H), 8.41 (t, J = 8.0 Hz, 1H), 8.38 (s, 1H), 8.22 (d, J = 7.5 Hz, 1H), 7.65 (d, J = 8.5 Hz, 1H), 7.32 (d, J = 2.0 Hz, 1H), 7.25 (dd, J = 8.5, 2.0 Hz, 1H), 7.16 (s, 1H), 5.06 (dd, J = 13.0, 5.5 Hz, 1H), 4.40 (br, 1H), 3.99 (s, 3H), 3.52-3.46 (m, 2H), 3.43-3.37 (m, 2H), 2.98-2.84 (m, 3H), 2.83-2.73 (m, 1H), 2.66- 2.56 (m, 2H), 2.14-1.89 (m, 9H), 1.70-1.55 (m, 5H), 1.50-1.43 (m, 1H). |
| II-13 | 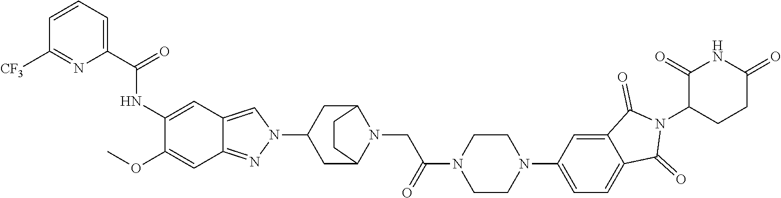 [SMILES: COc1cc2nn(C3CC4CCC(C3)N4CC(=O)N3CCN(c4ccc5c(c4)C(=O)N(C4CCC(=O)NC4=O)C5=O)CC3)cc2cc1NC(=O)c1cccc(C(F)(F)F)n1] II-13 | 828.6 | (500 MHz, CDCl₃) δ 10.62 (s, 1H), 8.73 (s, 1H), 8.42 (d, J = 8.0 Hz, 1H), 8.28 (s, 1H), 8.08-8.01 (m, 1H), 7.93 (s, 1H), 7.78 (d, J = 8.0 Hz, 1H), 7.65 (d, J = 8.0 Hz, 1H), 7.23 (s, 1H), 7.02 (d, J = 8.5 Hz, 1H), 6.99 (s, 1H), 4.89 (dd, J = 12.0, 5.0 Hz, 1H), 4.65- 4.58 (m, 1H), 3.95 (s, 3H), 3.85 (br, 2H), 3.75 (br, 2H), 3.52-3.44 (m, 3H), 3.43-3.37 (m, 2H), 3.36-3.25 (m, 2H), 2.86-2.74 (m, 2H), 2.73- 2.67 (m, 1H), 2.65-2.56 (m, 3H), 2.10-2.04 (m, 1H), 1.96-1.89 (m, 2H), 1.79-1.72 (m, 2H), 1.68-1.62 |
| | | | (m, 2H). |
| II-16 | 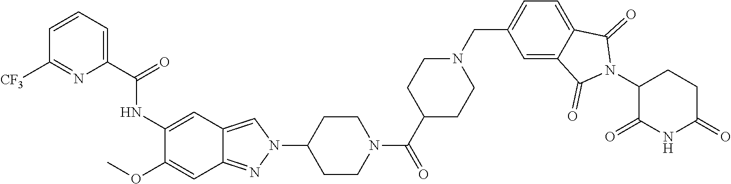 [SMILES: COc1cc2nn(C3CCN(C(=O)C4CCN(Cc5ccc6c(c5)C(=O)N(C5CCC(=O)NC5=O)C6=O)CC4)CC3)cc2cc1NC(=O)c1cccc(C(F)(F)F)n1] II-16 | 801.6 | (500 MHz, DMSO-d₆) δ 11.13 (s, 1H), 10.50 (s, 1H), 8.69 (s, 1H), 8.46 (d, J = 7.5 Hz, 1H), 8.43-8.39 (m, 1H), 8.38 (s, 1H), 8.22 (d, J = 7.5 Hz, 1H), 7.89 (d, J = 7.5 Hz, 1H), 7.85 (s, 1H), 7.82 (d, J = 7.5 Hz, 1H), 7.16 (s, 1H), 5.15 (dd, J = 13.0, 5.5 Hz, 1H), 4.72-4.66 (m, 1H), 4.58-4.52 (m, 1H), 4.13-4.07 (m, 1H), 3.98 (s, 3H), 3.68 (s, 2H), 2.93-2.83 (m, 3H), 2.80-2.74 (m, 1H), 2.70-2.58 (m, 3H), 2.19-2.05 (m, 5H), 2.03- 1.94 (m, 2H), 1.91-1.85 (m, 1H), 1.71-1.58 (m, 4H). |
| II-17 | 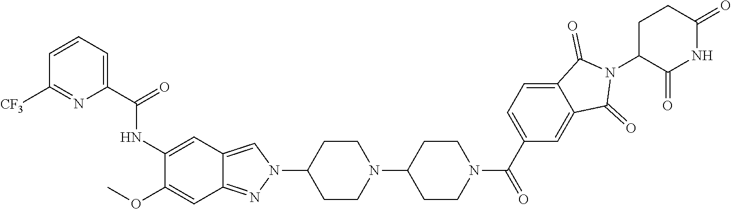 [SMILES: COc1cc2nn(C3CCN(C4CCN(C(=O)c5ccc6c(c5)C(=O)N(C5CCC(=O)NC5=O)C6=O)CC4)CC3)cc2cc1NC(=O)c1cccc(C(F)(F)F)n1] II-17 | 787.5 | (500 MHz, DMSO-d₆) δ 11.21 (s, 1H), 10.56 (s, 1H), 8.74 (s, 1H), 8.52 (d, J = 7.5 Hz, 1H), 8.46 (t, J = 8.0 Hz, 1H), 8.42 (s, 1H), 8.27 (d, J = 8.0 Hz, 1H), 8.05 (d, J = 7.5 Hz, 1H), 7.99 (s, 1H), 7.96-7.90 (m, 1H), 7.22 (s, 1H), 5.25 (dd, J = 13.0, 5.5 Hz, 1H), 4.66-4.55 (m, 1H), 4.48- 4.37 (m, 1H), 4.04 (s, 3H), 3.18- 3.05 (m, 3H), 3.00-2.86 (m, 2H), 2.74-2.65 (m, 2H), 2.63-2.59 (m, 1H), 2.49-2.41 (m, 2H), 2.21-2.08 (m, 5H), 1.99-1.91 (m, 1H), 1.80- 1.71 (m, 1H), 1.69-1.44 (m, 3H. |
| II-20 | 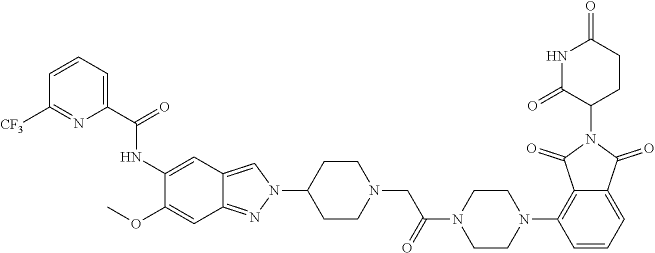 [SMILES: COc1cc2nn(C3CCN(CC(=O)N4CCN(c5cccc6c5C(=O)N(C5CCC(=O)NC5=O)C6=O)CC4)CC3)cc2cc1NC(=O)c1cccc(C(F)(F)F)n1] | 802.6 | (500 MHz, DMSO-d₆) δ 11.11 (s, 1H), 10.50 (s, 1H), 8.68 (s, 1H), 8.46 (d, J = 7.5 Hz, 1H), 8.40 (t, J = 8.0 Hz, 1H), 8.38 (s, 1H), 8.22 (dd, J = 7.5, 1.0 Hz, 1H), 7.74 (dd, J = 8.0, 1.5 Hz, 1H), 7.40 (dd, J = 9.0, 1.5 Hz, 2H), 7.16 (s, 1H), 5.12 (dd, J = 12.5, 5.5 Hz, 1H), 4.44-4.38 (m, 1H), 3.98 (s, 3H), 3.86-3.78 (m, 2H), 3.71-3.63 (m, 2H), 3.32-3.24 (m, 5H), 3.02-2.97 (m, 2H), 2.93- 2.85 (m, 1H), 2.66-2.55 (m, 2H), 2.55-2.52 (m, 1H), 2.33-2.26 (m, 2H), 2.15-2.08 (m, 4H), 2.07-2.03 (m, 1H). |
| | II-20 | | |
| II-21 | 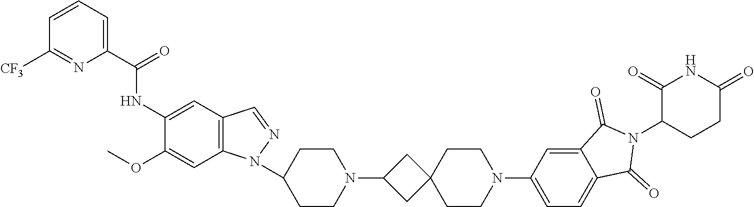 [SMILES: COc1cc2c(cnn2C2CCN(C3CC4(CCN(c5ccc6c(c5)C(=O)N(C5CCC(=O)NC5=O)C6=O)CC4)C3)CC2)cc1NC(=O)c1cccc(C(F)(F)F)n1] II-21 | 799.6 | (500 MHz, DMSO-d₆) δ 11.07 (s, 1H), 10.50 (s, 1H), 8.68 (s, 1H), 8.47 (d, J = 7.5 Hz, 1H), 8.41 (t, J = 8.0 Hz, 1H), 8.37 (s, 1H), 8.22 (d, J = 7.5 Hz, 1H), 7.66 (d, J = 8.5 Hz, 1H), 7.34 (d, J = 2.0 Hz, 1H), 7.29-7.24 (m, 1H), 7.16 (s, 1H) 5.06 (dd, J = 13.0, 5.5 Hz, 1H), 4.40 (br, 1H), 3.99 (s, 3H), 3.52-3.46 (m, 2H), 3.43- 3.37 (m, 2H), 2.98-2.84 (m, 3H), 2.83-2.73 (m, 1H), 2.66-2.56 (m, 2H), 2.14-1.89 (m, 9H), 1.70-1.55 (m, 5H), 1.50-1.43 (m, 1H) |

### Example 12: Synthesis of I-57 and I-58

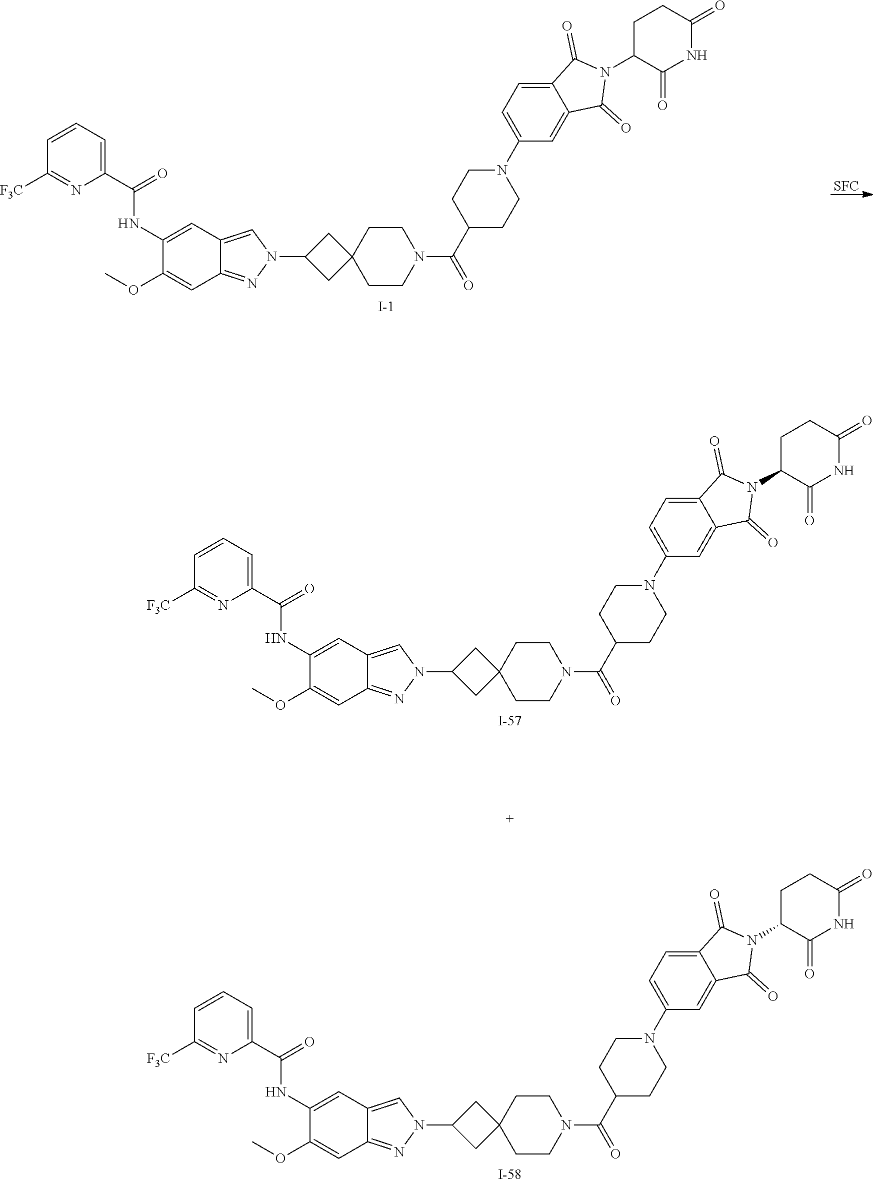

I-1 (100 mg) was chiral resolved by SFC (supercritical fluid chromatography) to obtain I-57 as a yellow solid (47 mg) and I-58 as a yellow solid (44 mg).

I-57 or I-58: Chiral H PLC: Optical purity (ee value)=100%, RT=2.262 min. MS (ESI) m/z: 827.8 [M+H]⁺. ¹H NMR (500 MHz, DMSO-d₆) δ 11.09 (s, 1H), 10.51 (s, 1H), 8.69 (s, 1H), 8.46 (d, J=8.0 Hz, 1H), 8.41 (t, J=8.0 Hz, 1H), 8.38 (s, 1H), 8.22 (d, J=8.0 Hz, 1H), 7.67 (d, J=8.5 Hz, 1H), 7.34 (s, 1H), 7.25 (d, J=8.5 Hz, 1H), 7.22 (s, 1H), 5.24-5.12 (m, 1H), 5.07 (dd, J=13.0, 5.0 Hz, 1H), 4.07 (d, J=12.5 Hz, 2H), 3.99 (s, 3H), 3.60-3.38 (m, 4H), 3.13-3.07 (m, 2H), 3.04-2.97 (m, 1H), 2.92-2.84 (m, 1H), 2.66-2.52 (m, 2H), 2.49-2.36 (m, 4H), 2.04-1.98 (m, 1H), 1.75-1.67 (m, 4H), 1.66-1.56 (m, 4H).

I-57 or I-58: Chiral HPLC: Optical purity (ee value)=99.64%, RT=3.319 min. MS (ESI) m/z: 827.8 [M+H]⁺. ¹H NMR (500 MHz, DMSO-d₆) δ 11.10 (s, 1H), 10.51 (s, 1H), 8.69 (s, 1H), 8.46 (d, J=8.0 Hz, 1H), 8.41 (t, J=8.0 Hz, 1H), 8.38 (s, 1H), 8.22 (d, J=8.0 Hz, 1H), 7.67 (d, J=8.5 Hz, 1H), 7.34 (s, 1H), 7.25 (d, J=8.5 Hz, 1H), 7.22 (s, 1H), 5.24-5.13 (m, 1H), 5.07 (dd, J=13.0, 5.0 Hz, 1H), 4.07 (d, J=12.5 Hz, 2H), 3.99 (s, 3H), 3.59-3.39 (m, 4H), 3.13-3.07 (m, 2H), 3.04-2.97 (m, 1H), 2.92-2.83 (m, 1H), 2.66-2.52 (m, 2H), 2.49-2.36 (m, 4H), 2.04-1.98 (m, 1H), 1.75-1.56 (m, 8H).

The conditions for SFC resolvation are as follows:
Instrument: SFC-150 (Waters)
Column: IH 25×250 mm, 10 um (Daicel)
Column temperature: 35° C.
Mobile phase: CO₂/(MeOH:MeCN=1:1)=50/50
Flow rate: 120 mL/min
Back pressure: 100 bar
Detection wavelength: 214 nm
Cycle time: 8.2 minutes
Sample solution: 100 mg of sample dissolved in 25 mL of methanol/dichloromethane
Injection volume: 4.0 mL

### Example 13: Synthesis of I-59 and I-60

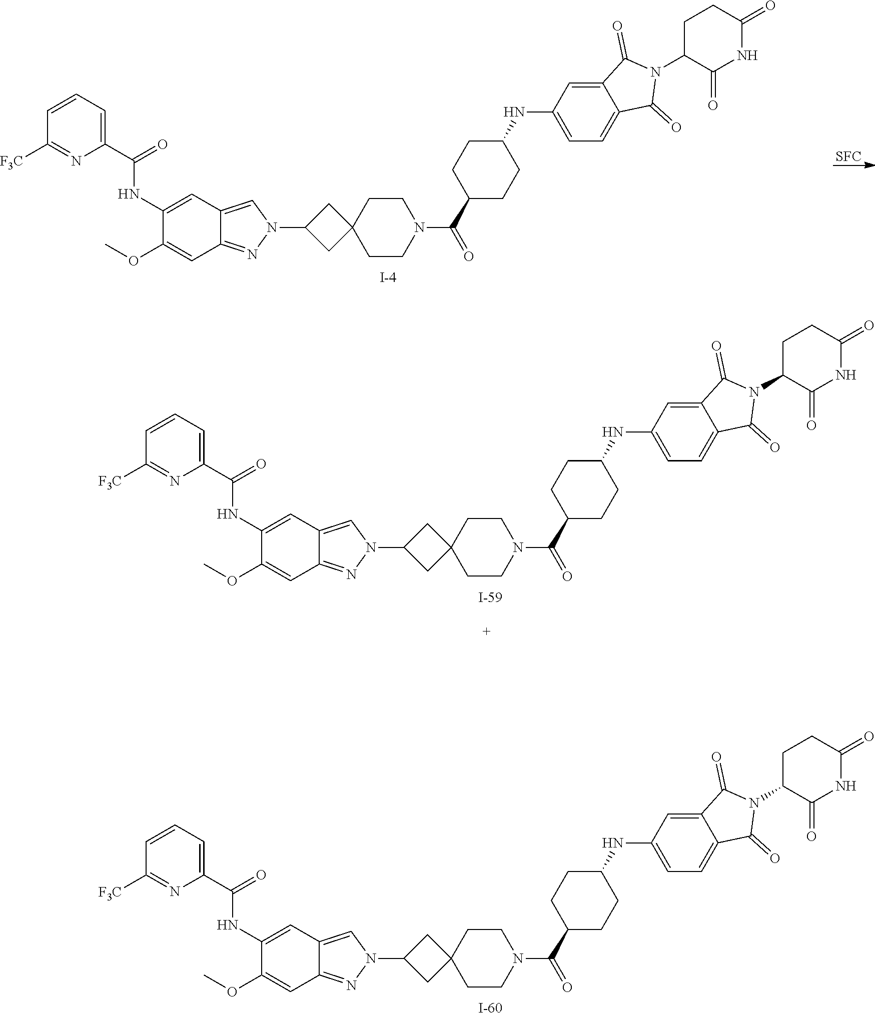

I-4 (50 mg) was chiral resolved by SFC to obtain I-59 as a yellow solid (17.4 mg) and I-60 as a yellow solid (15.9 mg).

I-59 or I-60: Chiral H PL C: Optical purity (ee value)=99.64%, RT=12.068 min. MS (ESI) m/z: 841.9[M+H]⁺. ¹H NMR (500 MHz, DMSO-d₆) δ 11.07 (s, 1H), 10.51 (s, 1H), 8.69 (s, 1H), 8.46 (d, J=8.0 Hz, 1H), 8.41 (t, J=8.0 Hz, 1H), 8.38 (s, 1H), 8.22 (d, J=8.0 Hz, 1H), 7.55 (d, J=8.0 Hz, 1H), 7.22 (s, 1H), 7.03 (d, J=7.0 Hz, 1H), 6.99 (s, 1H), 6.88 (d, J=8.0 Hz, 1H), 5.20-5.14 (m, 1H), 5.03 (dd, J=13.0, 5.5 Hz, 1H), 3.99 (s, 3H), 3.54-3.37 (m, 5H), 2.91-2.83 (m, 1H), 2.68-2.51 (m, 3H), 2.47-2.35 (m, 3H), 2.04-1.95 (m, 3H), 1.74-1.52 (m, 8H), 1.34-1.22 (m, 3H).

I-59 or I-60: Chiral H PL C: Optical purity (ee value)=97.76%, RT=14.283 min. MS (ESI) m/z: 841.9[M+H]⁺. ¹H NMR (500 MHz, DMSO-d₆) δ 11.07 (s, 1H), 10.51 (s, 1H), 8.69 (s, 1H), 8.46 (d, J=8.0 Hz, 1H), 8.41 (t, J=8.0 Hz, 1H), 8.38 (s, 1H), 8.22 (d, J=8.0 Hz, 1H), 7.55 (d, J=8.0 Hz, 1H), 7.22 (s, 1H), 7.04 (d, J=7.0 Hz, 1H), 6.99 (s, 1H), 6.88 (d, J=8.0 Hz, 1H), 5.20-5.14 (m, 1H), 5.03 (dd, J=13.0, 5.5 Hz, 1H), 3.99 (s, 3H), 3.52-3.37 (m, 5H), 2.91-2.83 (m, 1H), 2.68-2.51 (m, 3H), 2.47-2.35 (m, 3H), 2.04-1.95 (m, 3H), 1.75-1.53 (m, 8H), 1.32-1.25 (m, 3H).

The conditions for SFC resolvation are as follows:
Instrument: Gilson-281
Column: IC 25×250 mm, 10 um (Daicel)
Mobile phase: HEX (0.1% DEA):EtOH (0.1% DEA)=50:50
Flow rate: 50 mL/min
Single needle running time: 30 minutes
Injection volume: 3 mL
Sample solution: 50 mg of sample dissolved in 35 mL methanol

### Example 14: Synthesis of I-61 and I-62

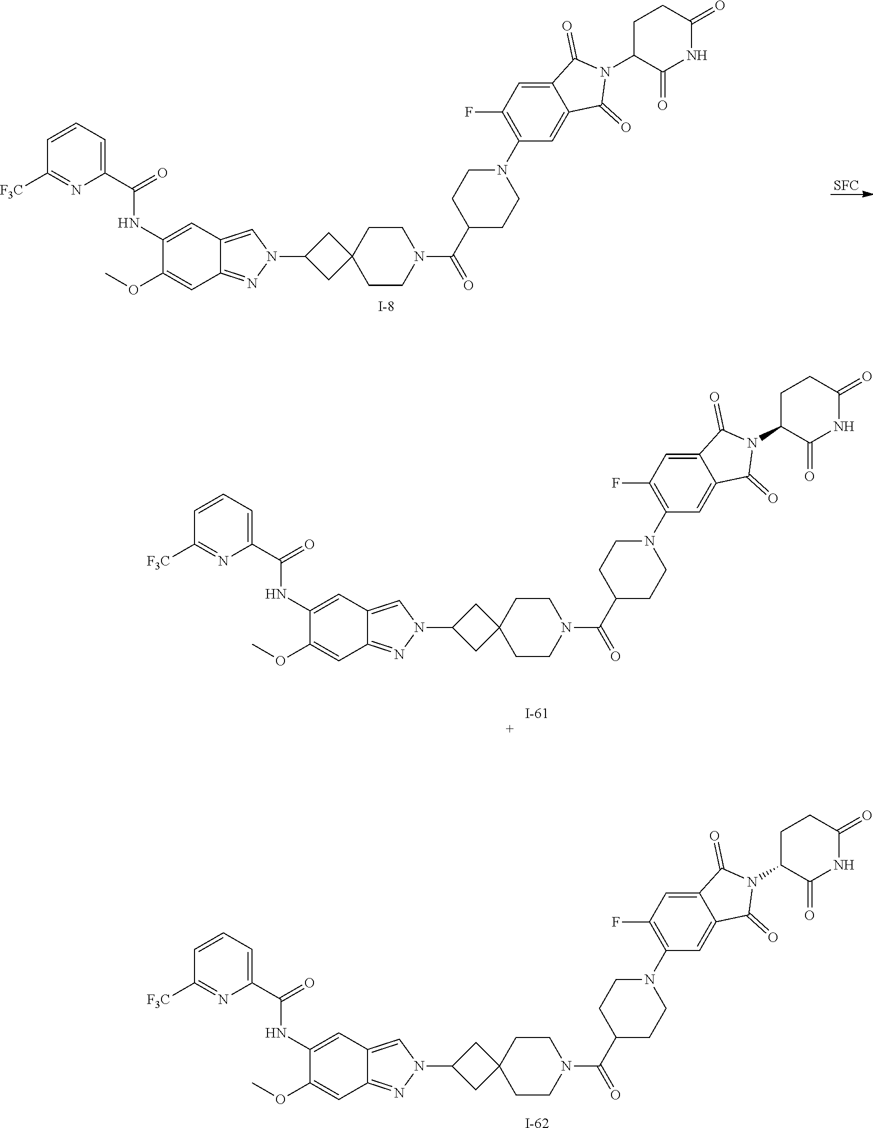

I-8 (100 mg) was chiral resolved by SFC to obtain I-61 as a yellow solid (40.6 mg) and I-62 as a yellow solid (41.2 mg).

I-61 or I-62: Chiral H PLC: Optical purity (ee value)=100%, RT=1.759 min. MS (ESI) m/z: 845.8 [M+H]⁺. ¹H NMR (500 MHz, DMSO-d₆) δ 11.12 (s, 1H), 10.51 (s, 1H), 8.69 (s, 1H), 8.46 (d, J=8.0 Hz, 1H), 8.41 (t, J=8.0 Hz, 1H), 8.38 (s, 1H), 8.22 (d, J=8.0 Hz, 1H), 7.71 (d, J=11.0 Hz, 1H), 7.44 (d, J=7.0 Hz, 1H), 7.22 (s, 1H), 5.21-5.15 (m, 1H), 5.11 (dd, J=13.0, 5.5 Hz, 1H), 3.99 (s, 3H), 3.64 (d, J=11.5 Hz, 2H), 3.58-3.39 (m, 4H), 3.05-2.97 (m, 2H), 2.94-2.86 (m, 2H), 2.64-2.53 (m, 2H), 2.49-2.36 (m, 4H), 2.10-2.01 (m, 1H), 1.79-1.57 (m, 8H).

I-61or I-62: Chiral H PLC: Optical purity (ee value)=99.74%, RT=2.520 min. MS (ESI) m/z: 845.8 [M+H]⁺. ¹H NMR (500 MHz, DMSO-d₆) δ 11.12 (s, 1H), 10.51 (s, 1H), 8.69 (s, 1H), 8.46 (d, J=8.0 Hz, 1H), 8.41 (t, J=8.0 Hz, 1H), 8.38 (s, 1H), 8.23 (d, J=8.0 Hz, 1H), 7.71 (d, J=11.0 Hz, 1H), 7.44 (d, J=7.0 Hz, 1H), 7.22 (s, 1H), 5.21-5.15 (m, 1H), 5.11 (dd, J=13.0, 5.5 Hz, 1H), 3.99 (s, 3H), 3.64 (d, J=11.5 Hz, 2H), 3.57-3.39 (m, 4H), 3.05-2.97 (m, 2H), 2.94-2.85 (m, 2H), 2.64-2.52 (m, 2H), 2.49-2.36 (m, 4H), 2.10-2.02 (m, 1H), 1.79-1.57 (m, 8H).

The conditions for SFC resolvation are as follows:
Instrument: SFC-150 (Waters)
Column: IH 25×250 mm, 10 um (Daicel)
Column temperature: 35° C.
Mobile phase: CO₂/(MeOH:MeCN=1:1)=50/50
Flow rate: 120 mL/min
Back pressure: 100 bar
Detection wavelength: 214 nm
Cycle time: 6.15 minutes
Sample solution: 100 mg of sample dissolved in 18 mL of methanol
Injection volume: 4.0 mL

### Example 15: Synthesis of I-63 and I-64

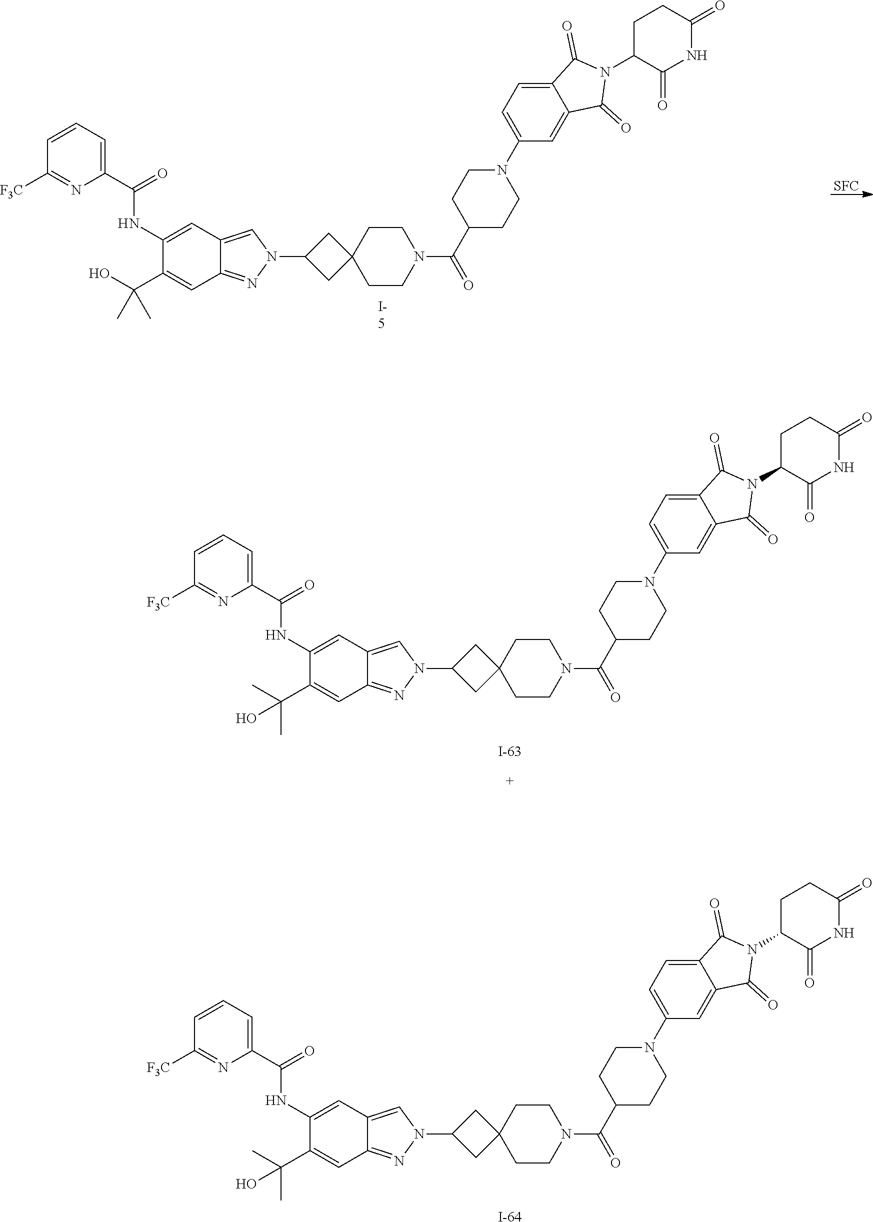

I-5 (20 mg) was chiral resolved by SFC to obtain I-63 as a yellow solid (5.7 mg) and I-64 as a yellow solid (3.9 mg).

I-63 or I-64: Chiral H PLC: Optical purity (ee value)=100%, RT=1.299 min. MS (ESI) m/z: 855.9 [M+H]⁺.

I-63 or I-64: Chiral H PLC: Optical purity (ee value)=99.98%, RT=1.936 min. MS (ESI) m/z: 855.9 [M+H]⁺.

The conditions for SFC resolvation are as follows:
Instrument: SFC-150 (Waters)
Column: AS 25×250 mm, 10 um (Daicel)
Column temperature: 35° C.
Mobile phase: Cl₂/[MeOH (0.2% NH₃(7M in MeOH):MeCN=1:1]=40/60
Flow rate: 100 mL/min
Back pressure: 100 bar
Detection wavelength: 214 nm
Cycle time: 7.1 minutes
Sample solution: 20 mg of sample dissolved in 35 mL of methanol
Injection volume: 4.9 mL

### Example 16: Synthesis of I-65 and I-66

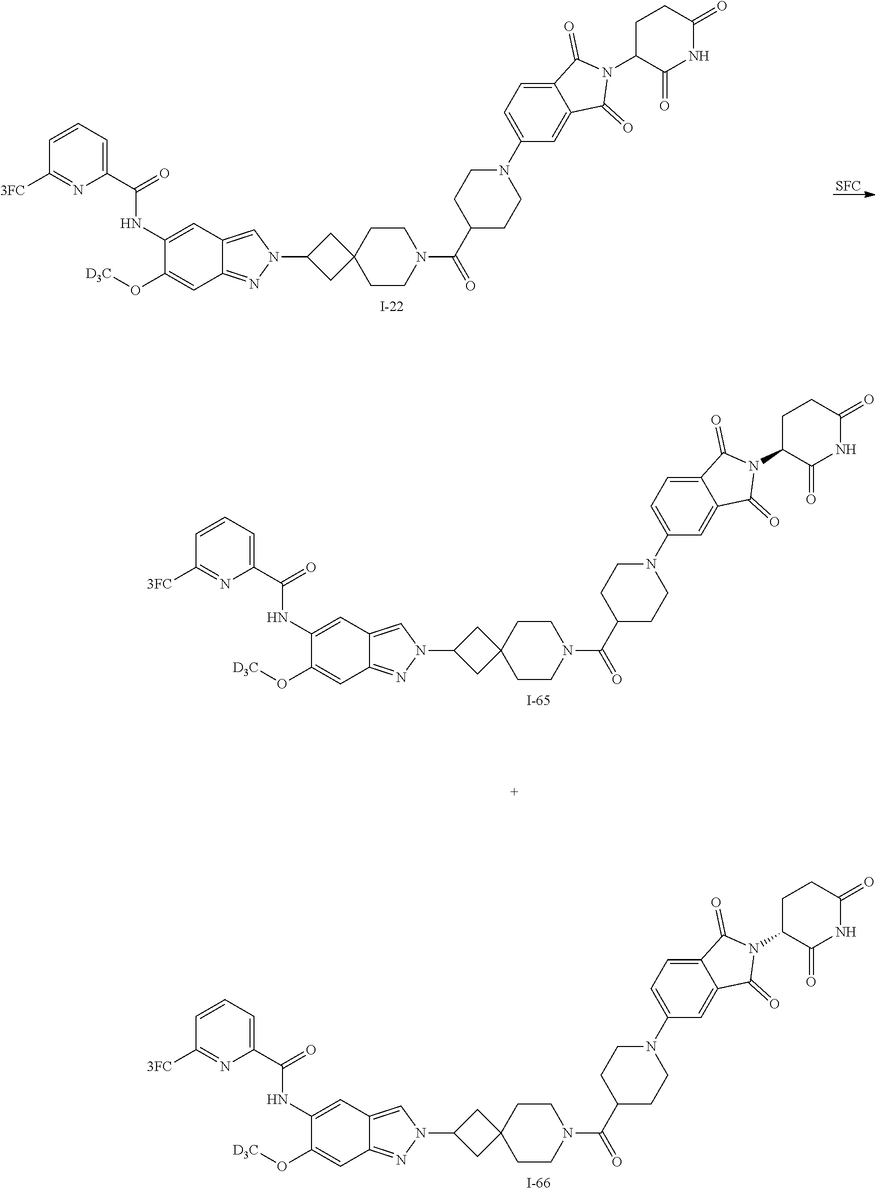

I-22 (100 mg) was chiral resolved by SFC to obtained I-65 as a yellow solid (48.2 mg) and I-66 as a yellow solid (46.6 mg).

I-65 or I-66: Chiral HPLC: Optical purity (ee value)=100%, RT=2.309 nm. MS (ESI) m/z: 830.8 [M+H]⁺. ¹H NMR (400 MHz, DMSO-d₆) δ 11.09 (s, 1H), 10.51 (s, 1H), 8.69 (s, 1H), 8.47-8.38 (m, 3H), 8.22 (d, J=7.6 Hz, 1H), 7.67 (d, J=8.4 Hz, 1H), 7.34 (s, 1H), 7.26-7.21 (m, 2H), 5.21-5.15 (m, 1H), 5.07 (dd, J=12.8, 5.2 Hz, 1H), 4.07 (d, J=13.2 Hz, 2H), 3.56-3.38 (m, 4H), 3.10-2.97 (m, 3H), 2.92-2.84 (m, 1H), 2.64-2.54 (m, 2H), 2.48-2.36 (m, 4H), 2.07-1.96 (m, 1H), 1.77-1.53 (m, 8H).

I-65 or I-66: Chiral H PL C: Optical purity (ee value)=99.70%, RT=3.384 min. MS (ESI) m/z: 830.8 [M+H]⁺. ¹H NMR (400 MHz, DMSO-d₆) δ 11.09 (s, 1H), 10.51 (s, 1H), 8.69 (s, 1H), 8.47-8.38 (m, 3H), 8.22 (d, J=7.6 Hz, 1H), 7.67 (d, J=8.4 Hz, 1H), 7.34 (s, 1H), 7.26-7.21 (m, 2H), 5.20-5.15 (m, 1H), 5.07 (dd, J=12.8, 5.2 Hz, 1H), 4.07 (d, J=13.2 Hz, 2H), 3.56-3.41 (m, 4H), 3.10-2.97 (m, 3H), 2.92-2.85 (m, 1H), 2.64-2.51 (m, 2H), 2.48-2.36 (m, 4H), 2.04-1.99 (m, 1H), 1.75-1.53 (m, 8H).

The conditions for SFC resolvation are as follows:
Instrument: SFC-150 (Waters)
Column: IH 25×250 mm, 10 um (Daicel)
Column temperature: 35° C.
Mobile phase: CO₂/[MeOH:MeCN=1:1]=50/50
Flow rate: 120 mL/min
Back pressure: 100 bar
Detection wavelength: 214 nm
Cycle time: 8.65 minutes
Sample solution: 100 mg of sample dissolved in 35 mL of methanol
Injection volume: 4.8 mL

### Application Examples

### 1. Evaluation of Inhibitory Effects of Compounds on Kinase Activity

Based on an experimental method of fluorescence microfluidic mobility detection, an IC₅₀ value of competitive binding of kinase IRAK4 to ATP by the compounds was determined. The initial detection concentration of the compounds was 10 μM, diluted 4-fold gradient down to 0.38 nM, and tested in duplicate. Among them, commercial staurosporine was the standard control substance in this experiment.

### 1.1 Reagents and Materials Information is as Follows:

| Name | Supplier | No. |
|---|---|---|
| kinase IRAK4 | Carna | 09-145 |
| substrate peptide FAM-P8 | GL Biochem | 112396 |
| Adenosine 5′-triphosphate disodium salt hydrate | Merck | A7699-1G |
| Dimethyl Sulfoxide (DMSO) | Merck | D2650 |
| Ethylene Diamine Tetraacetic Acid (EDTA) | Merck | E5134 |
| Staurosporine | Selleckchem | S1421 |
| 4-(2-hydroxyethyl)-1-piperazine ethanesulfonic | Gibco | 15630-080 |
| acid (HEPES) | | |
| Polyethylene oxide lauroyl ether (Brij-35 solution) | Merck | 9002-92-0 |
| 1,4-dimercaptothreitol (DDT) | Merck | D0632-20G |
| 0.2% Coating Reagent #3 | Platinum Elmer | 760050 |
| 96-well plate | Corning | 3365 |
| 384-well plate | Corning | 3573 |

### 1.2 Experimental Operation Method

1) IRAK4 kinase was dissolved in kinase buffer (50 mM H E PES pH 7.5, 10 mM MgCl₂, 2 mM DTT and 0.01% Brij-35) to a final concentration of 6 nM.

2) The substrate peptide FAM-P8 and ATP were dissolved in the above kinase buffer. The final concentrations of the IRAK4 substrate peptide FAM-P8 and ATP were 3 μM and 10 μM respectively.

3) Dilution of the compounds: the compound was first diluted to 50 μM, and then diluted down with a 4-fold gradient of DMSO. The solution without the compound and kinase is used as a blank control, corresponding to the “minimum value” shown below; and the solution without the compound but with kinase, adenosine 5′-triphosphate disodium salt hydrate, DMSO and buffer is used as a positive control, corresponding to the “maximum value” shown below.

4) Kinase reaction and termination: 10 μL of kinase buffer was added to a 384-well plate containing 5 μL of the compound to be tested, and incubated at room temperature for 10 minutes. Another 10 μL of buffer containing the substrate peptide and adenosine 5′-triphosphate disodium salt hydrate was added to the 384-well plate. After incubation at 28° C. for one hour, 25 μL stop solution (100 mM HEPES pH 7.5, 50 mM EDTA, 0.2% Coating Reagent #3 and 0.015% Brij-35) was added to each well to terminate the reaction.

5) Data reading: the CaliperEZ Reader II instrument was used to read the conversion rate data. Conditions: downstream voltage-500V, upstream voltage-2250V, base pressure-0.5 PSI, and screening pressure -1.2 PSI.

6) Data calculation: the conversion rate data was copied from CaliperEZ Reader II and converted into inhibition rate data. The calculation formula is as follows:

$$Inhibition percentage ( \% ) = ( maximum value - conversion rate ) \text{} / ( maximum value - minimum value ) * 100 \%$$

IC₅₀ values were fitted using XL Fit excel add-in version 5.4.0.8,
Fitting formula: Y=Bottom+(Top−Bottom)/(1+(IC₅₀/X)\hat{ }HillSlope)
Kinase activity data are shown in Table 1 or 2

TABLE 1

| | IRAK4 |
|---|---|
| | IC₅₀ |
| No. | (nM) |
| I-1 | ++++ |
| I-2 | ++++ |
| I-3 | ++++ |
| I-4 | ++++ |
| I-8 | ++++ |
| I-13 | ++++ |
| II-1 | ++++ |
| II-4 | ++++ |
| II-5 | ++++ |
| II-6 | ++++ |
| II-10 | ++++ |
| II-12 | ++++ |
| II-13 | ++++ |
| II-16 | ++++ |
| II-17 | ++++ |

TABLE 2

| | IRAK4 | | IRAK4 | | IRAK4 |
|---|---|---|---|---|---|
| | IC₅₀ | | IC₅₀ | | IC₅₀ |
| No. | (nM) | No. | (nM) | No. | (nM) |
| II-1 | 16.4 | II-2 | 34 | II-4 | 20 |
| II-5 | 11 | II-6 | 8.2 | II-7 | 3.57 |
| II-10 | 7.45 | II-12 | 14.24 | II-13 | 10.52 |
| II-16 | 14 | II-17 | 11 | II-20 | 5.5 |
| I-1 | 26 | I-2 | 11 | I-3 | 16 |
| I-4 | 36.6 | I-5 | 11.35 | I-7 | 8.1 |
| I-8 | 25.87 | I-9 | 32.9 | I-10 | 24 |
| I-12F | 57.6 | I-13 | 4.81 | I-14 | 16.6 |
| I-16 | 11.55 | I-17 | 31 | I-19 | 8.1 |
| I-20 | 23.6 | I-22 | 25 | I-23 | 10.2 |
| I-24 | 17.43 | I-25 | 20.4 | I-26 | 30 |
| I-27 | 42 | I-28 | 12.5 | I-29 | 74 |
| I-30 | 24 | I-31 | 34 | I-32 | 34.8 |
| I-33 | 26.5 | I-34 | 39.7 | I-35 | 33.4 |
| I-36 | 12.4 | I-37 | 29.9 | I-38 | 14.1 |
| | | | | I-41 | 49.8 |
| I-42 | 10.2 | I-43 | 20.7 | I-44 | 17 |
| I-45 | 39.1 | I-46 | 17.7 | I-47 | 9.17 |
| I-48 | 19.2 | I-49 | 13.4 | I-50 | 70.7 |
| I-51 | 5.33 | I-52 | 13.3 | I-53 | 138 |
| I-54 | 11.6 | I-55 | 34.3 | I-56 | 1955 |
| I-57 | 32.4 | I-58 | 35.1 | I-59 | 27.3 |
| I-60 | 40.5 | I-61 | 31.4 | I-62 | 19.0 |
| I-63 | 32.1 | I-64 | 22.7 | I-65 | 26.3 |
| I-66 | 25.3 | | | | |

IRAK4 assay: IC₅₀ <100 nM: ++++; ≥100 nM, <1 μM: +++; ≥1 μM, <10 μM: ++; ≥10 μM: +

2. LPS stimulates THP-1 to release cytokines

### 2.1 Experimental Materials

| Name | Supplier | No. |
|---|---|---|
| THP-1 | ATCC | TIB-202 |
| Dulbecco's Phosphate Buffer | Biosera | LM-S2041/500 |
| (DPBS) | | |
| RPMI1640 medium | Thermo Fisher | 11875119 |
| Fetal Calf Serum (FBS) | Biological | 04-002-1A |
| | Industries | |
| Penicillin-Streptomycin Solution | Invitrogen | 15140122 |
| (P/S) | | |
| β-Mercaptoethanol | Merck | M3148 |
| Dimethylsulfoxide (DMSO) | Sigma | D2650 |
| lipopolysaccharide (LPS) | Thermo Fisher | tlrl-pb5lps |
| 96-well cell culture plate | Corning | 3799 |
| Human TNF-α Duoset ELISA Kit | R&D | DY210 |

### 2. Detection of PROTAC-Induced IRAK4 Protein Degradation in THP-1

2.1 Based on the experimental method of Western Blot, specific antibodies were used to stain the THP-cell samples treated by gel electrophoresis, and the degrading activity of the compounds on the IRAK4 protein in THP-1 cells was determined by analyzing the position and depth of staining. The concentrations of compound for the detection included 0 μM, 0.3 μM, 1 μM and 3 μM, and the cells were treated by the compound for 8 hours, 16 hours, 24 hours, and 48 hours. Glyceraldehyde-3-phosphate dehydrogenase (GAPDH) is an internal reference protein in this experiment

### 2.2 Cells, Reagents and Materials Information is as Follows:

| Name | Supplier | No. |
|---|---|---|
| THP-1 cell | ATCC | TIB-202 |
| RPMI 1640 medium | Gibco | 22400-089 |
| Fetal Calf Serum, FBS | ExCell Bio | FSP500 |
| Penicillin-Streptomycin Solution (P/S) | HyClone | SV30010 |
| Pierce ™ BCA Protein Assay Kit | Thermo Fisher | 23227 |
| RIPA lysate | Merck | R0278 |
| Protease inhibitor (complete Tablets EDTA-free, | Roche | 4693132001 |
| EASYpack) | | |
| Phosphatase Inhibitor Cocktail 2 | Merck | P5726 |
| Phosphatase Inhibitor Cocktail 3 | Merck | P0044-5ML |
| Dimethylsulfoxide, DMSO | Merck | D2650 |
| NuPAGE ™ MOPS SDS electrophoresis buffer (20×) | Invitrogen | NP0001 |
| Pierce ™ 20× TBS Tween ™ 20 buffer | Thermo Fisher | 28360 |
| NuPAGE ™ 4-12% Bis-Tris, 1.5 mm, Mini protein gel, | Invitrogen | NP0336BOX |
| 15 wells | | |
| 1,4-dimercaptothreitol (DDT) | Invitrogen | P2325 |
| Bovine Serum Albumin (BSA) | Merck | B2064-100G |
| Difco ™ skim milk | BD Biosciences | 232100 |
| IBlot ™ 2 transfer membrane set, nitrocellulose, regular | Invitrogen | IB23001 |
| size | | |
| NuPAGE ® LDS sample buffer (4×) | Invitrogen | NP0007 |
| PageRuler Prestained Protein Ladder | Thermo Fisher | 26616 |
| SuperSignal ™ West Femto Maximum Sensitivity | Thermo Fisher | 34095 |
| Substrate | | |
| Anti-IRAK4 antibody | Abcam | ab5985 |
| Anti-Glyceraldehyde-3-Phosphate Dehydrogenase | Merck | MAB374 |
| Antibody, clone 6C5 (GAPDH) | | |
| Goat Anti-Rabbit IgG H&L (HRP) | Abcam | ab205718 |
| Goat Anti-Mouse IgG H&L (HRP) | Abcam | ab205719 |

### 2.3 Device

| Name of device | Supplier | No. |
|---|---|---|
| Cell counter | Count start | IC1000 |
| Multi-Mode Microplate Reader | Molecular Device | Molecular Device Flexstation III |
| Life technologies iBlot2 Gel | IB 21001 | 31252173 |
| Transfer Device | | |
| Image Quant LAS 4000 | 399699 | Image Quant LAS 4010 |

2.4 Method for preparing the reagents

Electrophoresis buffer: 50 mL MOPS SDS Running Buffer (20×) and 50 mL 20×TBS Tween-20 buffer were diluted with deionized water to 1 L as the electrophoresis buffer;

5% skim milk (w/v): 2.5 g skim milk was diluted with 50 mL 1×TBS Tween-20 buffer to prepare 5% skim milk;

5% BSA (w/v): 2.5 g BSA was diluted with 50 mL 1×TBS Tween-20 buffer to prepare 5% BSA;

Anti-IRAK4 antibody was diluted with 5% BSA at 1:1000 into a primary antibody working solution; Goat Anti-Rabbit IgG H&L (HRP) was diluted with 5% BSA at 1:2000 into a second antibody working solution; and

Goat Anti-Mouse IgG H&L (HRP) was diluted with 5% BSA at 1:2000 into a second antibody working solution.

THP-1 cells were plated in a 6-well plate at a density of 1.5×10⁶ cells per ml and incubated for 2 hours in a cell culture incubator under 37° C. and 5% CO₂. The compound was diluted with DMSO to 0.6 mM, 0.2 mM and 0.06 mM respectively. 10 μL of compound solution was added to the corresponding wells, and continued to incubate in the incubator for 16 hours, 24 hours and 48 hours respectively. The compound-treated THP-1 cells in the well were collected, added with 120 μL of RIPA lysate containing protease inhibitor, Phosphatase Inhibitor Cocktail 2 and Phosphatase Inhibitor Cocktail 3, lysed on wet ice for 30 minutes, and then the cells lysate was centrifuged at a high speed and a low temperature for 5 minutes to collect the supernatant. The protein concentration of the cell sample was determined according to the instructions in the Pierce™ BCA Protein Assay K it.

The concentration of the sample was adjusted to be consistent using lysis buffer and NuPAGE® LDS sample buffer containing 1M DTT. The samples were heated at 95° C. for 5 minutes and centrifuged at a low temperature and at a high speed. 20 μL of the prepared protein sample and 4 μL of PageRuler Prestained Protein Ladder were added into the gel well for running at 80V voltage for 0.5 hours, then the voltage was adjusted to 120V for further running for 1.5 hours. The gel was removed and the proteins in the gel were transferred to the Blot™ 2 transfer membrane set at 20V. After successful transfer, bands at 65 kDa-40 kDa and 40 kDa-30 kDa were cut respectively. The membranes were blocked with 5% skim milk for 1 hour at room temperature. The membranes were washed three times with 1× concentration of TBST, then incubated with IRAK4 antibody working solution overnight at 4° C. The IRAK4 antibody working solution was discarded, and the membranes were washed three times with 1× concentration of TBST, and incubated with Goat Anti-Rabbit IgG H&L (HRP) working solution and Goat Anti-Mouse IgG H&L (HRP) working solution at room temperature. After 1 hour of incubation, the antibody working solution was discarded and the membranes were washed three times with 1× concentration of TBST. According to the instructions of the kit SuperSignal™ West Femto Maximum Sensitivity Substrate, the reagents in the kit were mixed in equal volume proportions to prepare a luminescent liquid mixture. The membranes were incubated for 1 minute, then removed for exposure.

The results of Western blotting 24 hours after administration of the compounds with a concentration of 1 μM were shown in Table 3:

TABLE 3

| | IRAK4 degradation rate |
|---|---|
| | 24 hours after administration |
| | of the compounds with a |
| No. | concentration of 1 μM |
| I-1 | 75~100% |
| I-2 | 75~100% |
| I-3 | 75~100% |
| II-1 | 75~100% |
| II-4 | 50~75% |
| II-5 | 75~100% |
| II-6 | 75~100% |
| II-16 | <25% |
| II-17 | <25% |

The concentrations that degraded 50% of IRAK4 by Western blotting 24 hours after administration were shown in Table 4:

TABLE 4

| | IRAK4 | | IRAK4 | | IRAK4 |
|---|---|---|---|---|---|
| | DC₅₀ | | DC₅₀ | | DC₅₀ |
| No. | (nM) | No. | (nM) | No. | (nM) |
| II-1 | 4.33 | II-2 | 32.77 | II-4 | 12.4 |
| II-5 | 6.15 | II-6 | 9.99 | II-7 | 2.2 |
| II-10 | 5.98 | II-12 | 20.49 | II-13 | 478.5 |
| II-16 | 32.92 | II-17 | >1000 | II-20 | 14.06 |
| I-1 | 4.02 | I-2 | 4.41 | I-3 | 3.64 |
| I-4 | 2.06 | I-5 | 3.6 | I-7 | 3.6 |
| I-8 | 8.20 | I-9 | 12 | I-10 | 5.07 |
| I-12F | 1.08 | I-13 | 15.75 | I-14 | 12.61 |
| I-16 | 9.9 | I-17 | 25.9 | I-19 | 1.78 |
| I-20 | 11.2 | I-22 | 6.6 | I-23 | 7.2 |
| I-24 | 15.3 | I-25 | >1000 | I-26 | 44.6 |
| I-27 | 6.9 | I-28 | 8.3 | I-29 | 16.32 |
| I-30 | 26.21 | I-31 | 47.28 | I-32 | 33.8 |
| I-33 | 8.05 | I-34 | 22.72 | I-35 | 7.86 |
| I-36 | 1.16 | I-37 | 20.67 | I-38 | 2.61 |
| | | | | I-41 | 4.42 |
| I-42 | 1.20 | I-43 | >1000 | I-44 | 1.16 |
| I-45 | >1000 | I-46 | 9.15 | I-47 | 5.53 |
| I-48 | >1000 | I-49 | 9.79 | I-50 | 21.6 |
| I-51 | 148.9 | I-52 | 3.9 | I-53 | >1000 |
| I-54 | 6.2 | I-55 | 14.6 | | |
| I-57 | 12.15 | I-58 | 2.67 | I-59 | 8.05 |
| I-60 | 2.75 | I-61 | 7.62 | I-62 | 3.26 |
| I-63 | 2.41 | I-64 | 2.32 | I-65 | 3.71 |
| I-66 | 3.14 | | | | |

1. A compound of Formula I-d: 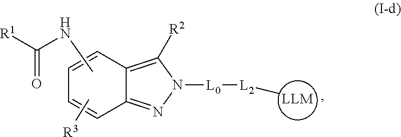
or a pharmaceutically acceptable salt thereof, wherein:
R¹ is a 5- to 10-membered heteroaryl that is unsubstituted or substituted by one of more R¹⁻¹, or a 6-10 membered aryl that is unsubstituted or substituted by one or more R¹⁻²; the heteroatoms of the 5- to 10-membered heteroaryl are selected from one or more of N, S and O, and the number of heteroatoms is 1, 2 or 3;
each R¹⁻¹ and R¹⁻² are independently halogen, hydroxyl, 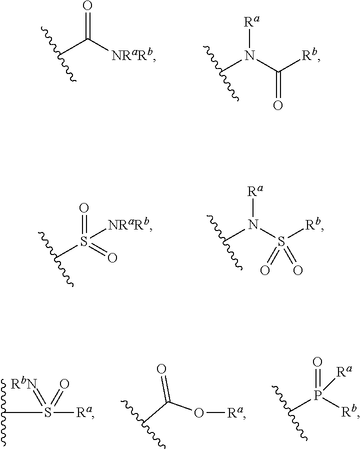
—SO₂—R^{a}, —SO—R^{a}, cyano, nitro, a 3- to 11-membered heterocycloalkyl that is unsubstituted or substituted by one or more R¹⁻¹⁻¹, C₁-C₆ alkoxy that is unsubstituted or substituted by one or more R¹⁻¹⁻³, C₁-C₆ alkyl that is unsubstituted or substituted by one or more R¹⁻¹⁻⁴, 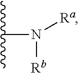
a 3- to 10-membered cycloalkyl that is unsubstituted or substituted by one or more R¹⁻¹⁻⁵, a 6- to 10-membered aryl that is unsubstituted or substituted by one or more R¹⁻¹⁻⁸, or a 5- to 10-membered heteroaryl that is unsubstituted or substituted by one or more R¹⁻¹⁻⁷; the heteroatoms of the 3- to 11-membered heterocycloalkyl are selected from one or more of N, S and O, and the number of heteroatoms is 1, 2 or 3; the heteroatoms of the 5- to 10-membered heteroaryl are selected from one or more of N, S and O, and the number of heteroatom is 1, 2 or 3;
each R¹⁻¹⁻¹, R¹⁻¹⁻³, R¹⁻¹⁻⁴, R¹⁻¹⁻⁵, R¹⁻¹⁻⁷, and R¹⁻¹⁻⁸ are independently deuterium, halogen, oxo, hydroxyl, 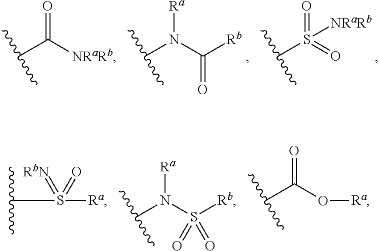
SO₂—R^{a}, —SO—R^{a}, C₁-C₆ alkoxy that is unsubstituted or substituted by one or more halogens, cyano, nitro, a 3- to 11-membered heterocycloalkyl that is unsubstituted or substituted by one or more R¹⁻¹⁻¹⁻¹, C₁-C₆ alkyl that is unsubstituted or substituted by one or more halogens or deuterium, a 3- to 10-membered cycloalkyl, a 6- to 10-membered aryl that is unsubstituted or substituted by one or more R¹⁻¹⁻¹⁻², or a 5- to 10-membered heteroaryl that is unsubstituted or substituted by one or more R¹⁻¹⁻¹⁻³; the heteroatoms of the 3- to 11-membered heterocycloalkyl are selected from one or more of N, S and O, and the number of heteroatoms is 1, 2 or 3; the heteroatoms of the 5- to 10-membered heteroaryl are selected from one or more of N, S and O, and the number of heteroatom is 1, 2 or 3;
each R¹⁻¹⁻¹⁻¹, R¹⁻¹⁻¹⁻², and R¹⁻¹⁻¹⁻³ are independently halogen, oxo, hydroxyl, C₁-C₆ alkoxy or C₁-C₆ alkyl;
R² is hydrogen, hydroxyl, cyano, halogen, C₁-C₆ alkyl that is unsubstituted or substituted by one or more R²⁻³, C₁-C₆ alkoxy that is unsubstituted or substituted by one or more R²⁻², 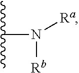
a 5- to 10-membered heteroaryl that is unsubstituted or substituted by one or more R²⁻⁴; the heteroatoms of the 5- to 10-membered heteroaryl are selected from one or more of N, S and O, and the number of heteroatoms is 1, 2 or 3; the heteroatoms of the 4- to 10-membered heterocycloalkyl are selected from one or more N, S and O, and the number of heteroatoms is 1, 2 or 3;
each R²⁻¹, R²⁻², R²⁻³ and R²⁻⁴ are independently halogen, hydroxyl, cyano, nitro, C₁-C₆ alkyl that is unsubstituted or substituted by one or more halogens, 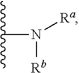
or C₁-C₆ alkoxy that is unsubstituted or substituted by one or more halogens;
R³ is hydrogen, deuterium, halogen, cyano, hydroxyl, nitro, 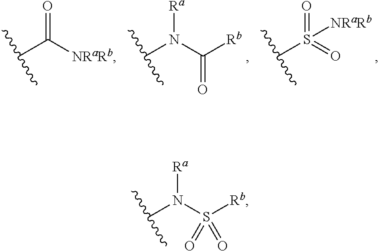
—SO₂—R^{a}, —SO—R^{a}, 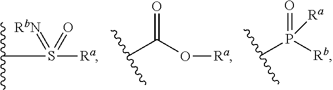
a 3- to 11-membered heterocycloalkyl that is unsubstituted or substituted by one or more R³⁻¹, a 3- to 10-membered cycloalkyl that is unsubstituted or substituted by one or more R³⁻², 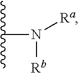
C₁-C₆ alkyl that is unsubstituted or substituted by one or more R³⁻⁴, a 6- to 10-membered aryl that is unsubstituted or substituted by one or more R³⁻⁵, a 5- to 10-membered heteroaryl that is unsubstituted or substituted by one or more R³⁻⁶, a hydroxyl substituted by R³⁻⁸, a C₁-C₆ alkoxy that is unsubstituted or substituted by one or more R³⁻⁷ or —O—COR^{a}; the heteroatoms of the 3- to 11-membered heterocycloalkyl are selected from one or more of N, S and O, and the number of the heteroatoms is 1, 2 or 3; the heteroatoms of the 5- to 10-membered heteroaryl are selected from one or more of N, S and O, and the number of heteroatoms is 1, 2 or 3;
each R³⁻¹, R³⁻², R³⁻⁴, R³⁻⁵, R³⁻⁶ and R³⁻⁷ are independently deuterium, halogen, oxo, a hydroxyl, a 3- to 11-membered heterocycloalkyl that is unsubstituted or substituted by one or more R³⁻¹⁻¹, 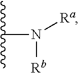
cyano, alkoxy that is unsubstituted or substituted by one or more R³⁻¹⁻³, 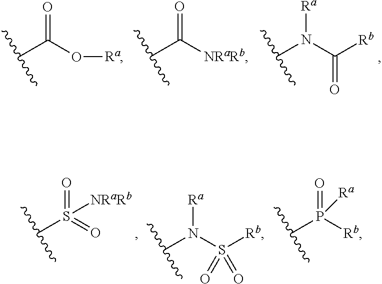
C₁-C₆ alkyl that is unsubstituted or substituted by one or more R³⁻¹⁻⁴, a 3- to 10-membered cycloalkyl that is unsubstituted or substituted by one or more R³⁻¹⁻⁵, —SO₂—R^{a}, —SO—R^{a}, 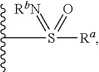
a 6- to 10-membered aryl, or a 5- to 10-membered heteroaryl; the heteroatoms of the 3- to 11-membered heterocycloalkyl are selected from one or more of N, S and O, and the number of heteroatoms is 1, 2 or 3; the heteroatoms of the 5- to 10-membered heteroaryl are selected from one or more of N, S and O, and the number of heteroatoms is 1, 2 or 3;
R³⁻⁸ is a 3- to 10-membered cycloalkyl, a 6- to 10-membered aryl, a 3- to 11-membered heterocycloalkyl, or a 5- to 10-membered heteroaryl; the heteroatoms of the 3- to 11-membered heterocycloalkyl are selected from one or more of N, S and O, and the number of heteroatoms is 1, 2 or 3; the heteroatoms of the 5- to 10-membered heteroaryl are selected from one or more N, S and O, and the number of heteroatoms is 1, 2 or 3;
each R³⁻¹⁻¹, R³⁻¹⁻³, R³⁻¹⁻⁴ and R³⁻¹⁻⁵ are independently C1-C6 alkyl that is unsubstituted or substituted by one or more halogens, halogen, oxo or hydroxyl;
each R^{a} and each R^{b} are independently H, C₁-C₆ alkyl that is unsubstituted or substituted by one or more R^{a-1}, a 3- to 10-membered cycloalkyl that is unsubstituted or substituted by one or more R^{a-2}, a 3- to 11-membered heterocycloalkyl that is unsubstituted or substituted by one or more R^{a-3}, a 6- to 10-membered aryl that is unsubstituted or substituted by one or more R^{a-4}, or a 5- to 10-membered heteroaryl that is unsubstituted or substituted by one or more R^{a-5}; or
R^{a} and R^{b} and the atoms connected thereto together form a 3- to 11-membered heterocycle; the heteroatoms of the 3- to 11-membered heterocycle are selected from one or more of N, S and O, and the number of heteroatoms is 1, 2 or 3;
each R^{a-1}, R^{a-2}, R^{a-3}, R^{a-4} and R^{a-5} are independently halogen, cyano, hydroxyl, nitro, C₁-C₆ alkyl, C₁-C₆ alkoxy, a 3- to 10-membered cycloalkyl, a 3- to 11-membered heterocycloalkyl, a 6- to 10-membered aryl, a 5- to 10-membered heteroaryl; the heteroatoms of the 3- to 11-membered heterocycloalkyl are selected from one or more of N, S and O, and the number of heteroatoms is 1, 2 or 3; the heteroatoms of the 5- to 10-membered heteroaryl are selected from one or more of N, S and O, and the number of heteroatoms is 1, 2 or 3;
L₀-L₂- is 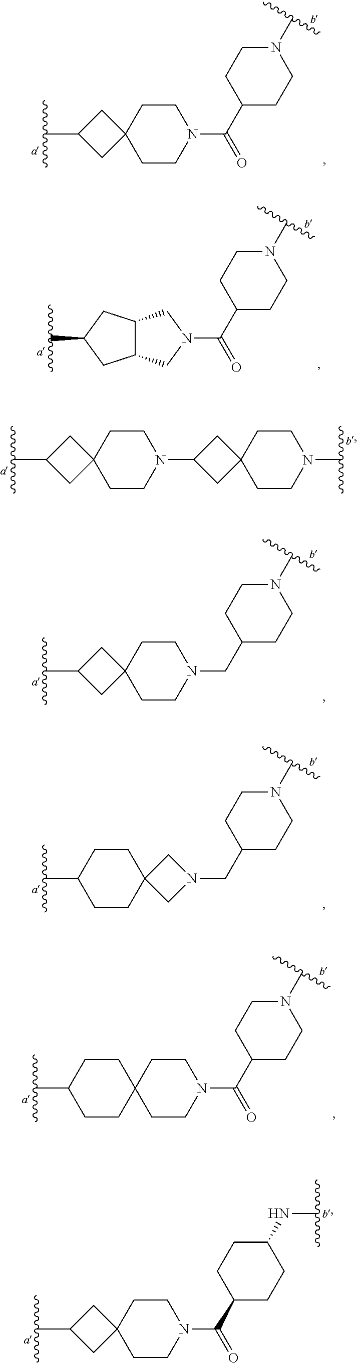 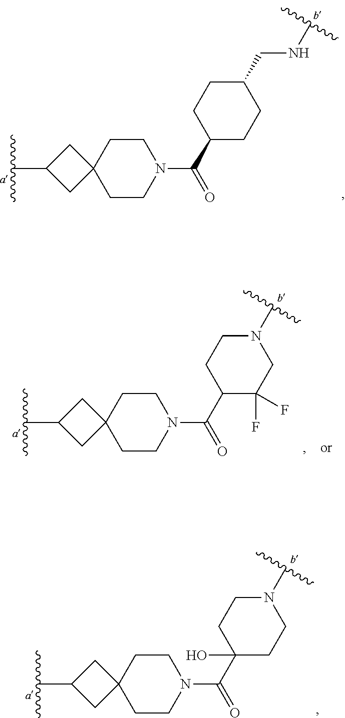 [SMILES: *NC[C@H]1CC[C@H](C(=O)N2CCC3(CC2)CC(*)C3)CC1 ; *C1CC2(CCN(C(=O)C3CCN(*)CC3(F)F)CC2)C1 ; *C1CC2(CCN(C(=O)C3(O)CCN(*)CC3)CC2)C1]
wherein the a′ end is connected to 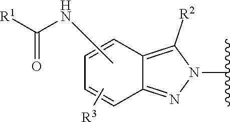
and the b′ end is connected to LLM;
LLM is 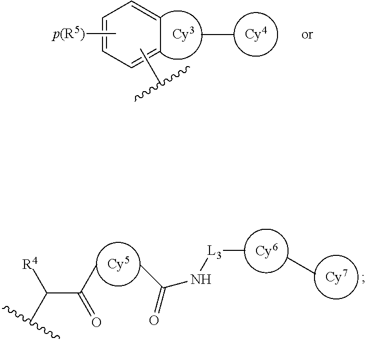
ring Cy³ is a 5- to 12-membered heterocycle that is unsubstituted or substituted by one or more Cy³⁻¹, the heteroatoms of the 5- to 12-membered heterocycle are selected from one or more of N, S and O, and the number of heteroatoms is 1, 2 or 3; Cy³⁻¹ is independently C₁-C₆ alkyl, halogen, hydroxyl or oxo;
ring Cy⁴ is a 5- to 12-membered heterocycloalkyl that is unsubstituted or substituted by one or more Cy⁴⁻¹, the heteroatoms of the 5- to 12-membered heterocycle are selected from one or more of N, S and O, and the number of heteroatoms is 1, 2 or 3; Cy⁴⁻¹ is independently C₁-C₆ alkyl, halogen, hydroxyl or oxo;
Ring Cy⁵ is a 5- to 12-membered heterocycle that is unsubstituted or substituted by one or more Cy⁵⁻¹, the heteroatoms of the 5- to 12-membered heterocycle are selected from one or more of N, S and O, and the number of heteroatoms is 1, 2 or 3; Cy⁵⁻¹ is independently C₁-C₆ alkyl, hydroxyl or oxo;
Ring Cy⁶ is a 6- to 10-membered aromatic ring that is unsubstituted or substituted by one or more Cy⁶⁻¹; Cy⁶⁻¹ is independently C₁-C₆ alkyl, hydroxyl or halogens;
Ring Cy⁷ is a 5- to 9-membered heteroaromatic ring that is unsubstituted or substituted by one or more Cy⁷⁻¹, the heteroatoms of the 5- to 9-membered heteroaromatic ring are selected from one or more of N, S and O, and the number of heteroatoms is 1, 2 or 3; Cy⁷⁻¹ is independently C₁-C₆ alkyl, hydroxyl or halogen;
R⁴ is independently hydrogen, halogen, hydroxyl or C₁-C₆ alkyl that is unsubstituted or substituted by one or more halogens;
p is 0, 1, 2 or 3;
each R^{S} is independently halogen;
L₃ is 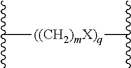
that is unsubstituted or substituted by one or more L₃^{- 1;}
wherein m is an integer from 1 to 4, q is an integer from 1 to 6, and X is absent or O; and L₃⁻¹ is independently halogen, or C₁-C₆ alkyl that is unsubstituted or substituted by one or more halogens.

2. The compound of claim 1, wherein LLM is 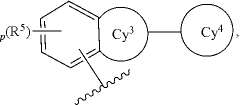
wherein:
Ring Cy³ is a 5-6 membered heterocycle that is unsubstituted or substituted by one or more Cy³⁻¹, the heteroatom of the 5- to 6-membered heterocycle is N, S or O, and the number of heteroatoms is 1;
Cy³⁻¹ is independently oxo;
Ring Cy⁴ is a 5- to 8-membered heterocycloalkyl that is unsubstituted or substituted by one or more Cy⁴-, the heteroatom of the 5- to 8-membered heterocycloalkyl is N, S or O, and the number of heteroatoms is 1;
Cy⁴⁻¹ is oxo;
R⁵ is halogen; and
p is 0, 1, 2 or 3.

3. The compound of claim 1, wherein the LLM is: 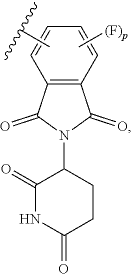
wherein p is 0 or 1; 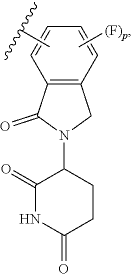
wherein p is 0 or 1; 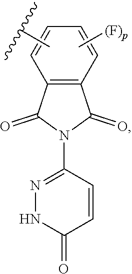
wherein p
is 0 or 1; or

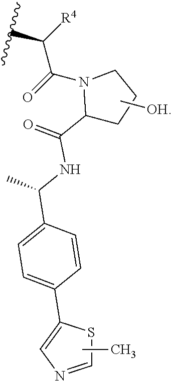

4. The compound of claim 3, wherein the LLM is

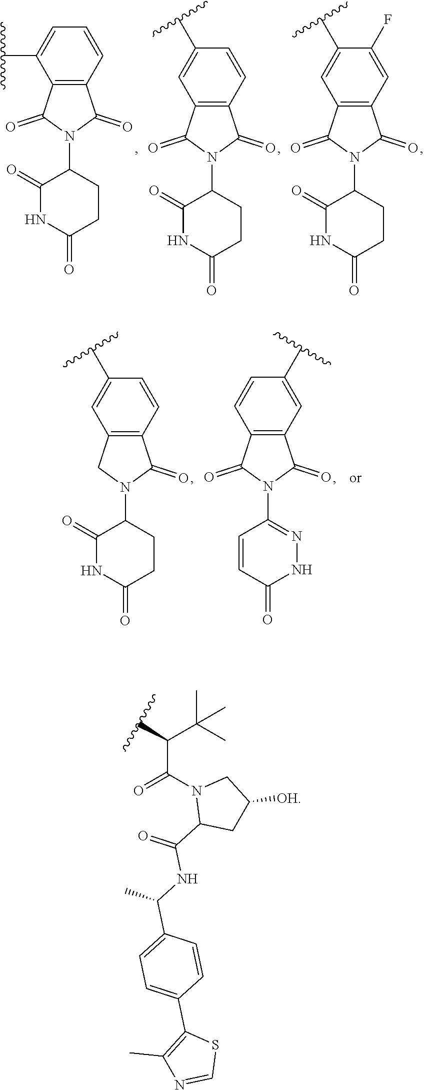

5. The compound of claim 1, wherein:
R¹ is a 5- to 10-membered heteroaryl that is unsubstituted or substituted by one of more R¹⁻¹, wherein the heteroatoms of the 5- to 10-membered heteroaryl are selected from one or more of N, S and O, and the number of heteroatoms is 1, 2 or 3; wherein,
R¹⁻¹ is a C₁-C₆ alkyl that is unsubstituted or substituted by one or more R¹⁻¹⁻⁴, a 3- to 11-membered heterocycloalkyl that is unsubstituted or substituted by one or more R¹⁻¹⁻¹ or a 5- to 10-membered heteroaryl group that is unsubstituted or substituted by one or more R¹⁻¹⁻⁷; wherein the heteroatoms of the 5- to 10-membered heteroaryl are selected from one or two of N, S and O, and the number of heteroatoms is 1 or 2; wherein the heteroatoms of the 3- to 11-membered heterocycloalkyl are selected from one or two of N, S and O, and the number of heteroatoms is 1 or 2; and
each of R¹⁻¹⁻¹, R¹⁻¹⁻⁴ and R¹⁻¹⁻⁷ are independently halogen or C₁-C₆ alkyl unsubstituted or substituted by one or more halogens.

6. The compound of claim 1, wherein R¹ is

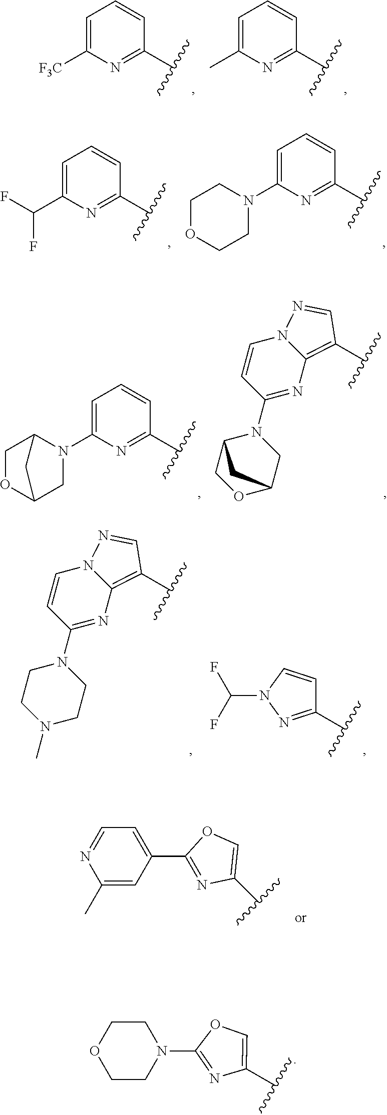

7. The compound of claim 1, wherein R³ is hydrogen, deuterium, halogen, 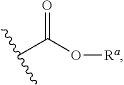
a 3- to 11-membered heterocycloalkyl, 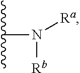
C₁-C₆ alkyl that is unsubstituted with or substituted by one or more R³⁻⁴, a C₁-C₆ alkoxy that is unsubstituted or substituted by one or more R³⁻⁷ or —O—COR^{a}, wherein:
each R^{a} and each R^{b} are independently H or C₁-C₆ alkyl that is unsubstituted or substituted by one or more R^{a}-1; or R^{a} and R^{b} and the atoms connected thereto together form a 3- to 11-membered heterocycle;
each R³⁻⁴ and R³⁻⁷ are independently deuterium, halogen, a hydroxyl or C₁-C₆ alkyl that is unsubstituted or substituted by one or more R³⁻¹⁻⁴; and
R³⁻¹⁻⁴ is halogen.

8. The compound of claim 7, wherein R³ is methoxy, isopropoxy, difluoromethoxy, trifluoromethoxy, carboxyl, fluorine,

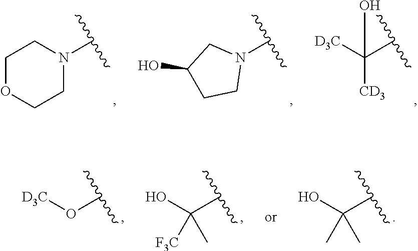

9. The compound of claim 8, wherein R³ is methoxy,

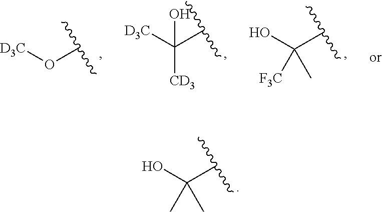

10. The compound of claim 1, wherein R² is hydrogen.

11. The compound of claim 1, wherein 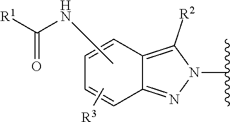
is:

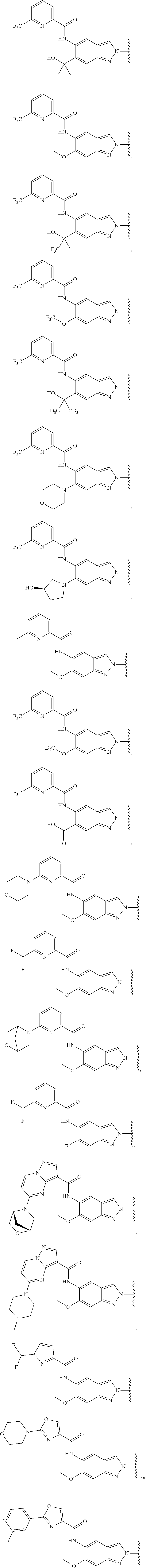

12. The compound of claim 1, wherein 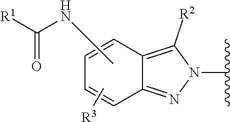
is: 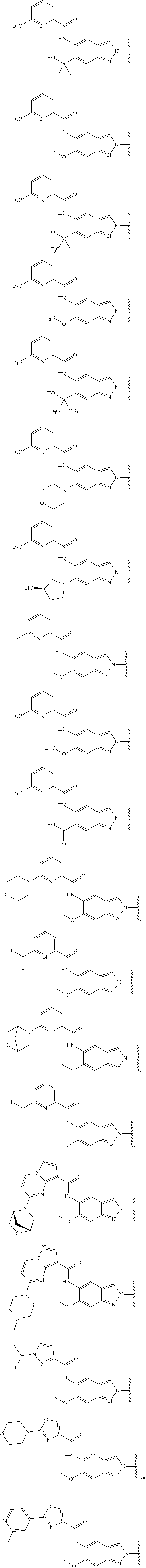
and
LLM is:

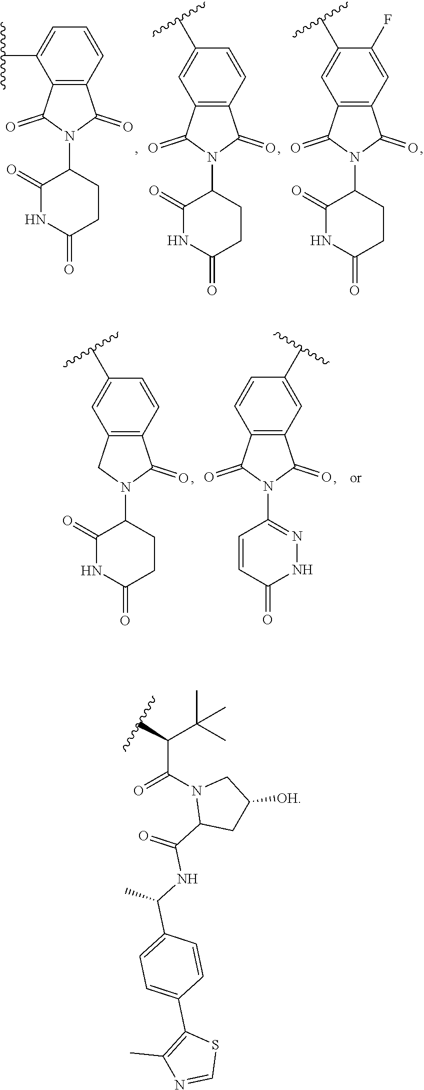

13. The compound of claim 1, wherein 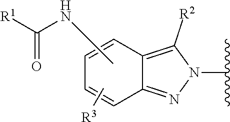
is 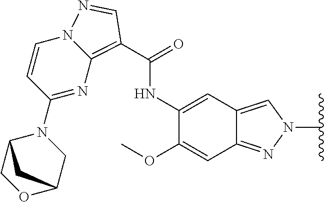 [SMILES: *n1cc2cc(NC(=O)c3cnn4ccc(N5C[C@H]6C[C@@H]5CO6)nc34)c(OC)cc2n1]
and -L₀-L₂- is 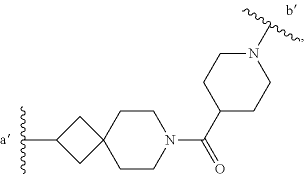 [SMILES: *C1CC2(CCN(C(=O)C3CCN(*)CC3)CC2)C1]
wherein the a′ end is connected 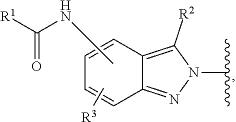
and the b′ end is connected to LLM.

14. The compound of claim 1, wherein t 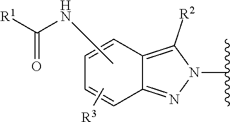
is 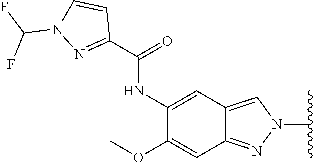 [SMILES: *n1cc2cc(NC(=O)c3ccn(C(F)F)n3)c(OC)cc2n1]
and -L₀-L₂- is, 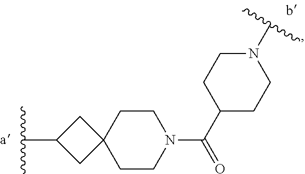 [SMILES: *C1CC2(CCN(C(=O)C3CCN(*)CC3)CC2)C1]
wherein the a′ end is connected to 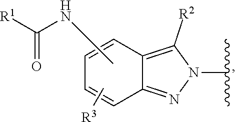
and the b′ end is connected to LLM.

15. A pharmaceutical composition comprising a compound according to claim 1, or a pharmaceutically acceptable salt thereof.

16. The pharmaceutical composition of claim 15, further comprising at least one pharmaceutically acceptable excipient.

17. A pharmaceutical composition comprising a compound according to claim 12, or a pharmaceutically acceptable salt thereof.

18. The pharmaceutical composition of claim 17, further comprising at least one pharmaceutically acceptable excipient.

19. The compound of claim 1, wherein the compound is selected from the group consisting of: 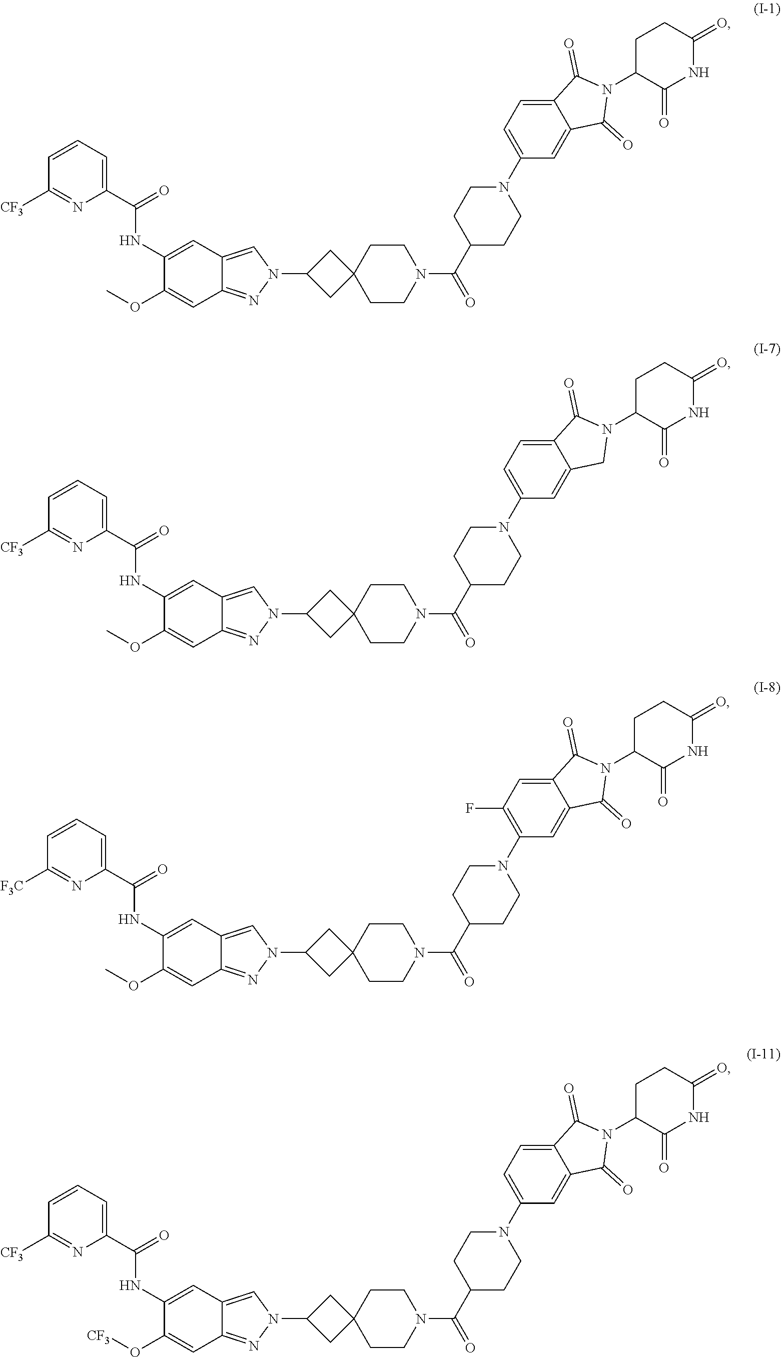 [SMILES: COc1cc2nn(C3CC4(CCN(C(=O)C5CCN(c6ccc7c(c6)C(=O)N(C6CCC(=O)NC6=O)C7=O)CC5)CC4)C3)cc2cc1NC(=O)c1cccc(C(F)(F)F)n1 ; COc1cc2nn(C3CC4(CCN(C(=O)C5CCN(c6ccc7c(c6)CN(C6CCC(=O)NC6=O)C7=O)CC5)CC4)C3)cc2cc1NC(=O)c1cccc(C(F)(F)F)n1 ; COc1cc2nn(C3CC4(CCN(C(=O)C5CCN(c6cc7c(cc6F)C(=O)N(C6CCC(=O)NC6=O)C7=O)CC5)CC4)C3)cc2cc1NC(=O)c1cccc(C(F)(F)F)n1 ; O=C1CCC(N2C(=O)c3ccc(N4CCC(C(=O)N5CCC6(CC5)CC(n5cc7cc(NC(=O)c8cccc(C(F)(F)F)n8)c(OC(F)(F)F)cc7n5)C6)CC4)cc3C2=O)C(=O)N1] 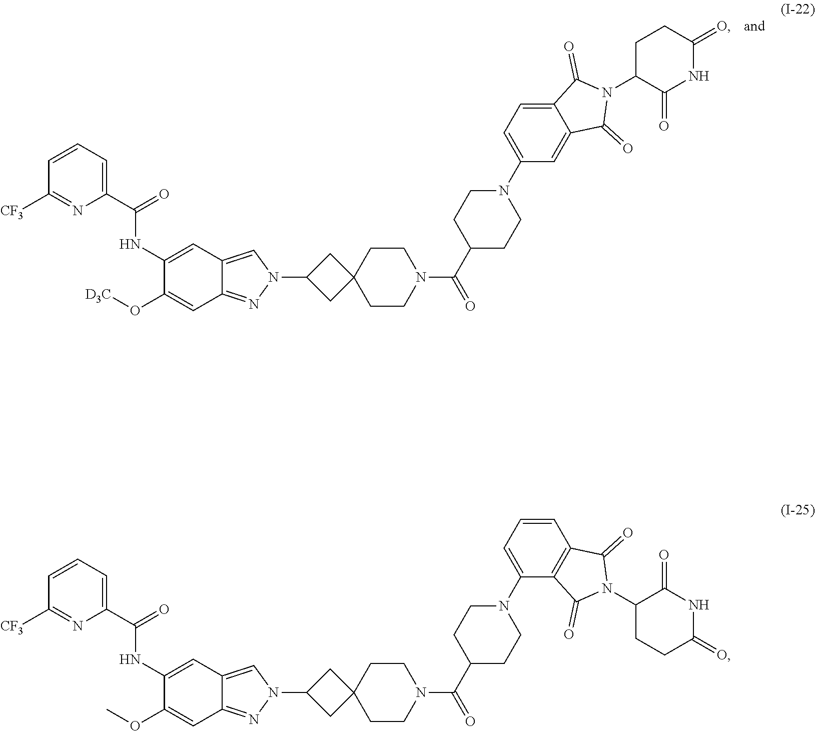 [SMILES: [2H]C([2H])([2H])Oc1cc2nn(C3CC4(CCN(C(=O)C5CCN(c6ccc7c(c6)C(=O)N(C6CCC(=O)NC6=O)C7=O)CC5)CC4)C3)cc2cc1NC(=O)c1cccc(C(F)(F)F)n1 ; COc1cc2nn(C3CC4(CCN(C(=O)C5CCN(c6cccc7c6C(=O)N(C6CCC(=O)NC6=O)C7=O)CC5)CC4)C3)cc2cc1NC(=O)c1cccc(C(F)(F)F)n1]
or a pharmaceutically acceptable salt thereof.

20. The compound of claim 1, wherein the compound is I-8: 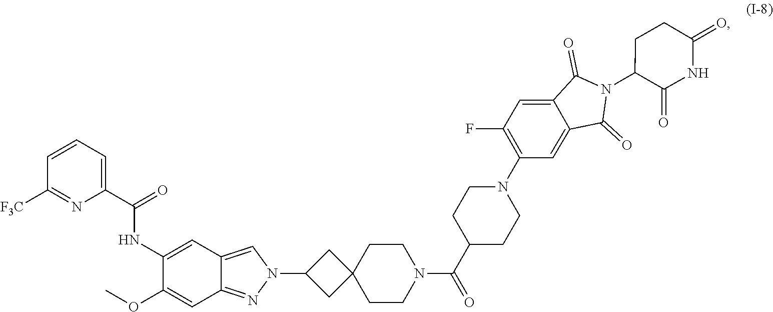 [SMILES: COc1cc2nn(C3CC4(CCN(C(=O)C5CCN(c6cc7c(cc6F)C(=O)N(C6CCC(=O)NC6=O)C7=O)CC5)CC4)C3)cc2cc1NC(=O)c1cccc(C(F)(F)F)n1]
or a pharmaceutically acceptable salt thereof.

21. The compound of claim 20, wherein the compound is: 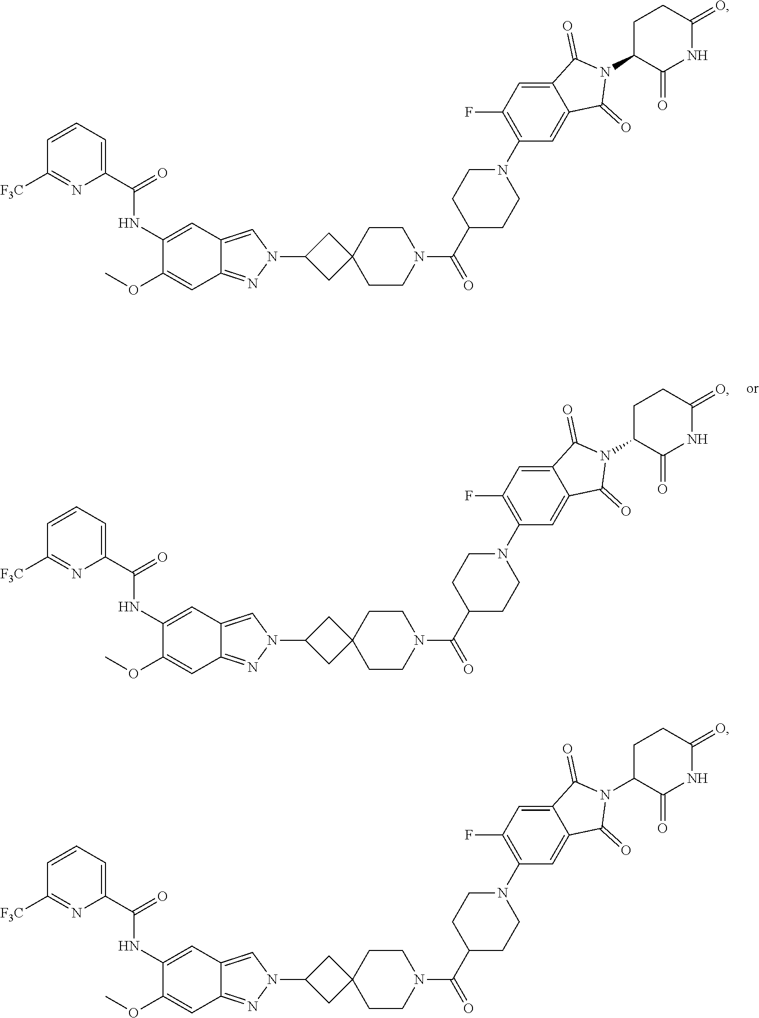 [SMILES: COc1cc2nn(C3CC4(CCN(C(=O)C5CCN(c6cc7c(cc6F)C(=O)N([C@H]6CCC(=O)NC6=O)C7=O)CC5)CC4)C3)cc2cc1NC(=O)c1cccc(C(F)(F)F)n1 ; COc1cc2nn(C3CC4(CCN(C(=O)C5CCN(c6cc7c(cc6F)C(=O)N([C@@H]6CCC(=O)NC6=O)C7=O)CC5)CC4)C3)cc2cc1NC(=O)c1cccc(C(F)(F)F)n1 ; "or" ; COc1cc2nn(C3CC4(CCN(C(=O)C5CCN(c6cc7c(cc6F)C(=O)N(C6CCC(=O)NC6=O)C7=O)CC5)CC4)C3)cc2cc1NC(=O)c1cccc(C(F)(F)F)n1]
or a pharmaceutically acceptable salt thereof, and
wherein the compound has a retention time of 1.759 min or 2.520 min under the following conditions: chromatographic column: IH 25×250 mm, 10 μm (Daicel); mobile phase: CO₂/[MeOH:MeCN=1:1]=50/50; flow rate:120 mL/min.

22. The compound of claim 1, wherein the compound is I-11: 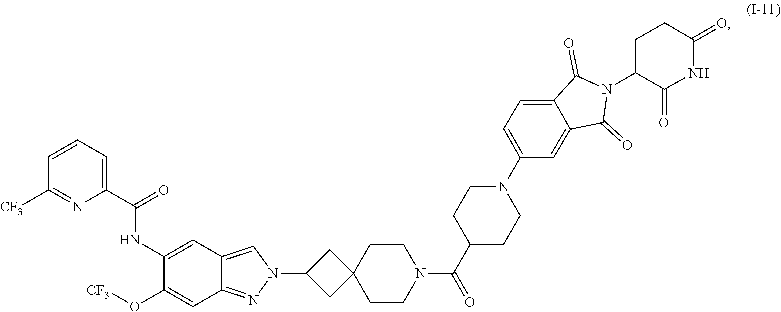 [SMILES: O=C1CCC(N2C(=O)c3ccc(N4CCC(C(=O)N5CCC6(CC5)CC(n5cc7cc(NC(=O)c8cccc(C(F)(F)F)n8)c(OC(F)(F)F)cc7n5)C6)CC4)cc3C2=O)C(=O)N1]
or a pharmaceutically acceptable salt thereof.

23. The compound of claim 1, wherein the compound is I-22: 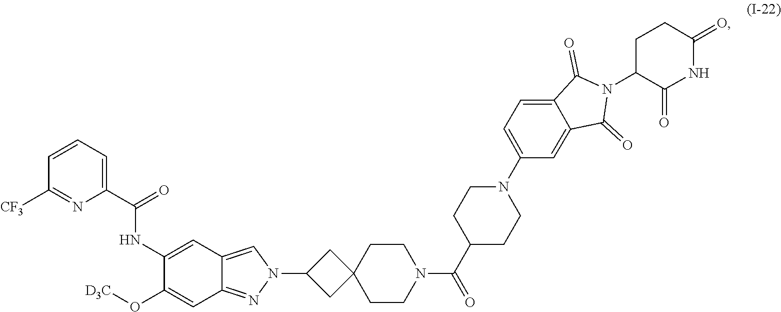 [SMILES: [2H]C([2H])([2H])Oc1cc2nn(C3CC4(CCN(C(=O)C5CCN(c6ccc7c(c6)C(=O)N(C6CCC(=O)NC6=O)C7=O)CC5)CC4)C3)cc2cc1NC(=O)c1cccc(C(F)(F)F)n1]
or a pharmaceutically acceptable salt thereof.

24. The compound of claim 23, wherein the compound is: 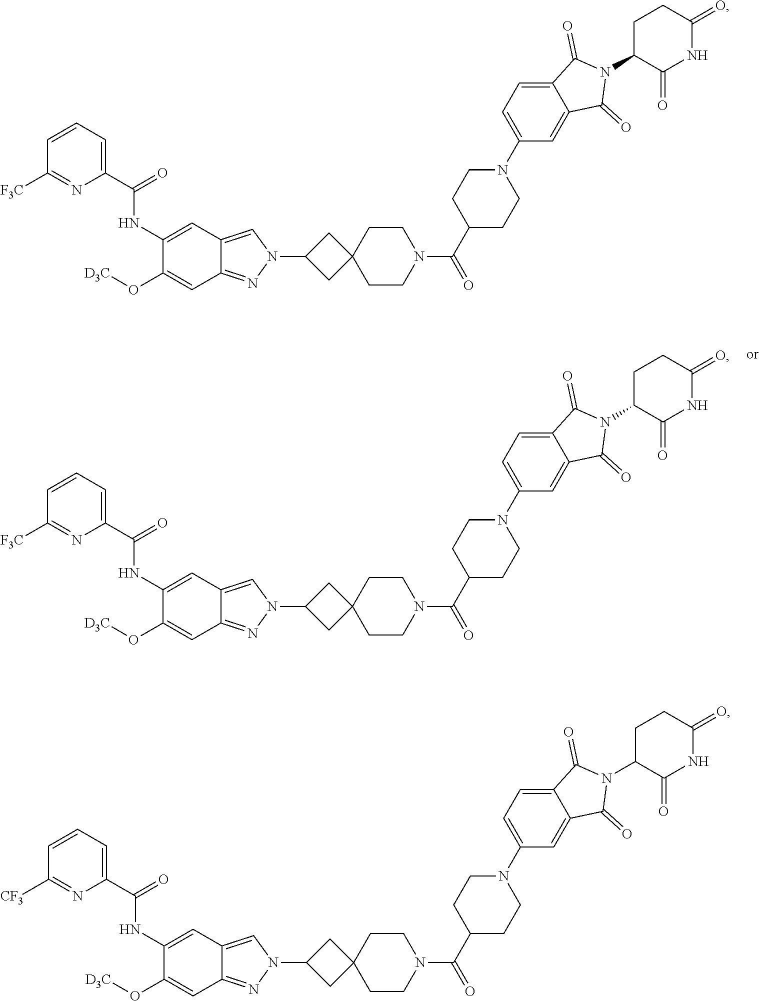 [SMILES: [2H]C([2H])([2H])Oc1cc2nn(C3CC4(CCN(C(=O)C5CCN(c6ccc7c(c6)C(=O)N([C@H]6CCC(=O)NC6=O)C7=O)CC5)CC4)C3)cc2cc1NC(=O)c1cccc(C(F)(F)F)n1 ; [2H]C([2H])([2H])Oc1cc2nn(C3CC4(CCN(C(=O)C5CCN(c6ccc7c(c6)C(=O)N([C@@H]6CCC(=O)NC6=O)C7=O)CC5)CC4)C3)cc2cc1NC(=O)c1cccc(C(F)(F)F)n1 ; "or" ; [2H]C([2H])([2H])Oc1cc2nn(C3CC4(CCN(C(=O)C5CCN(c6ccc7c(c6)C(=O)N(C6CCC(=O)NC6=O)C7=O)CC5)CC4)C3)cc2cc1NC(=O)c1cccc(C(F)(F)F)n1]
or a pharmaceutically acceptable salt thereof, and
wherein the compound has a retention time of 2.309 min or 3.384 min under the following conditions: chromatographic column: IH 25×250 mm, 10 μm (Daicel); mobile phase: CO₂/[MeOH:MeCN=1:1]=50/50; flow rate: 120 mL/min.
